(19) United States
(12) Patent Application Publication
(10) Pub. No.: US 2024/0344138 A1
(43) Pub. Date: Oct. 17, 2024

(54) TARGETED THERAPIES IN CANCER

(71) Applicant: Feng Biosciences, Inc, Waltham, MA (US)

(72) Inventors: Laura E. BENJAMIN, Waltham, MA (US); Kristen STRAND-TIBBITTS, Waltham, MA (US); Rafael ROSENGARTEN, Waltham, MA (US); Miha ŠTAJDOHAR, Ljubljana (SI); Robert CVITKOVIC, Ljubljana (SI)

(73) Assignee: Feng Biosciences, Inc, Waltham, MA (US)

(21) Appl. No.: 18/552,410

(22) PCT Filed: Mar. 24, 2022
(86) PCT No.: PCT/US2022/021806

§ 371 Date: Sep. 25, 2023

Related U.S. Application Data
(60) Provisional application No. 63/166,167, filed on Mar. 25, 2021.
(60) Provisional application No. 63/188,321, filed on May 13, 2021.

(51) Int. Cl. C12Q 1/6886; A61K 45/00; G16B 25/10; G16H 50/20

(52) U.S. Cl. CPC C12Q 1/6886; A61K 45/00; G16B 25/10; G16H 50/20; C12Q 2600/106; C12Q 2600/112; C12Q 2600/158

(57) ABSTRACT

The disclosure provides methods to categorize cancers and cancer patients using a classifier, TME Panel-1, which stratifies patients and cancers according to tumor microenvironments. Treatment decisions are then guided by the presence/absence of a particular TME phenotype class. Also provided are methods for treating a subject, e.g., a human subject, afflicted with gastric cancer, breast cancer, prostate cancer, liver cancer, carcinoma of head and neck, melanoma, colorectal cancer, or ovarian cancer comprising administering a particular therapy depending on the classification of the cancer's TME according to the TME Panel-1 classifier. Also provided are personalized treatments that can be administered to patients depending on the TME Panel-1 classification of a particular type of cancer, e.g., left or right colorectal cancer or dMMR colorectal cancer.

### CROSS-REFERENCE TO RELATED APPLICATIONS

This PCT application claims the priority benefit of U.S. Provisional Application No. 63/166,167, filed on Mar. 25, 2021, and 63/188,321, filed on May 13, 2021, both of which are herein incorporated by reference in their entireties.

### REFERENCE TO SEQUENCE LISTING SUBMITTED ELECTRONICALLY

The content of the electronically submitted sequence listing (Name: 4488_024PC01_Seqlisting_ST25.txt; Size: 20,480 Bytes; and Date of Creation: Mar. 11, 2022) is herein incorporated by reference in its entirety.

### FIELD

The present disclosure relates to methods for stratifying cancer patients suffering from colorectal cancer, gastric cancer, breast cancer, prostate cancer, liver cancer, carcinoma of head and neck, melanoma, ovarian cancer, glioma, glioblastoma, or lung cancer based on a diagnostic panel that uses gene expression data to classify patients based on the dominant biologies of the tumor microenvironment, methods for identifying subpopulations of cancer patients for treatment with particular therapies, and personalized therapies for treating patients having specific biologies of the tumor microenvironment.

### BACKGROUND

Few diagnostic tools exist to match an individual suffering from cancer to the therapy regime that provides optimal changes for survival, and for the majority of patients the clinician must choose therapies without the benefit of precision tools that would indicate the best course of treatment.

In 2015, a collaboration across six research groups produced the Consensus Molecular Subtypes (CMS) model for typing CRC patients (Guinney et al. 2015 Nat. Med. 21:1350-1356). The CMS model represents the synthesis of six different classification schemes, and based on gene expression data returns four distinct subtypes: CMS groups 1-4 (with a fraction of patients unclassifiable). These four groups have been further annotated based on analysis of additional molecular features: briefly, CMS1 is immunogenic and includes MSI-H; CMS2 is WNT & MYC active; CMS3 includes KRAS mutations and metabolic dysregulation; and CMS4 is stromal or angiogenic in nature. CMS subgroups by-and-large accord with known pathological features of the disease (Baran et al. 2018 Gastroenterol. Res. 11:264-273), and are prognostic for overall survival (OS) and progression free survival (PFS) (Lenz et al. 2019 J. Clin. Oncol. 37:1876-1885). Nevertheless, CMS has not proved to be predictive for targeted therapies such as bevacizumab, and has yielded some confounding results between different trials (e.g. CALBG/SWOG 80405 vs FIRE-3) (Lenz et al. 2019 J. Clin. Oncol. 37:1876-1885; Stintzing et al. 2019 Ann. Oncol. 30:1796-1803). Thus patients and clinicians are still in need of predictive diagnostic tools to guide precision treatment of colorectal cancer, gastric cancer, breast cancer, prostate cancer, liver cancer, carcinoma of head and neck, melanoma, or ovarian cancer.

### BRIEF SUMMARY

The present disclosure provides a method for treating a human subject afflicted with a cancer comprising administering a TME phenotype class-specific therapy to the subject, wherein, prior to the administration, a TME phenotype class is determined by applying an Artificial Neural Network (ANN) classifier to a plurality of RNA expression levels obtained from a gene panel from a cancer tumor sample obtained from the subject, wherein the cancer tumor is assigned a TME phenotype class selected from the group consisting of IS (immune suppressed), A (angiogenic), IA (immune active), ID (immune desert), and combinations thereof.

Also provided is a method for treating a human subject afflicted with a cancer comprising (i) applying an ANN classifier to a plurality of RNA expression levels obtained from a gene panel from a cancer tumor sample obtained from the subject, wherein the cancer tumor is assigned a TME phenotype class selected from the group consisting of IS, A, IA, ID, and combinations thereof, and, (ii) administering a TME phenotype class-specific therapy to the subject.

Also provided is a method for identifying a human subject afflicted with a cancer suitable for treatment with a TME phenotype class-specific therapy, the method comprising applying an ANN classifier to a plurality of RNA expression levels obtained from a gene panel from a cancer tumor sample obtained from the subject, wherein the cancer tumor is assigned a TME phenotype class selected from the group consisting of IS, A, IA, ID, and combinations thereof, and wherein the assigned TME phenotype class indicates that a TME phenotype class-specific therapy can be administered to treat the cancer. In some aspects, the ANN classifier comprises an input layer, a hidden layer, and an output layer. In some aspects, the input layer comprises between 2 and 100 nodes.

In some aspects, each node in the input layer corresponds to a gene in a gene panel selected from the genes presented in TABLE 1 and TABLE 2, wherein the gene panel comprises (i) between 1 and 63 genes selected from TABLE 1, and between 1 and 61 genes selected from TABLE 2, (ii) a gene panel comprising genes selected from TABLE 3 and TABLE 4, (iii) a gene panel of TABLE 5, or (iv) any of the gene panels (Genesets) disclosed in FIG. 9A-G. In some aspects, the sample comprises intratumoral tissue. In some aspects, the RNA expression levels are transcribed RNA expression levels determined using Next Generation Sequencing (NGS) such as RNA-Seq, EdgeSeq, PCR, Nanostring, WES, or combinations thereof. In some aspects, the hidden layer comprises 2 nodes and the output layer comprises 4 output nodes, wherein each one of the 4 output nodes in the output layer corresponds to a TME phenotype class, wherein the 4 TME phenotype classes are IA, IS, ID, and A. In some aspects, the method further comprises applying a logistic regression classifier comprising a Softmax function to the output of the ANN, wherein the Softmax function assigns probabilities to each TME phenotype class. In some aspects, the TME phenotype-class specific therapy is an IA, IS, ID or A TME phenotype class-specific therapy or a combination thereof. In some aspects, the TME phenotype class-specific therapy is an IA TME phenotype class-specific therapy comprising a checkpoint modulator therapy.

In some aspects, the checkpoint modulator therapy comprises administering (i) an activator of a stimulatory immune checkpoint molecule such as an antibody molecule against GITR, OX-40, ICOS, 4-1BB, or a combination thereof, (ii) a RORy agonist; or, (iii) an inhibitor of an inhibitory immune checkpoint molecule such as an antibody against PD-1 (such as nivolumab, pembrolizumab, cemiplimab, PDR001, CBT-501, CX-188, sintilimab, tislelizumab, TSR-042 or an antigen-binding portion thereof), an antibody against PD-L1 (such as avelumab, atezolizumab, durvalumab, CX-072, LY3300054, or an antigen-binding portion thereof), an antibody against PD-L2, or an antibody against CTLA-4, alone or a combination thereof, or in combination with an inhibitor of TIM-3, LAG-3, BTLA, TIGIT, VISTA, TGF-β, LAIR1, CD160, 2B4, GITR, OX40, 4-1BB, CD2, CD27, CDS, ICAM-1, LFA-1, ICOS, CD30, CD40, BAFFR, HVEM, CD7, LIGHT, NKG2C, SLAMF7, NKp80, or CD86.

In some aspects, the checkpoint modulator therapy comprises administering (i) an anti-PD-1 antibody selected from the group consisting of nivolumab, pembrolizumab, cemiplimab, PDR001, CBT-501, CX-188, sintilimab, tislelizumab, or TSR-042; (ii) an anti-PD-L1 antibody selected from the group consisting of avelumab, atezolizumab, CX-072, LY3300054, and durvalumab; or (iii) a combination thereof. In some aspects, the TME phenotype class-specific therapy is an IS-class TME therapy comprising administering (1) a checkpoint modulator therapy and an anti-immunosuppression therapy, and/or (2) an antiangiogenic therapy. In some aspects, the checkpoint modulator therapy comprises administering an inhibitor of an inhibitory immune checkpoint molecule. In some aspects, the inhibitor of an inhibitory immune checkpoint molecule is (i) an antibody against PD-1 selected from the group consisting of pembrolizumab, nivolumab, cemiplimab, PDR001, CBT-501, CX-188, sintilimab, tislelizumab, TSR-042, an antigen-binding portion thereof, and a combination thereof, (ii) an antibody against PD-L1 selected from the group consisting of avelumab, atezolizumab, CX-072, LY3300054, durvalumab, an antigen-binding portion thereof, and a combination thereof, (iii) an antibody against PD-L2 or an antigen binding portion thereof, (iv) an antibody against CTLA-4 selected from ipilimumab and the bispecific antibody XmAb20717 (anti PD-1/anti-CTLA-4); or (v) a combination thereof.

In some aspects, wherein the antiangiogenic therapy comprises administering (i) an anti-VEGF antibody selected from the group consisting of varisacumab, bevacizumab, navicixizumab (anti-DLL4/anti-VEGF bispecific), ABL101 (NOV1501) (anti-DLL4/anti-VEGF), ABT165 (anti-DLL4/anti-VEGF), and a combination thereof, (ii) an anti-VEGFR2 antibody, wherein the anti-VEGFR2 antibody comprises ramucirumab; or, (iii) a combination thereof. In some aspects, the anti-immunosuppression therapy comprises administering an anti-PS antibody, anti-PS targeting antibody, antibody that binds β2-glycoprotein 1, inhibitor of PI3Kγ, adenosine pathway inhibitor, inhibitor of IDO, inhibitor of TIM, inhibitor of LAG3, inhibitor of TGF-β, CD47 inhibitor, or a combination thereof, wherein (i) the anti-PS targeting antibody is bavituximab, or an antibody that binds β2-glycoprotein 1; (ii) the PI3Kγ inhibitor is LY3023414 (samotolisib) or IPI-549; (iii) the adenosine pathway inhibitor is AB-928; (iv) the TGFβ inhibitor is LY2157299 (galunisertib) or the TGFβR1 inhibitor is LY3200882; (v) the CD47 inhibitor is magrolimab (5F9); and, (vi) the CD47 inhibitor targets SIRPα.

In some aspects, the anti-immunosuppression therapy comprises administering an inhibitor of TIM-3, LAG-3, BTLA, TIGIT, VISTA, TGF-β or its receptors, an inhibitor of LAIR1, CD160, 2B4, GITR, OX40, 4-1BB, CD2, CD27, CDS, ICAM-1, LFA-1, ICOS, CD30, CD40, BAFFR, HVEM, CD7, LIGHT, NKG2C, SLAMF7, NKp80, an agonist of CD86, or a combination thereof. In some aspects, the TME phenotype class-specific therapy is an A TME phenotype class-specific therapy comprising administering a VEGF-targeted therapy, an inhibitor of angiopoietin 1 (Ang1), an inhibitor of angiopoietin 2 (Ang2), an inhibitor of DLL4, a bispecific of anti-VEGF and anti-DLL4, a TKI inhibitor, an anti-FGF antibody, an anti-FGFR1 antibody, an anti-FGFR2 antibody, a small molecule that inhibits FGFR1, a small molecule that inhibits FGFR2, an anti-PLGF antibody, a small molecule against a PLGF receptor, an antibody against a PLGF receptor, an anti-VEGFB antibody, an anti-VEGFC antibody, an anti-VEGFD antibody, an antibody to a VEGF/PLGF trap molecule such as aflibercept, or ziv-aflibercet, an anti-DLL4 antibody, an anti-Notch therapy such as an inhibitor of gamma-secretase, or any combination thereof.

In some aspects, the TKI inhibitor is selected from the group consisting of cabozantinib, vandetanib, tivozanib, axitinib, lenvatinib, sorafenib, regorafenib, sunitinib, fruquitinib, pazopanib, and any combination thereof.

In some aspects, the VEGF-targeted therapy comprises administering (i) an anti-VEGF antibody comprising varisacumab, bevacizumab, an antigen-binding portion thereof, or a combination thereof, (ii) an anti-VEGFR2 antibody comprising ramucirumab or an antigen-binding portion thereof, or, (iii) a combination thereof.

In some aspects, the A TME phenotype class-specific therapy comprises administering an angiopoietin/TIE2-targeted therapy comprising endoglin and/or angiopoietin. In some aspects, the A TME phenotype class-specific therapy comprises administering a DLL4-targeted therapy comprising navicixizumab, ABL101 (NOV1501), ABT165, or a combination thereof. In some aspects, the TME phenotype class-specific therapy is an ID TME phenotype class-specific therapy comprising administering a of a checkpoint modulator therapy concurrently or after the administration of a therapy that initiates an immune response. In some aspects, the therapy that initiates an immune response is a vaccine, a CAR-T, or a neo-epitope vaccine. In some aspects, the checkpoint modulator therapy comprises the administration of an inhibitor of an inhibitory immune checkpoint molecule. In some aspects, the inhibitor of an inhibitory immune checkpoint molecule is an antibody against PD-1, PD-L1, PD-L2, CTLA-4, or a combination thereof. In some aspects, the anti-PD-1 antibody comprises nivolumab, pembrolizumab, cemiplimab, PDR001, CBT-501, CX-188, sintilimab, tislelizumab, or TSR-042, or an antigen-binding portion thereof. In some aspects, the anti-PD-L1 antibody comprises avelumab, atezolizumab, CX-072, LY3300054, durvalumab, or an antigen-binding portion thereof. In some aspects, the anti-CTLA-4 antibody comprises ipilimumab or the bispecific antibody XmAb20717 (anti PD-1/anti-CTLA-4), or an antigen-binding portion thereof. In some aspects, the checkpoint modulator therapy comprises the administration of (i) an anti-PD-1 antibody selected from the group consisting of nivolumab, pembrolizumab, cemiplimab PDR001, CBT-501, CX-188, sintilimab, tislelizumab, and TSR-042; (ii) an anti-PD-L1 antibody selected from the group consisting of avelumab, atezolizumab, CX-072, LY3300054, and durvalumab; (iv) an anti-CTLA-4 antibody, which is ipilimumab or the bispecific antibody XmAb20717 (anti PD-1/anti-CTLA-4), or (iii) a combination thereof.

In some aspects, the method further comprises (a) administering chemotherapy; (b) performing surgery; (c) administering radiation therapy; or, (d) any combination thereof. In some aspects, the cancer is relapsed, refractory, metastatic, dMMR, or a combination thereof. In some aspects, the cancer is refractory following at least one prior therapy comprising administration of at least one anticancer agent. In some aspects, the cancer is selected from the group consisting of gastric cancer, breast cancer, prostate cancer, liver cancer, carcinoma of head and neck, melanoma, colorectal cancer, ovarian cancer, glioma, glioblastoma, or lung cancer. In some aspects, the gastric cancer is locally advanced, metastatic gastric cancer, or previously untreated gastric cancer). In some aspects, the breast cancer is locally advanced or metastatic Her2-negative breast cancer. In some aspects, the prostate cancer is castration-resistant metastatic prostate cancer. In some aspects, the liver cancer is hepatocellular carcinoma such as advanced metastatic hepatocellular carcinoma. In some aspects, the carcinoma of head and neck is recurrent or metastatic squamous cell carcinoma of head and neck. In some aspects, the colorectal cancer is advanced colorectal cancer metastatic to liver. In some aspects, the ovarian cancer is platinum-resistant ovarian cancer or platinum-sensitive recurrent ovarian cancer. In some aspects, the glioma is metastatic glioma. In some aspects, the lung cancer is NSCLC.

In some aspects, administering a TME phenotype class-specific therapy reduces the cancer burden by at least about 10%, 20%, 30%, 40%, or 50% compared to the cancer burden prior to the administration. In some aspects, the subject exhibits progression-free survival of at least about 1, 2, 3, 4, 5, 6, 7, 8, 9, 10, 11, or 12 months, or at least about 1, 2, 3, 4 or 5 years after the initial administration of the TME phenotype class-specific therapy. In some aspects, the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy. In some aspects, the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy. In some aspects, the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy.

In some aspects, administering the TME phenotype class-specific therapy improves progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject who has not received a TME phenotype class-specific therapy assigned using an ANN classifier such as TME Panel-1. In some aspects, administering the TME phenotype class-specific therapy improves overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject who has not received a TME phenotype class-specific therapy assigned using an ANN classifier such as TME Panel-1.

Also provided is a method of assigning a TME phenotype class to a cancer in a subject in need thereof, the method comprising (i) generating an ANN classifier by training an ANN with a training set comprising RNA expression levels for each gene in a gene panel in a plurality of samples obtained from a plurality of subjects, wherein each sample is assigned a TME phenotype classification; and, (ii) assigning, using the ANN classifier, a TME phenotype class to the cancer in the subject, wherein the input to the ANN classifier comprises RNA expression levels for each gene in the gene panel in a test sample obtained from the subject.

Also provided is a method of assigning a TME phenotype class to a cancer in a subject in need thereof, the method comprising generating an ANN classifier by training an ANN with a training set comprising RNA expression levels for each gene in a gene panel in a plurality of samples obtained from a plurality of subjects, wherein each sample is assigned a TME phenotype classification; wherein the ANN classifier assigns a TME phenotype class to the cancer in the subject using as input RNA expression levels for each gene in the gene panel in a test sample obtained from the subject. Also provided is a method of assigning a TME phenotype class to a cancer in a subject in need thereof, the method comprising using an ANN classifier to predict the TME phenotype class of the cancer in the subject, wherein the ANN classifier is generated by training an ANN with a training set comprising RNA expression levels for each gene in a gene panel in a plurality of samples obtained from a plurality of subjects, wherein each sample is assigned a TME phenotype class or combination thereof. In some aspects, the method is implemented in a computer system comprising at least one processor and at least one memory, the at least one memory comprising instructions executed by the at least one processor to cause the at least one processor to implement the machine-learning model. In some aspects the method further comprises (i) inputting, into the memory of the computer system, the ANN classifier code; (ii) inputting, into the memory of the computer system, the gene panel input data corresponding to the subject, wherein the input data comprises RNA expression levels; (iii) executing the ANN classifier code; or; (v) any combination thereof.

Also provided is a method to treat a subject having a locally advanced, metastatic gastric cancer with an IA TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a locally advanced, metastatic gastric cancer with an A TME phenotype comprising administering an A TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a locally advanced, metastatic gastric cancer with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a previously untreated gastric cancer with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a previously untreated gastric cancer with an A TME phenotype comprising administering an A TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a locally advanced/metastatic HER2-negative breast Cancer with an A TME phenotype comprising administering an A TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a locally advanced/metastatic HER2-negative breast cancer with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a castration-resistant metastatic prostate cancer with an A TME phenotype comprising administering an A TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a castration-resistant metastatic prostate cancer with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a advanced metastatic hepatocellular carcinoma with an IA TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a advanced metastatic hepatocellular carcinoma with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a recurrent/metastatic squamous cell carcinoma of head and neck with an IA TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a recurrent/metastatic squamous cell carcinoma of head and neck with an IS TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a melanoma with an IA TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a melanoma with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a advanced colorectal cancer metastatic to liver with an ID TME phenotype comprising administering an ID TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having a platinum resistant or platinum-sensitive recurrent ovarian cancer with an IA, IS or A TME phenotype comprising administering an IA, IS, or A TME phenotype class-specific therapy to the subject, wherein the TME phenotype class has been assigned by applying an ANN classifier to a plurality of RNA expression levels obtained from a gene panel from a cancer tumor sample obtained from the subject.

Also provided is method to treat a subject having platinum-resistant or platinum-sensitive recurrent triple negative breast Cancer with an IA, IS or A TME phenotype comprising administering an IA, IS or A TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having melanoma with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having metastatic colorectal cancer with an A or IS TME phenotype comprising administering an A or IS TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having glioma or glioblastoma with an IS or IA TME phenotype comprising administering an IS or IA TME phenotype class-specific therapy to the subject, wherein the TME phenotype class has been assigned by applying an ANN classifier to a plurality of RNA expression levels obtained from a gene panel from a cancer tumor sample obtained from the subject. Also provided is a method to treat a subject having non-small cell lung cancer with an IS or IA TME phenotype comprising administering an IS or IA TME phenotype class-specific therapy to the subject, wherein the TME phenotype class has been assigned by applying an ANN classifier to a plurality of RNA expression levels obtained from a gene panel from a cancer tumor sample obtained from the subject.

The present disclosure also provides a kit comprising (i) a plurality of oligonucleotide probes capable of specifically detecting an RNA encoding a gene biomarker from TABLE 1, and (ii) a plurality of oligonucleotide probes capable of specifically detecting an RNA encoding a gene biomarker from TABLE 2. Also provides is an article of manufacture comprising (i) a plurality of oligonucleotide probes capable of specifically detecting an RNA encoding a gene biomarker from TABLE 1 (or FIG. 9A-9G), and (ii) a plurality of oligonucleotide probes capable of specifically detecting an RNA encoding a gene biomarker from TABLE 2 (or FIG. 9A-9G), wherein the article of manufacture comprises a microarray.

The present disclosure provides an ANN classifier comprising
(a) an input layer comprising between 2 and 100 nodes, wherein each node in the input layer corresponds to a gene in a gene panel selected from the genes presented in TABLE 1 and TABLE 2, wherein the gene panel comprises (i) between 1 and 63 genes selected from TABLE 1, and between 1 and 61 genes selected from TABLE 2, (ii) a gene panel comprising genes selected from TABLE 3 and TABLE 4, (iii) a gene panel of TABLE 5, or (iv) any of the gene panels (Genesets) disclosed in FIG. 9A-G;
(b) a hidden layer comprising 2 nodes; and,
(c) an output layer comprising 4 output nodes, wherein each one of the 4 output nodes in the output layer corresponds to a TME phenotype class, wherein the 4 TME phenotype classes are IA, IS, ID, and A,
   and optionally further comprising applying a logistic regression classifier comprising a Softmax function to the output of the ANN, wherein the Softmax function assigns probabilities to each TME phenotype class.

The present disclosure provides an IA TME phenotype class-specific therapy comprising a checkpoint modulator therapy comprising administering:
(i) an activator of a stimulatory immune checkpoint molecule such as an antibody molecule against GITR, OX-40, ICOS, 4-1BB, or a combination thereof,
(ii) a RORy agonist;
(iii) an inhibitor of an inhibitory immune checkpoint molecule such as an antibody against PD-1 (such as nivolumab, pembrolizumab, cemiplimab, PDR001, CBT-501, CX-188, sintilimab, tislelizumab, TSR-042 or an antigen-binding portion thereof), an antibody against PD-L1 (such as avelumab, atezolizumab, durvalumab, CX-072, LY3300054, or an antigen-binding portion thereof), an antibody against PD-L2, or an antibody against CTLA-4, alone or a combination thereof, or in combination with an inhibitor of TIM-3, LAG-3, BTLA, TIGIT, VISTA, TGF-β, LAIR1, CD160, 2B4, GITR, OX40, 4-1BB, CD2, CD27, CDS, ICAM-1, LFA-1, ICOS, CD30, CD40, BAFFR, HVEM, CD7, LIGHT, NKG2C, SLAMF7, NKp80, or CD86; or
(iv) as combination thereof.

The present disclosure provides an IS-class TME therapy comprising administering
(1) a checkpoint modulator therapy and an anti-immunosuppression therapy, and/or
(2) an antiangiogenic therapy,
   wherein the checkpoint modulator therapy comprises administering an inhibitor of an inhibitory immune checkpoint molecule comprising
(i) an antibody against PD-1 selected from the group consisting of pembrolizumab, nivolumab, cemiplimab, PDR001, CBT-501, CX-188, sintilimab, tislelizumab, TSR-042, an antigen-binding portion thereof, and a combination thereof,
(ii) an antibody against PD-L1 selected from the group consisting of avelumab, atezolizumab, CX-072, LY3300054, durvalumab, an antigen-binding portion thereof, and a combination thereof,
(iii) an antibody against PD-L2 or an antigen binding portion thereof,
(iv) an antibody against CTLA-4 selected from ipilimumab and the bispecific antibody XmAb20717 (anti PD-1/anti-CTLA-4); or
(v) a combination thereof,
   wherein the antiangiogenic therapy comprises administering
(a) an anti-VEGF antibody selected from the group consisting of varisacumab, bevacizumab, navicixizumab (anti-DLL4/anti-VEGF bispecific), ABL101 (NOV1501) (anti-DLL4/anti-VEGF), ABT165 (anti-DLL4/anti-VEGF), and a combination thereof,
(b) an anti-VEGFR2 antibody, wherein the anti-VEGFR2 antibody comprises ramucirumab; or,
(c) a combination thereof,
   and wherein the anti-immunosuppression therapy comprises administering
(a) an anti-PS antibody, anti-PS targeting antibody, antibody that binds β2-glycoprotein 1, inhibitor of PI3Kγ, adenosine pathway inhibitor, inhibitor of IDO, inhibitor of TIM, inhibitor of LAG3, inhibitor of TGF-β, CD47 inhibitor, or a combination thereof, wherein
   the anti-PS targeting antibody is bavituximab, or an antibody that binds D2-glycoprotein 1;
   the PI3Kγ inhibitor is LY3023414 (samotolisib) or IPI-549; the adenosine pathway inhibitor is AB-928; the TGFβ inhibitor is LY2157299 (galunisertib) or the TGFβR1 inhibitor is LY3200882;
   the CD47 inhibitor is magrolimab (5F9); and, the CD47 inhibitor targets SIRPα;
(b) an inhibitor of TIM-3, LAG-3, BTLA, TIGIT, VISTA, TGF-β or its receptors, an inhibitor of LAIR1, CD160, 2B4, GITR, OX40, 4-1BB, CD2, CD27, CDS, ICAM-1, LFA-1, ICOS, CD30, CD40, BAFFR, HVEM, CD7, LIGHT, NKG2C, SLAMF7, NKp80, an agonist of CD86, or a combination thereof; or,
(c) a combination thereof.

The present disclosure provides an A TME phenotype class-specific therapy comprising administering
(i) a VEGF-targeted therapy, an inhibitor of angiopoietin 1 (Ang1), an inhibitor of angiopoietin 2 (Ang2), an inhibitor of DLL4, a bispecific of anti-VEGF and anti-DLL4, a TKI inhibitor, an anti-FGF antibody, an anti-FGFR1 antibody, an anti-FGFR2 antibody, a small molecule that inhibits FGFR1, a small molecule that inhibits FGFR2, an anti-PLGF antibody, a small molecule against a PLGF receptor, an antibody against a PLGF receptor, an anti-VEGFB antibody, an anti-VEGFC antibody, an anti-VEGFD antibody, an antibody to a VEGF/PLGF trap molecule such as aflibercept, or ziv-aflibercet, an anti-DLL4 antibody, an anti-Notch therapy such as an inhibitor of gamma-secretase, or any combination thereof, wherein the TKI inhibitor is selected from the group consisting of cabozantinib, vandetanib, tivozanib, axitinib, lenvatinib, sorafenib, regorafenib, sunitinib, fruquitinib, pazopanib, and any combination thereof, and wherein the VEGF-targeted therapy comprises administering (a) an anti-VEGF antibody comprising varisacumab, bevacizumab, an antigen-binding portion thereof, or a combination thereof, (b) an anti-VEGFR2 antibody comprising ramucirumab or an antigen-binding portion thereof, or, (c) a combination thereof;
(ii) an angiopoietin/TIE2-targeted therapy comprising endoglin and/or angiopoietin; or,
(iii) a DLL4-targeted therapy comprising navicixizumab, ABL101 (NOV1501), ABT165, or a combination thereof.

The present disclosure provides an ID TME phenotype class-specific therapy comprising administering a checkpoint modulator therapy concurrently or after the administration of a therapy that initiates an immune response, wherein the checkpoint modulator therapy comprises the administration of an inhibitor of an inhibitory immune checkpoint molecule such as an antibody against PD-1, PD-L1, PD-L2, CTLA-4, or a combination thereof, and wherein the therapy that initiates an immune response is a vaccine, a CAR-T, or a neo-epitope vaccine, wherein
(i) the anti-PD-1 antibody comprises nivolumab, pembrolizumab, cemiplimab, PDR001, CBT-501, CX-188, sintilimab, tislelizumab, or TSR-042, or an antigen-binding portion thereof;
(ii) the anti-PD-L1 antibody comprises avelumab, atezolizumab, CX-072, LY3300054, durvalumab, or an antigen-binding portion thereof; and,
(iii) the anti-CTLA-4 antibody comprises ipilimumab or the bispecific antibody XmAb20717 (anti PD-1/anti-CTLA-4), or an antigen-binding portion thereof.

In some aspects, the TME phenotype class-specific therapies disclosed herein are combined with (a) administering chemotherapy; (b) performing surgery; (c) administering radiation therapy; or, (d) any combination thereof.

In some aspects, the cancer is selected from the group consisting of (i) gastric cancer, such as locally advanced, metastatic gastric cancer, or previously untreated gastric cancer; (ii) breast cancer, such as locally advanced, triple negative breast cancer, or metastatic Her2-negative breast cancer; (iii) prostate cancer, such as castration-resistant metastatic prostate cancer; (iv) liver cancer, such as advanced metastatic hepatocellular carcinoma; (v) carcinoma of head and neck, such as recurrent or metastatic squamous cell carcinoma of head and neck; (vi) melanoma, such as metastatic melanoma; (vii) colorectal cancer, such as advanced colorectal cancer metastatic to liver; (viii) ovarian cancer, such as platinum-resistant ovarian cancer or platinum-sensitive recurrent ovarian cancer; (ix) glioma, such as metastatic glioma; (x) lung cancer, such non-small cell lung cancer (NSCLC); and, (xi) glioblastoma.

In some aspects, administering a TME phenotype class-specific therapy results in (i) reduction of the cancer burden by at least about 10%, 20%, 30%, 40%, or 50% compared to the cancer burden prior to the administration; (ii) progression-free survival of at least about 1, 2, 3, 4, 5, 6, 7, 8, 9, 10, 11, or 12 months, or at least about 1, 2, 3, 4 or 5 years after the initial administration of the TME phenotype class-specific therapy; (iii) stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy; (iv) partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy; (v) complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy; (vi) improved progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject who has not received a TME phenotype class-specific therapy assigned using an ANN classifier such as TME Panel-1; (vii) improved overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject who has not received a TME phenotype class-specific therapy assigned using an ANN classifier such as TME Panel-1; or, (viii) a combination thereof.

The present disclosure provides a method of assigning a TME phenotype class to a cancer in a subject in need thereof, the method comprising
(i) generating an ANN classifier by training an ANN with a training set comprising RNA expression levels for each gene in a gene panel in a plurality of samples obtained from a plurality of subjects, wherein each sample is assigned a TME phenotype classification; and, assigning, using the ANN classifier, a TME phenotype class to the cancer in the subject, wherein the input to the ANN classifier comprises RNA expression levels for each gene in the gene panel in a test sample obtained from the subject; or,
(ii) generating an ANN classifier by training an ANN with a training set comprising RNA expression levels for each gene in a gene panel in a plurality of samples obtained from a plurality of subjects, wherein each sample is assigned a TME phenotype classification; wherein the ANN classifier assigns a TME phenotype class to the cancer in the subject using as input RNA expression levels for each gene in the gene panel in a test sample obtained from the subject; or,
(iii) using an ANN classifier to predict the TME phenotype class of the cancer in the subject,
   wherein the ANN classifier is generated by training an ANN with a training set comprising RNA expression levels for each gene in a gene panel in a plurality of samples obtained from a plurality of subjects, wherein each sample is assigned a TME phenotype class or combination thereof.

The present disclosure provides a method to treat a subject having a cancer with a specific TME phenotype comprising administering a TME phenotype class-specific therapy to the subject wherein,
(i) the cancer is locally advanced, metastatic gastric cancer and the TME phenotype is IA, A, or IS;
(ii) the cancer is untreated gastric cancer and the TME phenotype is IS or A;
(iii) the cancer is advanced/metastatic HER2-negative breast Cancer and the TME phenotype is A or IS;
(iv) the cancer is castration-resistant metastatic prostate cancer and the TME phenotype is A or IS;
(v) the cancer is advanced metastatic hepatocellular carcinoma and the TME phenotype is IA or IS;
(vi) the cancer is recurrent/metastatic squamous cell carcinoma of head and neck and the TME phenotype is IA or IS;
(vii) the cancer is melanoma and the TME phenotype is IA or IS;
(viii) the cancer is advanced colorectal cancer metastatic to liver and the TME phenotype is ID;
(ix) the cancer is platinum resistant or platinum-sensitive recurrent ovarian cancer and the TME phenotype is IA, IS or A;
(x) the cancer is platinum-resistant or platinum-sensitive recurrent triple negative breast cancer and the TME phenotype is IA, IS or A;
(xi) the cancer is metastatic colorectal cancer and the TME phenotype is A or IS;
(xii) the cancer is glioma or glioblastoma and the TME phenotype is IS or IA; or,
(xiii) the cancer is non-small cell lung cancer and the TME phenotype is IS or IA;
   wherein the TME phenotype class has been assigned by applying an ANN classifier to a plurality of RNA expression levels obtained from a gene panel from a cancer tumor sample obtained from the subject, wherein the ANN classifier comprises
(a) an input layer comprising between 2 and 100 nodes, wherein each node in the input layer corresponds to a gene in a gene panel selected from the genes presented in TABLE 1 and TABLE 2, wherein the gene panel comprises (i) between 1 and 63 genes selected from TABLE 1, and between 1 and 61 genes selected from TABLE 2, (ii) a gene panel comprising genes selected from TABLE 3 and TABLE 4, (iii) a gene panel of TABLE 5, or (iv) any of the gene panels (Genesets) disclosed in FIG. 9A-G;
(b) a hidden layer comprising 2 nodes; and,
(c) an output layer comprising 4 output nodes, wherein each one of the 4 output nodes in the output layer corresponds to a TME phenotype class, wherein the 4 TME phenotype classes are IA, IS, ID, and A,
   and optionally further comprises a logistic regression classifier comprising a Softmax function to the output of the ANN, wherein the Softmax function assigns probabilities to each TME phenotype class.

### BRIEF DESCRIPTION OF THE DRAWINGS/FIGURES

FIG. 1 shows the four TME (tumor microenvironment) phenotype classes assigned by the TME Panel-1 Classifier. The angiogenic TME phenotype class, A, is characterized by high angiogenesis and low immune signature scores. Pathologic angiogenesis drives tumor growth and metastasis. The immune suppressed TME phenotype class, IS, is characterized by high angiogenesis and high immune signature score. The immune complement consists mostly of suppressive cells. The immune desert TME phenotype class, ID, is characterized by a low angiogenesis signature score and a low immune signature score. Immune cells are absent and vasculature is functional. The immune active TME phenotype class, IA, is characterized by a low angiogenesis and a high immune signature score. T-cells have infiltrated but may not be functioning optimally.

FIG. 2A shows the prevalence of TME phenotype classes in the CIT dataset and the Wood-Hudson CRC dataset. The CIT dataset was split into early- (0-2) and late-stage (3-4) disease, and compared to Wood Hudson for which 89 of 93 patients were stage 3-4. For each data subset, the proportion of patients classified as Angiogenic (A), Immune Active (IA), Immune Desert (ID) and Immune Suppressed (IS) was tabulated. TME phenotype classes are color coded according to the figure legend.

FIG. 2B shows the left and right-handed CRC composition of each TME phenotype class by stage, as classified by the TME Panel-1 Classifier. The data subsets presented in FIG. 2A were further split based on the side of tumor, Left (distal) or Right (proximal), and the TME phenotype class proportions were retabulated.

FIG. 3A is Kaplan-Meier plot of disease-free survival (DFS) of patients in early stage (0-2) in the CIT dataset as classified by the TME Panel-1 Classifier. Each survival curve represents a TME Panel-1 phenotype class, as indicated in the legend. Capital-N is number of patients, lower case-n is number of deaths. In the absence of anti-angiogenic treatment, patients with tumors in the A TME phenotype class had the worst prognosis, followed by the patients with tumors in the IS TME phenotype class. Patients in the IA TME phenotype class had the best outcome.

FIG. 3B is a Kaplan-Meier plot of overall survival (OS) of late stage (3-4) Wood-Hudson CRC patients as classified by the TME Panel-1 Classifier. Each survival curve represents a TME Panel-1 phenotype class, as indicated in the legend. Capital-N is number of patients, lower case-n is number of deaths. In the absence of anti-angiogenic treatment, patients with tumor in the A TME phenotype class had the worst prognosis, showing lowest median survival, followed by the patients with tumors in the IS TME phenotype class.

FIG. 4A is a diagram of the angiogenic and immune axes that underlies the latent space analysis shown in FIGS. 4B-4F. Each patient was plotted on the TME Panel-1 landscape as defined by the Immune signature (x-axis) and Angiogenesis signature (y-axis).

FIGS. 4B-4E are latent space plots of the CMS classes 1-4 after classification by the TME Panel-1 Classifier. The grayscale contours are probability bands that represent the probability of a particular TME phenotype classification by the TME Panel-1 Classifier.

FIG. 4F is a latent space plot of unclassified patients of the CIT dataset, after classification by the TME Panel-1 Classifier. The grayscale contours are probability bands that represent the probability of a particular TME phenotype classification by the TME Panel-1 Classifier.

FIG. 5A shows TME phenotype class distribution of the CIT dataset within each CMS group. For each CMS group, the proportion of patients of each TME class is shown, shaded according to the legend.

FIG. 5B shows CMS distribution of the CIT dataset within each TME phenotype class. For each TME class, the proportion of patients of each CMS group is shown, shaded according to the legend. FIGS. 5A and 5B represent the same but converse tabulation analysis.

FIGS. 6A and 6B show prevalence of DNA Mismatch Repair (dMMR) defective-patients among CMS groups and among TME, or stromal, phenotype classes. About three quarters of dMMR patients were captured by CMS1 (77%) (FIG. 6A), whereas 96% of dMMR patients were classified as high immune TME phenotypes (IA) and (IS) (FIG. 6B). Groups and classes are shaded according to the legends.

FIG. 7 shows a simplified view of the TME Panel-1 Classifier in the present disclosure. The TME Panel-1 Classifier comprises an input layer with inputs corresponding to each gene in the gene panel (e.g., a 124 gene panel, 105 gene panel, 98 gene panel, or alternatively an 87 gene panel), a hidden layer comprising two neurons (or alternatively 3, 4 or 5 neurons), and an output layer that would correspond to TME phenotype class assignments (i.e., stromal phenotype assignments).

FIG. 8 is a chart showing TME phenotype class assignments based on the application of the TME Panel-1 Classifier disclosed herein, as well as treatment classes assigned to each TME phenotype class.

FIG. 9A shows the presence (open cells) or absence (full cells) of 124 genes in Genesets 1 to 44.

FIG. 9B shows the presence (open cells) or absence (full cells) of 124 genes in Genesets 45 to 88.

FIG. 9C shows the presence (open cells) or absence (full cells) of 124 genes in Genesets 89 to 132.

FIG. 9D shows the presence (open cells) or absence (full cells) of 124 genes in Genesets 133 to 177.

FIG. 9E shows the presence (open cells) or absence (full cells) of 124 genes in Geneset 178 to 222.

FIG. 9F shows the presence (open cells) or absence (full cells) of 124 genes in Geneset 223 to 267.

FIG. 9G shows the presence (open cells) or absence (full cells) of 124 genes in Geneset 268 to 282.

FIG. 10 is a latent space plot corresponding to vidulotimod/CMP-001.

### DETAILED DESCRIPTION

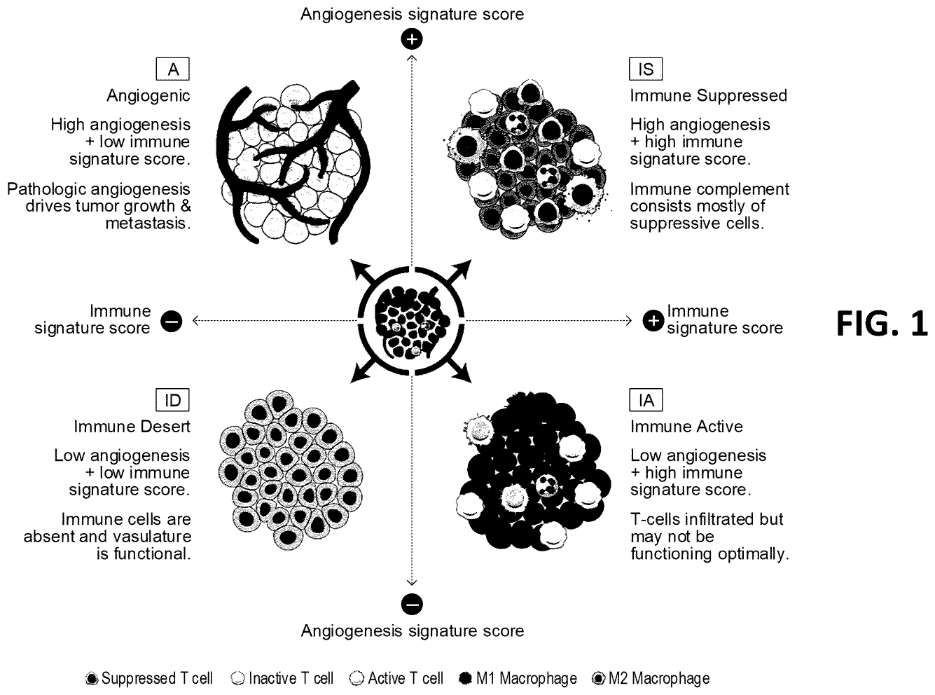

The present disclosure provides methods to stratify patients with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) according to a diagnostic panel that uses gene expression data to classify patients based on the dominant biologies of the tumor microenvironment. Strand-Tibbitts et al. 2020 Development of an RNA-based Diagnostic Platform Based on the Tumor Microenvironment Dominant Biology. SITC (2020). See FIG. 1.

The TME Panel-1 Classifier (“TME Panel-1”) used to stratify cancer patients disclosed herein employs a machine learning model that has learned two gene signatures, an Angiogenesis Signature and an Immune Signature, representing respectively the angiogenic and immune biologies that dominate the stroma of the tumor. The combinations of these biologies result in four different tumor microenvironment (TME), or stromal, phenotype classes: Angiogenic (A), Immune Suppressed (IS), Immune Active (IA) and Immune Desert (ID). In some aspects, the terms “immune desert” and “microenvironment desert” are used interchangeably. The TME Panel-1 Classifier assigns patients into one of these four TME phenotype classes based on gene expression from patient tumor samples, e.g., RNA expression data. These TME phenotype classes are independent of disease stage or demographics, and confer distinct prognostic risk. The TME Panel-1 Classifier is predictive of outcome for anti-angiogenic and checkpoint inhibitor therapies, including approved and investigational drugs. See, e.g., U.S. application Ser. No. 17/089,234, which is incorporated by reference herein in its entirety.

TME Panel-1 learns the (latent) gene expression patterns that classify an individual patient into specific TME phenotype classes. TME Panel-1 effectively compresses the high dimensional data (gene expressions of all genes in the input geneset) into a lower dimensional (latent) space. TME Panel-1 was originally trained and validated on gastric cancer. Analysis of over 2,000 biobanked patient samples indicated that the classifier can also be applied to other cancers, e.g., colorectal cancer. TME Panel-1 can stratify cancer patients, e.g., patients with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) predict therapeutic outcomes, and guide the selection of specific therapies.

In some aspects, the present disclosure provides methods for treating a subject, e.g., a human subject, afflicted with a particular type of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) comprising administering a particular therapy depending on the classification of the cancer or the patient in a specific TME phenotype class according to a classifier disclosed herein, e.g., the TME Panel-1 Classifier.

Also provided are personalized treatments that can be administered to a subject having a particular type of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) wherein the cancer or the subject have been classified into a particular TME phenotype class or determined not to have a cancer classified into a particular TME phenotype class according to a classifier disclosed herein, e.g., the TME Panel-1 Classifier.

The present disclosure also provides methods for treating a subject, e.g., a human subject, afflicted with a particular type of cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC), the methods comprising administering a particular therapy depending on the classification of the cancer or the patient in a specific TME phenotype class according to a classifier disclosed herein, e.g., the TME Panel-1 Classifier.

Also provided are personalized treatments that can be administered to a subject having a particular type of cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) wherein the cancer or the subject have been classified into a particular TME phenotype class or determined not to have a cancer classified into a particular TME phenotype class according to a classifier disclosed herein, e.g., the TME Panel-1 Classifier.

The application of the methods and compositions disclosed herein can improve clinical outcomes by matching cancer patients, e.g., patients having gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) to TME phenotype class-specific therapies with a mechanism of action that targets one or more specific TME phenotype classes.

Similarly, the application of the methods and compositions disclosed herein can improve clinical outcomes by matching cancer patients, e.g., patients having gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) to TME phenotype class-specific therapies with a mechanism of action that targets one or more specific TME phenotype classes.

Dominant TME phenotype classes can be directional but modified for any specific drug based on the complexity of the mechanism of action of drug, drugs, or clinical regimen. Combinations of drugs or clinical regimens (i.e., one or more TME phenotype class-specific therapies disclosed below) can be applied to multiple TME phenotype classes if relevant, e.g., to a patient having a tumor that is biomarker-positive for more than one TME phenotype class or is predominantly one TME phenotype class, but there is contribution of another TME phenotype class as seen in the probability function of the model, e.g., the TME Panel-1 Classifier disclosed herein. Thus, the term “predominantly,” as applied to a TME phenotype class disclosed herein indicates that a patient or sample is biomarker positive for a particular TME phenotype class (e.g., IA), but other TME phenotype classes (e.g., IS, ID or A) or combinations thereof also contribute to the biomarker signal as seen in the probability function of the model, e.g., the TME Panel-1 Classifier disclosed herein.

An advantage of the disclosed ANN classifiers, e.g., the TME Panel-1 Classifier, over other classifiers known in the art is that a sample from a patient who is, e.g., part of a clinical trial or a clinical regimen, can be correctly assigned to a specific TME phenotype class without reference to any other current patient data. Thus, while the availability of a latent plot with the probabilities for each TME phenotype class is useful, it is not required to correctly assign a specific TME phenotype class.

The ANN classifiers of the present disclosure, e.g., the TME Panel-1 Classifier, are particularly advantageous over classifiers known in the art such as CMS (Consensus Molecular Subtype) in the case of colorectal cancer, which has no clear predictive value. Prognostic biomarkers are used to foretell the course of a disease independent of treatment. For example, patients with hepatocellular carcinoma (HCC) and high levels of alpha fetoprotein (AFP) tend to have worse outcomes irrespective of therapy, and obesity is a known prognostic biomarker for outcomes of COVID-19 patients. CMS subtypes, which resulted from unsupervised clustering of input data (DNA, RNA, proteomics), are not predictive in the same way that the TME phenotypes classes A, IA, IS, and ID are. The sets of genes used to classify a tumor within a TME phenotype classes or classes based on an angiogenesis signature score and an immune signature score were developed empirically from the biology of the tumor microenvironment, and therefore the ANN method trained using those sets of genes, e.g., the TME Panel-1 classifier, is predictive of beneficial treatment.

The CMS approach in colorectal cancer addresses many cancer biologies. Stintzing et al., set out to differentiate cetuximab and bevacizumab in colorectal cancer, as both were approved for wild type KRAS colorectal cancer in their respective trials. Stintzing et al. (2019) Annals of Oncology 30: 1796-1803. The trial described in Stintzing, FIRE1, was inconclusive. Since CMS4 Mesenchymal is associated with stromal infiltration, TGFB activation, and angiogenesis, it would follow that anti-angiogenic therapies would benefit some of these patients. However, in Stintzing et al., bevacizumab did not help the CMS3 and CMS4 patients as much as cetuximab. Further, since CMS2 is associated with WNT and MYC activation, it would follow that EGFR inhibitors, including the anti-EGFR monoclonal antibody cetuximab, would benefit some of these patients. However, in Stintzing et al., the CMS2 patients benefitted more from the anti-angiogenic bevacizumab than from cetuximab.

The ANN classifiers of the present disclosure, e.g., the TME Panel-1 Classifier, are not limited to colorectal cancer and address two biologies, represented by two signatures. The empirically-determined genes of each signature represent genes related to angiogenesis or to immune processes, but the ANN method relies on inputs from genes related to angiogenesis (e.g., those in TABLE 1) or to immune processes (e.g., those in TABLE 2) for both of the hidden nodes or neurons. Yet, the classifier output can be simplified by calling one hidden node or neuron the angiogenic axis, corresponding to an angiogenic signature score, and the other the immune axis, corresponding to an immune signature score. See FIG. 1.

Application of the ANN classifiers of the present disclosure, e.g., the TME Panel-1 Classifier, to colorectal cancer patient data indicates that the TME Panel-1 classifier is superior to CMS subtypes in predictive power, e.g., to predict the response of tumors belonging to specific TME phenotype classes to bevacizumab (AVASTIN®) in colorectal cancer patients. Furthermore, the ANN classifiers of the present disclosure, e.g., the TME Panel-1 Classifier, identify TME differences between left and right colorectal cancers, which allows the selection of TME phenotype class-specific therapies matching the different phenotype observed in left and right colorectal cancer. The differences in TME phenotypes between left and right colorectal cancers provide an explanation for responses to bevacizumab (AVASTIN®), which could not be explained based on CMS classification. Quite generally, left-sided colorectal cancer has a more angiogenic stromal phenotype, and right-sided colorectal cancer has a more immune stromal phenotype.

The classifiers of the present disclosure, e.g., the TME Panel-1 Classifier, are capable of more effectively capturing the population of colorectal cancer patients with an A TME phenotype class than CMS4, and therefore permit a more accurate selection of the appropriate therapy, and are more effective predictors of therapeutic response. Similarly, the classifiers of the present disclosure, e.g., the TME Panel-1 Classifier, are capable of more effectively and completely capturing the population of patients with an IA TME phenotype class that are eligible for checkpoint inhibitor therapy even when they are not dMMR or MSI-H. Thus, the classifiers of the present disclosure, e.g., the TME Panel-1 Classifier, can stratify, e.g., colorectal cancer patients, in specific subpopulations based, for example, on whether the cancer is metastatic or not, the location of the cancer (e.g., left or right), or the presence or absence of specific molecular biomarkers or features (e.g., MMR status or MSI-H status), and assign personalized TME phenotype class-specific therapies to the patient more accurately than other classifiers known in the art. This stratification of cancer patients, e.g., colorectal cancer patients, in specific subpopulations with specific TME phenotypes allows for more accurately predicting the therapeutic response to each available therapy, allowing the clinician to design a course of treatment(s) that maximizes the chances of a positive outcome.

An important advantage of the classifiers of the present disclosure is that they are tumor agnostic, i.e., the same oncology predictive platform can be applied to multiple types of cancer. Whereas other biomarker-based approaches to classify different types of cancer rely of cancer-specific sets of biomarkers and probes (e.g., each type of tumor cell requires a specific set of RNA probes which are cancer-specific), the present approach is based on the use of a common set of genes (described as a “training set” or “defining set”) that can be applied to different types of cancer. In other words, a conventional biomarker-based tumor classification system would require a number of probes that would be specific to each cancer type (e.g., breast cancer, liver cancer, ovarian cancer, prostate cancer, etc.). Thus, for example, a set of probes (e.g., in a kit, array, etc.) to select a treatment for breast cancer could not be used to select a treatment for prostate cancer or liver cancer. In contrast, the current method relies on a proprietary set of genes expressed in the stroma of the tumor, i.e., cells, vasculature, etc. surrounding the cancer cells, not the cancer cells. Accordingly, it is possible to use a single set of RNA probes (e.g., in a kit, array, etc.) to obtain RNA expression data that can yield, after being processed by the Artificial Intelligence platform based on machine learning disclosed herein, a preferred treatment or a prediction of the therapeutic outcome for numerous types of cancer.

### Terms

In order that the present disclosure can be more readily understood, certain terms are first defined. As used in this disclosure, except as otherwise expressly provided herein, each of the following terms shall have the meaning set forth below. Additional definitions are set forth throughout the disclosure.

“Administering” refers to the physical introduction of a composition comprising a therapeutic agent (e.g., a monoclonal antibody) to a subject, using any of the various methods and delivery systems known to those skilled in the art. Preferred routes of administration include intravenous, intramuscular, subcutaneous, intraperitoneal, spinal or other parenteral routes of administration, for example by injection or infusion.

The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, intraocular, intravitreal, periorbital, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “antibody” includes, by way of example, monoclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody. As used herein, the term “antibody” does not include naturally occurring antibodies or polyclonal antibodies. As used herein, the term “naturally occurring antibodies” and “polyclonal antibodies” do not include antibodies resulting from an immune reaction induced by a therapeutic intervention, e.g., a vaccine.

An “isolated antibody” refers to an antibody that is substantially free of other antibodies having different antigenic specificities (e.g., an isolated antibody that binds specifically to VEGF-A is substantially free of antibodies that bind specifically to antigens other than VEGF-A). An isolated antibody that binds specifically to VEGF-A (e.g., bevacizumab, or an antigen binding portion thereof) can, however, have cross-reactivity to other antigens, such as VEGF-A molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

A “bispecific antibody” as used herein refers to an antibody comprising two antigen-binding sites, a first binding site having affinity for a first antigen or epitope and a second binding site having binding affinity for a second antigen or epitope distinct from the first.

An “anti-antigen antibody” refers to an antibody that binds specifically to the antigen. For example, an anti-VEGF-A antibody (e.g., bevacizumab, or an antigen binding portion thereof) binds specifically to VEGF-A.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-VEGF-A antibody (e.g., bevacizumab, or an antigen binding portion thereof) described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using available techniques in the art, and the fragments are screened for utility in the same manner as are intact antibodies. Antigen-binding portions can be produced by recombinant DNA techniques, or by enzymatic or chemical cleavage of intact immunoglobulins.

As used herein, the term “antibody,” when applied to a specific antigen, encompasses also antibody molecules comprising other binding moieties with different binding specificities. Accordingly, in one aspect, the term antibody also encompasses antibody drug conjugates (ADC). In another aspect, the term antibody encompasses multispecific antibodies, e.g., bispecific antibodies. Thus, for example, the term anti-VEGF-A antibody would also encompass ADCs comprising an anti-VEGF-A antibody or an antigen-binding portion thereof. Similarly, the term anti-VEGF-A antibody would encompass bispecific antibodies comprising an antigen-binding portion capable of specifically binding to VEGF-A.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. In some aspects, a cancer disclosed herein is selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC). In some aspects, the cancer is gastric cancer (e.g., locally advanced, metastatic gastric cancer, or previously untreated gastric cancer). In some aspects, the cancer is breast cancer (e.g., locally advanced or metastatic Her2-negative breast cancer). In some aspects, the cancer is prostate cancer (e.g., castration-resistant metastatic prostate cancer). In some aspects, the cancer is liver cancer (e.g., hepatocellular carcinoma such as advanced metastatic hepatocellular carcinoma). In some aspects, the cancer is carcinoma of head and neck (e.g., recurrent or metastatic squamous cell carcinoma of head and neck). In some aspects, the cancer is melanoma (e.g., metastatic melanoma). In some aspects, the cancer is colorectal cancer (e.g., advanced colorectal cancer metastatic to liver). In some aspects, the cancer is ovarian cancer (e.g., platinum-resistant ovarian cancer or platinum-sensitive recurrent ovarian cancer). In some aspects, the cancer is glioma (e.g., metastatic glioma). In some aspects, the cancer is glioblastoma. In some aspects, the cancer is lung cancer (e.g., NSCLC).

The term “tumor” refers to a solid cancer. The term “carcinoma” refers to a cancer of epithelial origin.

As used herein, the term “stroma” refers to a whole cell mixture comprising endothelial cells, smooth muscle cells, pericytes, immune cells (including lymphoid and myeloid cell types), supportive or connective tissue characteristic of that tissue located in or around a tissue or organ, particularly that connective and/or supportive tissue located in or around a tumor tissue or whole tumor as found in vivo. Stromal preparations may not be characterized by a single type or species of cells or proteins. For example, they can be instead characterized by a mixture of diverse molecular biomarker species characteristic of a whole stromal tissue preparation as observed in vivo in association with a whole organ or tumor. Tumor growth and spread are not only determined by the cancer cells, but also by the non-malignant constituents of the malignant lesion, which are subsumed under the term stroma. Thus, in some aspects, the term stroma refers to the non-malignant constituents of a tumor. In some aspects, the term stroma further includes malignant components of a tumor, i.e., cancer cells. In some aspects of the present disclosure, the terms stroma and tumor microenvironment (TME) are interchangeable.

The term “CMS” as used herein refers to a classification of colorectal cancer based on self-clustering of genes in a genomics DNA and RNA analysis of colorectal cancer. Guinney et al. (2015) Nature Medicine 21:1350-6. The classification resulted in four clusters of genes called CMS, or Consensus Molecular Subtypes. “CMS1” is called MSI Immune, and is characterized by MSI (microsatellite instability), CIMP (CpG Island Methylation Phenotype) high, hypermutation, BRAF mutations, immune infiltrations and activation, and worse survival after relapse. “CMS2” is called Canonical and is characterized by SCNA (somatic copy number alterations) high, and WNT and MYC activation. “CMS3” is called Metabolic, and is characterized by mixed MSI status, SCNA low, CIMP low, KRAS mutations, and metabolic deregulation. “CMS4” is called Mesenchymal, and is characterized by SCNA high, stromal infiltration, TGFβ activation, angiogenesis, and worse relapse-free and overall survival.

As used herein, the term “MSI-H” stands for microsatellite instability-high (MSH-High). In general, this describes cancer cells that have a greater than normal number of genetic markers called microsatellites. Microsatellites are short, repeated, sequences of DNA. Cancer cells that have large numbers of microsatellites may have defects in the ability to correct mistakes that occur when DNA is copied in the cell. Microsatellite instability is found most often in colorectal cancer, other types of gastrointestinal cancer, and endometrial cancer. It may also be found, e.g., in cancers of the breast, prostate, bladder, and thyroid.

The term “dMMR” refers to deficient mismatch repair. MSI-H/dMMR can occur when a cell is unable to repair mistakes made during the division process.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

In the context of the present disclosure, the terms “immunosuppressed” or “immunosuppression” describe the status of the immune response to the cancer. The patient's immune response to the cancer can be dampened by immune suppressive cells in the tumor microenvironment, thus blocking, preventing, or diminishing an immune system attack on the cancer. In immunosuppression therapy the goal is to relieve immunosuppression (as opposed to causing immunosuppression, e.g., as in the context of an organ transplant) by giving patients certain drugs, so that the immune system can attack the cancer.

The term “small molecule” refers to an organic compound having a molecular weight of less than about 900 Daltons, or less than about 500 Daltons. The term includes agents having the desired pharmacological properties, and includes compounds that can be taken orally or by injection. The term includes organic compounds that modulate the activity of TGF-β, and/or other molecules associated with enhancing or inhibiting an immune response.

“VEGF-A”, also known as vascular endothelial growth factor A, vascular permeability factor, VEGF, VPF or MVCD1, refers to a gene or the expressed polypeptide thereof that is a member of the PDGF/VEGF growth factor family. VEGF-A encodes a heparin-binding protein. It is a growth factor that induces proliferation and migration of vascular endothelial cells and is essential for both physiological and pathological angiogenesis. Disruption of this gene in mice resulted in abnormal embryonic blood vessel formation. This gene is up-regulated in many known tumors and its expression is correlated with tumor stage and progression. Variants of this gene has been reported, including, but not limited to, allelic variants associated with microvascular complications of diabetes 1 (MVCD1) and atherosclerosis, alternatively spliced transcript variants encoding different isoforms, alternative translation initiation from upstream non-AUG (CUG) codons (resulting in additional isoforms), and C-terminally extended isoforms produced by use of an alternative in-frame translation termination codon via a stop codon read-through mechanism (with this isoform being antiangiogenic). In some aspects, the term VEGF-A encompasses the sequence of Unipro Acc. No. P15692, NCBI Gene ID 7422, as well as its homologues and isoforms.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7. The human PD-L1 protein is encoded by the human CD274 gene (NCBI Gene ID: 29126).

As used herein, the term “subject” includes any human or nonhuman animal. The terms, “subject” and “patient” are used interchangeably herein. The term “nonhuman animal” includes, but is not limited to, vertebrates such as dogs, cats, horses, cows, pigs, boar, sheep, goat, buffalo, bison, llama, deer, elk and other large animals, as well as their young, including calves and lambs, and to mice, rats, rabbits, guinea pigs, primates such as monkeys and other experimental animals. Within animals, mammals are preferred, most preferably, valued and valuable animals such as domestic pets, racehorses and animals used to directly produce (e.g., meat) or indirectly produce (e.g., milk) food for human consumption, although experimental animals are also included. In specific aspects, the subject is a human. Thus, the present disclosure is applicable to clinical, veterinary and research uses.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition, or biochemical indicia associated with a disease or enhancing overall survival. Treatment can be of a subject having a disease or a subject who does not have a disease (e.g., for prophylaxis). As used here, the terms “treat,” “treating,” and “treatment” refer to the administration of an effective dose or effective dosage.

The term “effective dose” or “effective dosage” is defined as an amount sufficient to achieve or at least partially achieve a desired effect.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction.

A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease.

In addition, the terms “effective” and “effectiveness” with regard to a treatment disclosed herein includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

The ability of a therapeutic agent to promote disease regression, e.g., cancer regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” or combination thereof promotes cancer regression in a subject. In some aspects, a therapeutically effective amount of the therapeutic agent promotes cancer regression to the point of eliminating the cancer.

In some aspects of the present disclosure, the anticancer agents are administered as a combination of therapies, e.g., a therapy comprising the administration of (i) an anti-angiogenic therapy, e.g., an anti-VEGF-A antibody such as bevacizumab, and (ii) a checkpoint inhibitor therapy, e.g., an antibody against PD1 or PD-L1.

“Promoting cancer regression” means that administering an effective amount of the drug or combination thereof (administered together as a single therapeutic composition or as separate compositions in separate treatments as discussed above), results in a reduction in cancer burden, e.g., reduction in tumor growth or size, necrosis of the tumor, a decrease in severity of at least one disease symptom, an increase in frequency and duration of disease symptom-free periods, or a prevention of impairment or disability due to the disease affliction.

Notwithstanding these ultimate measurements of therapeutic effectiveness, evaluation of immunotherapeutic drugs must also make allowance for immune-related response patterns. The ability of a therapeutic agent to inhibit cancer growth, e.g., tumor growth, can be evaluated using assays described herein and other assays known in the art. Alternatively, this property of a composition can be evaluated by examining the ability of the compound to inhibit cell growth, such inhibition can be measured in vitro by assays known to the skilled practitioner.

“Tumor,” as used herein, refers to all neoplastic cell growth and proliferation and all pre-cancerous and cancerous cells and tissues. In some aspects, the cancer, e.g., colorectal cancer, gastric cancer, breast cancer, prostate cancer, liver cancer, carcinoma of head and neck, melanoma, or ovarian cancer is relapsed. The term “relapsed” refers to a situation where a subject, that has had a remission of cancer (e.g., colorectal cancer) after a therapy, has a return of cancer cells. In some aspects, the cancer, e.g., colorectal cancer, gastric cancer, breast cancer, prostate cancer, liver cancer, carcinoma of head and neck, melanoma, ovarian cancer, glioma, glioblastoma, or lung cancer is refractory. As used herein, the term “refractory” or “resistant” refers to a circumstance where a subject, even after intensive treatment, has residual cancer cells in the body. In some aspects, the cancer, e.g., colorectal cancer, gastric cancer, breast cancer, prostate cancer, liver cancer, carcinoma of head and neck, melanoma, ovarian cancer, glioma, glioblastoma, or lung cancer is refractory following at least one prior therapy comprising administration of at least one anticancer agent. In some aspects, the cancer, e.g., colorectal cancer, gastric cancer, breast cancer, prostate cancer, liver cancer, carcinoma of head and neck, melanoma, ovarian cancer, glioma, glioblastoma, or lung cancer is metastatic.

A “cancer” or “cancer tissue” can include a tumor at various stages. In certain aspects, the cancer or tumor is Stage 0, such that, e.g., the cancer or tumor is very early in development and has not metastasized. In some aspects, the cancer or tumor is Stage I, such that, e.g., the cancer or tumor is relatively small in size, has not spread into nearby tissue, and has not metastasized. In other aspects, the cancer or tumor is Stage II or Stage III, such that, e.g., the cancer or tumor is larger than in Stage 0 or Stage I, and it has grown into neighboring tissues but it has not metastasized, except potentially to the lymph nodes. In other aspects, the cancer or tumor is Stage IV, such that, e.g., the cancer or tumor has metastasized. Stage IV can also be referred to as advanced or metastatic cancer.

The terms “biological sample” or “sample” as used herein refers to biological material isolated from a subject. The biological sample can contain any biological material suitable for determining gene expression, for example, by sequencing nucleic acids.

The biological sample can be any suitable biological tissue, for example, cancer tissue. In one aspect, the sample is a tumor tissue biopsy, e.g., a formalin-fixed, paraffin-embedded (FFPE) tumor tissue or a fresh-frozen tumor tissue or the like. In another aspect, an intratumoral sample is used. In another aspect, biological fluids can be present in a tumor tissue biopsy, but the biological sample will not be a biological fluid per se.

In some aspects, the sample, e.g., a biopsy (e.g., a tumor biopsy, peritumoral biopsy, or a combination thereof), tissue section, or tissue sample can be obtained from a primary tumor. In some aspects, the sample, e.g., a biopsy (e.g., a tumor biopsy, peritumoral biopsy, or a combination thereof), tissue section, or tissue sample can be obtained from a metastasis or metastases tumor. In some aspects, the sample, e.g., a biopsy (e.g., a tumor biopsy, peritumoral biopsy, or a combination thereof), tissue section, or tissue sample can be obtained from any alternative site beyond the original diagnostic location.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about,” “comprising essentially of,” or “consisting essentially of,” refer to a value or composition that is within an acceptable error range for the particular value or composition as determined by one of ordinary skill in the art, which will depend in part on how the value or composition is measured or determined, i.e., the limitations of the measurement system. For example, “about,” “comprising essentially of,” or “consisting essentially of,” can mean within 1 or more than 1 standard deviation per the practice in the art. Alternatively, “about,” “comprising essentially of,” or “consisting essentially of,” can mean a range of up to 10%. Furthermore, particularly with respect to biological systems or processes, the terms can mean up to an order of magnitude or up to 5-fold of a value. When particular values or compositions are provided in the specification and claims, unless otherwise stated, the meaning of “about,” “comprising essentially of,” or “consisting essentially of,” should be assumed to be within an acceptable error range for that particular value or composition.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

It is understood that wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

### I. TME Phenotype Class Specific Cancer Treatments

The present disclosure provides methods for the classification of cancer patients or cancer tumors into specific tumor microenvironment (TME) phenotype classes, which can be used to guide therapy choices, to determine the eligibility of a cancer patient for a specific treatment, or to predict the therapeutic response to a specific treatment, wherein the cancer is selected, e.g., from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC).

The TME, also known as stroma, encompasses the non-malignant constituents of a tumor including endothelial cells, smooth muscle cells, pericytes, fibroblasts, immune cells (including lymphoid and myeloid cell types), and supportive and/or connective tissue characteristic of that tissue in which the tumor is located and/or connective and/or supportive tissue located in or around a tumor tissue or whole tumor as found in vivo.

The TME Panel-1 Classifier of the present disclosure can classify patients having, for example, gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) into TME phenotypes classes that reflect appreciated molecular biological characteristics of the disease, as discussed below. The TME Panel-1 Classifier of the present disclosure successfully classifies colorectal cancer patients into TME phenotypes classes that reflect appreciated molecular biological characteristics of the disease, namely enrichment for angiogenic and immune processes across disease stages and tumor size. TME Panel-1 identifies similar prevalence of TME phenotype classes in colorectal cancer as it does in gastric cancer, with similar implications for survival. Thus, TME Panel-1 is prognostic for disease free and overall survival in cancer, and for predicting outcome of targeted therapy in cancer. When applied to colorectal cancer, TME Panel-1 is consistent with the Consensus Molecular Subtypes (CMS) model's general annotations of CMS1 as immune enriched and CMS4 as angiogenic. However, the CMS group designations do not completely capture either of these biologies, nor their interactions. TME Panel-1 was specifically developed to capture these dominant biological processes and yields more granular predictions as to the appropriate pairing of targeted therapy to patient.

The classifiers of the present disclosure, e.g., the TME Panel-1 Classifier, are based on the application of a predictive model generated by machine-learning using an artificial neural network (ANN). In some aspects, the classifier, e.g., the TME Panel-1 Classifier, is generated using a training set comprising expression data (e.g., RNA expression data) preprocessed according to a population-based classifier as training set. See U.S. application Ser. No. 17/089,234, which is herein incorporated by reference in its entirety.

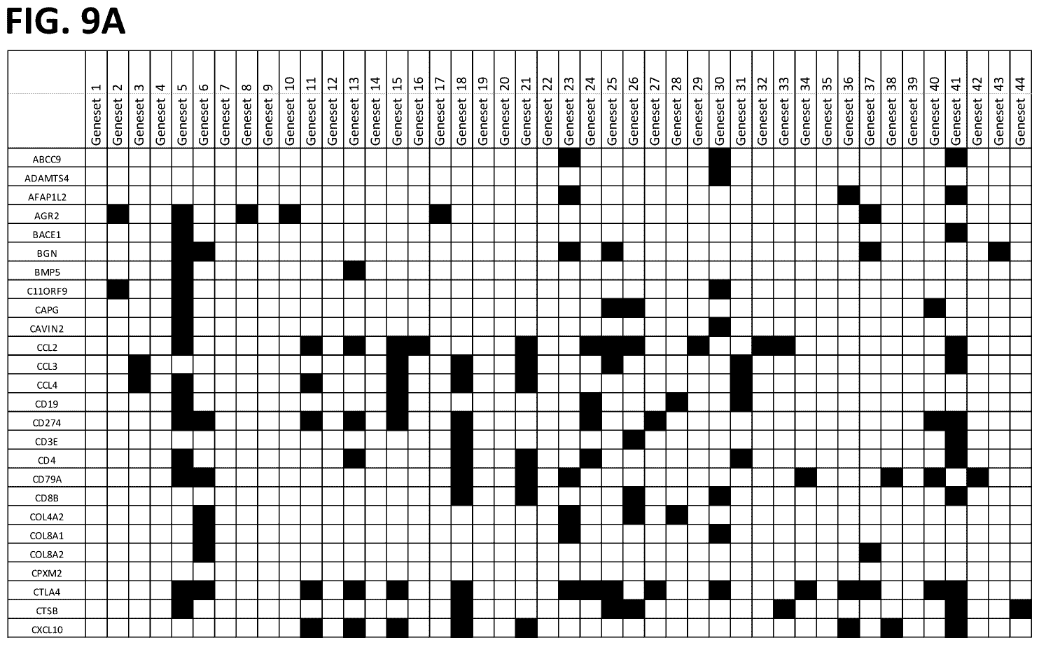

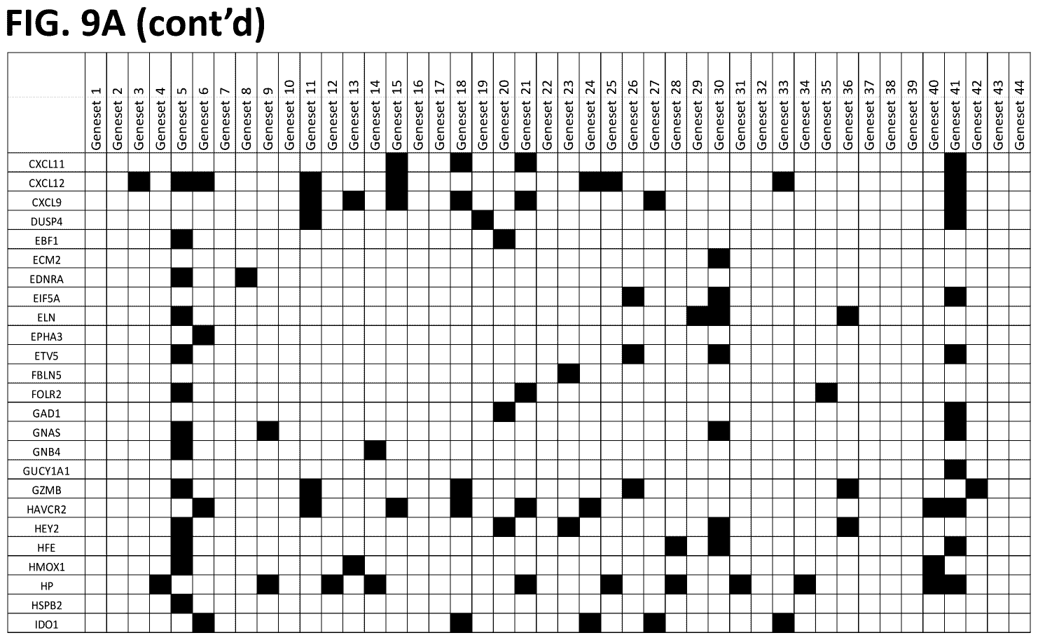

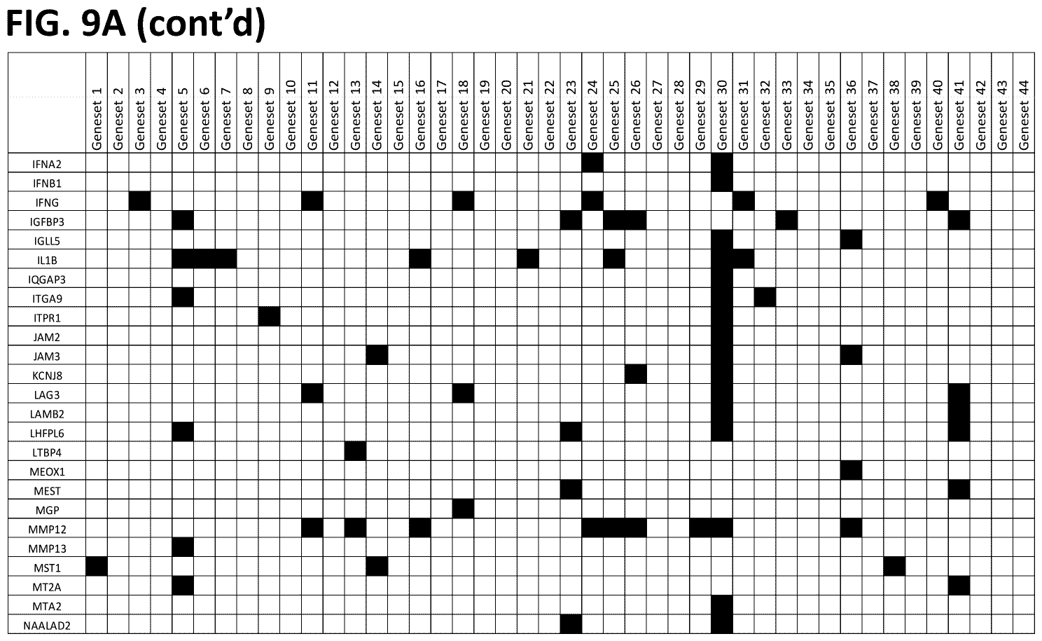

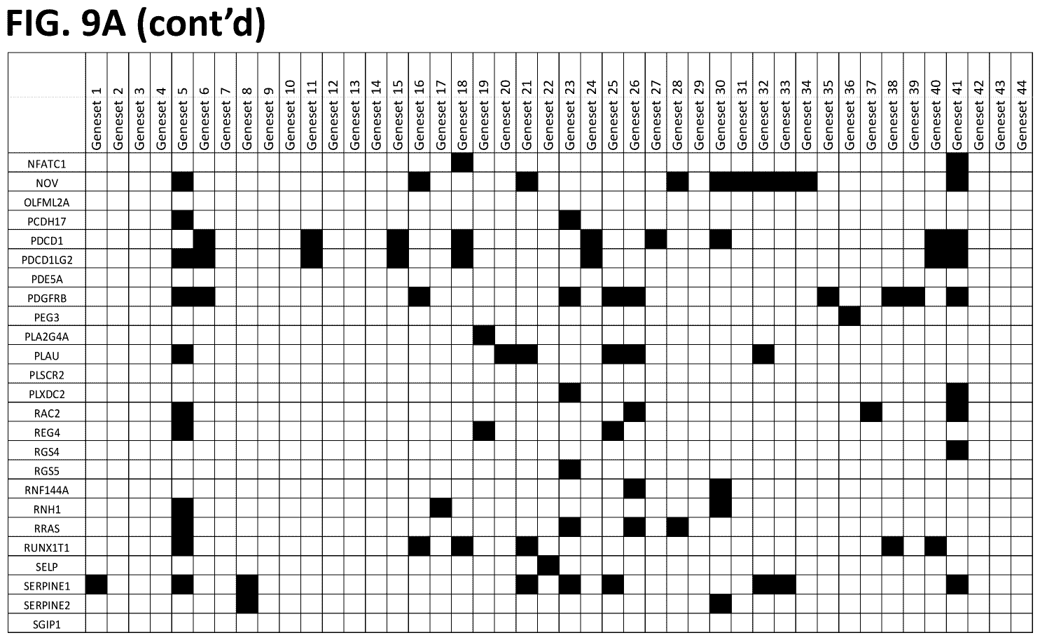

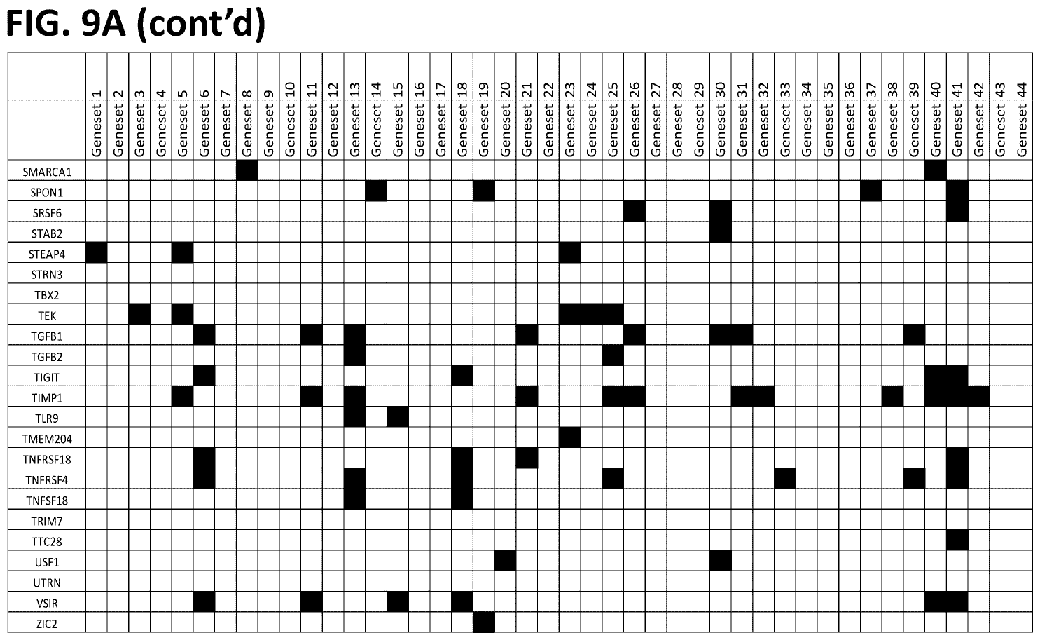

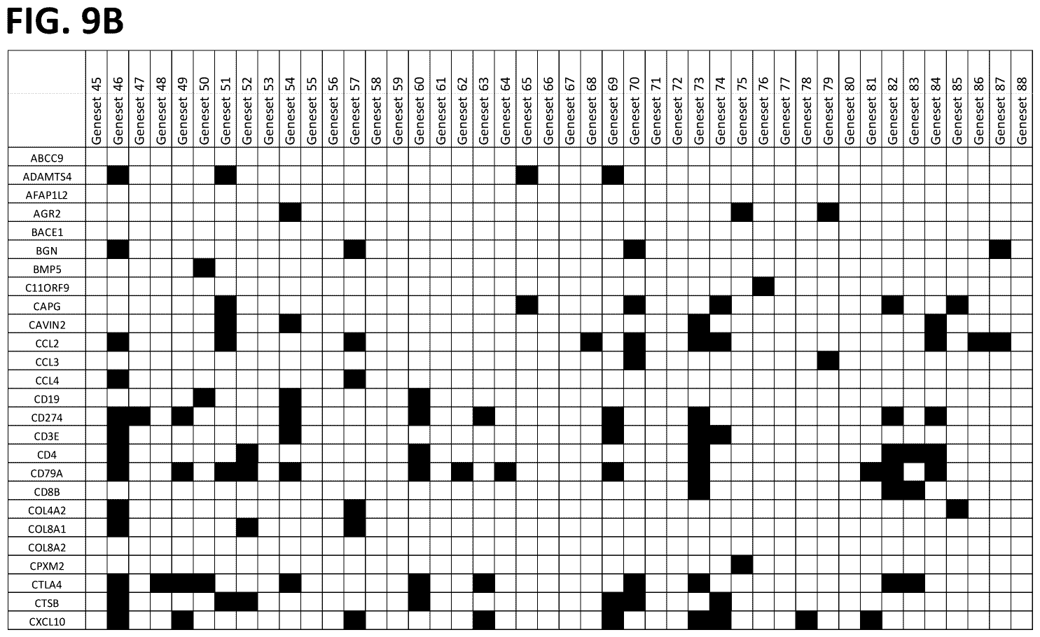

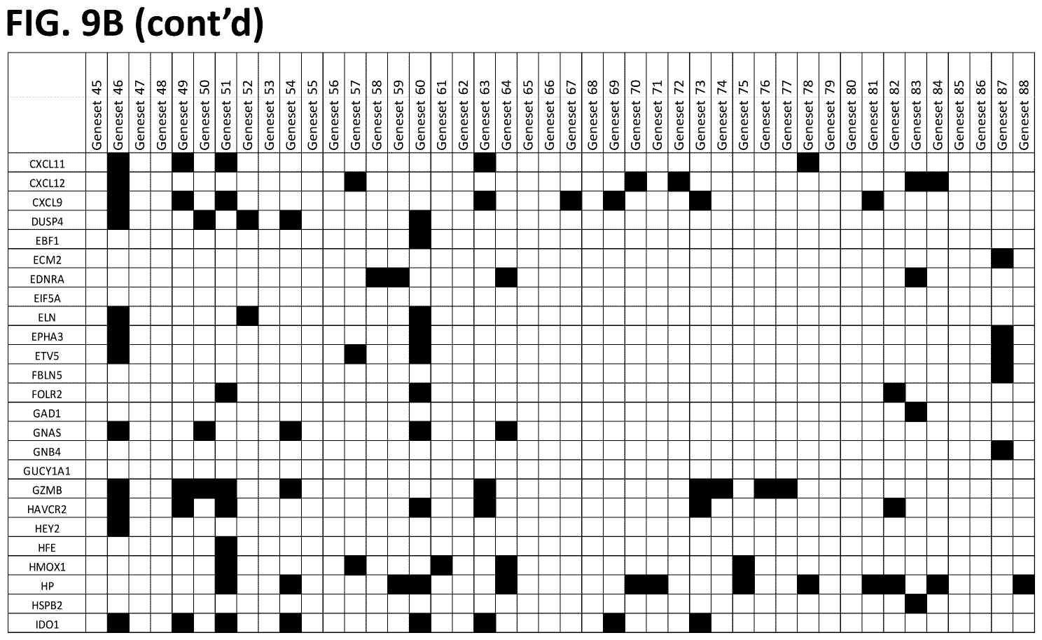

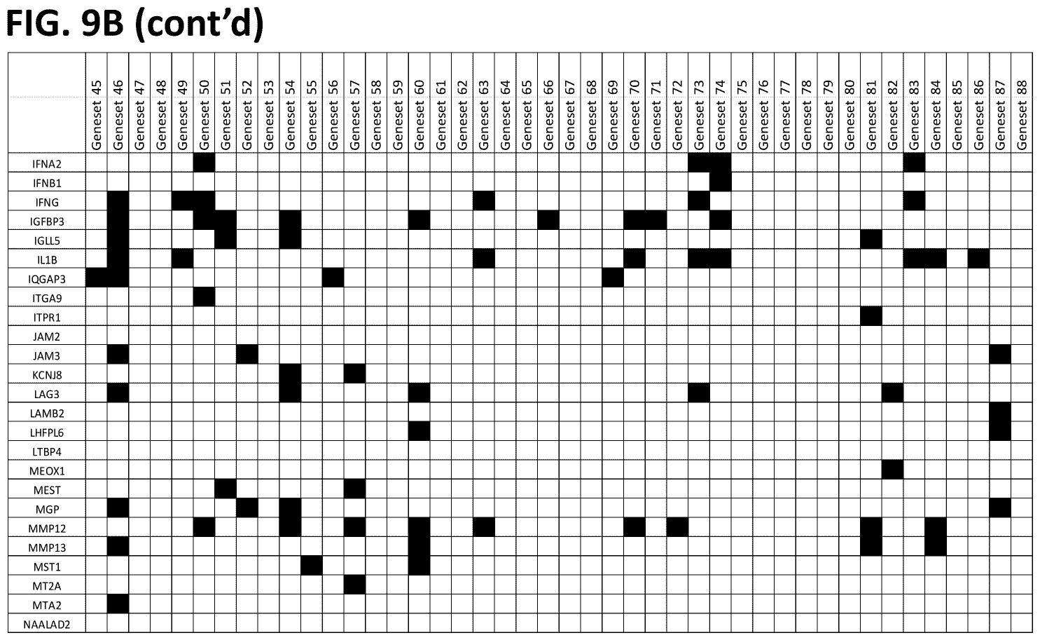

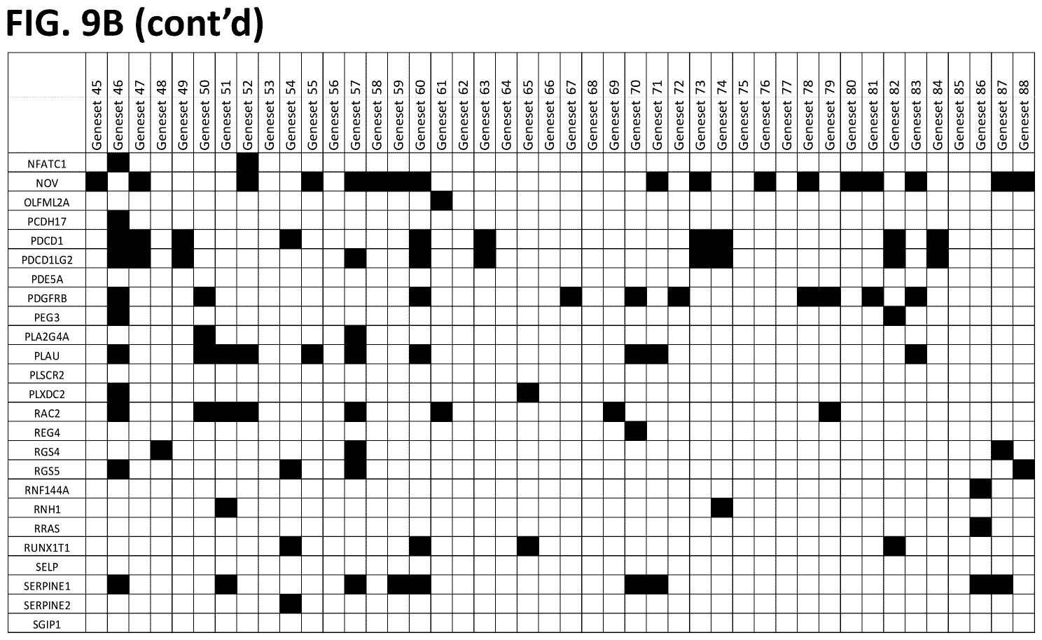

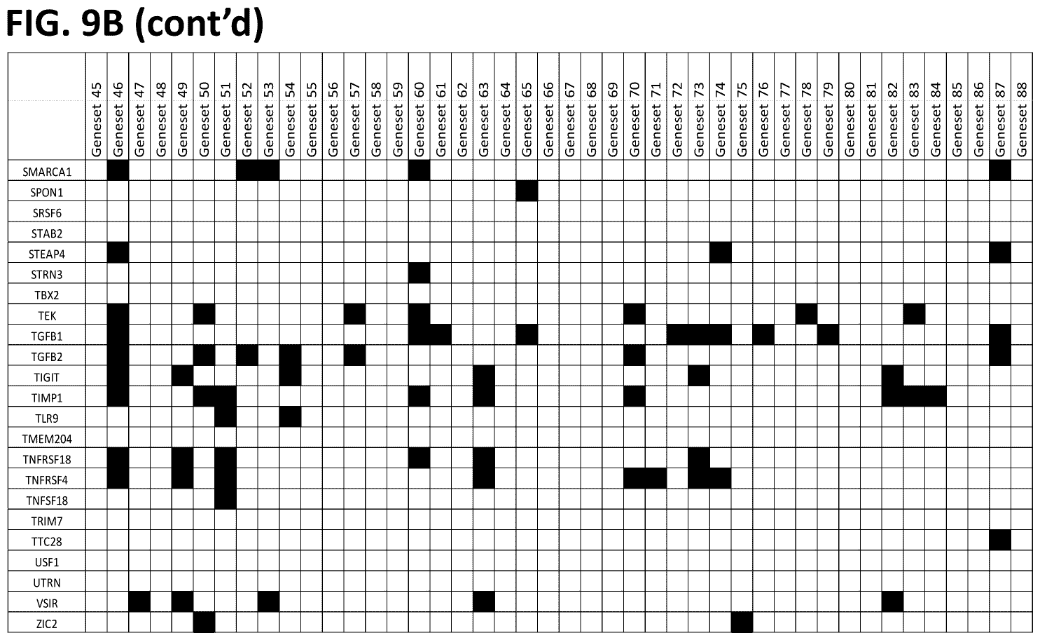

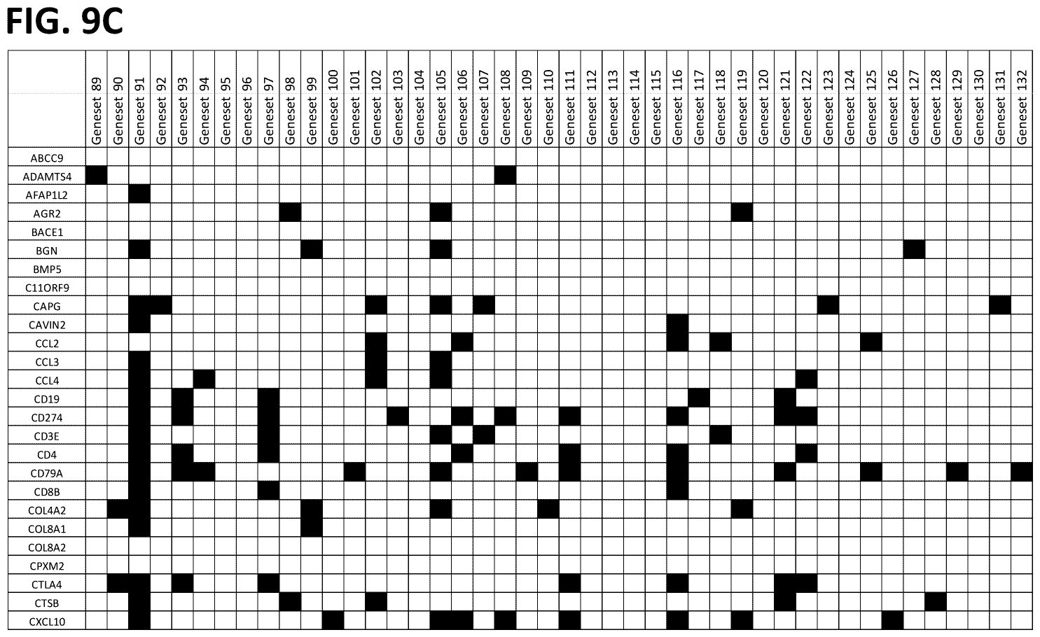

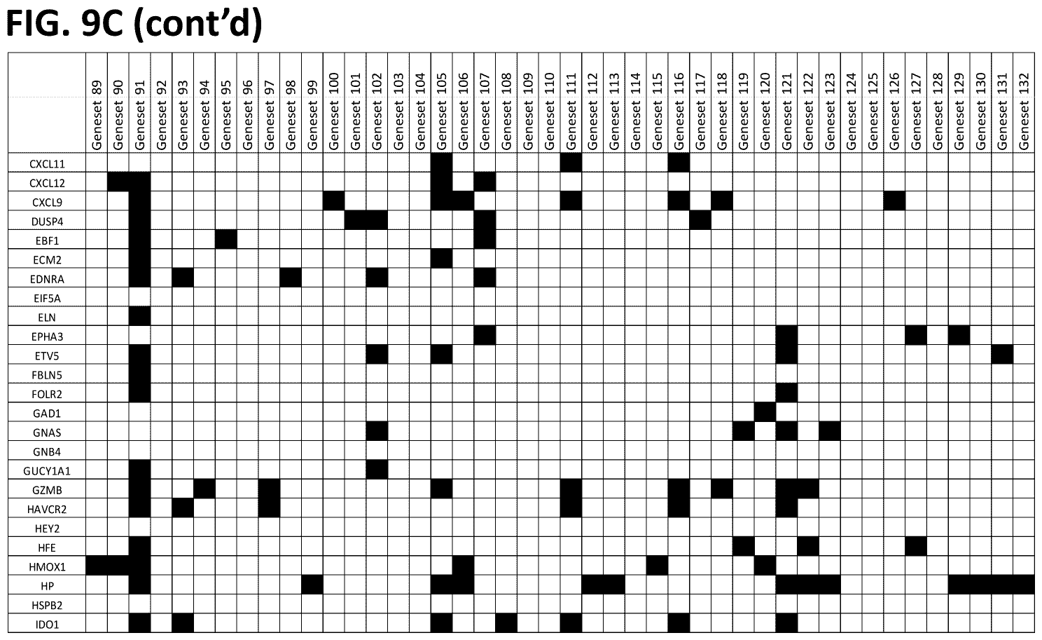

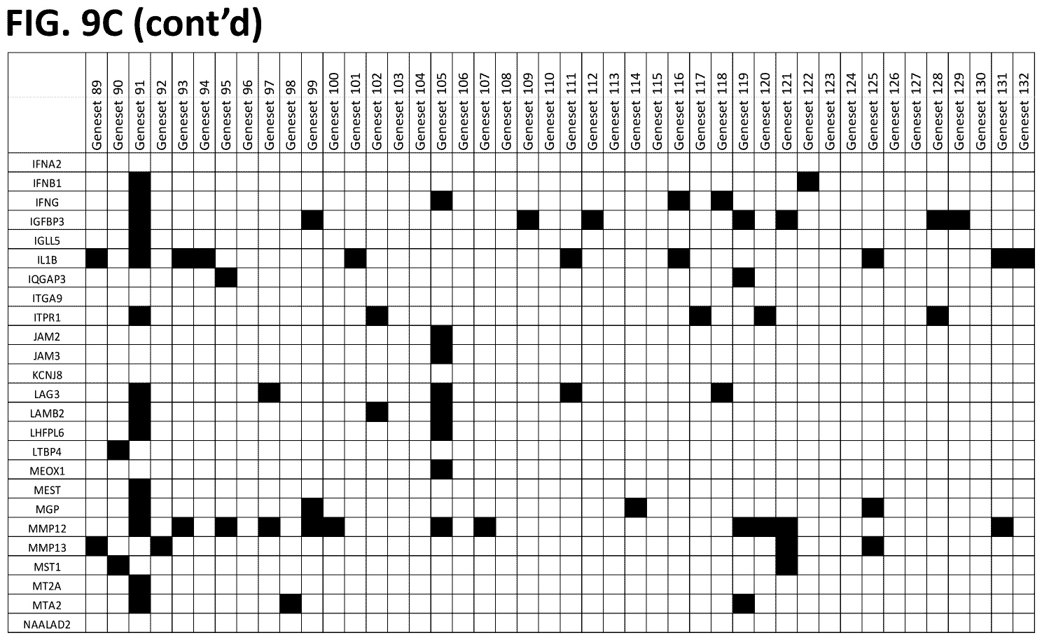

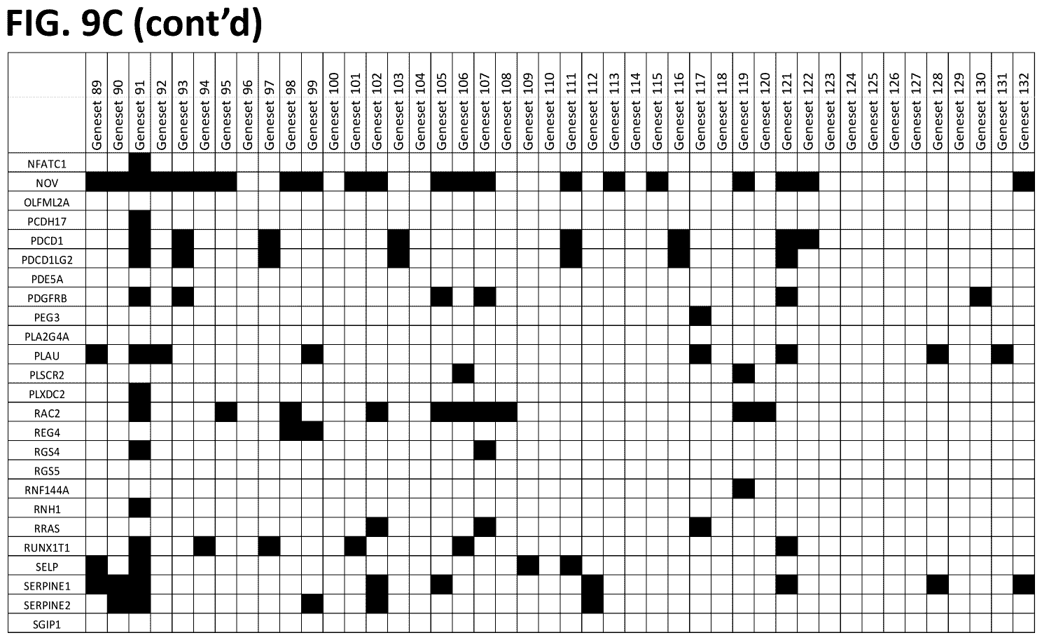

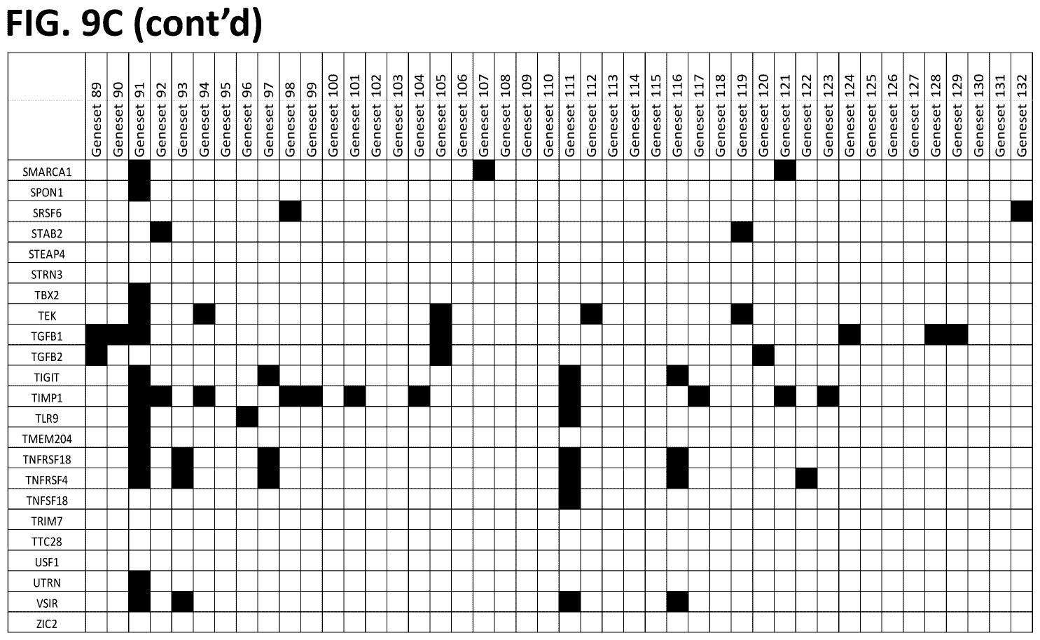

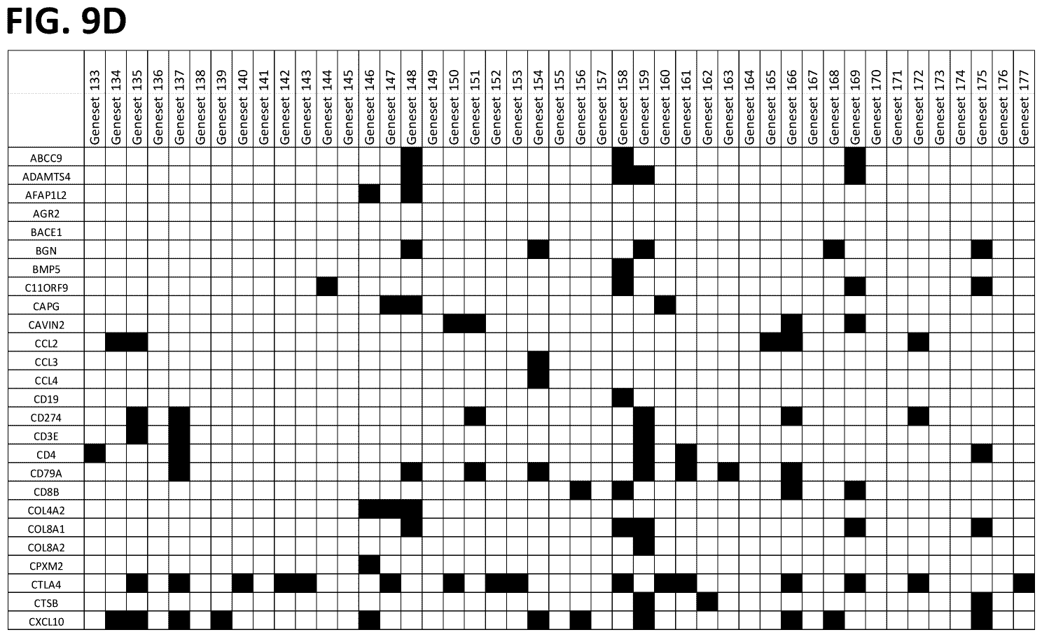

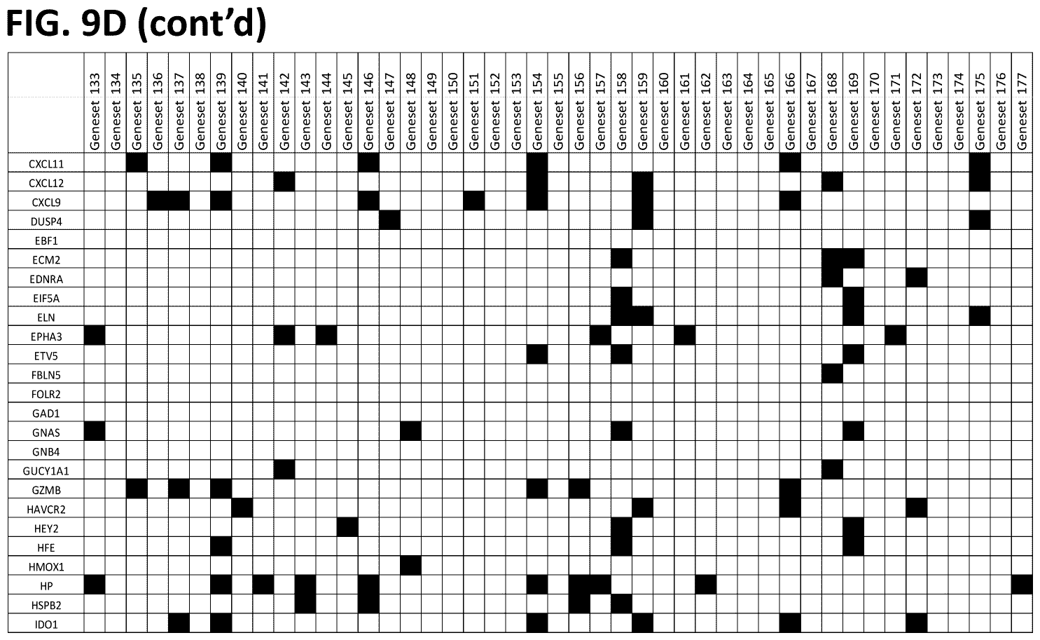

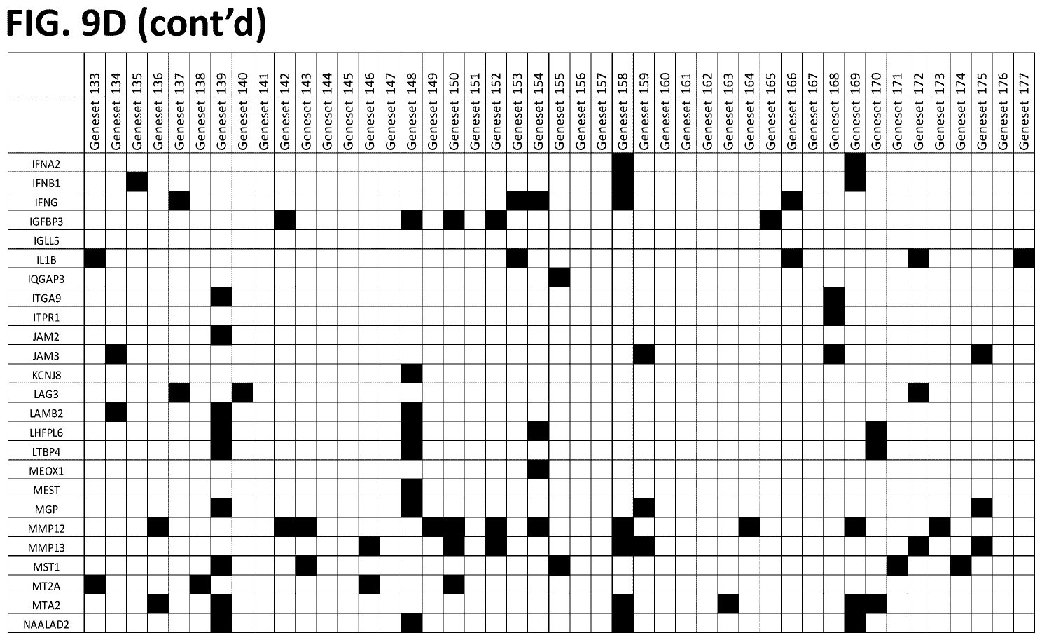

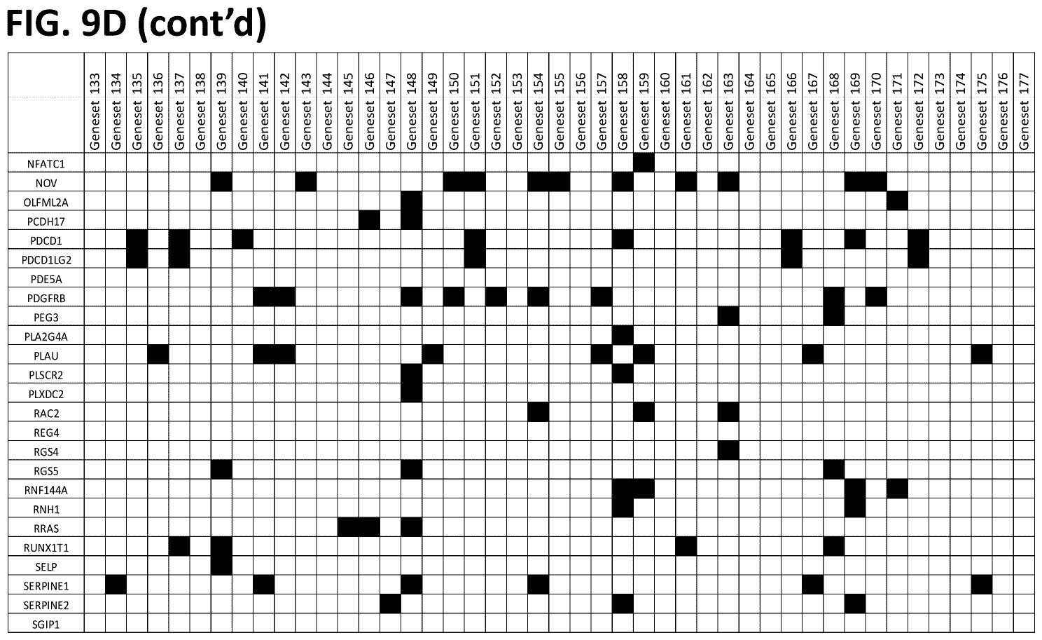

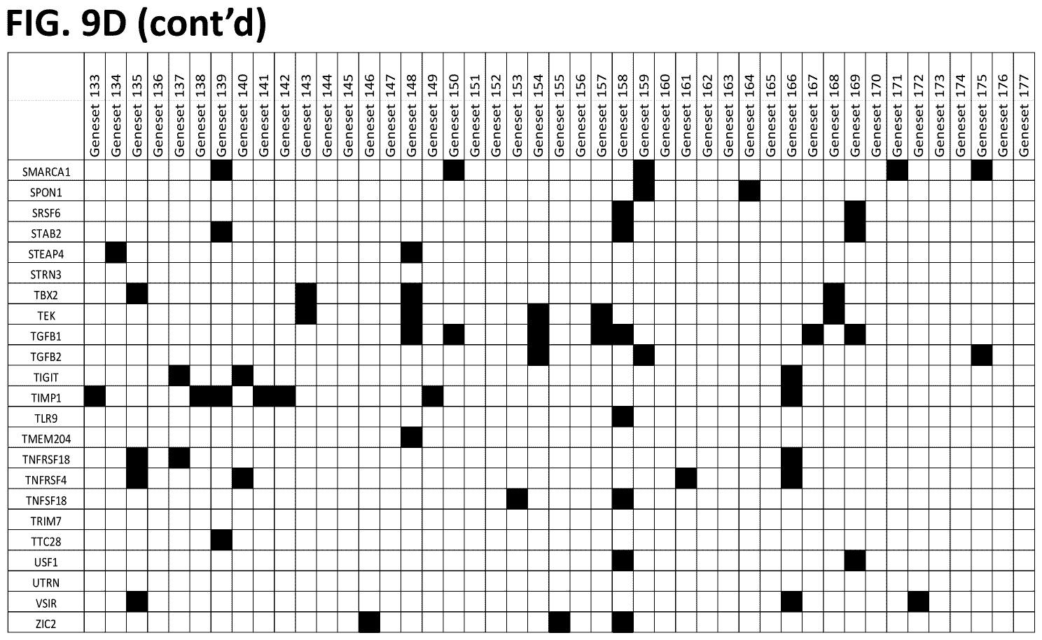

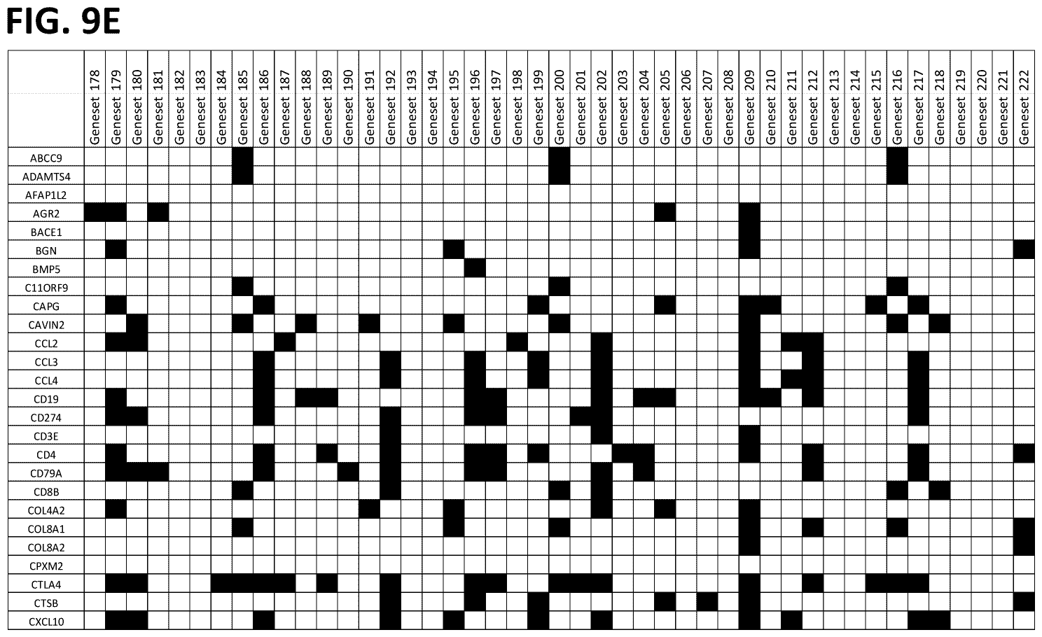

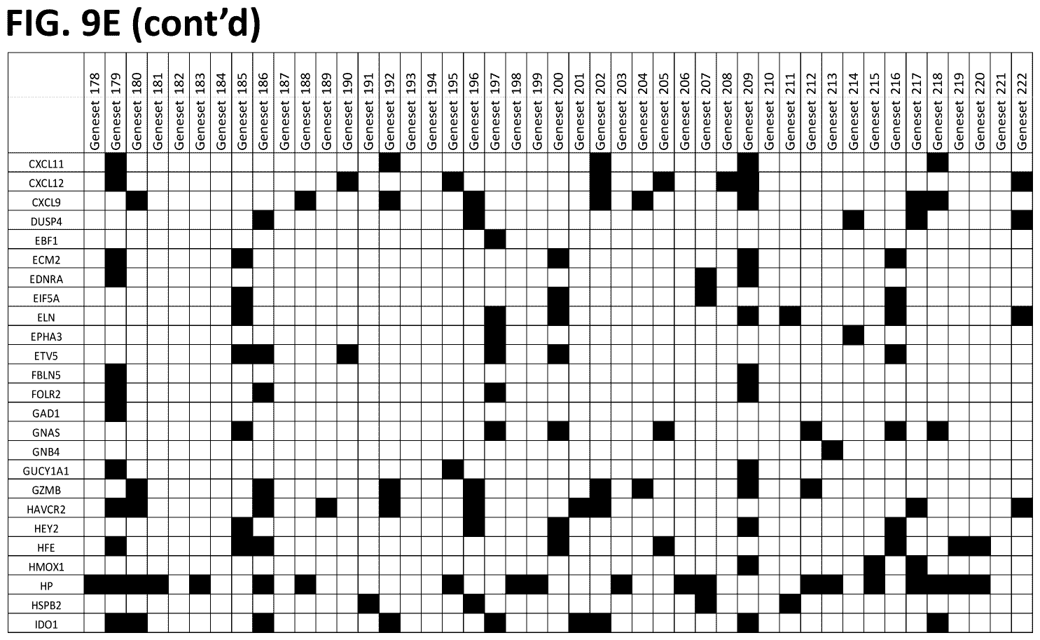

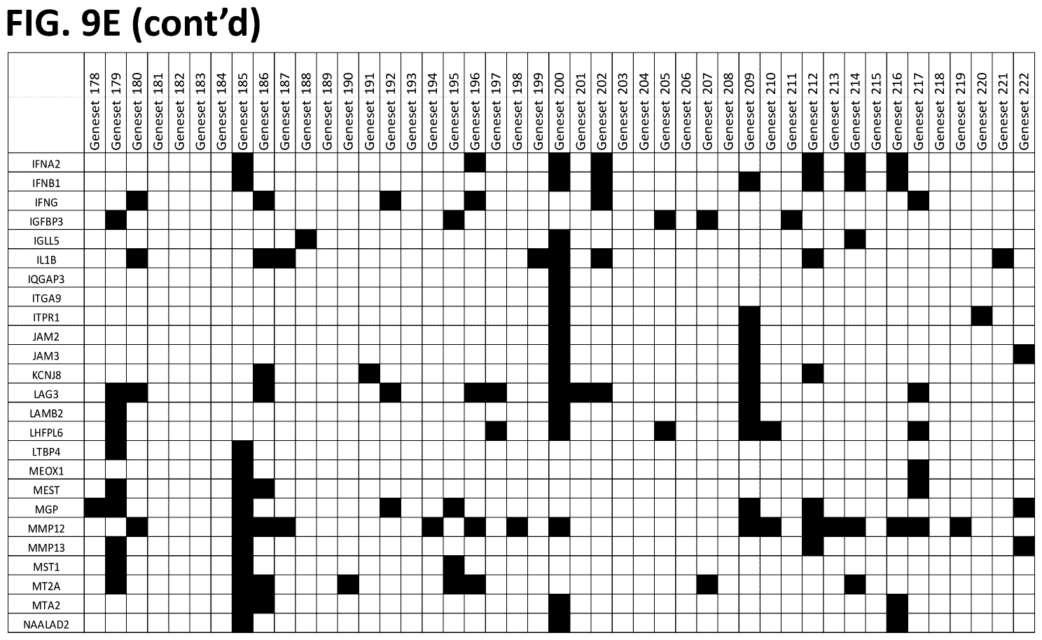

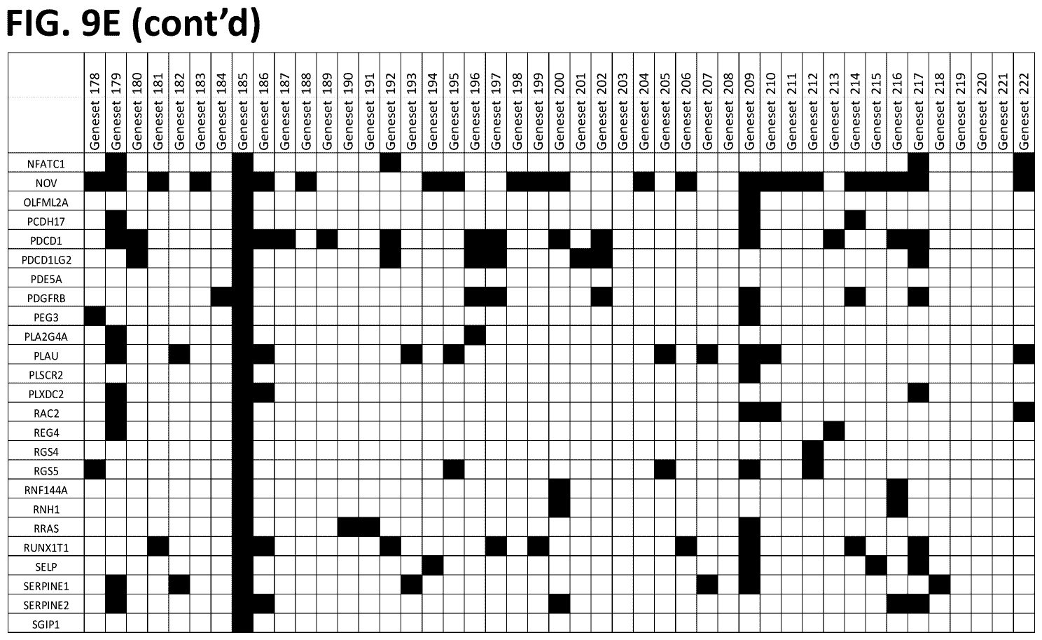

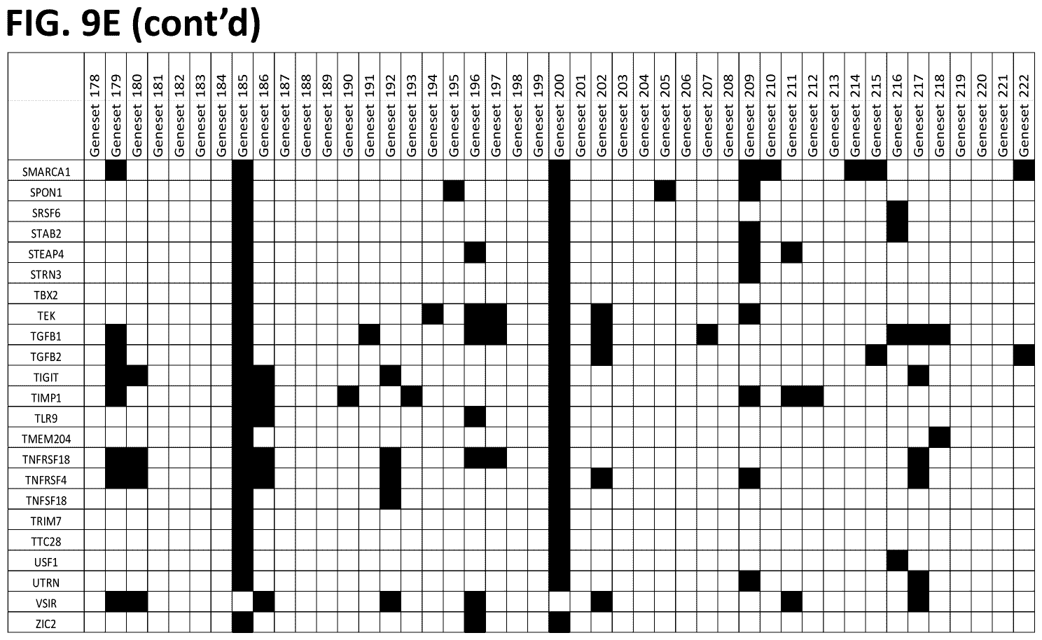

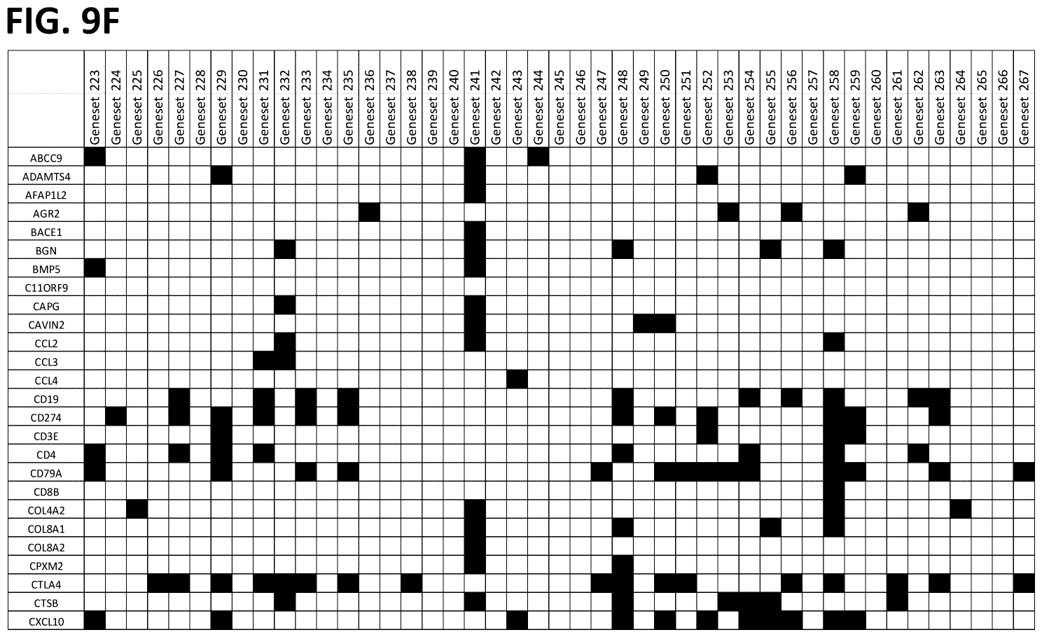

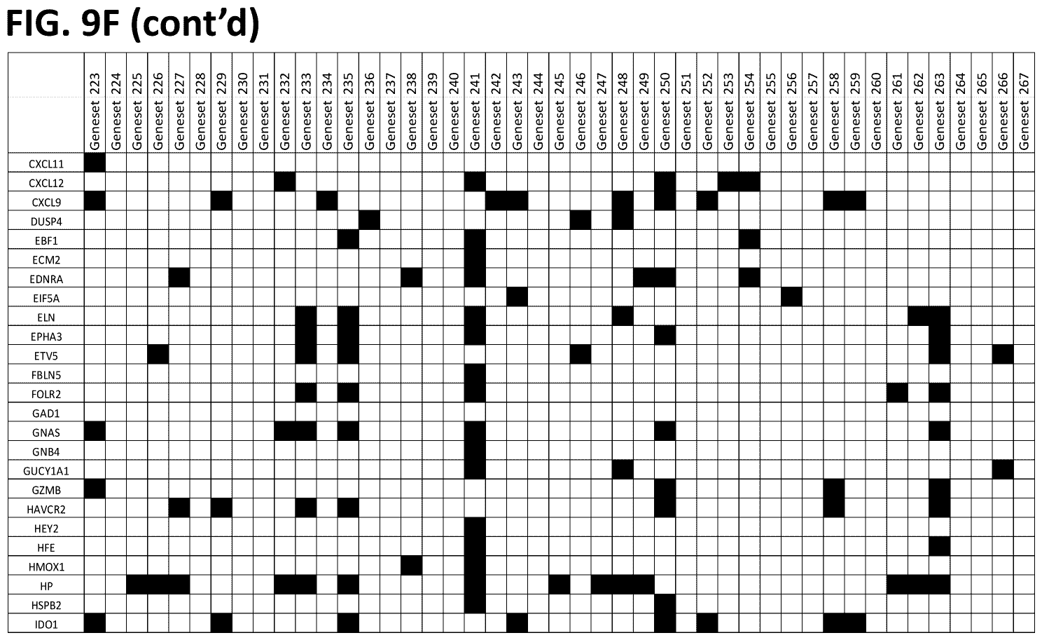

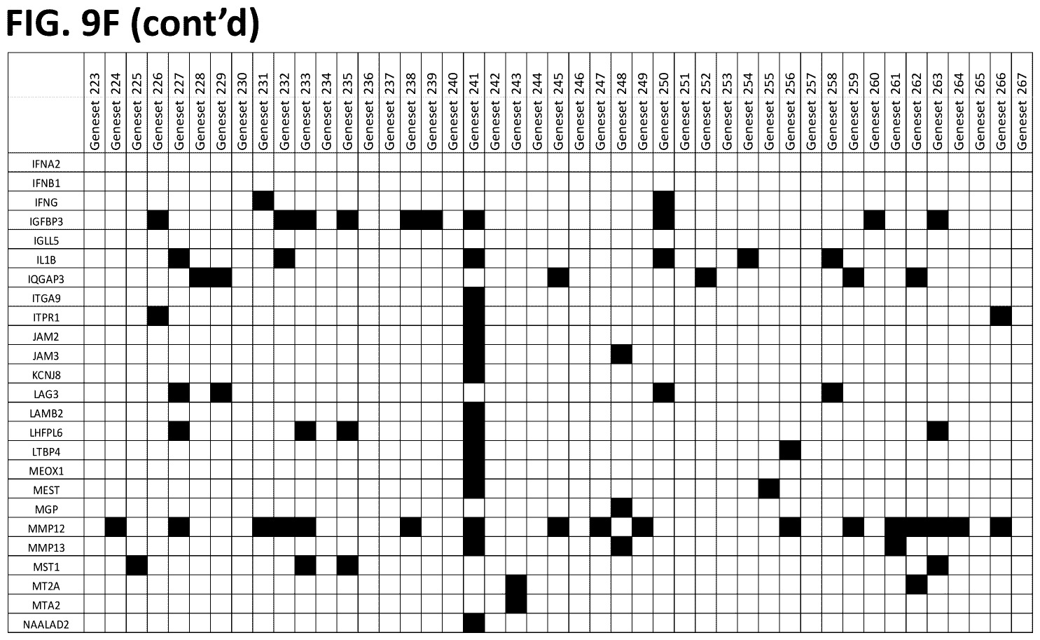

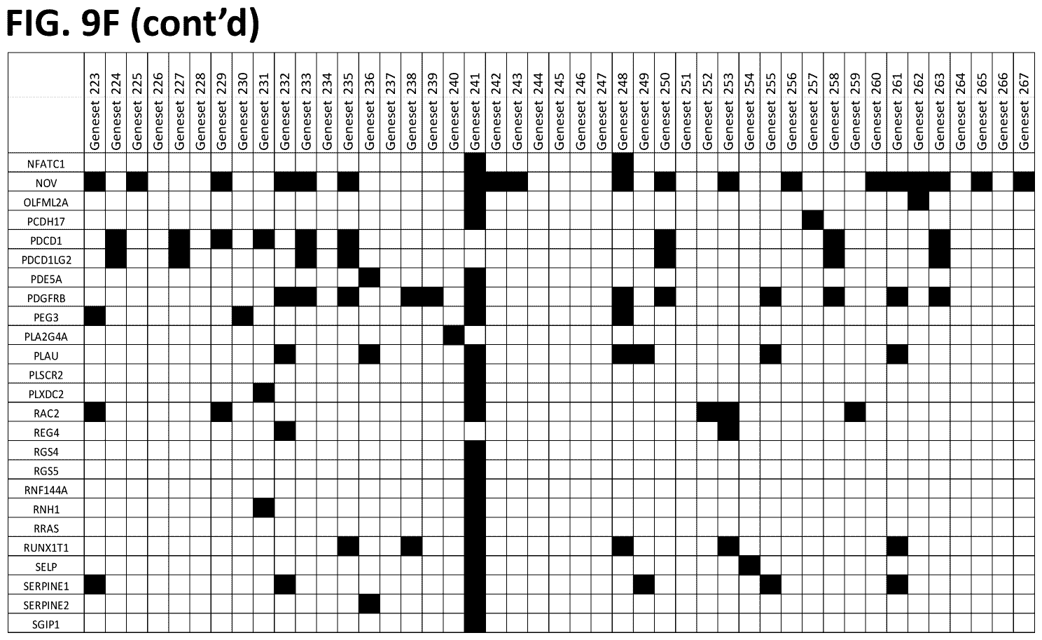

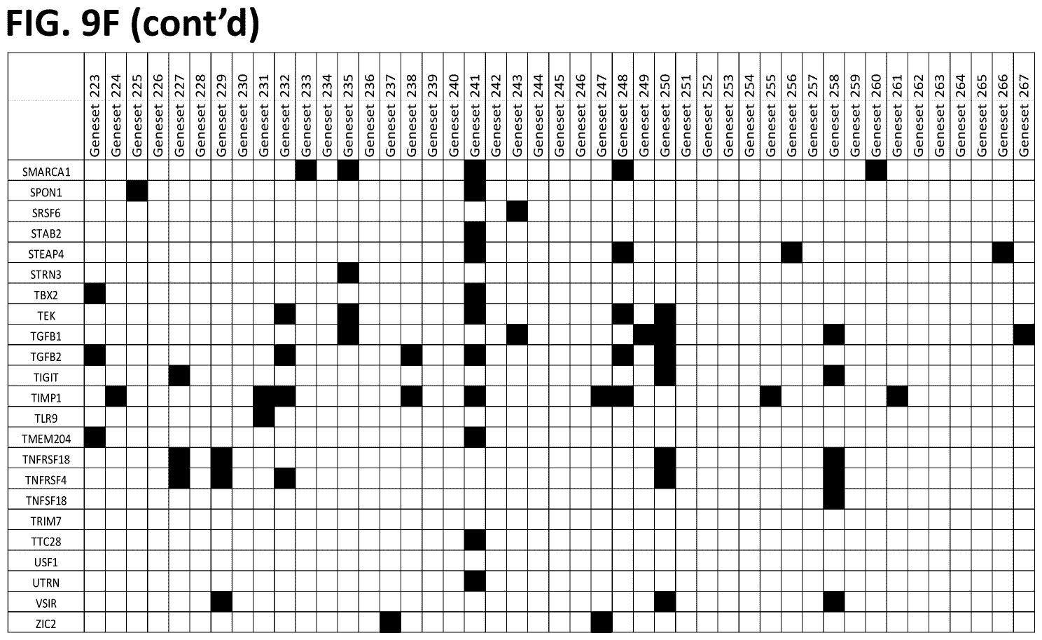

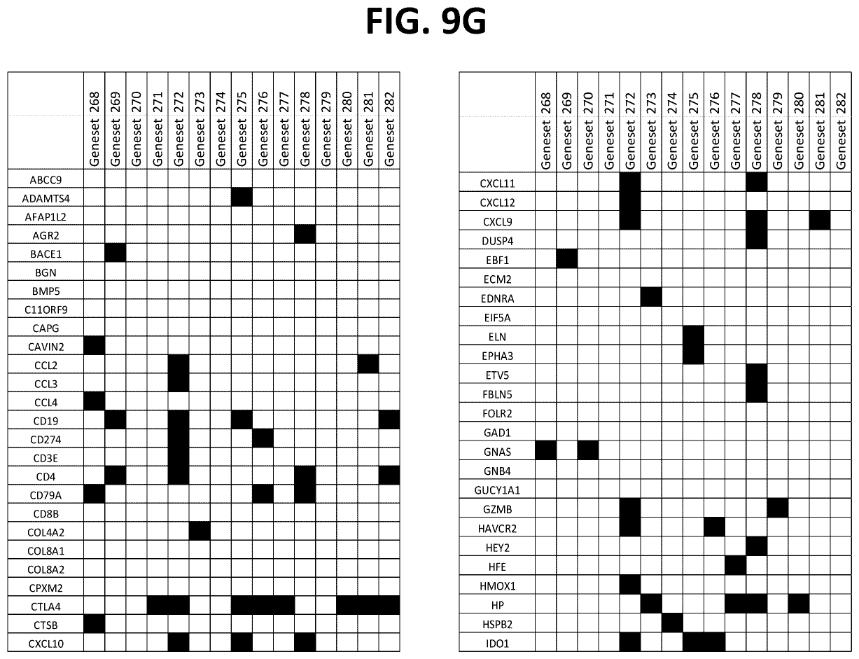

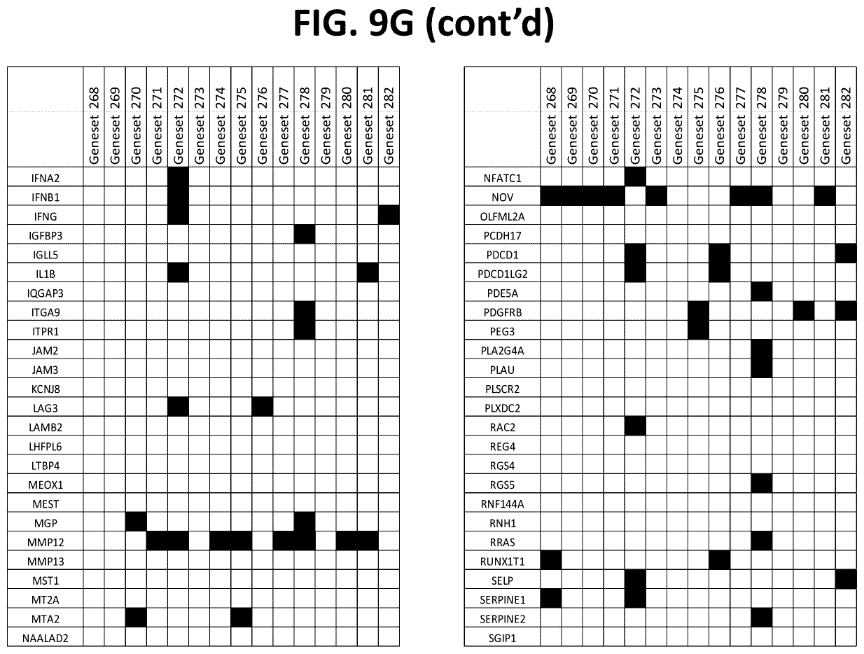

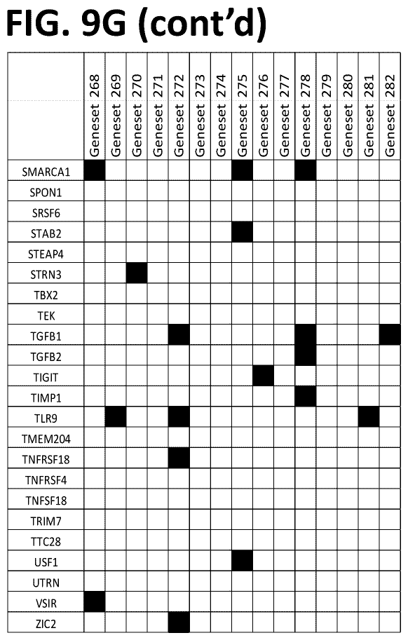

The application of a ANN classifier of the present disclosure, e.g., the TME Panel-1 Classifier, comprises measuring the expression levels (e.g., mRNA expression levels) of a gene panel (e.g., a gene panel comprising at least one gene from TABLE 1 and one gene from TABLE 2, a gene panel comprising a set of genes from TABLE 3 and a set of genes from TABLE 4, gene panel from TABLE 5, or any of the gene panels (Genesets) disclosed in FIG. 9A-G) in a sample obtained from a cancer patient; and applying the classifier to the measured expression levels. The classifier e.g., the TME Panel-1 Classifier, assigns the patient's cancer, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) to a particular TME phenotype class or a combination thereof.

Afterwards, the output of an ANN classifier of the present disclosure, e.g., the TME Panel-1 Classifier, assigning the subject's cancer to a particular TME phenotype class or to a combination thereof would guide the selection and administration of a specific treatment or treatments which have been determined to be effective to treat a cancer assigned to the same TME phenotype class in other subjects, i.e., a TME phenotype class-specific therapy disclosed below or a combination thereof.

As used herein, the terms “tumor microenvironment” and “TME” refer to the environment surrounding tumor cells, including, e.g., blood vessels, immune cells, endothelial cells, fibroblasts, other stromal cells, signaling molecules, and the extracellular matrix. In some aspects, the terms “stromal subtype,” “stromal phenotype,” and grammatical variants thereof are used interchangeably with the term “TME phenotype.” As used herein, the term “TME phenotype class” refers to the output of classifier of the present disclosure, e.g., the TME Panel-1 Classifier, assigning the subject's cancer to a particular TME phenotype.

The tumor cells and the surrounding microenvironment are closely related and interact constantly. In general, tumor microenvironment (also known as, e.g., stromal phenotype) encompasses any structural and/or functional characteristic of the stroma of a tumor and tumoral environment. Numerous non-tumoral cell types can exist in a TME, e.g., carcinoma associated fibroblasts, myeloid-derived suppressor cells, tumor-associated macrophages, neutrophils, or tumor infiltrating lymphocytes. In some aspects, the classification of a particular TME can include the analysis of the cell types present in the stroma. A TME can also be characterized by specific functional characteristics, e.g., by abnormal oxygenation levels, abnormal blood vessel permeability, or abnormal levels of particular proteins such as collagens, elastin, glycosaminoglycans, proteoglycans, or glycoproteins.

The output of the ANN classifiers of the present disclosure, e.g., the TME Panel-1 Classifier, is a combined biomarker, i.e., it is a biomarker derived from discrete biomarkers integrated into a combination of signature scores, namely, an angiogenic signature score and an immune signature score.

Assignment, non-assignment, discontinuation, interruption or modification of a TME phenotype class-specific therapy can be based on the presence, absence, magnitude, or change of a specific TME phenotype class. For example, if a subject has a cancer tumor, e.g., a tumor from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) classified by an ANN classifier of the present disclosure, e.g., the TME Panel-1 Classifier, in the IA TME phenotype class, an IA TME phenotype class-specific therapy would be administered. In some aspects, assignment of a TME phenotype class-specific therapy is based on the absence of a specific TME phenotype, i.e., if a subject has a cancer tumor that is not classified by an ANN classifier of the present disclosure, e.g., the TME Panel-1 Classifier, in the IA TME phenotype class, an IA TME phenotype class-specific therapy would not be administered. Thus, identification of a patient or tumor from the patient as belonging to a TME phenotype class could be used to discard potential therapeutic options. Similarly, identification of a patient or tumor from the patient as belonging to a TME phenotype class that does not match a current therapy, could be used to cease or interrupt the therapy or to modify the therapy, for example, by including or excluding additional therapeutic agents. For example, identifying a mismatch between patient or tumor TME phenotype class and current therapy could be used to include adjuvant therapies, resulting in a TME phenotype class-specific treatment that would match the TME phenotype classification of the patient.

In some aspects, the classification of a patient or cancer sample into a TME phenotype class, and assignment of a TME phenotype class-specific therapy to the patient or cancer is not biunivocal. In other words, a patient or cancer sample can be classified as biomarker-positive and/or biomarker-negative for more than one TME phenotype class, and more than one TME phenotype class therapy or a combination thereof can be used to treat that patient. For example, the classification of a patient or cancer sample as biomarker-positive for two different TME phenotype classes could be used to select a treatment comprising a combination of pharmacological approaches in the TME phenotype class-specific therapies corresponding to the TME phenotype classes for which the patient or cancer sample is biomarker-positive. Furthermore, if the patient or cancer sample is biomarker-negative for a particular TME phenotype class, such knowledge can be used to exclude specific pharmacological approaches in the TME phenotype class therapy corresponding to the TME phenotype class for which the patient or cancer sample is biomarker-negative. Thus, drugs or combinations thereof, treatments or combinations thereof, and/or clinical regimens or combinations that are useful to treat a specific type of cancer classified as biomarker-positive for a particular TME phenotype class, can be combined to treat patients having more than one biomarker-positive signal, i.e., having a cancer sample classified as biomarker-positive for more than one TME phenotype class.

In some aspects, depending on the mechanism of action of a drug or a clinical regimen, different classification parameters, e.g., different gene panel subsets, different thresholds, different ANN architectures, different activation functions, or different post-processing functions, can be used to yield different TME phenotype classes, which in turn would be used to select appropriate TME phenotype class-specific therapies. Thus, depending on the mechanism of action of a drug or a clinical regimen, different classification parameters, e.g., different gene panel subsets, different variants of the ANN classifiers disclosed herein, e.g., the TME Panel-1 Classifier, could be developed. Accordingly, each drug or drug regimen may have different diagnostic gene panels and differently configured ANN classifiers to inform the clinician, e.g., a medical doctor, to decide whether a patient should be selected for treatment, whether treatment should be initiated, whether treatment should be suspended, or whether treatment should be modified.

In some aspects, a clinician can account for co-variates of biomarker status of a patient, and combine the probability of the TME phenotype class with MSI/MSS (Microsatellite Instability/Microsatellite Stability-High) status, EBV (Epstein-Barr virus) status, PD-1/PD-L1 status (such as CPS, i.e., combined positive score), neutrophil-leukocyte ratio (NLR), dMMR status, presence or absence of mutations in specific molecular biomarkers (e.g., KRAS, NRAS, BRAF), tumor location (e.g., left tumor or right tumor in the case of colorectal cancer), tumor size, tumor shape, tumor surface to volume ratio, invasiveness (e.g., whether cancer cells are present is lymphatic nodes in the case of breast cancer), or confounding variables such as prior treatment history.

In some aspects, the clinician is given a binary result from the classifier, and the decision to treat or not treat as described herein is made. In one aspect, the clinician is given, e.g., a plot of the patient's cancer classification results superimposed on a latent space and interpreted with probability thresholds, or a linear or polynomial logistic regression.

Classification of a dMMR tumor in the IA TME phenotype class by using an ANN classifier disclosed herein, e.g., TME Panel-1, correlates with improved clinical outcomes in treatments with checkpoint inhibitors. Accordingly, patients with dMMR cancer with an IA TME phenotype can be administered a therapy comprising checkpoint inhibitors selected from the IA TME phenotype class-specific therapies disclosed below. The present disclosure provides a method to treat a patient having a dMMR cancer with an IA TME phenotype comprising administering IA TME phenotype class-specific therapy to the patient. Also provided is a method of selecting a patient for treatment with IA TME phenotype class-specific therapy if the patient has a dMMR cancer with an IA TME phenotype.

Classification of a dMMR tumor in the IS TME phenotype class by using an ANN classifier disclosed herein, e.g., TME Panel-1, correlates with improved clinical outcomes in treatments combining checkpoint inhibitors and phosphatidylserine inhibitors. Accordingly, patients with dMMR cancer with an IS TME phenotype can be administered a combined therapy comprising checkpoint inhibitors and phosphatidylserine inhibitors selected from the IS TME phenotype class-specific therapies disclosed below. The present disclosure provides a method to treat a patient having a dMMR cancer with an IS TME phenotype comprising administering a treatment combining checkpoint inhibitors and phosphatidylserine inhibitors to the patient. Also provided is a method of selecting a patient for a treatment combining checkpoint inhibitors and phosphatidylserine inhibitors if the patient has a dMMR cancer with an IS TME phenotype.

Classification of a MSI-H tumor in the IA TME phenotype class by using an ANN classifier disclosed herein, e.g., TME Panel-1, correlates with improved clinical outcomes in treatments with checkpoint inhibitors. Accordingly, patients with MSI-H cancer with an IA TME phenotype can be administered a therapy comprising checkpoint inhibitors selected from the IA TME phenotype class-specific therapies disclosed below. The present disclosure provides a method to treat a patient having a MSI-H cancer with an IA TME phenotype comprising administering IA TME phenotype class-specific therapy to the patient. Also provided is a method of selecting a patient for treatment with IA TME phenotype class-specific therapy if the patient has a dMMR cancer with an IA TME phenotype.

Classification of a MSI-H tumor in the IS TME phenotype class by using an ANN classifier disclosed herein, e.g., TME Panel-1, correlates with improved clinical outcomes in treatments combining checkpoint inhibitors and phosphatidylserine inhibitors. Accordingly, patients with MSI-H cancer with an IS TME phenotype can be administered a combined therapy comprising checkpoint inhibitors and phosphatidylserine inhibitors selected from the IS TME phenotype class-specific therapies disclosed below. The present disclosure provides a method to treat a patient having a MSI-H cancer with an IS TME phenotype comprising administering a treatment combining checkpoint inhibitors and phosphatidylserine inhibitors to the patient. Also provided is a method of selecting a patient for a treatment combining checkpoint inhibitors and phosphatidylserine inhibitors if the patient has a MSI-H cancer with an IS TME phenotype.

The methods and compositions disclosed herein can be used for the treatment of multiple types cancer, e.g., to identify patients for treatment with specific therapies, to predict disease free probability and overall survival, or to predict the outcome of targeted therapies. In some aspects, the cancer is, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC).

In some aspects, the methods and compositions disclosed herein are used to reduce or decrease a size of a cancer tumor or inhibit a cancer tumor growth in a subject in need thereof, wherein the cancer is, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC).

Classification of a metastatic tumor, e.g., a tumor from gastric cancer, breast cancer, prostate cancer, liver cancer, carcinoma of head and neck, melanoma, colorectal cancer, ovarian, glioma, glioblastoma, or lung cancer in the A or IS TME phenotype classes by using an ANN classifier disclosed herein, e.g., TME Panel-1, correlates with improved clinical outcomes in treatments with angiogenesis inhibitors. Accordingly, patients with metastatic cancer with an A or IS TME phenotype can be administered a therapy comprising angiogenesis inhibitors selected from the A TME phenotype class-specific therapies disclosed below. The present disclosure provides a method to treat a patient having metastatic cancer with an A or IS TME phenotype comprising administering a treatment with angiogenesis inhibitors to the patient. Also provided is a method of selecting a patient for a treatment with angiogenesis inhibitors if the patient has a metastatic cancer with an A or IS TME phenotype.

Classification of a tumor e.g., a tumor from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) in the IA TME phenotype class by using an ANN classifier disclosed herein, e.g., TME Panel-1, can be used to select a checkpoint inhibitor, e.g., pembrolizumab, as an adjuvant therapy. The present disclosure provides a method to treat a patient having a cancer e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) with an IA TME phenotype comprising administering a treatment comprising a checkpoint inhibitor, e.g., pembrolizumab, as an adjuvant therapy. Also provided is a method of selecting a patient for a treatment comprising a checkpoint inhibitor, e.g., pembrolizumab, as an adjuvant therapy if the patient has a colorectal cancer with an IA TME phenotype. Classification of a tumor, e.g., a tumor from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) in the A TME phenotype class by using an ANN classifier disclosed herein, e.g., TME Panel-1, can be used to select an anti-angiogenic therapy, e.g., with bevacizumab, as an adjuvant therapy. The present disclosure provides a method to treat a patient having a cancer, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) with an A TME phenotype comprising administering a treatment comprising an anti-angiogenic therapy, e.g., with bevacizumab, as an adjuvant therapy. Also provided is a method of selecting a patient for a treatment comprising an anti-angiogenic therapy, e.g., with bevacizumab, as an adjuvant therapy if the patient has a colorectal cancer with an A TME phenotype.

Classification of a tumor from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) as having a dominant TME phenotype class by using an ANN classifier disclosed herein, e.g., TME Panel-1 can be used to select a therapy disclosed below that would match, for example, the dominant TME phenotype class. The present disclosure provides a method to treat a patient having a gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) with a specific TME phenotype class comprising administering a TME phenotype class-specific treatment to the patient, wherein the treatment matches the specific TME phenotype class. Also provided is a method of selecting a patient for a treatment with a TME phenotype class-specific treatment if the patient has gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) with a specific TME phenotype, wherein the TME phenotype class-specific treatment matches the specific TME phenotype class.

The present disclosure provides a method to treat a patient having locally advanced/metastatic gastric cancer with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having locally advanced/metastatic gastric cancer to an IA TME phenotype class. In some aspects, the ANN classifier (e.g., the TME Panel-1 Classifier) assigns the tumor sample from the patient having locally advanced/metastatic gastric cancer to an A TME phenotype class. In some aspects, the ANN classifier (e.g., the TME Panel-1 Classifier) assigns the tumor sample from the patient having locally advanced/metastatic gastric cancer to an IS TME phenotype class. In some aspects, a patient having locally advanced/metastatic gastric cancer assigned to an IA TME phenotype class can be treated with a therapy comprising or consisting of an immune checkpoint inhibitor therapy (e.g., an anti-PD(L)1 therapy). In some aspects, a patient having locally advanced/metastatic gastric cancer assigned to an IA TME phenotype class can be treated with a combination therapy comprising or consisting of chemotherapy, an immune checkpoint inhibitor therapy (e.g., an anti-PD(L)1 therapy), and navicixizumab. In some aspects, a patient having locally advanced/metastatic gastric cancer assigned to an IA TME phenotype class can be treated with a combination therapy comprising or consisting of chemotherapy, an immune checkpoint inhibitor therapy (e.g., an anti-PD(L)1 therapy), and bavituximab. In some aspects, a patient having locally advanced/metastatic gastric cancer assigned to an A TME phenotype class can be treated with a combination therapy comprising or consisting of chemotherapy and an anti-angiogenic therapy. In some aspects, a patient having locally advanced/metastatic gastric cancer assigned to an A TME phenotype class can be treated with a combination therapy comprising or consisting of chemotherapy, an immune checkpoint inhibitor therapy (e.g., an anti-PD(L)1 therapy), and navicixizumab. In some aspects, a patient having locally advanced/metastatic gastric cancer assigned to an IS TME phenotype class can be treated with a combination therapy comprising or consisting of chemotherapy, an immune checkpoint inhibitor therapy (e.g., an anti-PD(L)1 therapy), and bavituximab. In some aspects, a patient having locally advanced/metastatic gastric cancer assigned to an IS TME phenotype class can be treated with a combination therapy comprising or consisting of chemotherapy, an immune checkpoint inhibitor therapy (e.g., an anti-PD(L)1 therapy), and navicixizumab.

The present disclosure provides a method to treat a patient having previously untreated gastric cancer with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having previously untreated gastric cancer to an IS TME phenotype class. In some aspects, the ANN classifier (e.g., the TME Panel-1 Classifier) assigns the tumor sample from the patient having previously untreated gastric cancer to an A TME phenotype class. In some aspects, a patient having previously untreated gastric cancer assigned to an IS TME phenotype class can be treated with a combination therapy comprising or consisting of chemotherapy, and an anti-angiogenic therapy. In some aspects, a patient having previously untreated gastric cancer assigned to an A TME phenotype class can be treated with a combination therapy comprising or consisting of chemotherapy, and an anti-angiogenic therapy.

The present disclosure provides a method to treat a patient having breast cancer (e.g., locally advanced or metastatic Her2-negative breast cancer) with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having breast cancer (e.g., locally advanced or metastatic Her2-negative breast cancer) to an A TME phenotype class. In some aspects, the ANN classifier (e.g., the TME Panel-1 Classifier) assigns the tumor sample from the patient having breast cancer (e.g., locally advanced or metastatic Her2-negative breast cancer) to an IS TME phenotype class. In some aspects, a patient having breast cancer (e.g., locally advanced or metastatic Her2-negative breast cancer) assigned to an A TME phenotype class can be treated with a combination therapy comprising navicixizumab. In some aspects, a patient having breast cancer (e.g., locally advanced or metastatic Her2-negative breast cancer) assigned to an IS TME phenotype class can be treated with a combination therapy comprising navicixizumab. In some aspects, the combination therapy comprising navicixizumab comprises or consists of navicixizumab plus chemotherapy (e.g., docetaxel or cabazitaxel). In some aspects, the combination therapy comprising navicixizumab comprises or consists of navicixizumab plus PARP inhibitor therapy (e.g., rucaparib or olaparib).

The present disclosure provides a method to treat a patient having prostate cancer (e.g., castration-resistant metastatic prostate cancer) with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having prostate cancer (e.g., castration-resistant metastatic prostate cancer) to an A TME phenotype class. In some aspects, the ANN classifier (e.g., the TME Panel-1 Classifier) assigns the tumor sample from the patient having prostate cancer (e.g., castration-resistant metastatic prostate cancer) to an IS TME phenotype class. In some aspects, a patient having prostate cancer (e.g., castration-resistant metastatic prostate cancer) assigned to an A TME phenotype class can be treated with a combination therapy comprising navicixizumab. In some aspects, a patient having prostate cancer (e.g., castration-resistant metastatic prostate cancer) assigned to an IS TME phenotype class can be treated with a combination therapy comprising navicixizumab. In some aspects, the combination therapy comprising navicixizumab comprises or consists of navicixizumab plus chemotherapy (e.g., docetaxel or cabazitaxel). In some aspects, the combination therapy comprising navicixizumab comprises or consists of navicixizumab plus PARP inhibitor therapy (e.g., rucaparib or olaparib).

The present disclosure provides a method to treat a patient having liver cancer (e.g., hepatocellular carcinoma such as advanced metastatic hepatocellular carcinoma) with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having liver cancer (e.g., hepatocellular carcinoma such as advanced metastatic hepatocellular carcinoma) to an IA TME phenotype class. In some aspects, the ANN classifier (e.g., the TME Panel-1 Classifier) assigns the tumor sample from the patient having liver cancer (e.g., hepatocellular carcinoma such as advanced metastatic hepatocellular carcinoma) to an IS TME phenotype class. In some aspects, a patient having liver cancer (e.g., hepatocellular carcinoma such as advanced metastatic hepatocellular carcinoma) assigned to an IA TME phenotype class can be treated with a combination therapy comprising or consisting of bavituximab and an immune checkpoint inhibitor therapy (e.g., an anti-PD(L)1 therapy). In some aspects, a patient having liver cancer (e.g., hepatocellular carcinoma such as advanced metastatic hepatocellular carcinoma) assigned to an IS TME phenotype class can be treated with a combination therapy comprising or consisting of bavituximab and an immune checkpoint inhibitor therapy (e.g., an anti-PD(L)1 therapy).

The present disclosure provides a method to treat a patient having carcinoma of head and neck (e.g., recurrent or metastatic squamous cell carcinoma of head and neck) with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having carcinoma of head and neck (e.g., recurrent or metastatic squamous cell carcinoma of head and neck) to an IA TME phenotype class. In some aspects, the ANN classifier (e.g., the TME Panel-1 Classifier) assigns the tumor sample from the patient having carcinoma of head and neck (e.g., recurrent or metastatic squamous cell carcinoma of head and neck) to an IS TME phenotype class. In some aspects, a patient having carcinoma of head and neck (e.g., recurrent or metastatic squamous cell carcinoma of head and neck) assigned to an IA TME phenotype class can be treated with a combination therapy comprising or consisting of bavituximab and an immune checkpoint inhibitor therapy (e.g., an anti-PD(L)1 therapy). In some aspects, a patient having carcinoma of head and neck (e.g., recurrent or metastatic squamous cell carcinoma of head and neck) assigned to an IS TME phenotype class can be treated with a combination therapy comprising or consisting of bavituximab and an immune checkpoint inhibitor therapy (e.g., an anti-PD(L)1 therapy).

The present disclosure provides a method to treat a patient having melanoma with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having melanoma to an IA TME phenotype class. In some aspects, the ANN classifier (e.g., the TME Panel-1 Classifier) assigns the tumor sample from the patient having melanoma to an IS TME phenotype class. In some aspects, a patient having melanoma assigned to an IA TME phenotype class can be treated with a combination therapy comprising or consisting of bavituximab and radiation therapy. In some aspects, a patient having melanoma assigned to an IS TME phenotype class can be treated with a combination therapy comprising or consisting of bavituximab and radiation therapy.

The present disclosure provides a method to treat a patient having colorectal cancer (e.g., advanced colorectal cancer metastatic to liver) with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having colorectal cancer (e.g., advanced colorectal cancer metastatic to liver) to an ID TME phenotype class. In some aspects, a patient having colorectal cancer (e.g., advanced colorectal cancer metastatic to liver) assigned to an ID TME phenotype class can be treated with a combination therapy comprising or consisting of navicixizumab, an anti-PD(L)1 therapy, and an innate immune stimulating agent, such as the Dectin agonist Imprime PGG, the STING agonist BMS-986301, or the NLR agonist BMS-986299.

The present disclosure provides a method to treat a patient having ovarian cancer (e.g., platinum-resistant ovarian cancer or platinum-sensitive recurrent ovarian cancer) with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having ovarian cancer (e.g., platinum-resistant ovarian cancer or platinum-sensitive recurrent ovarian cancer) to an IS or A TME phenotype class. In some aspects, a patient having ovarian cancer (e.g., platinum-resistant ovarian cancer or platinum-sensitive recurrent ovarian cancer) assigned to an IS or A TME phenotype class can be treated with a combination therapy comprising or consisting of PARP inhibitor (Olaparib, Rucaparib, Niraparib, etc.) plus an immune checkpoint inhibitor therapy (e.g., anti-PD-(L)1, i.e., an inhibitor to PD-1 or PD-L1) plus navicixizumab, and represents a non-chemotherapeutic treatment option for ovarian cancer.

The present disclosure provides a method to treat a patient having breast cancer (e.g., platinum-resistant or platinum-sensitive recurrent triple negative breast cancer) with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having breast cancer (e.g., platinum-resistant or platinum-sensitive recurrent triple negative breast cancer) to an IA, IS or A TME phenotype class. In some aspects, a patient having breast cancer (e.g., platinum-resistant or platinum-sensitive recurrent triple negative breast cancer) assigned to an IA, IS or A TME phenotype class can be treated with a PARP inhibitor, an immune checkpoint inhibitors and navicixizumab.

The present disclosure provides a method to treat a patient having melanoma with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having melanoma to an IS TME phenotype class. In some aspects, a patient having melanoma assigned to an IS TME phenotype class can be treated with an immune modulator (such as vidutolimod) and CPI combination therapy. Example additional immune modulators in this class are ProMune CpG 7909 (PF3512676), SD-101, 1018 ISS, IMO-2123, Litenimod, MIS416, Cobitolimod, ImprimePGG (odetiglucan), imiquimod, fingolimod, tilsotolimod, and BL-7040.

The present disclosure provides a method to treat a patient having colorectal cancer (e.g., metastatic colorectal cancer) with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having colorectal cancer (e.g., metastatic colorectal cancer) to an A or IS TME phenotype class. In some aspects, a patient having colorectal cancer (e.g., metastatic colorectal cancer) assigned to an A or IS TME phenotype class can be treated with anti-DLL4/anti-VEGF antagonist in combination with a chemotherapeutic agent (e.g., FOLFOX, FOLFIRI, or irinotecan).

The present disclosure provides a method to treat a patient having glioma or glioblastoma with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having glioma or glioblastoma to an IS or IA TME phenotype class. In some aspects, a patient having glioma or glioblastoma assigned to an IS or IA TME phenotype class can be treated with a bavituximab, a checkpoint inhibitor, and radiation.

The present disclosure provides a method to treat a patient having non-small cell lung cancer with a specific TME phenotype class comprising administering a TME phenotype class-specific treatment to the patient, wherein the treatment matches the specific TME phenotype class. In some aspects, the ANN classifier (e.g., the TME Panel-1 Classifier) assigns the tumor sample from the patient having non-small cell lung cancer to an IS or IA TME phenotype class. In some aspects, a patient having glioma or glioblastoma assigned to an IS or IA TME phenotype class can be treated with a combination therapy of tislelizumab and chemotherapy

The present disclosure provides methods to treat tumors that are biomarker positive, comprised of the immune active (IA), immune suppressed (IS) and angiogenic (A) phenotypes with anti-DLL4/anti-VEGF antagonist, such as navicixizumab, ABT-165, or CTX-009, or an anti-VEGF antagonist, such as bevacizumab, ramucirumab, or varisacumab, in combination with an anti-PD-1 or an anti-PD-L1 checkpoint inhibitor (CPI), or a bispecific immunoglobulin or modified immunoglobulin of an anti-VEGF antagonist and a CPI.

The present disclosure provides a method to monitor the progression of disease, to select a specific treatment, to selection a patient for treatment, or to determine whether to continue or discontinue a treatment comprising (i) treating an immune desert (ID) patient with an investigator's choice of standard of care chemotherapeutics and/or tumor vaccines, the latter such as AST-301(pNGVL3-hICD), NeoVax, Proscavax, a personalized vaccine, α-lactalbumin vaccine, P10s-PADRE, OncoVax, PVX-410, Galinpepimut-S, GRT-C903/GRT-C904, KRAS peptide vaccine, pING-hHER3FL, GVAX, INCAGN01876, or a non-genetically-manipulated, living immune cell immunotherapy, a non-limiting example is AlloStim, (ii) taking a biopsy, e.g., 2 months after treatment, and (ii) reassessing the patient's TME Panel-1 status, wherein patients with transition from ID to IA are treated with an immunotherapy and responds, and patients that remain in the ID group are spared from more-futile therapies to which they are unlikely to respond, such as immunotherapy or antiangiogenic therapy.

The present disclosure also provides stratification strategies comprising a prespecified randomization ratio or prioritizing biomarkers. In some aspects, the prespecified randomization ratio uses a reverse prevalence ratio in which patients who have low-prevalence biomarkers have a greater likelihood of being assigned to a substudy for the lower prevalence population. In some aspects, the biomarker-prioritizing approach comprises ranking biomarker groups based on their predictive value and assigning patients to the treatment group for which the patients' biomarker profile has the highest predictive value. In some aspects, the TME phenotype or biomarker status (i.e., IA, IS, ID, A, A+IA, A+IS, or biomarker positive) is prioritized over other biomarkers, or used in combination with other biomarkers such as MSS status or PD-L1.

The present disclosure provides methods for classifying/stratifying cancer patients and/or cancer or tumor samples from those patients according to a TME phenotype determination resulting from applying an ANN classifier derived from a combined biomarker, e.g., a set of gene expression data corresponding to a gene panel, wherein the cancer is, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC). In some aspects, the ANN classifier is the TME Panel-1 Classifier.

In one aspect, the present disclosure provides a method for treating a human subject afflicted with a cancer, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) comprising administering an IA TME phenotype class-specific therapy to the subject, wherein, prior to the administration, an ANN classifier disclosed herein, e.g., TME Panel-1, is applied to a set of data comprising RNA expression levels of a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G), in a tumor sample obtained from the subject, and the ANN classifier assigns the tumor sample to an IA TME phenotype class.

The present disclosure also provides a method for treating a human subject afflicted with a cancer e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC), wherein the method comprises (A) identifying via an ANN disclosed herein, e.g., TME Panel-1, prior to the administration, a subject exhibiting an IA TME phenotype as determined by measuring RNA expression levels of a gene panel a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G), in a sample obtained from the subject; and, (B) administering to the subject an IA TME phenotype class-specific therapy.

In some aspects, the IA TME phenotype class-specific therapy can be administered in combination with additional TME phenotype class-specific therapies disclosed herein if the subject is biomarker-positive for additional TME phenotypes.

Also provided is a method for identifying a human subject afflicted with a cancer e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) suitable for treatment with an IA TME phenotype class-specific therapy, the method comprising applying an ANN classifier disclosed herein, e.g., TME Panel-1, to RNA expression levels of a gene panel (e.g., a gene panel comprising at least one gene from TABLE 1 and one gene from TABLE 2, a gene panel comprising a set of genes from TABLE 3 and a set of genes from TABLE 4, gene panel from TABLE 5, or any of the gene panels (Genesets) disclosed in FIG. 9A-G), in a sample obtained from a tumor from the subject; wherein the classification of the tumor in the IA TME phenotype class indicates that an IA TME phenotype class-specific therapy can be administered to the subject to treat the cancer.

In some aspects, the IA TME phenotype class-specific therapy comprises a checkpoint modulator therapy.

In some aspects, the checkpoint modulator therapy comprises administering an activator of a stimulatory immune checkpoint molecule. In some aspects, the activator of a stimulatory immune checkpoint molecule is, e.g., an antibody molecule against GITR (glucocorticoid-induced tumor necrosis factor receptor, TNFRSF18), OX-40 (TNFRSF4, ACT35, CD134, IMD16, TXGP1L, tumor necrosis factor receptor superfamily member 4, TNF receptor superfamily member 4), ICOS (Inducible T Cell Costimulator), 4-1BB (TNFRSF9, CD137, CDw137, ILA, tumor necrosis factor receptor superfamily member 9, TNF receptor superfamily member 9), or a combination thereof. In some aspects, the checkpoint modulator therapy comprises the administration of a RORy (RORC, NR1F3, RORG, RZR-GAMMA, RZRG, TOR, RAR-related orphan receptor gamma, IMD42, RAR related orphan receptor C) agonist.

In some aspects, the checkpoint modulator therapy comprises the administration of an inhibitor, modulator, agonist, or antagonist of an inhibitory immune checkpoint molecule. As used herein, the term “modulator,” refers to a molecule that interacts with a target either directly or indirectly, and imparts an effect on a biological or chemical process or mechanism. For example, a modulator can increase, facilitate, upregulate, activate, inhibit, decrease, block, prevent, delay, desensitize, deactivate, down regulate, or the like, a biological or chemical process or mechanism. Accordingly, a modulator can be an “agonist” or an “antagonist” of the target. The term “agonist” refers to a compound that increases at least some of the effect of the endogenous ligand of a protein, receptor, enzyme or the like. The term “antagonist” refers to a compound that inhibits at least some of the effect of the endogenous ligand of a protein, receptor, enzyme or the like.

In some aspects, the inhibitor of an inhibitory immune checkpoint molecule is, e.g., an antibody against PD-1 (PDCD1, CD279, SLEB2, hPD-1, hPD-1, hSLE1, Programmed cell death 1), e.g., sintilimab, tislelizumab, pembrolizumab, or an antigen binding portion thereof, an antibody against PD-L1 (CD274, B7-H, B7H1, PDCD1L1, PDCD1LG1, PDL1, CD274 molecule, Programmed cell death ligand 1, hPD-L1), an antibody against PD-L2 (PDCD1LG2, B7DC, Btdc, CD273, PDCD1L2, PDL2, bA574F11.2, programmed cell death 1 ligand 2), an antibody against CTLA-4 (CTLA4, ALPS5, CD, CD152, CELIAC3, GRD4, GSE, IDDM12, cytotoxic T-lymphocyte associated protein 4), a bispecific antibody comprising at least a binding specificity for PD-L1, PD-L2, or CTLA-4, alone or a combination thereof.

In some aspects, the inhibitor of an inhibitory immune checkpoint molecule is, e.g., any of the antibodies disclosed above in combination with an inhibitor, modulator, antagonist or agonist of TIM-3 (T-cell immunoglobulin and mucin-domain containing-3), LAG-3 (Lymphocyte-activation gene 3), BTLA (B- and T-lymphocyte attenuator), TIGIT (T cell immunoreceptor with Ig and ITIM domains), VISTA (V-domain Ig suppressor of T cell activation), TGF-β (transforming growth factor beta) or its receptors, a CD86 (Cluster of Differentiation 86) agonist, LAIR1 (Leukocyte-associated immunoglobulin-like receptor 1), CD160 (Cluster of Differentiation 160), 2B4 (Natural Killer Cell Receptor 2B4; Cluster of Differentiation 244), GITR, OX40, 4-1BB (CD137), CD2 (Cluster of Differentiation 2), CD27 (Cluster of Differentiation 27), CDS (CDP-Diacylglycerol Synthase 1), ICAM-1 (Intercellular Adhesion Molecule 1), LFA-1 (Lymphocyte function-associated antigen 1; CD11a/CD18), ICOS (Inducible T-cell COStimulator; CD278), CD30 (Cluster of Differentiation 30), CD40 (Cluster of Differentiation 40), BAFFR (B-cell activating factor receptor), HVEM (Herpesvirus entry mediator), CD7 (Cluster of Differentiation 7), LIGHT (tumor necrosis factor superfamily member 14; TNFSF14), NKG2C (killer cell lectin like receptor C2; KLRC2, CD159c), SLAMF7 (SLAM family member 7), NKp80 (Activating Coreceptor NKp80; Lectin-Like Receptor F1; KLRF1; Killer Cell Lectin Like Receptor F1), or any combination thereof.

In some aspects, the anti-PD-1 antibody comprises, e.g., nivolumab, pembrolizumab, cemiplimab, sintilimab, tislelizumab, or an antigen-binding portion thereof. In some aspects, the anti-PD-1 antibody cross-competes with nivolumab, pembrolizumab, cemiplimab, sintilimab, or tislelizumab for binding to human PD-1, or binds to the same epitope as nivolumab, pembrolizumab, cemiplimab, sintilimab, or tislelizumab.

In some aspects, the anti-PD-L1 antibody comprises, e.g., avelumab, atezolizumab, durvalumab, or an antigen-binding portion thereof. In some aspects, the anti-PD-1 antibody cross-competes with avelumab, atezolizumab, or durvalumab for binding to human PD-1, or binds to the same epitope as avelumab, atezolizumab, or durvalumab.

In some aspects, the checkpoint modulator therapy comprises the administration of (i) an anti-PD-1 antibody, e.g., an antibody selected from the group consisting of nivolumab, pembrolizumab, sintilimab, tislelizumab, and cemiplimab; (ii) an anti-PD-L1 antibody, e.g., an antibody selected from the group consisting of avelumab, atezolizumab, and durvalumab; or (iii) a combination thereof.

In one aspect, the present disclosure provides a method for treating a human subject afflicted with a cancer e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) comprising administering an IS TME phenotype class-specific therapy to the subject, wherein, prior to the administration, an ANN classifier disclosed herein, e.g., TME Panel-1, is applied to a set of data comprising RNA expression levels of a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G) in a tumor sample obtained from the subject, and the ANN classifier assigns the tumor sample to an IS TME phenotype class.

In one aspect, the present disclosure provides a method for treating a human subject afflicted with a cancer e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) comprising administering an IS TME phenotype class-specific therapy to the subject, wherein, prior to the administration, an ANN classifier disclosed herein, e.g., TME Panel-1, is applied to a set of data comprising RNA expression levels of a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G) in a tumor sample obtained from the subject, and the ANN classifier assigns the tumor sample to an IS TME phenotype class.

The present disclosure also provides a method for treating a human subject afflicted with a cancer, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) comprising (A) identifying via an ANN disclosed herein, e.g., TME Panel-1, prior to the administration, a subject exhibiting an IS TME phenotype as determined by measuring RNA expression levels of a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G), in a sample obtained from the subject; and, (B) administering to the subject an IS TME phenotype class-specific therapy.

In some aspects, the IS TME phenotype class-specific therapy can be administered in combination with additional TME phenotype class-specific therapies disclosed herein if the subject is biomarker-positive for additional TME phenotypes.

Also provided is a method for identifying a human subject afflicted with a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) suitable for treatment with an IS TME phenotype class-specific therapy, the method comprising applying an ANN classifier disclosed herein, e.g., TME Panel-1, to RNA expression levels of a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G), in a sample obtained from a tumor from the subject; wherein the classification of the tumor in the IS TME phenotype class indicates that an IS TME phenotype class-specific therapy can be administered to the subject to treat the cancer.

In some aspects, the IS TME phenotype class-specific therapy comprises, e.g., the administration of (1) a checkpoint modulator therapy and an anti-immunosuppression therapy (e.g., a combination therapy comprising the administration of pembrolizumab and bavituximab) and/or (2) an antiangiogenic therapy. In some aspects, the checkpoint modulator therapy comprises, e.g., the administration of an inhibitor of an inhibitory immune checkpoint molecule. In some aspects, the inhibitor of an inhibitory immune checkpoint molecule is, e.g., an antibody against PD-1 (e.g., sintilimab, tislelizumab, pembrolizumab, or an antigen binding portion thereof), PD-L1, PD-L2, CTLA-4, or a combination thereof.

In some aspect, the anti-PD-1 antibody comprises, e.g., nivolumab, pembrolizumab, cemiplimab, spartalizumab (PDR001), sintilimab, tislelizumab, or geptanolimab (CBT-501), or an antigen-binding portion thereof. In some aspects, the anti-PD-1 antibody cross-competes with nivolumab, pembrolizumab, cemiplimab, PDR001, sintilimab, tislelizumab, or CBT-501, for binding to human PD-1, or binds to the same epitope as nivolumab, pembrolizumab, cemiplimab, sintilimab, tislelizumab, PDR001, or CBT-501.

In some aspects, the anti-PD-L1 antibody comprises, e.g., avelumab, atezolizumab, durvalumab, or an antigen-binding portion thereof. In some aspects, the anti-PD-L1 antibody cross-competes with avelumab, atezolizumab, or durvalumab for binding to human PD-L1 or binds to the same epitope as avelumab, atezolizumab, or durvalumab.

In some aspects, the anti-CTLA-4 antibody comprises ipilimumab, or an antigen-binding portion thereof. In some aspects, the anti-CTLA-4 antibody cross-competes with ipilimumab for binding to human CTLA-4 or binds to the same epitope as ipilimumab.

In some aspects, the checkpoint modulator therapy comprises, e.g., the administration of (i) an anti-PD-1 antibody selected, e.g., from the group consisting of nivolumab, pembrolizumab, sintilimab, tislelizumab, and cemiplimab; (ii) an anti-PD-L1 antibody selected, e.g., from the group consisting of avelumab, atezolizumab, and durvalumab; (iii) an anti-CTLA-4 antibody, e.g., ipilimumab, or (iii) a combination thereof.

In some aspects, the antiangiogenic therapy comprises, e.g., the administration of an anti-VEGF (Vascular endothelial growth factor) antibody selected from the group consisting of varisacumab, bevacizumab, navicixizumab (an anti-DLL4/anti-VEGF bispecific antibody), and a combination thereof. In some aspects, the antiangiogenic therapy comprises, e.g., the administration of an anti-VEGFR antibody. In some aspects, the anti-VEGFR antibody is an anti-VEGFR2 Vascular endothelial growth factor receptor 2) antibody. In some aspects, the anti-VEGFR2 antibody comprises ramucirumab. In some aspects, the antiangiogenic therapy comprises, e.g., navicixizumab, ABL101 (NOV1501), or dilpacimab (ABT165).

In some aspects, the anti-immunosuppression therapy comprises, e.g., the administration of an anti-PS (phosphatidylserine) antibody, anti-PS targeting antibody, antibody that binds β2-glycoprotein 1, inhibitor of PI3Kγ (phosphatidylinositol-4,5-bisphosphate 3-kinase catalytic subunit gamma isoform), adenosine pathway inhibitor, inhibitor of IDO, inhibitor of TIM, inhibitor of LAG3, inhibitor of TGF-β, CD47 inhibitor, or a combination thereof.

In some aspects, the anti-PS targeting antibody is, e.g., bavituximab, 1N11, or an antibody that binds β2-glycoprotein 1 (β2GP1, or Apo-H). In some aspects, the anti-PS targeting antibody is bavituximab. In some aspects, the anti-PS targeting antibody is an antibody that binds β2-glycoprotein 1 (β2GP1, or Apo-H. In some aspects, the anti-PS targeting antibody is 1N11. See, e.g., Schad et al. (2020) J. Immunol. 201 (S1):170.5; Yin et al. (2009) Cancer Res. 69 (S9):5463; Zohar & Shoenfeld (2018) Immunotargets Ther. 7:51-53, all of which are herein incorporated by reference in their entireties.

In some aspects, the PI3Kγ inhibitor is, e.g., LY3023414 (samotolisib) or IPI-549 (eganelisib). In some aspects, the adenosine pathway inhibitor is, e.g., AB-928. In some aspects, the TGFβ inhibitor is, e.g., LY2157299 (galunisertib) or the TGFβR1 inhibitor LY3200882. In some aspects, the CD47 inhibitor is, e.g., magrolimab (5F9). In some aspects, the CD47 inhibitor targets SIRPα.

In some aspects, the anti-immunosuppression therapy comprises the administration of an inhibitor, modulator, agonist or antagonist of TIM-3, LAG-3, BTLA, TIGIT, VISTA, TGF-β or its receptor, CD86, LAIR1, CD160, 2B4, GITR, OX40, 4-1BB (CD137), CD2, CD27, CDS, ICAM-1, LFA-1 (CD11a/CD18), ICOS (CD278), CD30, CD40, BAFFR, HVEM, CD7, LIGHT, NKG2C, SLAMF7, NKp80, or a combination thereof.

In one aspect, the present disclosure provides a method for treating a human subject afflicted with a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) wherein the method comprises administering an A TME phenotype class-specific therapy to the subject, wherein, prior to the administration, an ANN classifier disclosed herein, e.g., TME Panel-1, is applied to a set of data comprising RNA expression levels of a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G) in a tumor sample obtained from the subject, and the ANN classifier assigns the tumor sample to an A TME phenotype class.

The present disclosure also provides a method for treating a human subject afflicted with a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) wherein the method comprises (A) identifying via an ANN disclosed herein, e.g., TME Panel-1, prior to the administration, a subject exhibiting an A TME phenotype as determined by measuring RNA expression levels of a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G), in a sample obtained from the subject; and, (B) administering to the subject an A TME phenotype class-specific therapy.

In some aspects, the A TME phenotype class-specific therapy can be administered in combination with additional TME phenotype class-specific therapies disclosed herein if the subject is biomarker-positive for additional TME phenotypes.

Also provided is a method for identifying a human subject afflicted with a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) suitable for treatment with an A TME phenotype class-specific therapy, the method comprising applying an ANN classifier disclosed herein, e.g., TME Panel-1, to RNA expression levels of a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G), in a sample obtained from a tumor from the subject; wherein the classification of the tumor in the A TME phenotype class indicates that an A TME phenotype class-specific therapy can be administered to the subject to treat the cancer.

In some aspects, the A TME phenotype class-specific therapy comprises a VEGF-targeted therapy and other anti-angiogenics, Angiopoietin 1 and 2 (Ang1 and Ang2), DLL4 (Delta Like Canonical Notch Ligand 4), bispecifics of anti-VEGF and anti-DLL4, TKI (tyrosine kinase inhibitors) such as fruquintinib, anti-FGF (Fibroblast growth factor) antibodies and antibodies or small molecules that inhibit the FGF receptor family (FGFR1 and FGFR2); anti-PLGF (Placental growth factor) antibodies and small molecules and antibodies against PLGF receptors, anti-VEGF-B (Vascular endothelial growth factor B) antibodies, anti-VEGF-C (Vascular endothelial growth factor C) antibodies, anti-VEGF-D (Vascular endothelial growth factor D); antibodies to VEGF/PLGF trap molecules such as aflibercept, or ziv-aflibercet; anti-DLL4 antibodies or anti-Notch therapies, such as inhibitors of gamma-secretase. In some aspects, the anti-angiogenic therapy comprises that administration of antagonists to endoglin, e.g., carotuximab (TRC105).

As used herein the term “VEGF-targeted therapy” refers to targeting the ligands, i.e., VEGF-A (vascular endothelial growth factor A), VEGF-B (vascular endothelial growth factor B), VEGF-C (vascular endothelial growth factor C), VEGF-D (vascular endothelial growth factor D), or PLGF (placental growth factor); the receptors, e.g., VEGFR1 (vascular endothelial growth factor receptor 1), VEGFR2 (vascular endothelial growth factor receptor 2), or VEGFR3 (vascular endothelial growth factor receptor 3); or any combination thereof.

In some aspects, the VEGF-target therapy comprises the administration of an anti-VEGF antibody or an antigen-binding portion thereof. In some aspects, the anti-VEGF antibody comprises, e.g., varisacumab, bevacizumab, or an antigen-binding portion thereof. In some aspects, the anti-VEGF antibody cross-competes with varisacumab or bevacizumab for binding to human VEGF-A, or binds to the same epitope as varisacumab or bevacizumab.

In some aspects, the VEGF-targeted therapy comprises the administration of an anti-VEGFR antibody. In some aspects, the anti-VEGFR antibody is an anti-VEGFR2 antibody. In some aspects, the anti-VEGFR2 antibody comprises ramucirumab or an antigen-binding portion thereof.

In some aspects, the A TME phenotype class-specific therapy comprises the administration of an angiopoietin/TIE2 (TEK receptor tyrosine kinase; CDC202B)-targeted therapy. In some aspects, the angiopoietin/TIE2-target therapy comprises the administration of endoglin and/or angiopoietin. In some aspects, the A TME phenotype class-specific therapy comprises the administration of a DLL4-targeted therapy. In some aspects, the DLL4-targeted therapy comprises the administration of navicixizumab, ABL101 (NOV1501), or ABT165.

In all methods disclosed above, e.g., methods of treating a subject with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC), or methods for selecting a subject with a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) for treatment with a TME phenotype class-specific therapy, wherein the TME phenotype class-specific therapy is selected according to the classification of the cancer's TME phenotype into one or more TME phenotype classes using an ANN classifier disclosed herein, e.g., TME Panel-1, the administration of the specific therapy, e.g., a TME phenotype class-specific therapy disclosed herein or a combination thereof, can effectively treat the cancer.

In some aspects, the administration of a TME phenotype class-specific therapy disclosed herein or a combination thereof to a subject with a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) reduces the cancer burden.

In some aspects, the administration of a TME phenotype class-specific therapy disclosed herein or a combination thereof to a subject with colorectal cancer reduces the cancer burden by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the cancer burden prior to the administration of the therapy.

In some aspects, the administration of a TME phenotype class-specific therapy disclosed herein or a combination thereof results in progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration of the therapy.

In some aspects, the subject exhibits stable disease after the administration of TME phenotype class-specific therapy disclosed herein or a combination thereof. The term “stable disease” refers to a diagnosis for the presence of a cancer, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC), however the cancer has been treated and remains in a stable condition, i.e. one that that is not progressive, as determined, e.g., by imaging data and/or best clinical judgment.

The term “progressive disease” refers to a diagnosis for the presence of a highly active state of the cancer, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC), i.e., one that has not been treated and is not stable or has been treated and has not responded to therapy, or has been treated and active disease remains, as determined by imaging data and/or best clinical judgment.

“Stable disease” can encompass a (temporary) tumor shrinkage/reduction in tumor volume during the course of the treatment compared to the initial tumor volume at the start of the treatment (i.e. prior to treatment). In this context, “tumor shrinkage” can refer to a reduced volume of the tumor upon treatment compared to the initial volume at the start of (i.e. prior to) the treatment. A tumor volume of, for example, less than 100% (e.g., of from about 99% to about 66% of the initial volume at the start of the treatment) can represent a “stable disease”.

“Stable disease” can alternatively encompass a (temporary) tumor growth/increase in tumor volume during the course of the treatment compared to the initial tumor volume at the start of the treatment (i.e. prior to treatment). In this context, “tumor growth” can refer to an increased volume of the tumor upon treatment inhibitor compared to the initial volume at the start of (i.e. prior to) the treatment. A tumor volume of, for example, more than 100% (e.g. of from about 101% to about 135% of the initial volume, preferably of from about 101% to about 110% of the initial volume at the start of the treatment) can represent a “stable disease”.

The term “stable disease” can include the following aspects. For example, the tumor volume does, for example, either not shrink after treatment (i.e. tumor growth is halted) or it does, for example, shrink at the start of the treatment but does not continue to shrink until the tumor has disappeared, i.e. tumor growth is first reverted but, before the tumor has, for example, less than 65% of the initial volume, the tumor grows again.

The term “response” when used in reference to the patients or the tumors to a TME phenotype class-specific therapy disclosed herein or a combination thereof can be reflected in a “complete response” or “partial response” of the patients or the tumors. The term “complete response” as used herein can refer to the disappearance of all signs of cancer in response to a TME phenotype class-specific therapy disclosed herein or a combination thereof. The term “complete response” and the term “complete remission” can be used interchangeably herein. For example, a “complete response” can be reflected in the continued shrinkage of the tumor (as shown in the appended example) until the tumor has disappeared. A tumor volume of, for example, 0% compared to the initial tumor volume (100%) at the start of (i.e. prior to) the treatment can represent a “complete response”.

Treatment with a TME phenotype class-specific therapy disclosed herein or a combination thereof can result in a “partial response” (or partial remission; e.g. a decrease in the size of a tumor, or in the extent of cancer in the body, in response to the treatment). A “partial response” can encompass a (temporary) tumor shrinkage/reduction in tumor volume during the course of the treatment compared to the initial tumor volume at the start of the treatment (i.e. prior to treatment). Thus, in some aspects, the subject exhibits a partial response after the administration of a TME phenotype class-specific therapy disclosed herein or a combination. In other aspects, the subject exhibits a complete response after the administration of a TME phenotype class-specific therapy disclosed herein or a combination thereof.

The term “response” can refer to a “tumor shrinkage.” Accordingly, the administration of TME phenotype class-specific therapy disclosed herein or a combination thereof to a subject in need thereof can result in a reduction in volume or shrinkage of the tumor.

In some aspects, following the administration of a TME phenotype class-specific therapy disclosed herein or a combination thereof, the tumor can be reduced in size by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to the tumor's volume prior to the treatment.

In some aspects, the volume of the tumor following the administration of a TME phenotype class-specific therapy disclosed herein or a combination thereof, is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% of the original volume of the tumor prior to the treatment.

In some aspects, the administration of a TME phenotype class-specific therapy disclosed herein or a combination thereof can reduce the growth rate of the tumor by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to the growth rate of the tumor's prior to the treatment.

The term “response” can also refer to a reduction in the number of tumors, for example, when a cancer has metastasized.

In some aspects, the administration of a TME phenotype class-specific therapy disclosed herein or a combination thereof improves progression-free survival probability of the subject by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 12%, at least about 130%, at least about 135%, at least about 140%, at least about 145%, or at least about 150%, compared to the progression-free survival probability of a subject not exhibiting the TME phenotype, or a subject not treated with a specific therapy disclosed herein, e.g., a TME phenotype class-specific therapy disclosed herein or a combination thereof.

In some aspects, the administration of a TME phenotype class-specific therapy disclosed herein or a combination thereof improves overall survival probability by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 125%, at least about 130%, at least about 140%, at least about 150%, at least about 160%, at least about 170%, at least about 175%, at least about 180%, at least about 190%, at least about 200%, at least about 210%, at least about 220%, at least about 225%, at least about 230%, at least about 240%, at least about 250%, at least about 260%, at least about 270%, at least about 275%, at least about 280%, at least about 290%, at least about 300%, at least about 310%, at least about 320%, at least about 325%, at least about 330%, at least about 340%, at least about 350%, at least about 360%, at least about 370%, at least about 375%, at least about 380%, at least about 390%, or at least about 400%, compared to the overall survival probability of a subject not exhibiting the TME phenotype or a subject not treated with a TME phenotype class-specific therapy disclosed herein or a combination thereof.

The present disclosure also provides a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G), for use in assigning a tumor e.g., a tumor in a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC), in a cancer patient to a specific TME phenotype class via an ANN classifier disclosed herein, e.g., TME Panel-1, wherein the assignment or non-assignment of the tumor to a specific TME phenotype class or a combination thereof is used for (i) identifying a patient as suitable for an anticancer therapy; (ii) determining the prognosis of a patient undergoing anticancer therapy; (iii) initiating, suspending, or modifying the administration of an anticancer therapy to the patient; or, (iv) a combination thereof. In some aspects, the gene panel is used according to the methods disclosed here, e.g., to classify a colorectal cancer tumor from a patient (e.g., to determine whether a tumor is biomarker-positive or biomarker-negative for a TME phenotype class disclosed herein or a combination thereof) and to administer a specific therapy (e.g., a TME phenotype class-specific therapy disclosed herein or a combination thereof) based on that classification.

The present disclosure also provides a combined biomarker for identifying via an ANN classifier, e.g., TME Panel-1, a human subject afflicted with a cancer, e.g., a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) suitable for treatment with an anticancer therapy, wherein the cancer's TME phenotype class is determined by measuring the expression levels, e.g., mRNA expression levels, of the genes in a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G), in a sample obtained from the subject, and wherein (a) the therapy is an IA TME phenotype class-specific therapy if the TME phenotype class assigned is IA; (b) the therapy is an IS TME phenotype class-specific therapy if the TME phenotype assigned is IS; (c) the therapy is an ID TME phenotype class-specific therapy if the TME phenotype assigned is ID; or (d) the therapy is an A TME phenotype class-specific therapy if the TME phenotype assigned is A.

In some aspects, e.g., when the subject is identified via an ANN classifier disclosed herein, e.g., TME Panel-1, as biomarker-positive or biomarker-negative for more than one of the TME phenotype classes disclosed herein, e.g., the subject is biomarker-positive for the IA and IS TME phenotype classes, the subject can be administered a combination therapy corresponding to TME phenotype class-specific therapies corresponding to TME phenotype classes for which the subject is biomarker positive, e.g., a combination therapy comprising an IA TME phenotype class-specific therapy and a IS TME phenotype class-specific therapy.

The present disclosure also provides an anticancer therapy for treating a cancer, e.g., a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) in a human subject in need thereof, wherein the subject is identified via an ANN classifier disclosed herein, e.g., the TME Panel-1 Classifier, as exhibiting or not exhibiting a specific TME phenotype determined by measuring the expression levels, e.g., mRNA expression levels, of the genes in a gene panel (e.g., a gene panel comprising at least one gene from TABLE 1 and one gene from TABLE 2, a gene panel comprising a set of genes from TABLE 3 and a set of genes from TABLE 4, a gene panel from TABLE 5, or any of the gene panels (Genesets) disclosed in FIG. 9A-G) in a sample obtained from the subject, and wherein (a) the therapy is an IA TME phenotype class-specific therapy if the TME phenotype class assigned is IA; (b) the therapy is an IS TME phenotype class-specific therapy if the TME phenotype class assigned is IS; or (c) the therapy is an A TME phenotype class-specific therapy if the TME phenotype class assigned is A. In some aspects, if the patient is biomarker-positive for more than one TME phenotype classes, the patient can receive a therapy combining TME phenotype class-specific therapies corresponding to each of the TME phenotype classes for which the patient is biomarker-positive.

In some aspects, the term “administering” can also comprise commencing a therapy, discontinuing or suspending a therapy, temporarily suspending a therapy, or modifying a therapy (e.g., increasing dosage or frequency of doses, or adding one of more therapeutic agents in a combination therapy).

In some aspects, samples can, for example, be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to the original healthcare provider or yet another healthcare provider, healthcare benefits provider or the patient. Similarly, the quantification of the expression level of a biomarker disclosed herein; comparisons between biomarker scores or protein expression levels; evaluation of the absence or presence of biomarkers; determination of biomarker levels with respect to a certain threshold; treatment decisions; or combinations thereof, can be performed by one or more healthcare providers, healthcare benefits providers, and/or clinical laboratories.

As used herein, the term “healthcare provider” refers to individuals or institutions that directly interact with and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer a therapy disclosed herein to treat a cancer, e.g., a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC).

A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification of a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition, a healthcare benefits provides can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a cancer tumor sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities, wherein the sample is from a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC).

The assignment of a gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) patient or cancer tumor to a specific TME phenotype class or classes disclosed herein can be applied, in addition to the treatment of patients or to the selection of a patient for treatment, to other therapeutic or diagnostic uses.

For example, to devise new methods of treatment (e.g., by selecting patients as candidates for a certain therapy or for participation in a clinical trial), to methods to monitor the efficacy of therapeutic agents, or to methods to adjust a treatment (e.g., formulations, dosage regimens, or routes of administration).

The methods disclosed herein can also include additional steps such as prescribing, initiating, and/or altering prophylaxis and/or treatment, based at least in part on the determination of the presence or absence of a particular TME phenotype in a subject's tumor from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) through the application of a classifier disclosed herein, e.g., the TME Panel-1 Classifier.

The present disclosure also provides a method of determining whether to treat with a certain TME phenotype class-specific therapy disclosed herein or a combination thereof a colorectal cancer patient having a tumor with a particular TME phenotype identified through the application of a classifier disclosed herein, e.g., the TME Panel-1 Classifier. Also provided are methods of selecting a patient diagnosed with a specific type of colorectal cancer (e.g., a left colorectal cancer, a right colorectal cancer, dMMR colorectal cancer, MSI-H colorectal cancer, or metastatic colorectal cancer) as a candidate for treatment with a certain TME phenotype class-specific therapy disclosed herein or a combination thereof based on the presence and/or absence of a particular TME phenotype identified through the application of a classifier disclosed herein, e.g., the TME Panel-1 classifier.

The present disclosure also provides a method of determining whether to treat with a certain TME phenotype class-specific therapy disclosed herein or a combination thereof a cancer patient, e.g., a patient with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) having a tumor with a particular TME phenotype identified through the application of a classifier disclosed herein, e.g., the TME Panel-1 Classifier.

Also provided are methods of selecting a patient diagnosed with a specific type of colorectal cancer (e.g., a left colorectal cancer, a right colorectal cancer, dMMR colorectal cancer, MSI-H colorectal cancer, or metastatic colorectal cancer) as a candidate for treatment with a certain TME phenotype class-specific therapy disclosed herein or a combination thereof based on the presence and/or absence of a particular TME phenotype identified through the application of a classifier disclosed herein, e.g., the TME Panel-1 classifier.

Also provided are methods of selecting a patient diagnosed with a specific type of cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) as a candidate for treatment with a certain TME phenotype class-specific therapy disclosed herein or a combination thereof based on the presence and/or absence of a particular TME phenotype identified through the application of a classifier disclosed herein, e.g., the TME Panel-1 classifier.

In one aspect, the methods disclosed herein include making a diagnosis, which can be a differential diagnosis, based at least in part on the assignment of a cancer tumor in a subject with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) to a specific TME phenotype class based on the application of a classifier, e.g., the TME Panel-1 Classifier, to mRNA expression levels for a panel of genes disclosed herein obtained from a sample from the tumor. This diagnosis can be recorded in a patient medical record. For example, in various aspects, the classification of the cancer's TME phenotype class, the diagnosis of the patient as treatable with a certain TME phenotype class-specific therapy disclosed below or a combination thereof, or the selected treatment, can be recorded in a medical record. The medical record can be in paper form and/or can be maintained in a computer-readable medium. The medical record can be maintained by a laboratory, physician's office, a hospital, a healthcare maintenance organization, an insurance company, and/or a personal medical record website.

In some aspects, a diagnosis, TME classification, selected therapy, etc., based on the application of classifier disclosed herein, e.g., the TME Panel-1 Classifier, can be recorded on or in a medical alert article such as a card, a worn article, and/or a radio-frequency identification (RFID) tag. As used herein, the term “worn article” refers to any article that can be worn on a subject's body, including, but not limited to, a tag, bracelet, necklace, or armband.

In some aspects, the sample can be obtained by a healthcare professional treating or diagnosing a cancer patient with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) for measurement of the biomarker levels (e.g., mRNA levels corresponding to the gene panels disclosed herein) in the sample according to the healthcare professional's instructions (e.g., using a particular assay as described herein).

In some aspects, the clinical laboratory performing the assay can advise the healthcare provider as to whether a cancer patient with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) can benefit from treatment with a specific TME phenotype class-specific therapy disclosed herein or a combination thereof based on whether the patient's cancer is classified as belonging to a particular TME phenotype class, e.g., by applying a classifier disclosed herein such as the TME Panel-1 Classifier.

In some aspects, results of a TME phenotype classification conducted by applying a classifier disclosed herein, e.g., the TME Panel-1 Classifier, can be submitted to a healthcare benefits provider for determination of whether the cancer patient's insurance will cover treatment with a specific TME phenotype class-specific therapy disclosed herein or a combination thereof. In some aspects, the clinical laboratory performing the assay can advise the healthcare provide as to whether a cancer patient with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) can benefit from treatment with a specific TME phenotype class-specific therapy disclosed herein or combination thereof based on the cancer's TME phenotype classification, e.g., using the TME Panel-1 Classifier disclosed herein.

The treatments with checkpoint inhibitors disclosed above and through the specification can comprise any checkpoint inhibitors selected from the group consisting of nivolumab (PD-1), pembrolizumab (PD-1), durvalumab (PD-L1), atezolizumab (PD-L1), ABBV-181 (PD-1), AMG 404 (PD-1), BI 754091 (PD-1), dostarlimab (PD-1), TSR-075 (PD-1/LAG-3 bi-specific), cetrelimab (PD-1), spartalizumab (PD-1), camrelizumab (PD-1), ATA2271 (PD-1), CDX-527 (PD-L1/CD27 bi-specific), cosibelimab (PD-L1), CX-072 (PD-1/PD-L1 probody), FS222 (PD-L1/CD137 bi-specific), FS118 (PD-L1/LAG-3 bi-specific), GEN1046 (PD-L1/CD137 bi-specific), JTX-4014 (PD-1), KY1043 (PD-L1), IMC-001 (PD-L1), TG-1501 (PD-L1), XmAb20717 (PD-1/CTLA-4 bi-specific), XmAb23104 (PD-1/ICOS bi-specific), genolimzumab (PD-1), APL-502 (PD-L1), cadonilimab (PD-1/CTLA-4 bi-specific), AK112 (PD-1/VEGF bi-specific), penpulimab (PD-1), KN046 (PD-L1/CTLA-4 bi-specific), SHR-1316 (PD-L1), BI-1206 (PD-1/FcγRIIB), BI-1808 (PD-1/TNFR2), PM8001 (PD-L1 bi-specific), CDX-527 (PD-L1/CD27 bi-specific), IBI315 (PD-1/HER2 bi-specific), HBM9167 (PD-L1), HLX10 (PD-1), LAE005 (PD-L1), LZM009 (PD-1), YBL-013 (PD-L1/CD3 bi-specific), avelumab (PD-L1), cemiplimab (PD-1), sintilimab (PD-1), tislelizumab (PD-1), toripalimab (PD-1), balstilimab (PD-1), zimberelimab (PD-1), sugemalimab (PD-L1), CS1003 (PD-1), GS-4224 (PD-L1), retifanlimab (PD-1), tebotelimab (PD-1/LAG-3 DART), MGD019 (PD-1/CTLA-4 DART), M7824 (PD-L1/TGFB bi-functional), sasanlimab (PD-1), envafolimab (PD-L1), ABSK043, ACE1708 (PD-L1), AN4005 (small molecule against PD-1/PD-L1), ALPN-202 (PD-L1/CTLA-4 w/CD28), AVA004 (PD-L1), BN-101A (PD-L1), prolgolimab (PD-1), BCD-217 (PD-1/CTLA-4 bi-specific), CCX-559 (PD-L1), CTX-8371 (PD-1/PD-L1 bi-specific), CB213 (PD-1/LAG-3 bi-specific), CA-170 (PD-L1/VISTA), CA-327 (PD-L1/TIM-3), Aurigene's PD-L1/TIGIT, GNR-051 (PD-1), GS19 (PD-L1/TGF-βR2 dual-targetin), HX008, IGM-7354 (PD-L1/IL-15 bi-specific), IMGS-001 (PD-1), MVR-T3011 (IL-12), INCB-086550 (PD-L1), INCB106385 (PD-1/A2A/A2B/CD73), INBRX-105 (PD-L1/CD137 tetravalent bi-specific), IBI322 (PD-L1/CD46 recombinant), IO103 (PD-L1), JS201 (PD-1/TGF-β bi-functional), KD033 (PD-L1/IL-15 bi-functional), GT90008 (PD-L1/TGF-β bi-specific), socazolimab (PD-L1), MCLA-145 (PD-L1/CD137), MT-6402 (PD-L1), ND021 (PD-L1/CD137/HSA tri-specific), OSE-279 (PD-1/IL-7 bi-specific), PH-762-ACT (PD-1), PH-762-TME (PD-1), PRS-344 (PD-L1/CD137 bi-specific), QL1604, RG6139 (PD-1/LAG-3 bi-specific), RG6279 (PD-1 IL-2 variant), RG7769 (PD-1/TIM-3 bi-specific), SL-279252 (PD-L1/OX40), SCT-I10A, STI-A1014 (PD-L1), PSB205 (PD-1/CTLA4 bi-functional), STM418 (PD-1), Sym021 (PD-1), MASCT-I (PD-1), TC-510 (PD-1), MSB2311 (PD-L1), VG-161 (PD-L1/IL-12/IL-15), Y111 (PD-L1/CD3 bi-specific), XmAb20717 (PD-1/CTLA-4 bi-specific), XmAb23104 (PD-1/ICOS bi-specific), YBL-013 (PD-L1/CD3 bi-specific), and combinations thereof. The target of the checkpoint inhibitor, if known, is presented between parentheses after the name of the checkpoint inhibitor.

### II. ANN Classifiers: TME Panel-1 Classifier

In some aspects, the present disclosure provides the methodology to create an artificial neural network (ANN) classifier that is able to stratify (or classify) gene expression samples obtained from a tumor from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) into several TME phenotype classes. The underlying tumor biology of the four TME phenotype classes (i.e., stromal subtypes or phenotypes): IA (immune active), ID (immune desert), A (angiogenic) and IS (immune suppressed), discussed above, can be revealed by application of an ANN. In some aspects, application of the methods disclosed herein can classify a tumor sample or patient into more than one of the TME phenotype classes disclosed herein, e.g., a patient or sample can be biomarker positive for two or more TME phenotype classes.

The ANN takes as input the gene expression values of the genes or subset thereof disclosed herein (i.e. features), and based on the pattern of expression identifies patient samples (i.e., patients) with either predominantly angiogenic expression, predominantly activated immune gene expression, a mixture of both or neither of these expression patterns. These four phenotypic types are predictive of the response to certain types of treatment.

The ANN classifiers disclosed herein can be trained with data corresponding to a set of samples for which gene expression data, e.g., mRNA expression data, corresponding to a gene panel has been obtained. For example, the training set comprises expression data from the genes presented in TABLE 1 and TABLE 2, and any combination thereof. In some aspects, the gene panel comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 84, 86, 87, 88, 89, 90, 91, 92, 93, 94, 95, 96, 97, 98, 99, or 100 genes. In some aspects, the gene panel comprises more than 100 genes. In some aspects, the gene panel comprises between about 10 and about 20, about 20 and about 30, about 30 and about 40, about 40 and about 50, about 50 and about 60, about 60 and about 70, about 70 and about 80, about 80 and about 90, or about 90 and about 100 genes selected from TABLE 1 and TABLE 2. In some aspects, the gene panel comprises a set of genes from TABLE 3 and set of genes from TABLE 4. In some aspects, the gene panel is a gene panel selected from TABLE 5. In some aspects, the gene panel is a gene panel (Geneset) disclosed in FIG. 9A-G.

The classifier of the present disclosure relies on the selection of a specific gene panel as the source of the input data used by the classifier. In some aspects, each one of the genes in a gene panel of the present disclosure is referred to as a “biomarker.” The terms “geneset” and “gene panel” are used interchangeably. In some aspects, the biomarker is a nucleic acid biomarker. The term “nucleic acid biomarker,” as used herein, refers to a nucleic acid (e.g., a gene in a gene panel disclosed herein) that can be detected (e.g., quantified) in a subject or a sample therefrom, e.g., a sample comprising tissues, cells, stroma, cell lysates, and/or constituents thereof, e.g., from a tumor. In some aspects, the term nucleic acid biomarker refers to the presence or absence of a specific sequence of interest (e.g., a nucleic acid variant or a single nucleotide polymorphism) in a nucleic acid (e.g., a gene in a gene panel disclosed herein) that can be detected (e.g., quantified) in a subject or a sample therefrom, e.g., a sample comprising tissues, cells, stroma, cell lysates, and/or constituents thereof, e.g., from a tumor.

The “level” of a nucleic acid biomarker can, in some aspects, refer to the “expression level” of the biomarker, e.g., the level of an RNA or DNA encoded by the nucleic acid sequence of the nucleic acid biomarker in a sample. For example, in some aspects, the expression level of a particular gene disclosed in TABLE 1 or TABLE 2, refers to the amount of mRNA encoding such gene present in a sample obtained from a subject.

In some aspects, the “level” of a nucleic acid biomarker, e.g., an RNA biomarker, can be determined by measuring a downstream output (e.g., an activity level of a target molecule or an expression level of an effector molecule that is modulated, e.g., activated or inhibited, by the nucleic acid biomarker or an expression product, e.g., RNA or DNA, thereof).

In some aspects, the nucleic acid biomarker is an RNA biomarker. An “RNA biomarker,” as used herein, refers to an RNA comprising the nucleic acid sequence of a nucleic acid biomarker of interest, e.g., RNA encoding a gene disclosed in TABLE 1 or TABLE 2.

The “expression level” of an RNA biomarker generally refers to a detected quantity of RNA molecules comprising the nucleic acid sequence of interest present in the subject or sample therefrom, e.g., the quantity of RNA molecules expressed from a DNA molecule (e.g., the genome of the subject or the subject's cancer) comprising the nucleic acid sequence.

In some aspects, the expression level of an RNA biomarker is the quantity of the RNA biomarker in a tumor stromal sample. In some aspects, an RNA biomarker is quantified using PCR (e.g., real-time PCR), sequencing (e.g., deep sequencing or next generation sequencing, e.g., RNA-Seq), or microarray expression profiling or other technologies that utilize RNAse protection in combination with amplification or amplification and new quantitation methods such as RNA-Seq or other methods.

The methods disclosed herein comprise measuring the expression levels of a gene panel selected from a sample, e.g., a biological sample obtained from a subject. See U.S. application Ser. No. 17/089,234, which is incorporated herein by reference in its entirety. Biomarker levels (e.g., expression levels of genes in a gene panel of the present disclosure) can be measured in any biological sample that contains or is suspected to contain one or more of the biomarkers (e.g., RNA biomarkers) disclosed herein, including any tissue sample or biopsy from a subject or patient, e.g., cancer tissue, tumor, and/or stroma of a subject. The source of the tissue sample can be solid tissue, e.g., from a fresh, frozen and/or preserved organ, tissue sample, biopsy, or aspirate. In some aspects, the sample is a cell-free sample, e.g., comprising cell-free nucleic acids (e.g., DNA or RNA). A sample can, in some aspects, comprise compounds that are not naturally intermixed with the tissue in nature such as preservatives, anticoagulants, buffers, fixatives, nutrients, antibiotics or the like.

In some aspects, fresh samples are preferred to archival samples. As used herein, the terms “fresh sample,” “non-archival sample,” and grammatical variants thereof refer to a sample (e.g., a tumor sample from colorectal cancer, gastric cancer, breast cancer, prostate cancer, liver cancer, carcinoma of head and neck, melanoma, ovarian cancer, glioma, glioblastoma, or lung cancer) which has been processed (e.g., to determine mRNA expression) before a predetermined period of time, e.g., one week, after extraction from a subject. In some aspects, a fresh sample has not been frozen. In some aspects, a fresh sample has not been fixed. In some aspects, a fresh sample has been stored for less than about two weeks, less than about one week, or less than six, five, four, three, or two days before processing.

As used herein, the term “archival sample” and grammatical variants thereof refers to a sample (e.g., a tumor sample from colorectal cancer, gastric cancer, breast cancer, prostate cancer, liver cancer, carcinoma of head and neck, melanoma, ovarian cancer, glioma, glioblastoma, or lung cancer) which has been processed (e.g., to determine RNA) after a predetermined period of time, e.g., a week, after extraction from a subject. In some aspects, an archival sample has been frozen. In some aspects, an archival sample has been fixed. In some aspect, an archival sample has a known diagnostic and/or a treatment history. In some aspects, an archival sample has been stored for at least one week, at least one month, at least six months, or at least one year, before processing.

Biomarker levels can, in some instances, be derived from fixed tumor tissue. In some aspects, the sample is preserved as a frozen sample or as formalin-, formaldehyde-, or paraformaldehyde-fixed paraffin-embedded (FFPE) tissue preparation. For example, the sample can be embedded in a matrix, e.g., an FFPE block or a frozen sample. In some aspects, a sample can comprise, e.g., tissue biopsy specimens or surgical specimens. In some aspects, a sample is or comprises cells obtained from a patient.

In some aspects, the sample can be obtained, e.g., from surgical material or from biopsy (e.g., a recent biopsy, a recent biopsy since last progression, or a recent biopsy since the last failed therapy). In some aspects, the biopsy can be archival tissue from a previous line of therapy. In some aspects, the biopsy can be from tissue that is therapy naïve. In some aspects, biological fluids are not used as samples.

The level of expression of the genes in the gene panels described herein can be determined using any method in the art. In some aspects, the RNA levels are determined using sequencing methods, e.g., Next Generation Sequencing (NGS). In some aspects, the NGS is RNA-Seq, EdgeSeq, PCR, Nanostring, or combinations thereof, or any technologies that measure RNA. In some aspects, the RNA measurement methods comprise nuclease protection. Specific methods to determine expression levels of the genes in the gene panels described herein are detailed in the U.S. application Ser. No. 17/089,234, which is incorporated herein by reference in its entirety.

In the ANN classifiers disclosed herein, expression levels for genes in a gene panel acquired from a population of samples (e.g., samples from a clinical study) and their assignments to a TME phenotype class (or a combination thereof, i.e., a sample can be classified not only as biomarker-positive for a single TME phenotype class, but also can be classified as biomarker-positive for two or more TME phenotype classes) obtained according to the populations classifiers disclosed herein can be used as a training set for the ANN. The machine-learning process would yield a model, e.g., an ANN model. Subsequently, expression levels for genes in a gene panel obtained from a sample or samples from a test subject would be used as input for the model, which would classify the subject's tumors into a particular TME phenotype class (or a combination thereof, i.e., a sample can be classified not only as biomarker-positive for a single TME phenotype class, but also can be classified as biomarker-positive for two or more TME phenotype classes).

Standard names, aliases, etc. of proteins and genes designated by identifiers used throughout this disclosure can be identified, for example, via Genecards (www.genecards.org) or Uniprot (www.uniprot.org).

TABLE 1
Angiogenesis signature genes and accession numbers (n = 63)

| Gene | |
|---|---|
| Symbol | Gene Description |
| ABCC9 | ATP binding cassette subfamily C member 9 |
| AFAP1L2 | actin filament associated protein 1 like 2 |
| BACE1 | beta-secretase 1 |
| BGN | Biglycan |
| BMP5 | bone morphogenetic protein 5 |
| COL4A2 | collagen type IV alpha 2 chain |
| COL8A1 | collagen type VIII alpha 1 chain |
| COL8A2 | collagen type VIII alpha 2 chain |
| CPXM2 | carboxypeptidase X, M14 family member 2 |
| CXCL12 | C-X-C motif chemokine ligand 12 |
| EBF1 | early B cell factor 1 |
| ECM2 | extracellular matrix protein 2 |
| EDNRA | endothelin receptor type A |
| ELN | Elastin |
| EPHA3 | EPH receptor A3 |
| FBLN5 | fibulin 5 |
| GNAS | GNAS complex locus |
| GNB4 | G protein subunit beta 4 |
| GUCY1A3 | guanylate cyclase 1 soluble subunit alpha 1 |
| HEY2 | HES related family bHLH transcription factor |
| | with YRPW motif 2 |
| HSPB2 | heat shock protein family B (small) member 2 |
| IL1B | interleukin 1 beta |
| ITGA9 | integrin subunit alpha 9 |
| ITPR1 | inositol 1,4,5-trisphosphate receptor type 1 |
| JAM2 | junctional adhesion molecule 2 |
| JAM3 | junctional adhesion molecule 3 |
| KCNJ8 | potassium voltage-gated channel subfamily J member 8 |
| LAMB2 | laminin subunit beta 2 |
| LHFP | LHFPL tetraspan subfamily member 6 |
| LTBP4 | latent transforming growth factor beta binding protein 4 |
| MEOX1 | mesenchyme homeobox 1 |
| MGP | matrix Gla protein |
| MMP12 | matrix metallopeptidase 12 |
| MMP13 | matrix metallopeptidase 13 |
| NAALAD2 | N-acetylated alpha-linked acidic dipeptidase 2 |
| NFATC1 | nuclear factor of activated T cells 1 |
| NOV | nephroblastoma overexpressed |
| OLFML2A | olfactomedin like 2A |
| PCDH17 | protocadherin 17 |
| PDE5A | phosphodiesterase 5A |
| PDGFRB | platelet derived growth factor receptor beta |
| PEG3 | paternally expressed 3 |
| PLSCR2 | phospholipid scramblase 2 |
| PLXDC2 | plexin domain containing 2 |
| RGS4 | regulator of G protein signaling 4 |
| RGS5 | regulator of G protein signaling 5 |
| RNF144A | ring finger protein 144A |
| RRAS | RAS related |
| RUNX1T1 | RUNX1 translocation partner 1 |
| CAV2 | caveolae associated protein 2 |
| SELP | selectin P |
| SERPINE2 | serpin family E member 2 |
| SGIP1 | SH3 domain GRB2 like endophilin interacting protein 1 |
| SMARCA1 | SWI/SNF related, matrix associated, actin dependent |
| | regulator of chromatin, subfamily a, member 1 |
| SPON1 | spondin 1 |
| STAB2 | stabilin 2 |
| STEAP4 | STEAP4 metalloreductase |
| TBX2 | T-box 2 |
| TEK | TEK receptor tyrosine kinase |
| TGFB2 | transforming growth factor beta 2 |
| TMEM204 | transmembrane protein 204 |
| TTC28 | tetratricopeptide repeat domain 28 |
| UTRN | Utrophin |

TABLE 2
Immune signature genes and accession numbers (n = 61)

| Gene | |
|---|---|
| Symbol | Gene Description |
| AGR2 | anterior gradient 2, protein disulphide |
| | isomerase family member |
| C11orf9 | myelin regulatory factor |
| DUSP4 | dual specificity phosphatase 4 |
| EIF5A | eukaryotic translation initiation factor 5A |
| ETV5 | ETS variant 5 |
| GAD1 | glutamate decarboxylase 1 |
| IQGAP3 | IQ motif containing GTPase activating protein 3 |
| MST1 | macrophage stimulating 1 |
| MT2A | metallothionein 2A |
| MTA2 | metastasis associated 1 family member 2 |
| PLA2G4A | phospholipase A2 group IVA |
| REG4 | regenerating family member 4 |
| SRSF6 | serine and arginine rich splicing factor 6 |
| STRN3 | striatin 3 |
| TRIM7 | tripartite motif containing 7 |
| USF1 | upstream transcription factor 1 |
| ZIC2 | Zic family member 2 |
| C10orf54 | V-set immunoregulatory receptor |
| CCL3 | C-C motif chemokine ligand 3 |
| CCL4 | C-C motif chemokine ligand 4 |
| CD19 | CD19 molecule |
| CD274 | CD274 molecule |
| CD3E | CD3e molecule |
| CD4 | CD4 molecule |
| CD8B | CD8b molecule |
| CTLA4 | cytotoxic T-lymphocyte associated protein 4 |
| CXCL10 | C-X-C motif chemokine ligand 10 |
| IFNA2 | interferon alpha 2 |
| IFNB1 | interferon beta 1 |
| IFNG | interferon gamma |
| LAG3 | lymphocyte activating 3 |
| PDCD1 | programmed cell death 1 |
| PDCD1LG2 | programmed cell death 1 ligand 2 |
| TGFB1 | transforming growth factor beta 1 |
| TIGIT | T cell immunoreceptor with Ig and ITIM domains |
| TNFRSF18 | TNF receptor superfamily member 18 |
| TNFRSF4 | TNF receptor superfamily member 4 |
| TNFSF18 | TNF superfamily member 18 |
| TLR9 | toll like receptor 9 |
| HAVCR2 | hepatitis A virus cellular receptor 2 |
| CD79A | CD79a molecule |
| CXCL11 | C-X-C motif chemokine ligand 11 |
| CXCL9 | C-X-C motif chemokine ligand 9 |
| GZMB | granzyme B |
| IDO1 | indoleamine 2,3-dioxygenase 1 |
| IGLL5 | immunoglobulin lambda like polypeptide 5 |
| ADAMTS4 | ADAM metallopeptidase with thrombospondin |
| | type 1 motif 4 |
| CAPG | capping actin protein, gelsolin like |
| CCL2 | C-C motif chemokine ligand 2 |
| CTSB | cathepsin B |
| FOLR2 | folate receptor beta |
| HFE | homeostatic iron regulator |
| HMOX1 | heme oxygenase 1 |
| HP | Haptoglobin |
| IGFBP3 | insulin like growth factor binding protein 3 |
| MEST | mesoderm specific transcript |
| PLAU | plasminogen activator, urokinase |
| RAC2 | Rac family small GTPase 2 |
| RNH1 | ribonuclease/angiogenin inhibitor 1 |
| SERPINE1 | serpin family E member 1 |
| TIMP1 | TIMP metallopeptidase inhibitor 1 |

TABLE 3
Angiogenesis Signature gene sets

| Panel | N | Gene Symbols |
|---|---|---|
| S1A | 63 | ABCC9, AFAP1L2, BACE1, BGN, BMP5, COL4A2, |
| | | COL8A1, COL8A2, CPXM2, CXCL12, EBF1, |
| | | ECM2, EDNRA, ELN, EPHA3, FBLN5, GNAS, |
| | | GNB4, GUCY1A3, HEY2, HSPB2, IL1B, ITGA9, |
| | | ITPR1, JAM2, JAM3, KCNJ8, LAMB2, LHFP, |
| | | LTBP4, MEOX1, MGP, MMP12, MMP13, |
| | | NAALAD2, NFATC1, NOV, OLFML2A, PCDH17, |
| | | PDE5A, PDGFRB, PEG3, PLSCR2, PLXDC2, |
| | | RGS4, RGS5, RNF144A, RRAS, RUNX1T1, CAV2, SELP, |
| | | SERPINE2, SGIP1, SMARCA1, SPON1, STAB2, STEAP4, |
| | | TBX2, TEK, TGFB2, TMEM204, TTC28, UTRN |
| S1B | 50 | ELN, EPHA3, FBLN5, GNAS, GNB4, GUCY1A3, HEY2, |
| | | HSPB2, IL1B, ITGA9, ITPR1, JAM2, JAM3, |
| | | KCNJ8, LAMB2, LHFP, LTBP4, MEOX1, MGP, |
| | | MMP12, MMP13, NAALAD2, NFATC1, NOV, OLFML2A, |
| | | PCDH17, PDE5A, PDGFRB, PEG3, PLSCR2, |
| | | PLXDC2, RGS4, RGS5, RNF144A, RRAS, |
| | | RUNX1T1, CAV2, SELP, SERPINE2, SGIP1, SMARCA1, |
| | | SPON1, STAB2, STEAP4, TBX2, TEK, |
| | | TGFB2, TMEM204, TTC28, UTRN |
| S1C | 40 | ITPR1, JAM2, JAM3, KCNJ8, LAMB2, LHFP, |
| | | LTBP4, MEOX1, MGP, MMP12, MMP13, NAALAD2, |
| | | NFATC1, NOV, OLFML2A, PCDH17, PDE5A, |
| | | PDGFRB, PEG3, PLSCR2, PLXDC2, RGS4, RGS5, |
| | | RNF144A, RRAS, RUNX1T1, CAV2, SELP, |
| | | SERPINE2, SGIP1, SMARCA1, SPON1, STAB2, |
| | | STEAP4, TBX2, TEK, TGFB2, TMEM204, TTC28, UTRN |
| S1D | 30 | MMP13, NAALAD2, NFATC1, NOV, OLFML2A, PCDH17, |
| | | PDE5A, PDGFRB, PEG3, PLSCR2, PLXDC2, |
| | | RGS4, RGS5, RNF144A, RRAS, RUNX1T1, CAV2, |
| | | SELP, SERPINE2, SGIP1, SMARCA1, SPON1, STAB2, |
| | | STEAP4, TBX2, TEK, TGFB2, TMEM204, TTC28, UTRN |
| S1E | 20 | PLXDC2, RGS4, RGS5, RNF144A, RRAS, RUNX1T1, |
| | | CAV2, SELP, SERPINE2, SGIP1, SMARCA1, SPON1, |
| | | STAB2, STEAP4, TBX2, TEK, TGFB2, TMEM204, |
| | | TTC28, UTRN |
| S1F | 10 | SMARCA1, SPON1, STAB2, STEAP4, TBX2, TEK, |
| | | TGFB2, TMEM204, TTC28, UTRN |

TABLE 4
Immune Signature gene sets

| Panel | N | Gene Symbols |
|---|---|---|
| S2A | 61 | AGR2, C11orf9, DUSP4, EIF5A, ETV5, GAD1, |
| | | IQGAP3, MST1, MT2A, MTA2, PLA2G4A, REG4, |
| | | SRSF6, STRN3, TRIM7, USF1, ZIC2, C10orf54, |
| | | CCL3, CCL4, CD19, CD274, CD3E, CD4, CD8B, |
| | | CTLA4, CXCL10, IFNA2, IFNB1, IFNG, LAG3, |
| | | PDCD1, PDCD1LG2, TGFB1, TIGIT, TNFRSF18, |
| | | TNFRSF4, TNFSF18, TLR9, HAVCR2, CD79A, |
| | | CXCL11, CXCL9, GZMB, IDO1, IGLL5, ADAMTS4, |
| | | CAPG, CCL2, CTSB, FOLR2, HFE, HMOX1, HP, |
| | | IGFBP3, MEST, PLAU, RAC2, RNH1, SERPINE1, TIMP1 |
| S2B | 50 | REG4, SRSF6, STRN3, TRIM7, USF1, ZIC2, C10orf54, |
| | | CCL3, CCL4, CD19, CD274, CD3E, CD4, CD8B, CTLA4, |
| | | CXCL10, IFNA2, IFNB1, IFNG, LAG3, PDCD1, |
| | | PDCD1LG2, TGFB1, TIGIT, TNFRSF18, TNFRSF4, |
| | | TNFSF18, TLR9, HAVCR2, CD79A, CXCL11, CXCL9, |
| | | GZMB, IDO1, IGLL5, ADAMTS4, CAPG, CCL2, CTSB, |
| | | FOLR2, HFE, HMOX1, HP, IGFBP3, |
| | | MEST, PLAU, RAC2, RNH1, SERPINE1, TIMP1 |
| S2C | 40 | CD274, CD3E, CD4, CD8B, CTLA4, CXCL10, IFNA2, |
| | | IFNB1, IFNG, LAG3, PDCD1, PDCD1LG2, TGFB1, |
| | | TIGIT, TNFRSF18, TNFRSF4, TNFSF18, TLR9, |
| | | HAVCR2, CD79A, CXCL11, CXCL9, GZMB, IDO1, |
| | | IGLL5, ADAMTS4, CAPG, CCL2, CTSB, FOLR2, HFE, |
| | | HMOX1, HP, IGFBP3, MEST, PLAU, RAC2, |
| | | RNH1, SERPINE1, TIMP1 |
| S2D | 30 | PDCD1, PDCD1LG2, TGFB1, TIGIT, TNFRSF18, |
| | | TNFRSF4, TNFSF18, TLR9, HAVCR2, CD79A, |
| | | CXCL11, CXCL9, GZMB, IDO1, IGLL5, |
| | | ADAMTS4, CAPG, CCL2, CTSB, FOLR2, HFE, |
| | | HMOX1, HP, IGFBP3, MEST, PLAU, RAC2, |
| | | RNH1, SERPINE1, TIMP1 |
| S2E | 20 | CXCL11, CXCL9, GZMB, IDO1, IGLL5, ADAMTS4, |
| | | CAPG, CCL2, CTSB, FOLR2, HFE, HMOX1, HP, |
| | | IGFBP3, MEST, PLAU, RAC2, RNH1, |
| | | SERPINE1, TIMP1 |
| S2F | 10 | HFE, HMOX1, HP, IGFBP3, MEST, PLAU, RAC2, |
| | | RNH1, SERPINE1, TIMP1 |

In some aspects, a gene panel to be used as part of the training set or model input in the ANN classifier disclosed herein comprises ABCC9, ADAMTS4, AFAP1L2, AGR2, BACE1, BGN, BMP5, C11ORF9, CAPG, CAVIN2, CCL2, CCL3, CCL4, CD19, CD274, CD3E, CD4, CD79A, CD8B, COL4A2, COL8A1, COL8A2, CPXM2, CTLA4, CTSB, CXCL10, CXCL11, CXCL12, CXCL9, DUSP4, EBF1, ECM2, EDNRA, EIF5A, ELN, EPHA3, ETV5, FBLN5, FOLR2, GAD1, GNAS, GNB4, GUCY1A1, GZMB, HAVCR2, HEY2, HFE, HMOX1, HP, HSPB2, IDO1, IFNA2, IFNB1, IFNG, IGFBP3, IGLL5, IL1B, IQGAP3, ITGA9, ITPR1, JAM2, JAM3, KCNJ8, LAG3, LAMB2, LHFPL6, LTBP4, MEOX1, MEST, MGP, MMP12, MMP13, MST1, MT2A, MTA2, NAALAD2, NFATC1, NOV, OLFML2A, PCDH17, PDCD1, PDCD1LG2, PDE5A, PDGFRB, PEG3, PLA2G4A, PLAU, PLSCR2, PLXDC2, RAC2, REG4, RGS4, RGS5, RNF144A, RNH1, RRAS, RUNX1T1, SELP, SERPINE1, SERPINE2, SGIP1, SMARCA1, SPON1, SRSF6, STAB2, STEAP4, STRN3, TBX2, TEK, TGFB1, TGFB2, TIGIT, TIMP1, TLR9, TMEM204, TNFRSF18, TNFRSF4, TNFSF18, TRIM7, TTC28, USF1, UTRN, VSIR, and ZIC2. In some aspects, a gene panel to be used as part of the training set or model input in the ANN classifier disclosed herein consists of ABCC9, ADAMTS4, AFAP1L2, AGR2, BACE1, BGN, BMP5, C11ORF9, CAPG, CAVIN2, CCL2, CCL3, CCL4, CD19, CD274, CD3E, CD4, CD79A, CD8B, COL4A2, COL8A1, COL8A2, CPXM2, CTLA4, CTSB, CXCL10, CXCL11, CXCL12, CXCL9, DUSP4, EBF1, ECM2, EDNRA, EIF5A, ELN, EPHA3, ETV5, FBLN5, FOLR2, GAD1, GNAS, GNB4, GUCY1A1, GZMB, HAVCR2, HEY2, HFE, HMOX1, HP, HSPB2, IDO1, IFNA2, IFNB1, IFNG, IGFBP3, IGLL5, IL1B, IQGAP3, ITGA9, ITPR1, JAM2, JAM3, KCNJ8, LAG3, LAMB2, LHFPL6, LTBP4, MEOX1, MEST, MGP, MMP12, MMP13, MST1, MT2A, MTA2, NAALAD2, NFATC1, NOV, OLFML2A, PCDH17, PDCD1, PDCD1LG2, PDE5A, PDGFRB, PEG3, PLA2G4A, PLAU, PLSCR2, PLXDC2, RAC2, REG4, RGS4, RGS5, RNF144A, RNH1, RRAS, RUNX1T1, SELP, SERPINE1, SERPINE2, SGIP1, SMARCA1, SPON1, SRSF6, STAB2, STEAP4, STRN3, TBX2, TEK, TGFB1, TGFB2, TIGIT, TIMP1, TLR9, TMEM204, TNFRSF18, TNFRSF4, TNFSF18, TRIM7, TTC28, USF1, UTRN, VSIR, and ZIC2.

In some aspects, a gene panel to be used as part of the training set or model input in the ANN classifier disclosed herein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124 genes selected from the group consisting of ABCC9, ADAMTS4, AFAP1L2, AGR2, BACE1, BGN, BMP5, C11ORF9, CAPG, CAVIN2, CCL2, CCL3, CCL4, CD19, CD274, CD3E, CD4, CD79A, CD8B, COL4A2, COL8A1, COL8A2, CPXM2, CTLA4, CTSB, CXCL10, CXCL11, CXCL12, CXCL9, DUSP4, EBF1, ECM2, EDNRA, EIF5A, ELN, EPHA3, ETV5, FBLN5, FOLR2, GAD1, GNAS, GNB4, GUCY1A1, GZMB, HAVCR2, HEY2, HFE, HMOX1, HP, HSPB2, IDO1, IFNA2, IFNB1, IFNG, IGFBP3, IGLL5, IL1B, IQGAP3, ITGA9, ITPR1, JAM2, JAM3, KCNJ8, LAG3, LAMB2, LHFPL6, LTBP4, MEOX1, MEST, MGP, MMP12, MMP13, MST1, MT2A, MTA2, NAALAD2, NFATC1, NOV, OLFML2A, PCDH17, PDCD1, PDCD1LG2, PDE5A, PDGFRB, PEG3, PLA2G4A, PLAU, PLSCR2, PLXDC2, RAC2, REG4, RGS4, RGS5, RNF144A, RNH1, RRAS, RUNX1T1, SELP, SERPINE1, SERPINE2, SGIP1, SMARCA1, SPON1, SRSF6, STAB2, STEAP4, STRN3, TBX2, TEK, TGFB1, TGFB2, TIGIT, TIMP1, TLR9, TMEM204, TNFRSF18, TNFRSF4, TNFSF18, TRIM7, TTC28, USF1, UTRN, VSIR, and ZIC2.

In some aspects, a gene panel to be used as part of the training set or model input in the ANN classifier disclosed herein consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124 genes selected from the group consisting of ABCC9, ADAMTS4, AFAP1L2, AGR2, BACE1, BGN, BMP5, C11ORF9, CAPG, CAVIN2, CCL2, CCL3, CCL4, CD19, CD274, CD3E, CD4, CD79A, CD8B, COL4A2, COL8A1, COL8A2, CPXM2, CTLA4, CTSB, CXCL10, CXCL11, CXCL12, CXCL9, DUSP4, EBF1, ECM2, EDNRA, EIF5A, ELN, EPHA3, ETV5, FBLN5, FOLR2, GAD1, GNAS, GNB4, GUCY1A1, GZMB, HAVCR2, HEY2, HFE, HMOX1, HP, HSPB2, IDO1, IFNA2, IFNB1, IFNG, IGFBP3, IGLL5, IL1B, IQGAP3, ITGA9, ITPR1, JAM2, JAM3, KCNJ8, LAG3, LAMB2, LHFPL6, LTBP4, MEOX1, MEST, MGP, MMP12, MMP13, MST1, MT2A, MTA2, NAALAD2, NFATC1, NOV, OLFML2A, PCDH17, PDCD1, PDCD1LG2, PDE5A, PDGFRB, PEG3, PLA2G4A, PLAU, PLSCR2, PLXDC2, RAC2, REG4, RGS4, RGS5, RNF144A, RNH1, RRAS, RUNX1T1, SELP, SERPINE1, SERPINE2, SGIP1, SMARCA1, SPON1, SRSF6, STAB2, STEAP4, STRN3, TBX2, TEK, TGFB1, TGFB2, TIGIT, TIMP1, TLR9, TMEM204, TNFRSF18, TNFRSF4, TNFSF18, TRIM7, TTC28, USF1, UTRN, VSIR, and ZIC2.

In some aspects, an ANN classifier disclosed herein, e.g., the TME Panel-1 Classifier, has been trained using a geneset provided in the table below.

TABLE 5
Genesets (gene panels) for use in ANN training, e.g., to
train the TME Panel-1 Classifier and variants thereof.

| | GENES |
|---|---|
| Training set 1 | ABCC9, ADAMTS4, AFAP1L2, AGR2, BACE1, |
| (n = 124) | BGN, BMP5, C11ORF9, CAPG, CAVIN2, |
| | CCL2, CCL3, CCL4, CD19, CD274, CD3E, |
| | CD4, CD79A, CD8B, COL4A2, COL8A1, |
| | COL8A2, CPXM2, CTLA4, CTSB, CXCL10, |
| | CXCL11, CXCL12, CXCL9, DUSP4, EBF1, |
| | ECM2, EDNRA, EIF5A, ELN, EPHA3, ETV5, |
| | FBLN5, FOLR2, GAD1, GNAS, GNB4, |
| | GUCY1A1, GZMB, HAVCR2, HEY2, HFE, |
| | HMOX1, HP, HSPB2, IDO1, IFNA2, |
| | IFNB1, IFNG, IGFBP3, IGLL5, IL1B, |
| | IQGAP3, ITGA9, ITPR1, JAM2, JAM3, |
| | KCNJ8, LAG3, LAMB2, LHFPL6, |
| | LTBP4, MEOX1, MEST, MGP, MMP12, |
| | MMP13, MST1, MT2A, MTA2, NAALAD2, |
| | NFATC1, NOV, OLFML2A, PCDH17, |
| | PDCD1, PDCD1LG2, PDE5A, PDGFRB, |
| | PEG3, PLA2G4A, PLAU, PLSCR2, PLXDC2, |
| | RAC2, REG4, RGS4, RGS5, RNF144A, |
| | RNH1, RRAS, RUNX1T1, SELP, SERPINE1, |
| | SERPINE2, SGIP1, SMARCA1, SPON1, SRSF6, |
| | STAB2, STEAP4, STRN3, TBX2, TEK, TGFB1, |
| | TGFB2, TIGIT, TIMP1, TLR9, TMEM204, |
| | TNFRSF18, TNFRSF4, TNFSF18, TRIM7, |
| | TTC28, USF1, UTRN, VSIR, ZIC2 |
| Training set 2 | ABCC9, ADAMTS4, AFAP1L2, AGR2, BACE1, |
| (n = 119) | BGN, BMP5, C11ORF9, CAPG, CAVIN2, CCL2, |
| | CCL3, CCL4, CD19, CD274, CD3E, CD4, |
| | CD79A, CD8B, COL8A1, COL8A2, CPXM2, |
| | CTLA4, CTSB, CXCL10, CXCL11, CXCL12, |
| | CXCL9, DUSP4, EBF1, ECM2, EDNRA, |
| | EIF5A, ELN, EPHA3, ETV5, FBLN5, |
| | GAD1, GNAS, GNB4, GUCY1A1, GZMB, |
| | HAVCR2, HEY2, HFE, HMOX1, HP, HSPB2, |
| | IDO1, IFNA2, IFNB1, IFNG, IGFBP3, |
| | IGLL5, IL1B, IQGAP3, ITGA9, JAM2, |
| | JAM3, KCNJ8, LAG3, LAMB2, LHFPL6, |
| | LTBP4, MEOX1, MEST, MGP, MMP12, |
| | MMP13, MST1, MT2A, MTA2, NAALAD2, |
| | NFATC1, NOV, OLFML2A, PCDH17, PDCD1, |
| | PDCD1LG2, PDE5A, PDGFRB, PEG3, |
| | PLAU, PLSCR2, PLXDC2, RAC2, REG4, |
| | RGS4, RGS5, RNF144A, RNH1, RRAS, |
| | RUNX1T1, SELP, SERPINE1, SERPINE2, |
| | SGIP1, SMARCA1, SPON1, SRSF6, STAB2, |
| | STEAP4, STRN3, TBX2, TEK, TGFB1, |
| | TGFB2, TIGIT, TIMP1, TLR9, TNFRSF18, |
| | TNFRSF4, TNFSF18, TRIM7, TTC28, |
| | USF1, UTRN, VSIR, ZIC2 |
| Training set 3 | ABCC9, ADAMTS4, AFAP1L2, AGR2, BACE1, |
| (n = 114) | BGN, BMP5, C11ORF9, CAPG, CAVIN2, |
| | CCL2, CCL3, CCL4, CD19, CD274, |
| | CD3E, CD4, CD79A, CD8B, COL8A2, |
| | CPXM2, CTLA4, CTSB, CXCL10, CXCL11, |
| | CXCL12, CXCL9, DUSP4, EBF1, ECM2, |
| | EDNRA, EIF5A, ELN, EPHA3, ETV5, |
| | FBLN5, GAD1, GNAS, GNB4, GUCY1A1, |
| | GZMB, HAVCR2, HEY2, HFE, HMOX1, |
| | HP, IDO1, IFNA2, IFNB1, IFNG, |
| | IGFBP3, IGLL5, IL1B, IQGAP3, ITGA9, |
| | JAM2, JAM3, KCNJ8, LAG3, LAMB2, |
| | LHFPL6, LTBP4, MEOX1, MEST, MGP, |
| | MMP12, MMP13, MST1, MT2A, MTA2, |
| | NAALAD2, NFATC1, NOV, PCDH17, PDCD1, |
| | PDCD1LG2, PDE5A, PDGFRB, PEG3, |
| | PLAU, PLSCR2, PLXDC2, RAC2, REG4, |
| | RGS4, RGS5, RNF144A, RNH1, RRAS, |
| | RUNX1T1, SELP, SERPINE2, SGIP1, |
| | SMARCA1, SPON1, SRSF6, STAB2, |
| | STEAP4, STRN3, TBX2, TEK, TGFB1, |
| | TGFB2, TIGIT, TIMP1, TLR9, |
| | TNFRSF18, TNFRSF4, TRIM7, TTC28, |
| | USF1, UTRN, VSIR, ZIC2 |
| Training set 4 | ABCC9, ADAMTS4, AFAP1L2, AGR2, |
| (n = 106) | BACE1, BGN, BMP5, C11ORF9, CAPG, |
| | CAVIN2, CCL2, CCL3, CCL4, CD19, |
| | CD274, CD3E, CD4, CD79A, CD8B, |
| | CPXM2, CTLA4, CTSB, CXCL10, CXCL11, |
| | CXCL12, CXCL9, DUSP4, EBF1, ECM2, |
| | EDNRA, EIF5A, ELN, EPHA3, ETV5, |
| | FBLN5, GAD1, GNAS, GNB4, GZMB, |
| | HAVCR2, HEY2, HFE, HMOX1, HP, IDO1, |
| | IFNA2, IFNB1, IFNG, IGFBP3, IGLL5, |
| | IL1B, IQGAP3, ITGA9, JAM2, JAM3, |
| | KCNJ8, LAG3, LAMB2, LTBP4, MEOX1, |
| | MEST, MGP, MMP12, MMP13, MST1, |
| | MT2A, MTA2, NFATC1, NOV, PCDH17, |
| | PDCD1, PDE5A, PDGFRB, PEG3, PLAU, |
| | PLSCR2, PLXDC2, RAC2, REG4, RGS4, |
| | RGS5, RNH1, RRAS, RUNX1T1, SELP, |
| | SGIP1, SMARCA1, SPON1, SRSF6, |
| | STAB2, STEAP4, STRN3, TBX2, TEK, |
| | TGFB1, TGFB2, TIGIT, TIMP1, TLR9, |
| | TNFRSF4, TRIM7, TTC28, USF1, |
| | UTRN, VSIR, ZIC2 |
| Training set 5 | ABCC9, AFAP1L2, BACE1, BGN, BMP5, |
| (n = 98) | COL4A2, COL8A1, COL8A2, CPXM2, CXCL12, |
| | EBF1, ECM2, EDNRA, ELN, EPHA3, FBLN5, |
| | GNAS, GNB4, GUCY1A3, HEY2, HSPB2, |
| | IL1B, ITGA9, ITPR1, JAM2, JAM3, |
| | KCNJ8, LAMB2, LHFP, LTBP4, |
| | MEOX1, MGP, MMP12, MMP13, NAALAD2, |
| | NFATC1, NOV, OLFML2A, PCDH17, PDE5A, |
| | PDGFRB, PEG3, PLSCR2, PLXDC2, RGS4, |
| | RGS5, RNF144A, RRAS, RUNX1T1, CAV2, |
| | SELP, SERPINE2, SGIP1, SMARCA1, |
| | SPON1, STAB2, STEAP4, TBX2, TEK, |
| | TGFB2, TMEM204, TTC28, UTRN, AGR2, |
| | C11orf9, DUSP4, EIF5A, ETV5, GAD1, |
| | IQGAP3, MST1, MT2A, MTA2, PLA2G4A, |
| | REG4, SRSF6, STRN3, TRIM7, USF1, |
| | ZIC2, C10orf54, CCL3, CCL4, CD19, |
| | CD274, CD3E, CD4, CD8B, CTLA4, |
| | CXCL10, IFNA2, IFNB1, IFNG, LAG3, |
| | PDCD1, PDCD1LG2, TGFB1, TIGIT |
| Training set 6 | ELN, EPHA3, FBLN5, GNAS, GNB4, |
| (n = 98) | GUCY1A3, HEY2, HSPB2, IL1B, ITGA9, |
| | ITPR1, JAM2, JAM3, KCNJ8, LAMB2, |
| | LHFP, LTBP4, MEOX1, MGP, MMP12, |
| | MMP13, NAALAD2, NFATC1, NOV, |
| | OLFML2A, PCDH17, PDE5A, PDGFRB, |
| | PEG3, PLSCR2, PLXDC2, RGS4, RGS5, |
| | RNF144A, RRAS, RUNX1T1, CAV2, SELP, |
| | SERPINE2, SGIP1, SMARCA1, SPON1, |
| | STAB2, STEAP4, TBX2, TEK, TGFB2, |
| | TMEM204, TTC28, UTRN, REG4, SRSF6, |
| | STRN3, TRIM7, USF1, ZIC2, C10orf54, |
| | CCL3, CCL4, CD19, CD274, CD3E, CD4, |
| | CD8B, CTLA4, CXCL10, IFNA2, IFNB1, |
| | IFNG, LAG3, PDCD1, PDCD1LG2, TGFB1, |
| | TIGIT, TNFRSF18, TNFRSF4, TNFSF18, |
| | TLR9, HAVCR2, CD79A, CXCL11, CXCL9, |
| | GZMB, IDO1, IGLL5, ADAMTS4, CAPG, |
| | CCL2, CTSB, FOLR2, HFE, HMOX1, HP, |
| | IGFBP3, MEST, PLAU, RAC2, RNH1 |
| Training set 7 | ITPR1, JAM2, JAM3, KCNJ8, LAMB2, |
| (n = 97) | LHFP, LTBP4, MEOX1, MGP, MMP12, |
| | MMP13, NAALAD2, NFATC1, NOV, OLFML2A, |
| | PCDH17, PDE5A, PDGFRB, PEG3, PLSCR2, |
| | PLXDC2, RGS4, RGS5, RNF144A, RRAS, |
| | RUNX1T1, CAV2, SELP, SERPINE2, SGIP1, |
| | SMARCA1, SPON1, STAB2, STEAP4, TBX2, |
| | TEK, TGFB2, TMEM204, TTC28, UTRN, |
| | AGR2, C11orf9, DUSP4, EIF5A, ETV5, |
| | GAD1, IQGAP3, MST1, MT2A, MTA2, |
| | PLA2G4A, REG4, SRSF6, STRN3, TRIM7, |
| | USF1, ZIC2, C10orf54, CCL3, CCL4, |
| | CD19, CD274, CD3E, CD4, CD8B, CTLA4, |
| | CXCL10, IFNA2, IFNB1, IFNG, LAG3, |
| | PDCD1, PDCD1LG2, TGFB1, TIGIT, |
| | TNFRSF18, TNFRSF4, TNFSF18, TLR9, |
| | HAVCR2, CD79A, CXCL11, CXCL9, GZMB, |
| | IDO1, IGLL5, ADAMTS4, CAPG, CCL2, |
| | CTSB, FOLR2, HFE, HMOX1, HP, |
| | IGFBP3, MEST, PLAU |
| Training set 8 | CD19, CD274, CD3E, CD4, EDNRA, |
| (n = 97) | EPHA3, FBLN5, FOLR2, GAD1, GNB4, |
| | GUCY1A3, GZMB, HAVCR2, HMOX1, |
| | HP, HSPB2, IDO1, IFNG, IGFBP3, |
| | IGLL5, IL1B, IQGAP3, ITGA9, ITPR1, |
| | JAM2, JAM3, KCNJ8, LAG3, LAMB2, |
| | LHFP, CD79A, COL4A2, COL8A2, CPXM2, |
| | CTSB, CXCL10, CXCL11, CXCL12, |
| | CXCL9, DUSP4, EBF1, LTBP4, |
| | MEOX1, AFAP1L2, SMARCA1, SPON1, |
| | STEAP4, STRN3, TBX2, TEK, TGFB2, |
| | TIGIT, TIMP1, TLR9, TMEM204, AGR2, |
| | BACE1, BGN, BMP5, C10orf54, CAPG, |
| | CAV2, CCL2, CCL3, CCL4, MEST, MGP, |
| | MMP13, MST1, MT2A, NFATC1, OLFML2A, |
| | PCDH17, PDCD1LG2, PDE5A, PDGFRB, |
| | PEG3, PLA2G4A, PLAU, PLSCR2, PLXDC2, |
| | RAC2, REG4, RGS4, RGS5, RRAS, |
| | RUNX1T1, SELP, SERPINE1, SGIP1, |
| | TNFRSF18, TNFRSF4, TNFSF18, TRIM7, |
| | TTC28, UTRN, ZIC2 |
| Training set 9 | MMP13, NAALAD2, NFATC1, NOV, OLFML2A, |
| (n = 87) | PCDH17, PDE5A, PDGFRB, PEG3, PLSCR2, |
| | PLXDC2, RGS4, RGS5, RNF144A, RRAS, |
| | RUNX1T1, CAV2, SELP, SERPINE2, SGIP1, |
| | SMARCA1, SPON1, STAB2, STEAP4, TBX2, TEK, |
| | TGFB2, TMEM204, TTC28, UTRN, REG4, SRSF6, |
| | STRN3, TRIM7, USF1, ZIC2, C10orf54, CCL3, |
| | CCL4, CD19, CD274, CD3E, CD4, CD8B, CTLA4, |
| | CXCL10, IFNA2, IFNB1, IFNG, LAG3, PDCD1, |
| | PDCD1LG2, TGFB1, TIGIT, TNFRSF18, TNFRSF4, |
| | TNFSF18, TLR9, HAVCR2, CD79A, CXCL11, |
| | CXCL9, GZMB, IDO1, IGLL5, ADAMTS4, CAPG, |
| | CCL2, CTSB, FOLR2, HFE, HMOX1, HP, IGFBP3, |
| | MEST, PLAU, RAC2, RNH1, SERPINE1, TIMP1, |
| | AGR2, C11orf9, DUSP4, EIF5A, ETV5, |
| | GAD1, IQGAP3 |
| Training set 10 | CPXM2, CTSB, CXCL10, CXCL11, CXCL12, |
| (n = 86) | CXCL9, DUSP4, EBF1, EDNRA, EPHA3, FBLN5, |
| | FOLR2, GAD1, GNB4, GUCY1A3, GZMB, HAVCR2, |
| | HMOX1, HP, HSPB2, IDO1, IFNG, IGFBP3, |
| | LTBP4, MEOX1, MEST, MGP, MMP13, AFAP1L2, |
| | OLFML2A, PCDH17, PDCD1LG2, PDE5A, |
| | SMARCA1, SPON1, STEAP4, STRN3, TBX2, |
| | TEK, TGFB2, TIGIT, AGR2, BACE1, BGN, |
| | BMP5, C10orf54, CAPG, CAV2, CCL2, |
| | CCL3, CCL4, CD19, PDGFRB, PEG3, |
| | PLA2G4A, PLAU, PLSCR2, PLXDC2, |
| | RAC2, REG4, RGS4, RGS5, RRAS, RUNX1T1, |
| | SELP, SERPINE1, SGIP1, CD274, CD3E, |
| | CD4, CD79A, COL4A2, COL8A2, MST1, |
| | MT2A, NFATC1, TIMP1, TLR9, TMEM204, |
| | TNFRSF18, TNFRSF4, TNFSF18, TRIM7, |
| | TTC28, UTRN, ZIC2 |
| Training set 11 | EPHA3, FBLN5, FOLR2, GAD1, GNB4, GUCY1A3, |
| (n = 79) | GZMB, HAVCR2, HMOX1, HP, HSPB2, IDO1, |
| | IFNG, IGFBP3, IGLL5, IL1B, IQGAP3, |
| | ITGA9, ITPR1, CD3E, CD4, CD79A, COL4A2, |
| | COL8A2, CPXM2, CTSB, CXCL10, CXCL11, |
| | CXCL12, CXCL9, DUSP4, EBF1, EDNRA, |
| | NFATC1, OLFML2A, PCDH17, PDCD1LG2, |
| | PDE5A, PDGFRB, PEG3, PLA2G4A, PLAU, |
| | PLSCR2, JAM2, JAM3, KCNJ8, LAG3, LAMB2, |
| | LHFP, LTBP4, MEOX1, MEST, MGP, MMP13, |
| | MST1, MT2A, AFAP1L2, AGR2, BACE1, BGN |
| | BMP5, C10orf54, CAPG, CAV2, CCL2, CCL3, |
| | CCL4, CD19, CD274, PLXDC2, RAC2, REG4, |
| | RGS4, RGS5, RRAS, RUNX1T1, SELP, |
| | SERPINE1, SGIP1 |
| Training set 12 | LAG3, LAMB2, LHFP, PCDH17, PDCD1LG2, |
| (n = 68) | PDE5A, PDGFRB, PEG3, PLA2G4A, PLAU, |
| | PLSCR2, PLXDC2, RAC2, REG4, RGS4, CCL4, |
| | CXCL11, CXCL12, CXCL9, DUSP4, EBF1, |
| | EDNRA, EPHA3, FBLN5, FOLR2, GAD1, IGLL5, |
| | IL1B, IQGAP3, ITGA9, ITPR1, JAM2, JAM3, |
| | RGS5, RRAS, RUNX1T1, SELP, SERPINE1, |
| | SGIP1, SMARCA1, SPON1, STEAP4, STRN3, |
| | TBX2, TEK, TGFB2, TIGIT, TIMP1, TLR9, |
| | AFAP1L2, AGR2, BACE1, BGN, BMP5, |
| | C10orf54, CAPG, CAV2, CCL2, CCL3, KCNJ8, |
| | TMEM204, TNFRSF18, TNFRSF4, TNFSF18, |
| | TRIM7, TTC28, UTRN, ZIC2 |
| Training set 13 | FBLN5, FOLR2, GAD1, GNB4, GUCY1A3, |
| (n = 68) | GZMB, HAVCR2, HMOX1, HP, HSPB2, IDO1, |
| | IFNG, IGFBP3, LTBP4, MEOX1, MEST, MGP, |
| | CCL3, CCL4, CD19, CD274, CD3E, CD4, |
| | CD79A, COL4A2, COL8A2, CPXM2, CTSB, |
| | CXCL10, CXCL11, CXCL12, CXCL9, DUSP4, |
| | EBF1, EDNRA, EPHA3, OLFML2A, PCDH17, |
| | PDCD1LG2, PDE5A, PDGFRB, PEG3, PLA2G4A, |
| | MMP13, MST1, MT2A, NFATC1, AFAP1L2, |
| | AGR2, BACE1, BGN, BMP5, C10orf54, |
| | CAPG, CAV2, CCL2, PLAU, PLSCR2, |
| | PLXDC2, RAC2, REG4, RGS4, RGS5, |
| | RRAS, RUNX1T1, SELP, SERPINE1, SGIP1 |
| Training set 14 | GAD1, GNB4, GUCY1A3, GZMB, HAVCR2, |
| (n = 61) | HMOX1, HP, HSPB2, IDO1, IFNG, IGFBP3, |
| | IGLL5, IL1B, IQGAP3, ITGA9, ITPR1, |
| | CD19, CD274, CD3E, CD4, CD79A, |
| | COL4A2, COL8A2, CPXM2, CTSB, CXCL10, |
| | CXCL11, CXCL12, CXCL9, DUSP4, EBF1, |
| | EDNRA, EPHA3, FBLN5, JAM2, JAM3, |
| | KCNJ8, LAG3, AFAP1L2, AGR2, BACE1, |
| | BGN, BMP5, C10orf54, CAPG, CAV2, |
| | CCL2, CCL3, CCL4, FOLR2, LAMB2, |
| | LHFP, LTBP4, MEOX1, MEST, MGP, |
| | MMP13, MST1, MT2A, NFATC1, |
| | OLFML2A |
| Training set 15 | COL8A2, CPXM2, CTSB, GZMB, HAVCR2, |
| (n = 51) | HMOX1, HP, HSPB2, IDO1, IFNG, IGFBP3, |
| | IGLL5, IL1B, IQGAP3, ITGA9, ITPR1, |
| | JAM2, AFAP1L2, AGR2, CXCL10, CXCL11, |
| | CXCL12, CXCL9, DUSP4, EBF1, EDNRA, |
| | EPHA3, FBLN5, FOLR2, GAD1, GNB4, |
| | GUCY1A3, BACE1, BGN, BMP5, C10orf54, |
| | CAPG, CAV2, CCL2, CCL3, CCL4, CD19, |
| | CD274, CD3E, CD4, CD79A, COL4A2, |
| | JAM3, KCNJ8, LAG3, LAMB2, LHFP |
| Training set 16 | CTSB, CXCL10, CXCL11, HMOX1, HP, |
| (n = 41) | HSPB2, IDO1, AFAP1L2, AGR2, BACE1, |
| | BGN, BMP5, C10orf54, CAPG, CAV2, |
| | CCL2, CCL3, CCL4, CD19, CXCL12, |
| | CXCL9, DUSP4, EBF1, EDNRA, EPHA3, |
| | FBLN5, FOLR2, GAD1, GNB4, GUCY1A3, |
| | GZMB, HAVCR2, CD274, CD3E, CD4, |
| | CD79A, COL4A2, COL8A2, CPXM2, |
| | IFNG, IGFBP3 |
| Training set 17 | CD79A, COL4A2, CD19, CD274, CAV2, |
| (n = 31) | CCL2, CCL3, CCL4, CXCL11, CXCL12, |
| | CXCL9, DUSP4, EBF1, EDNRA, EPHA3, |
| | FBLN5, FOLR2, CD3E, CD4, CXCL10, |
| | COL8A2, CPXM2, CTSB, AFAP1L2, AGR2, |
| | BACE1, BGN, BMP5, C10orf54, |
| | CAPG, GAD1 |

In some aspects, the training dataset comprises further variables for each sample, for example the sample classification according to a population-based classifier disclosed in U.S. application Ser. No. 17/089,234, which is incorporated herein by reference in its entirety. In other aspects, the training data comprises data about the sample such as type of treatment administered to the subject, dosage, dose regimen, administration route, presence or absence of co-therapies, response to the therapy (e.g., complete response, partial response or lack of response), age, body weight, gender, ethnicity, tumor size, tumor stage, presence or absence of biomarkers, etc.

In some aspects, the gene panel (e.g., a gene panel to determine an Angiogenesis Signature score or an Immune Signature score in an ANN classifier disclosed herein, e.g., TME Panel-1, or a gene panel to be used as part of the training set or model input in an ANN classifier herein, e.g., TME Panel-1) comprises the genes present in a geneset disclosed in FIG. 9A-G. In some aspects, the gene panel (e.g., a gene panel to determine an Angiogenesis Signature score or an Immune Signature score in an ANN classifier disclosed herein, e.g., TME Panel-1, or a gene panel to be used as part of the training set or model input in an ANN classifier herein, e.g., TME Panel-1) consists of the genes present in a geneset disclosed in FIG. 9A-G. In some aspects, the gene panel (e.g., a gene panel to determine an Angiogenesis Signature score or an Immune Signature score in an ANN classifier disclosed herein, e.g., TME Panel-1, or a gene panel to be used as part of the training set or model input in an ANN classifier herein, e.g., TME Panel-1) does not comprise the genes present in a geneset disclosed in FIG. 9A-G. In some aspects, the gene panel (e.g., a gene panel to determine an Angiogenesis Signature score or an Immune Signature score in an ANN classifier disclosed herein, e.g., TME Panel-1, or a gene panel to be used as part of the training set or model input in an ANN classifier herein, e.g., TME Panel-1) does not consist of the genes present in a geneset disclosed in FIG. 9A-G.

In some aspects, it is helpful to select genes for the training dataset on the basis of a combination of factors including p value, fold change, and coefficient of variation as would be understood by a person skilled in the art. In some aspects, the use of one or more selection criteria and subsequent rankings permits the selection of the top 2.5%, 5%, 7.5%, 10%, 12.5%, 15%, 17.5%, 20%, 30%, 40%, 50% or more of the ranked genes in a gene panel for input into the model. As would be understood, one can select therefore all of the individually identified gene or subsets of the genes in TABLE 1 and TABLE 2, and test all possible combinations of the selected genes to identify useful combinations of genes to generate a predictive model. A selection criterion to determine the number of selected individual genes to test in combination, and to select the number of possible combinations of genes will depend upon the resources available for obtaining the gene data and/or the computer resources available for calculating and evaluating classifiers resulting from the model.

In some aspects, genes can appear to be driver genes, based on the results of the training of the machine learning model. The term “driver gene” as used herein, refers to a gene which includes a driver gene mutation. In some aspects, a driver gene is a gene in which one or more acquired mutations, e.g., driver gene mutations, can be causally linked to cancer progression. In some aspects, a driver gene can modulate one or more cellular processes including: cell fate determination, cell survival and genome maintenance. A driver gene can be associated with (e.g., can modulate) one or more signaling pathways, e.g., a TGF-beta pathway, a MAPK pathway, a STAT pathway, a PI3K pathway, a RAS pathway, a cell cycle pathway, an apoptosis pathway, a NOTCH pathway, a Hedgehog (HH) pathway, a APC pathway, a chromatin modification pathway, a transcriptional regulation pathway, a DNA damage control pathway, or a combination thereof. Exemplary driver genes include oncogenes and tumor suppressors. In some aspects, a driver gene provides a selective growth advantage to the cell in which it occurs. In some aspects, a driver gene provides a proliferative capacity to the cell in which it occurs, e.g., allows for cell expansion, e.g., clonal expansion. In some aspects, a driver gene is an oncogene. In some aspects, a driver gene is a tumor suppressor gene (TSG).

The presence of noisy, low-expression genes in a geneset can decrease the sensitivity of the model. Accordingly, in some aspects, low-expression genes can be down-weighted or filtered (eliminated) from the machine-learning model. In some aspects, low-expression gene filtering is based on a statistic calculated from gene expression (e.g., RNA levels). In some aspects, low-expression gene filtering is based on minimum (min), maximum (max), average (mean), variance (sd), or combinations thereof of, e.g., raw read counts for each gene in the geneset. For each geneset, an optimal filtering threshold can be determined. In some aspects, the filtering threshold is optimized to maximize the number of differentially expressed genes in the geneset

The ANN classifier can be subsequently evaluated by determining the ability of the classifier to correctly call each test subject. In some aspects, the subjects of the training population used to derive the model are different from the subjects of the testing population used to test the model. As would be understood by a person skilled in the art, this allows one to predict the ability of the geneset used to train the classifier as to their ability to properly characterize a subject whose stromal phenotype trait characterization (e.g., TME phenotype class) is unknown.

The data which is input into the mathematical model (ANN) can be any data which is representative of the expression level of the product of the gene being evaluated, e.g., mRNA. Mathematical models useful in accordance with the present disclosure include those using supervised and/or unsupervised learning techniques. In some aspect of the disclosure, the mathematical model chosen uses supervised learning in conjunction with a “training population” to evaluate each of the possible combinations of biomarkers.

Classifiers (e.g., ANN models) generated according to the methods disclosed herein can be used to test an unknown or test subject. In one aspect, the model generated by an ANN identified herein can detect whether a subject or a cancer sample belongs to a particular TME phenotype class. In some aspects, the ANN model can predict whether a subject will respond to a particular therapy. In other aspects, the ANN model can select or be used to select a subject for administration of a particular therapy.

In one aspect of the disclosure, each ANN classifier is evaluated for its ability to properly characterize each subject of the training population using methods known to a person skilled in the art. For example, one can evaluate the ANN classifier using cross validation, Leave One Out Cross Validation (LOOCV), n-fold cross validation, or jackknife analysis using standard statistical methods. In another aspect, each ANN classifier is evaluated for its ability to properly characterize those subjects of the training population which were not used to generate the classifier.

In some aspects, one can train the ANN classifier using one dataset, and evaluate the ANN classifier on another distinct dataset. Accordingly, since the testing dataset is distinct from the training dataset, there is no need for cross validation.

In one aspect, the method used to evaluate the classifier for its ability to properly characterize each subject of the training population is a method which evaluates the classifier's sensitivity (TPF, true positive fraction) and 1-specificity (FPF, false positive fraction). In one aspect, the method used to test the classifier is Receiver Operating Characteristic (“ROC”) which provides several parameters to evaluate both the sensitivity and specificity of the result of the model generated, e.g., a model derived from the application of an ANN.

In some aspects, the metrics used to evaluate the classifier for its ability to properly characterize each subject of the training population comprise classification accuracy (ACC), Area Under the Receiver Operating Characteristic Curve (AUC ROC), Sensitivity (True Positive Fraction, TPF), Specificity (True Negative Fraction, TNF), Positive Predicted Value (PPV), Negative Predicted Value (NPV), or any combination thereof. In one specific aspect, the metrics used to evaluate the classifier for its ability to properly characterize each subject of the training population are classification accuracy (ACC), Area Under the Receiver Operating Characteristic Curve (AUC ROC), Sensitivity (True Positive Fraction, TPF), Specificity (True Negative Fraction, TNF), Positive Predicted Value (PPV), and Negative Predicted Value (NPV).

In some aspects, the training set includes a reference population of at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 250, at least about 300, at least about 350, at least about 400, at least about 450, at least about 500, at least about 600, at least about 700, at least about 800, at least about 900, or at least about 1000 subjects.

In some aspects, the expression, e.g., mRNA levels, measured for each of the biomarker genes in a gene panel of the present disclosure can be used to train a neural network. A neural network is a two-stage regression or classification model. A neural network can be binary or non-binary. A neural network has a layered structure that includes a layer of input units (and the bias) connected by a layer of weights to a layer of output units. For regression, the layer of output units typically includes just one output unit. However, neural networks can handle multiple quantitative responses in a seamless fashion. As such a neural network can be applied to allow identification of biomarkers which differentiate as between more than two populations (i.e., more than two phenotypic traits), e.g., the four TME phenotype classes disclosed herein.

In one specific example, a neural network can be trained using expression data from the products, e.g., mRNA, of the biomarker genes disclosed in TABLE 1 and TABLE 2 for a set of samples obtained from a population of subjects to identify those combinations of biomarkers which are specific for a particular TME phenotype. Neural networks are described in Duda et al., 2001, Pattern Classification, Second Edition, John Wiley & Sons, Inc., New York; and Hastie et al., 2001, The Elements of Statistical Learning, Springer-Verlag, New York.

In some aspects, an ANN classifier disclosed herein, such as TME Panel-1, comprises a back-propagation neural network (see, for example Abdi, 1994, “A neural network primer”, J. Biol System. 2, 247-283) containing a single input layer with, e.g., 98 or 87 genes from TABLES 1 and 2, a single hidden layer of 2 neurons, and 4 outputs in a single output layer. An ANN classifier disclosed herein, such as TME Panel-1, can be implemented using the EasyNN-Plus version 4.0g software package (Neural Planner Software Inc.), scikit-learn (scikit-learn.org), or any other machine learning package or program known in the art.

In some aspects, the ANN classifier is a feed-forward neural network. A feed-forward neural network is an artificial network wherein connection between the input and output nodes do not form a cycle. As used here in the context of an ANN, the terms “node” and “neuron” are used interchangeably. Thus, it is different from recurrent neural networks. In this network, the information moves in only one direction, forward, from the input nodes, through the hidden nodes (if any) and to the output nodes. There are no cycles or loops in the network. Except for the input nodes, each node is a neuron that uses a nonlinear activation function, which is developed to model the frequency of action potential, or firing, of biological neurons.

In some aspects, the ANN classifier is a single-layer perceptron network, which consists of a single layer of output nodes; the inputs are fed directly to the outputs via a series of weights. The sum of the products of the weights and the inputs is calculated in each node, and if the value is above some threshold (typically 0) the neuron fires and takes the activated value (typically 1).

In some aspects, the ANN is a multi-layer perceptron (MLP). This class of networks consists of multiple layers of computational units, usually interconnected in a feed-forward way. Each neuron in one layer has directed connections to the neurons of the subsequent layer. In many applications, the units of these networks apply an activation function, e.g., a sigmoid function. An MLP comprises at least three layers of nodes: an input layer, a hidden layer and an output layer.

In some aspects, the activation function is a sigmoid function described according to the formula y(vi)=tanh(vi), i.e., a hyperbolic tangent that ranges from −1 to +1. In some aspects, the activation function is a sigmoid function described according to the formula y(vi)=(1+e^{-vi})⁻¹, i.e., a logistic function similar in shape to the tanh function but ranges from 0 to +1. In these formulas, yᵢ is the output of the ith node (neuron) and vᵢ is the weighted sum of the input connections.

In some aspects, the activation function is a rectifier linear unit (ReLU) or a variant thereof, e.g., a noisy ReLU, a leaky ReLU, a parametric ReLU, or an exponential LU. In some aspects, the ReLU is defined by the formula f(x)=x⁺=max (0, x), wherein x is the input to a neuron. The ReLU activation function enables better training of deep neural networks (DNN) compared to the hyperbolic tangent or the logistic sigmoid. A DNN is an ANN with multiple layers between the input and output layers. DNNs are typically feed-forward networks in which data flows from the input layer to the output layer without looping back. DNNs are prone to over-fitting because of the added layers of abstraction, which allow them to model rare dependencies in the training data. In some aspects, the activation function is the softplus or smoothReLU function, a smooth approximation of the ReLU, which is described by the formula f(x)=ln(1+e^{x}). The derivative of softplus is the logistic function.

In some aspects, the ANN is a MLP comprising three or more layers (an input and an output layer with one or more hidden layers) of nonlinearly-activating nodes. Its multiple layers and non-linear activation distinguish MLP from a linear perceptron. It can distinguish data that is not linearly separable. Since the ANN is fully connected, each node in one layer connects with a certain weight wᵢⱼ to every node in the following layer. Learning occurs in the perceptron by changing connection weights after each piece of data is processed, based on the amount of error in the output compared to the expected result. This is an example of supervised learning, and is carried out through backpropagation.

In some aspects, the ANN has 3 layers. In other aspects, the ANN has more than 3 layers. In some aspects, the ANN has a single hidden layer. In other aspects, the ANN has more than one hidden layer.

In some aspects, the input layer comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 neurons.

In some aspects, the input layer comprises between 70 and 100 neurons. In some aspects, the input layer comprises between 70 and 80 neurons. In some aspects, the input layer comprises between 80 and 90 neurons. In some aspects, the input layer comprises between 90 and 100 neurons. In some aspects, the input layer comprises between 70 and 75 neurons. In some aspects, the input layer comprises between 75 and 80 neurons. In some aspects, the input layer comprises between 80 and 85 neurons. In some aspects, the input layer comprises between 85 and 90 neurons. In some aspects, the input layer comprises between 90 and 95 neurons. In some aspects, the input layer comprises between 95 and 100 neurons.

In some aspects, the input layer comprises between at least about 1 to at least about 5, between at least about 5 and at least about 10, between at least about 10 and at least about 15, between at least about 15 and at least about 20, between at least about 20 and at least about 25, between at least about 25 and at least about 30, between at least about 30 and at least about 35, between at least about 35 and at least about 40, between at least about 40 and at least about 45, between at least about 45 and at least about 50, between at least about 50 and at least about 55, between at least about 55 and at least about 60, between at least about 60 and at least about 65, between at least about 65 and at least about 70, between at least about 70 and at least about 75, between at least about 75 and at least about 80, between at least about 80 and at least about 85, between at least about 85 and at least about 90, between at least about 90 and at least about 95, between at least about 95 and at least about 100, between at least about 100 and at least about 105, between at least about 105 and at least about 110, between at least about 110 and at least about 115, between at least about 115 and at least about 120, between at least about 120 and at least about 125, between at least about 125 and at least about 130, between at least about 130 and at least about 135, between at least about 135 and at least about 140, between at least about 140 and at least about 145, or between at least about 145 and at least about 150 neurons.

In some aspects, the input layer comprises between at least about 1 and at least about 10, between at least about 10 and at least about 20, between at least about 20 and at least about 30, between at least about 30 and at least about 40, between at least about 40 and at least about 50, between at least about 50 and at least about 60, between at least about 60 and at least about 70, between at least about 70 and at least about 80, between at least about 80 and at least about 90, between at least about 90 and at least about 100, between at least about 100 and at least about 110, between at least about 110 and at least about 120, between at least about 120 and at least about 130, between at least about 130 and at least about 140, or between at least about 140 and at least about 150 neurons.

In some aspects, the input layer comprises between at least about 1 and at least about 20, between at least about 20 and at least about 40, between at least about 40 and at least about 60, between at least about 60 and at least about 80, between at least about 80 and at least about 100, between at least about 100 and at least about 120, between at least about 120 and at least about 140, between at least about 10 and at least about 30, between at least about 30 and at least about 50, between at least about 50 and at least about 70, between at least about 70 and at least about 90, between at least about 90 and at least about 110, between at least about 110 and at least about 130, or between at least about 130 and at least about 150 neurons.

In some aspects, the input layer comprises more than about 1, more than about 5, more than about 10, more than about 15, more than about 20, more than about 25, more than about 30, more than about 35, more than about 40, more than about 45, more than about 50, more than about 55, more than about 60, more than about 65, more than about 70, more than about 75, more than about 80, more than about 85, more than about 90, more than about 95, more than about 100, more than about 105, more than about 110, more than about 115, more than about 120, more than about 125, more than about 130, more than about 135, more than about 140, more than about 145, or more than about 150 neurons.

In some aspects, the input layer comprises less than about 1, less than about 5, less than about 10, less than about 15, less than about 20, less than about 25, less than about 30, less than about 35, less than about 40, less than about 45, less than about 50, less than about 55, less than about 60, less than about 65, less than about 70, less than about 75, less than about 80, less than about 85, less than about 90, less than about 95, less than about 100, less than about 105, less than about 110, less than about 115, less than about 120, less than about 125, less than about 130, less than about 135, less than about 140, less than about 145, or less than about 150 neurons. In some aspects, a weight is applied to the input of each one of the neurons in the input layer.

In some aspects, the ANN comprises a single hidden layer. In some aspects, the ANN comprises 1, 2, 3, 4, 5, 6, 7, 8, 9 or 10 hidden layers. In some aspects, the single hidden layer comprises 1, 2, 3, 4, 5, 6, 7, 8, 9, or 10 neurons. In some aspects, the single hidden layer comprises at least 1, at least 2, at least 3, at least 4, at least 5, at least 6, at least 7, at least 8, at least 9, or at least 10 neurons. In some aspects, the single hidden layer comprises less than 10, less than 9, less than 8, less than 7, less than 6, less than 5, less than 4, or less than 3 neurons. In some aspects, the single hidden layer comprises 2 neurons. In some aspects, the single hidden layer comprises 3 neurons. In some aspects, the single hidden layer comprises 4 neurons. In some aspects, the single hidden layer comprises 5 neurons. In some aspects, a bias is applied to the neurons in the hidden layer.

In some aspects, the ANN comprises four neurons in the output layer corresponding to different TME phenotypes. In some aspects, the four neurons in the output layer correspond to the four TME phenotype classes disclosed above, i.e., IA (immune active), IS (immune suppressed), ID (immune desert), and A (angiogenic).

In some aspects, the classification of the output layer is normalized to a probability distribution over predicted output classes, and the components will add up to 1, so that they can be interpreted as probabilities.

In some aspects, the multi-class classification of the output layer values into four TME phenotype classes (IA, ID, A, and IS) is supported by applying a logistic regression function. In some aspects, the multi-class TME classification of the output layer values into four TME phenotype classes (IA, ID, A, and IS) is supported by applying a logistic regression classifier, e.g., the Softmax function. Softmax assigns decimal probabilities to each class that adds up to 1.0. In some aspects, the use of a logistic regression classifier such as the Softmax function helps training converge more quickly. In some aspects, the logistic regression classifier comprising a Softmax function is implemented through a neural network layer just before the output layer. In some aspects, such neural network layer just before the output layer has the same number of nodes as the output layer.

In some aspects, various cut-offs are applied to the results of the logistic regression classifier (e.g., Softmax function) depending on the particular dataset used (see, e.g., cut-offs applied to select a particular population of subjects, e.g., those responding to a particular therapy). Thus, applying different sets of cut-offs can classify a cancer or a patient in not only one of the four TME phenotype classes disclosed above, IA (immune active), IS (immune suppressed), ID (immune desert), or A (angiogenic), but also classify a cancer or a patient in more than one TME phenotype class disclosed above. Accordingly, in some aspects, a cancer or a patient can be classified as being biomarker-positive for the IA, IS, ID, or A TME phenotype classes or any combination thereof. Conversely, in some aspects, a cancer or a patient can be classified as being biomarker-negative for the IA, IS, ID, or A TME phenotype classes or any combination thereof.

In some aspects, all, or a subset of genes of the Angiogenesis Signature, and all, or a subset of genes of the Immune Signature, have positive or negative gene weights in the ANN model for each hidden layer.

The practical behavior of the ANN model of the present disclosure is to represent high dimensional data in a compressed form. The compressed data can be represented visually in what is known as the latent space. A common example of this is a two dimensional graph (X & Y axes), where each patient is plotted as the value of some vector X and vector Y. Thus, the latent space is a projection of the signatures generated by the method of the present disclosure, e.g., whether is a projection of the Z-scores or the values of the hidden neurons. In some aspects, the latent space can be plotted in three-dimensions.

Disease score values of each patient can be plotted in the latent space (i.e., the probability result of the ANN model). Over time, patient data can be accumulated, or the results of a retrospective analysis of patient data with disease scores can be used as a reference plot, on which the subject patient's ANN probability result is plotted.

In some aspects, the latent space is a plot of the hidden neurons of the ANN model, and could include all 2-way combinations of those neurons. In some aspects, the ANN model predicts four TME phenotype classes based on the data compressed in the two hidden neurons, and plotting those neurons in the latent space also serves as a projection of the four output TME phenotype classes. In some aspects, the TME phenotype class assignments of each patient are visualized in the Neuron 1 versus Neuron 2 latent space.

The latent space projection may be enhanced by displaying the probability contours of the output TME phenotype assignments. In this way, the projection can show not only where subjects fall in the latent space, but also the confidence of each TME phenotype classification. In some aspects, clinical reporting can use the TME phenotype class as the biomarker logic—that is, IA=positive, or IA+IS=positive—then report out to the clinician the probability of the TME phenotype assignment, which is already an output of the model. The latent space plot can also be used to visualize the distance of that patient from the decision boundary to assist clinical decision makers in evaluating edge cases and exceptions.

In some aspects, the boundaries between the TME phenotype classes are not on the cartesian axes (x=0, y=0), but elsewhere in the plot.

In some aspects, a second model can learn the biomarker boundary from the ANN model latent space. In some aspects, that second model can be a logistic regression model. In some aspects it could be any other kind of regression or machine learning algorithm. In some aspects, a logistic regression function may be applied to the latent space. In some aspects, combining TME phenotypes to define the biomarker positive class, i.e. IA+IS, the confidence of the individual phenotype assignments does not equal the confidence of the combined class assignment. A logistic regression function is used to learn what it means to be biomarker positive and directly reports statistics on being biomarker positive. A logistic regression function can be used to fine-tune the biomarker positive/negative decision boundary based on real patient outcome data. In some aspects, the accuracy of the ANN model can be improved by slicing the latent space according to a secondary model.

In some aspects, the probability function can be plotted in two dimensions, one axis representing the probability that the signal is dominated by the genes of the Angiogenesis Signature, and the other axis representing the probability that the that the signal is dominated by the genes of Immune Signature. In some aspects, genes that play a role in angiogenesis and in immune functions contribute to each of the probability functions. Each quadrant of the latent space plot represents a stromal phenotype. In a further aspect, the threshold is applied by using a logistic regression. In some aspects, the logistic regression can be linear or polynomial. After a threshold is set, individual patient results can be analyzed according to the methods described herein.

### III. TME Phenotype Class-Specific Therapies

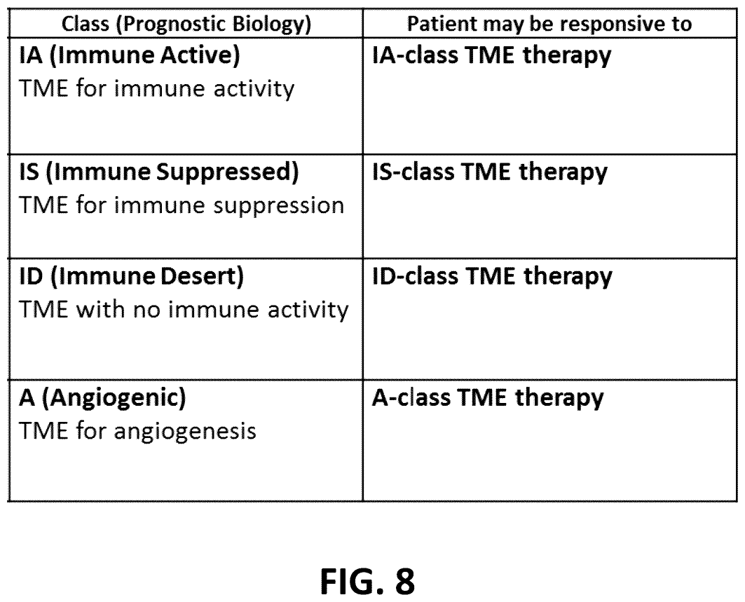

The analysis of RNA expression data from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) indicates that specific subpopulations of cancer patients can be effectively treated with IA (Immune Active), IS (Immune Suppressed), A (Angiogenic) or ID (Immune desert) TME phenotype class-specific therapies according to the methods disclosed above. Using the classifiers disclosed herein, e.g., the TME Panel-1 Classifier, to assign a patient or cancer tumor to one of four TME phenotypes classes can be used to predict which therapies are more effective to treat a specific subpopulation of patients, e.g., those having a left or right colorectal cancer, a mismatch repair deficient (dMMR), or having a MSI-H colorectal cancer. See, e.g., FIG. 8.

Classification of a dMMR or MSI-H tumor, e.g., a colorectal cancer tumor, in the IA TME phenotype class correlates with improved clinical outcomes in treatments with checkpoint inhibitors. Accordingly, patients with dMMR or MSI-H tumors, e.g., colorectal cancer tumors, with an IA TME phenotype would be administered a therapy comprising checkpoint inhibitors selected from the IA TME Phenotype Class-Specific Therapies disclosed below. Classification of a dMMR or MSI-H tumor, e.g., a colorectal cancer tumor, in the IS TME phenotype class correlates with improved clinical outcomes in treatments combining checkpoint inhibitors and phosphatidylserine inhibitors. Accordingly, patients with dMMR or MSI-H tumors, e.g., colorectal cancer tumors, with an IS TME phenotype would be administered a combined therapy comprising checkpoint inhibitors and phosphatidylserine inhibitors selected from the IS TME Phenotype Class-Specific Therapies disclosed below. Classification of a metastatic tumor, e.g., a metastatic colorectal tumor, in the A or IS TME phenotype classes correlates with improved clinical outcomes in treatments with angiogenesis inhibitors. Accordingly, patients with metastatic cancer with an A or IS TME phenotype would be administered a therapy comprising angiogenesis inhibitors selected from the A TME Phenotype Class-Specific Therapies disclosed below. Classification of a tumor in the IA TME phenotype class could be used to select a checkpoint inhibitor, e.g., pembrolizumab as an adjuvant therapy. Classification of a tumor in the A TME phenotype class could be used to select an anti-angiogenic therapy, e.g., with bevacizumab, as an adjuvant therapy. Furthermore, in the case of colorectal cancer, classification of a left or right colorectal tumor as having a dominant TME phenotype class, could be used to select a therapy disclosed below that would match, for example, the dominant TME phenotype class.

### III.A IA TME Phenotype Class-Specific Therapies

A TME that is dominated by immune activity, such as the TME of a tumor from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) classified in the IA (Immune Active) TME phenotype class by a classifier of the present disclosure such as the TME Panel-1 Classifier (i.e., an IA biomarker-positive patient) is likely to be responsive to immune checkpoint inhibitors (CPIs) such as anti-PD-1 (e.g., sintilimab, tislelizumab, pembrolizumab, or an antigen binding portion thereof), anti-PD-L1, or anti-CTLA-4, or to RORy agonist therapeutics.

Checkpoint inhibitors: In some aspects, the immune checkpoint inhibitors are blocking antibodies that bind to PD-1, e.g., nivolumab, cemiplimab (REGN2810), geptanolimab (CBT-501), pacmilimab (CX-072), dostarlimab (TSR-042), sintilimab, tislelizumab, and pembrolizumab; PD-L1, e.g., durvalumab (MEDI4736), avelumab, lodapolimab (LY-3300054), CX-188, and atezolizumab; or CTLA-4, e.g., ipilimumab and tremelimumab. In some aspect, a combination of one or more of such antibodies can be used.

Tremelimumab, nivolumab, durvalumab and atezolizumab are described, for example, in U.S. Pat. Nos. 6,682,736, 8,008,449, 8,779,108 and 8,217,149, respectively. In some aspects, atezolizumab can be replaced by another immune checkpoint antibody, such as another blocking antibody that binds to CTLA-4, PD-1 (e.g., sintilimab, tislelizumab, pembrolizumab, or an antigen binding portion thereof), PD-L1, or a bispecific blocking antibody that binds to any checkpoint inhibitor. In selecting a different blocking antibody, those of ordinary skill in the art will know the suitable dose and administration schedule from the literature. Suitable examples of anti-CTLA-4 antibodies are those described in U.S. Pat. No. 6,207,156. Other suitable examples of anti-PD-L1 antibodies are those described in U.S. Pat. No. 8,168,179, which particularly concerns treating PD-L1 over-expressing cancers with human anti-PD-L1 antibodies, including chemotherapy combinations; U.S. Pat. No. 9,402,899, which particularly concerns treating tumors with antibodies to PD-L1, including chimeric, humanized and human antibodies; and U.S. Pat. No. 9,439,962, which particularly concerns treating cancers with anti-PD-L1 antibodies and chemotherapy.

Further suitable antibodies to PD-L1 are those in U.S. Pat. Nos. 7,943,743, 9,580,505 and 9,580,507, kits thereof (U.S. Pat. No. 9,580,507) and nucleic acids encoding the antibodies (U.S. Pat. No. 8,383,796). Such antibodies bind to PD-L1 and compete for binding with a reference antibody; are defined by V_{H} and V_{L} genes; or are defined by heavy and light chain CDR3 (U.S. Pat. No. 7,943,743), or heavy chain CDR3 (U.S. Pat. No. 8,383,796), of defined sequences or conservative modifications thereof; or have 90% or 95% sequence identity to reference antibodies. These anti-PD-L1 antibodies also include those with defined quantitative (including binding affinity) and qualitative properties, immunoconjugates and bispecific antibodies. Further included are methods of using such antibodies, and those with defined quantitative (including binding affinity) and qualitative properties, including antibodies in single chain format and those that are in the format of an isolated CDR, in enhancing an immune response (U.S. Pat. No. 9,102,725). Enhancing an immune response, as in U.S. Pat. No. 9,102,725, can be used to treat cancer.

Further suitable antibodies to PD-L1 are those in U.S. Patent Application No. 2016/0009805, which concerns antibodies to particular epitopes on PD-L1, including antibodies of defined CDR sequences and competing antibodies; nucleic acids, vectors, host cells, immunoconjugates; detection, diagnostic, prognostic and biomarker methods; and treatment methods.

Specific treatments comprising ipilimumab are disclosed, e.g., in U.S. Pat. Nos. 7,605,238; 8,318,916; 8,784,815; and 8,017,114. Treatments comprising tremelimumab are disclosed, e.g., in U.S. Pat. Nos. 6,682,736, 7,109,003, 7,132,281, 7,411,057, 7,807,797, 7,824,679, 8,143,379, 8,491,895, and 8,883,984. Treatments with nivolumab are disclosed, e.g., in U.S. Pat. Nos. 8,008,449, 8,779,105, 9,387,247, 9,492,539, 9,492,540, 8,728,474, 9,067,999, 9,073,994, and 7,595,048. Treatments with pembrolizumab are disclosed, e.g., in U.S. Pat. Nos. 8,354,509, 8,900,587, and 8,952,136. Treatments with cemiplimab are disclosed, e.g., in US20150203579A1. Treatment with durvalumab are disclosed, e.g., in U.S. Pat. Nos. 8,779,108 and 9,493,565. Treatment with atezolizumab are disclosed, e.g., in U.S. Pat. No. 8,217,149. Treatments with CX-072 are disclosed, e.g., in Ser. No. 15/069,622. Treatments with LY300054 are disclosed, e.g., in U.S. Ser. No. 10/214,586B2. Treating of tumors with combination of antibodies to PD-1 and CTLA-4 is disclosed, e.g., in U.S. Pat. Nos. 9,084,776, 8,728,474, 9,067,999 and 9,073,994. Treating tumors with antibodies to PD-1 and CTLA-4, including sub-therapeutic doses and PD-L1 negative tumors is disclosed, e.g., in U.S. Pat. No. 9,358,289. Treating tumors with antibodies to PD-L1 and CTLA-4 is disclosed, e.g., in U.S. Pat. Nos. 9,393,301 and 9,402,899. All these patents and publication are incorporated herein by reference in their entireties.

Specific therapeutic agents and whether they are approved for the treatment of solid tumors are identified in the table below.

TABLE 6

| Target | Generic Name | Other name | Target |
|---|---|---|---|
| PD-1 | Nivolumab | OPDIVO ™ | |
| | Pembrolizumab | KEYTRUDA ™ | Solid Tumors; |
| | | | Colorectal Cancer |
| | Cemiplimab | REGN2810 | |
| | Spartalizumab | PDR001 | |
| | Geptanolimab | CBT-501 | Solid Tumors |
| | Sintilimab | TYVYT ™, IBI308 | |
| | Tislelizumab | BGB-A317 | Solid tumors |
| PD-L1 | Atezolizumab | TECENTRIQ ™ | Colorectal Cancer |
| | | MPDL3280A | |
| | Avelumab | BAVENCIO ™ | |
| | Durvalumab | MEDI4736 | |
| | Pacmilimab | CX-072, PROBODY ™ | Solid Tumors |
| | Lodapolimab | LY-3300054 | Solid Tumors |
| CTLA-4 | Ipilimumab | YERVOY ™ | |
| | | MDX-010 | |
| | Tremelimumab | AZD9150 | |

RORγ agonist therapeutics: In some aspects, RORγ agonist therapeutics are small molecule agonists of RORγ (Retinoid-related orphan receptor gamma), which belongs to the nuclear hormone receptor family. RORγ plays a critical role in control apoptosis during thymopoiesis and T cell homeostasis. Small molecule agonists in clinical development include LYC-55716 (cintirorgon).

### Tislelizumab

Tislelizumab (BGB-A317) is a humanized monoclonal antibody directed against PD-1. It prevents PD-1 from binding to the ligands PD-L1 and PD-L2 (hence it is a checkpoint inhibitor). Tislelizumab can be used for the treatment of solid cancers, e.g., Hodgkin's lymphoma (alone or in combination with an adjuvant therapy such as platinum-containing chemotherapy), urothelial cancer, NSCLC, or hepatocellular carcinoma. Sequences relating to tislelizumab are provided in the table below. In some aspects of the present disclosure, tislelizumab or an antigen binding portion thereof can be administered in combination with bavituximab.

TABLE 7
Tislelizumab Sequences

| SEQ ID | | |
|---|---|---|
| NO | Description | Sequence |
| 28 | VH CDR1 | GFSLTSYG |
| 29 | VH CDR2 | IYADGST |
| 30 | VH CDR3 | ARAYGNYWYIDV |
| 31 | VL CDR1 | ESVSND |
| 32 | VL CDR2 | YAF |
| 33 | VL CDR3 | HQAYSSPYT |
| 34 | VH | QVQLQESGPGLVKPSETLSLTCTVSGFSL |
| | | TSYGVHWIRQPPGKGLEWIGVIYADGSTN |
| | | YNPSLK.SRVTISKDTSKNQVSLKLSSVT |
| | | AADTAVYYCARAYGNYWYIDVWGQGTTVT |
| | | VSS |
| 35 | VL | DIVMTQSPDSLAVSLGERATINCKSSESV |
| | | SNDVAWYQQKPGQPPKLLINYAFHRFTGV |
| | | PDRFSGSGYGTDFTLTISSLQAEDVAVYY |
| | | CHQAYSSPYTFGQGTKLEIK |

### Sintilimab

Sintilimab (TYVYT®) is a fully human IgG4 monoclonal antibody directed against PD-1. It prevents PD-1 from binding to the ligands PD-L1 and PD-L2 (hence it is a checkpoint inhibitor). Sintilimab can be used for the treatment of solid cancers, e.g., Hodgkin's lymphoma, alone or in combination with an adjuvant therapy. Sequences relating to sintilimab are provided in the table below. In some aspects of the present disclosure, sintilimab or an antigen binding portion thereof can be administered in combination with bavituximab.

TABLE 8
Sintilimab Sequences

| SEQ ID | | |
|---|---|---|
| NO | Description | Sequence |
| 36 | VH CDR1 | GGTFSSYA |
| 37 | VH CDR2 | IIPMFDTA |
| 38 | VH CDR3 | ARAEHSSTGTFDY |
| 39 | VL CDR1 | QGISSW |
| 40 | VL CDR2 | AAS |
| 41 | VL CDR3 | QQANHLPFT |
| 42 | VH | QVQLVQSGAEVKKPGSSVKVSCKASGGTF |
| | | SSYAISWVRQAPGQGLEWMGLIIPMFDTA |
| | | GYAQKFQ.GRVAITVDESTSTAYMELSSL |
| | | RSEDTAVYYCARAEHSSTGTFDYWGQGTL |
| | | VTVSS |
| 43 | VL | DIQMTQSPSSVSASVGDRVTITCRASQGI |
| | | SSWLAWYQQKPGKAPKLLISAASSLQSGV |
| | | P.SRFSGSGSGTDFTLTISSLQPEDFATY |
| | | YCQQANHLPFTFGGGTKVEIK |

### III.B IS TME Phenotype Class-Specific Therapies

A TME that is dominated by immune suppression, such as the TME of a tumor from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) classified in the IS (Immune Suppressed) TME phenotype class by a classifier of the present disclosure such as the TME Panel-1 Classifier (i.e., an IS biomarker-positive patient) can be resistant to checkpoint inhibitors unless also given a drug to reverse immunosuppression such as anti-phosphatidylserine (anti-PS) and anti-phosphatidylserine-targeting therapeutics, PI3Kγ inhibitors, adenosine pathway inhibitors, IDO, TIMs, LAG3, TGFβ, and CD47 inhibitors.

Bavituximab is a preferred anti-PS-targeting therapeutic. A patient with this biology may also have underlying angiogenesis and can also get benefit from anti-angiogenics, such as those used for the A TME phenotype.

Specific therapeutics for patients with cancer tumors assigned to the IS TME phenotype class by a classifier disclosed herein, e.g., the TME Panel-1 classifier, are now discussed. Anti-PS and PS-targeting antibodies, include, but are not limited to bavituximab; PI3Kγ inhibitors such as LY3023414 (samotolisib), IPI-549; Adenosine Pathway inhibitors such as AB-928 (an oral antagonist of the adenosine 2a and 2b receptors); IDO inhibitors; anti-TIMs, both TIMs and TIM-3; anti-LAG3; TGFβ inhibitors, such as LY2157299 (galunisertib); CD47 inhibitors, such as Forty Seven's magrolimab (5F9).

Specific therapeutics for patients with cancer tumors assigned to the IS TME phenotype class by a classifier disclosed herein, e.g., the TME Panel-1 classifier, also include: Anti-TIGIT drugs, which are immunosuppressive through triggering of CD155 (Cluster of Differentiation 155) on dendritic cells (among other activities) and expression of subset of Tregs in tumors. A preferred anti-TIGIT antibody is AB-154. Another preferred anti-TIGIT antibody is BGB-A1217 (ociperlimab). Anti-activin A therapeutics, because Activin A promotes differentiation of M2-like tumor macrophages and inhibits generation of NK cells. Anti-BMP therapeutics are useful, because bone morphogenic protein (BMP) also promotes differentiation of M2-like tumor macrophages and inhibits CTLs and DCs.

Further specific therapeutics for patients with cancer tumors assigned to the IS TME phenotype class by a classifier disclosed herein, e.g., the TME Panel-1 classifier, also include: TAM (Tyro3, Axl, and Mer receptors) inhibitors or TAM product inhibitors; anti-IL-10 (interleukin) or anti-IL-10R (interleukin 10 receptor), since IL-10 is immunosuppressive; anti-M-CSF, as macrophage-colony stimulating factor (M-CSF) antagonism has been shown to deplete TAMs; anti-CCL2 (C-C Motif Chemokine Ligand 2) or anti-CCL2R (C-C Motif Chemokine Ligand 2 receptor), the particular pathway targeted by those drugs recruits myeloid cells to tumors; MERTK (Tyrosine-protein kinase Mer) antagonists, as inhibition of this receptor tyrosine kinase triggers a pro-inflammatory TAM phenotype and increases tumor CD8+ cells.

Other therapeutics for patients with cancer tumors assigned to the IS TME phenotype class by a classifier disclosed herein, e.g., the TME Panel-1 classifier, include: STING agonists, as cytosolic DNA sensing by Stimulator of Interferon Genes (STING) enhances DC-stimulation of anti-tumor CD8+ T cells, and agonists are part of STINGVAX®; antibodies to CCL3 (C-C motif chemokine 3), CCL4 (C-C motif chemokine 4), CCL5 (C-C motif chemokine 5) or their common receptor CCR5 (C-C motif chemokine receptor type 5), as these chemokines are products of myeloid-derived suppressor cells (MDSCs) and activate CCR5 on regulatory T cells (Tregs); inhibitors of arginase-1 because arginase-1 is produced by M2-like TAMs, decreases production of tumor infiltrating lymphocytes (TILs) and increases production of Tregs; antibodies to CCR4 (C-C motif chemokine receptor type 4) can be used to deplete Tregs; antibodies to CCL 17 (C-C motif chemokine 17) or CCL22 (C-C motif chemokine 22) can inhibit CCR4 (C-C motif chemokine receptor type 4) activation on Tregs; antibodies to GITR (glucocorticoid-induced TNFR-related protein) can be used to deplete Tregs; inhibitors of DNA methyltransferases (DNMTs) or histone deacetylases (HDACs) that cause the reversal of epigenetic silencing of immune genes, such as entinostat.

In pre-clinical models, inhibitors of phosphodiesterase-5, sildenafil, and tadalafil significantly inhibited the MDSC functions, which can provide benefit patients with colorectal cancer tumors having an IS TME phenotype. All-trans retinoic acid (ATRA) used to differentiate MDSCs into mature dendritic cells (DCs) and macrophages may provide benefit to patients with an IS phenotype. VEGF and c-kit signaling is reported to be involved in the generation of MDSC. Sunitinib treatment of metastatic renal cell carcinoma patients was reported to decrease the number of circulating MDSC, which may provide benefit to patients with colorectal cancer tumors having an IS TME phenotype.

Cancer tumors classified into the IS TME phenotype class represent the target population for bavituximab treatment in combination with a checkpoint inhibitor such as an anti-PD-1 (e.g., sintilimab, tislelizumab, pembrolizumab, or an antigen binding portion thereof), anti-PD-L1, or anti-CTLA-4. This is because the present disclosure notes that immune responses that take place in the presence of angiogenesis show signs of immunosuppression, and bavituximab can restore immune activity to immunosuppressed cells.

For single agent bavituximab to work, the ongoing immune response would have to be highly active to the extent that blocking immunosuppression would be sufficient to unleash the full potential of the patient's immune response. However, most late stage cancer patients are in need of keeping their immune response going, and are likely to need a combination with bavituximab and checkpoint inhibitors. Thus, the IS TME phenotype class disclosed herein can be used to determine which colorectal cancer patients that are likely to respond to bavituximab and checkpoint inhibitors.

### Bavituximab

Bavituximab is a PS-targeting antibody. Bavituximab binding to phosphatidylserine (PS) is mediated by β2-glycoprotein 1 (β2GPI), a serum protein. β2GPI is also known as apolipoprotein H (Apo-H). Bavituximab has been used in clinical trials for breast cancer, liver cancer (hepatocellular carcinoma), malignant melanoma, colorectal cancer, and prostate cancer.

In some aspects, bavituximab can be administered to a subject, e.g., a patient with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), or ovarian cancer (e.g., platinum-resistant ovarian cancer or platinum-sensitive recurrent ovarian cancer), in accordance with methods described herein. Sequences relating to bavituximab are provided in the table below.

TABLE 9
Bavituximab Sequences

| SEQ ID | | |
|---|---|---|
| NO | Description | Sequence |
| 1 | VH CDR1 | GYNMN |
| 2 | VH CDR2 | HIDPYYG |
| 3 | VH CDR3 | YCVKGGYY |
| 4 | VL CDR1 | RASQDIGSSLN |
| 5 | VL CDR2 | ATSSLDS |
| 6 | VL CDR3 | LQYVSSPPT |
| 22 | VH | EVQLQQSGPELEKPGASVKLSCKASGYSF |
| | | TGYNMNWVKQSHGKSLEWIGHIDPYYGDT |
| | | SYNQKFRGKATLTVDKSSSTAYMQLKSLT |
| | | SEDSAVYYCVKGGYYGHWYFDVWGAGTTV |
| | | TVSS |
| 23 | VL | DIQMTQSPSSLSASLGERVSLTCRASQDI |
| | | GSSLNWLQQGPDGTIKRLIYATSSLDSGV |
| | | PKRFSGSRSGSDYSLTISSLESEDFVDYY |
| | | CLQYVSSPPTFGAGTKLELK |

In some aspects, bavituximab is administered to a cancer patient in combination with an anti-PD-1 antibody (e.g., sintilimab, tislelizumab, pembrolizumab, or an antigen binding portion thereof). In some aspects, bavituximab is administered in combination with pembrolizumab. In some aspects, bavituximab is administered in combination with sintilimab. In some aspects, bavituximab is administered in combination with tislelizumab.

In some aspects, bavituximab is administered to a patient with gastric cancer (e.g., locally advances or metastatic gastric cancer) in a combination treatment comprising chemotherapy and an immune checkpoint inhibitor, e.g., an anti-PD-1 antibody (e.g., sintilimab, tislelizumab, pembrolizumab, or an antigen binding portion thereof).

In some aspects, bavituximab is administered to a patient with liver cancer (e.g., advanced metastatic hepatocellular carcinoma) or with a carcinoma of head and neck (e.g., recurrent or metastatic squamous cell carcinoma of head and neck) in a combination treatment comprising an immune checkpoint inhibitor, e.g., an anti-PD-1 antibody (e.g., sintilimab, tislelizumab, pembrolizumab, or an antigen binding portion thereof).

In some aspects, bavituximab is administered to a patient with melanoma in a combination therapy comprising radiation.

### II.C A TME Phenotype Class-Specific Therapies

For the A TME phenotype class, which is dominated by angiogenic activity, a patient can be responsive to VEGF-targeted therapies, DLL4-targeted therapies, Angiopoietin/TIE2-targeted therapies, anti-VEGF/anti-DLL4 bispecific antibodies, such as navicixizumab, and anti-VEGF or anti-VEGF receptor antibodies such as varisacumab, ramucirumab, bevacizumab, etc.

In some aspects, a dual-variable domain immunoglobulin molecule, drug, or therapy with anti-angiogenic effects, such as those that have anti-DLL4 and/or anti-VEGF activity, can be selected to treat a patient with a cancer classified within the A TME phenotype class, e.g., by applying the TME Panel-1 Classifier. In some aspects, the dual-variable domain immunoglobulin molecule, drug, or therapy is dilpacimab (ABT165). In some aspects, a dual-targeting protein, drug, or therapy with anti-angiogenic effects, such as those that have anti-DLL4 and/or anti-VEGF activity, can be selected to treat a patient with a cancer identified as having the A TME phenotype, e.g., by applying the TME Panel-1 Classifier. In some aspects, the dual-targeting protein, drug, or therapy is ABL001 (NOV1501, TR009), as taught by U.S. Publication No. 2016/0159929, which is herein incorporated by reference in its entirety.

### Bevacizumab

Bevacizumab, sold under the brand name AVASTIN® is a medication used to treat a number of types of cancer, e.g., colorectal cancer, breast cancer, or ovarian cancer. Bevacizumab is given by slow injection into a vein (intravenous). Bevacizumab is a monoclonal antibody that functions as an angiogenesis inhibitor. It works by slowing the growth of new blood vessels by inhibiting vascular endothelial growth factor A (VEGF-A), in other words anti-VEGF therapy. Bevacizumab was approved in the United States in 2004, for use in metastatic colorectal cancer when used with standard chemotherapy treatment (as first-line treatment). In 2006, it was approved with 5-fluorouracil-based therapy for second-line metastatic colorectal cancer. It has also been approved by the European Medicines Agency (EMA) for use in colorectal cancer. Bevacizumab has also been examined as an add-on to other chemotherapy drugs in people with non-metastatic colon cancer. In the European Union, bevacizumab in combination with fluoropyrimidine-based chemotherapy is indicated for treatment of adults with metastatic carcinoma of the colon or rectum. In the EU, bevacizumab in combination with paclitaxel is indicated for first-line treatment of adults with metastatic breast cancer. Bevacizumab in combination with capecitabine is indicated for first-line treatment of adults with metastatic breast cancer in whom treatment with other chemotherapy options including taxanes or anthracyclines is not considered appropriate.

In 2018, the U.S. Food and Drug Administration (FDA) approved bevacizumab in combination with chemotherapy for stage III or IV of ovarian cancer after initial surgical operation, followed by single-agent bevacizumab. The approval was based on a study of the addition of bevacizumab to carboplatin and paclitaxel. Progression-free survival was increased to 18 months from 13 months. In the EU, bevacizumab, in combination with carboplatin and paclitaxel is indicated for the front-line treatment of adults with advanced (International Federation of Gynecology and Obstetrics (FIGO) stages IIIB, IIIC and IV) epithelial ovarian, fallopian tube, or primary peritoneal cancer. Bevacizumab, in combination with carboplatin and gemcitabine or in combination with carboplatin and paclitaxel, is indicated for treatment of adults with first recurrence of platinum-sensitive epithelial ovarian, fallopian tube or primary peritoneal cancer who have not received prior therapy with bevacizumab or other VEGF inhibitors or VEGF receptor-targeted agents. In May 2020, the Food and Drug Administration expanded the indication of olaparib to include its combination with bevacizumab for first-line maintenance treatment of adults with advanced epithelial ovarian, fallopian tube, or primary peritoneal cancer who are in complete or partial response to first-line platinum-based chemotherapy and whose cancer is associated with homologous recombination deficiency positive status defined by either a deleterious or suspected deleterious BRCA mutation, and/or genomic instability.

Bevacizumab biosimilars: The FDA and the European Union have approved Amgen's biosimilar (generic name bevacizumab-awwb, product name Mvasi), Zirabev (Pfizer), Aybintio (Samsung Bioepis), and Equidacent (Centus Biotherapeutics) have been approved for use in the European Union. On 28 Jan. 2021, the Committee for Medicinal Products for Human Use (CHMP) of the European Medicines Agency (EMA) adopted a positive opinion, recommending the granting of a marketing authorization for the medicinal product Alymsys (Mabxience Research) for the treatment of colorectal cancer.

TABLE 10
Bevacizumab Sequences

| SEQ ID | | |
|---|---|---|
| NO | Description | Sequence |
| 44 | VH CDR1 | SGYTFTNYG |
| 45 | VH CDR2 | INTYTGEP |
| 46 | VH CDR3 | CAKYPHYYGSSHWYFDV |
| 47 | VL CDR1 | QDISNY |
| 48 | VL CDR2 | FTS |
| 49 | VL CDR3 | QQYSTVPWT |
| 50 | VH | EVQLVESGGGLVQPGGSLRLSCAASGYTF |
| | | TNYGMNWVRQAPGKGLEWVGWINTYTGEP |
| | | TYAADFKRRFTFSLDTSKSTAYLQMNSLR |
| | | AEDTAVYYCAKYPHYYGSSHWYFDVWGQG |
| | | TLVTVSS |
| 51 | VL | DIQMTQSPSSLSASVGDRVTITCSASQDI |
| | | SNYLNWYQQKPGKAPKVLIYFTSSLHSGV |
| | | PSRFSGSGSGTDFTLTISSLQPEDFATYY |
| | | CQQYSTVPWTFGQGTKVEIK |
| 52 | Heavy chain | EVQLVESGGGLVQPGGSLRLSCAASGYTF |
| | | TNYGMNWVRQAPGKGLEWVGWINTYTGEP |
| | | TYAADFKRRFTFSLDTSKSTAYLQMNSLR |
| | | AEDTAVYYCAKYPHYYGSSHWYFDVWGQG |
| | | TLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQT |
| | | YICNVNHKPSNTKVDKKVEPKSCDKTHTC |
| | | PPCPAPELLGGPSVFLFPPKPKDTLMISR |
| | | TPEVTCVVVDVSHEDPEVKFNWYVDGVEV |
| | | HNAKTKPREEQYNSTYRVVSVLTVLHQDW |
| | | LNGKEYKCKVSNKALPAPIEKTISKAKGQ |
| | | PREPQVYTLPPSREEMTKNQVSLTCLVKG |
| | | FYPSDIAVEWESNGQPENNYKTTPPVLDS |
| | | DGSFFLYSKLTVDKSRWQQGNVFSCSVMH |
| | | EALHNHYTQKSLSLSPGK |
| 53 | Light chain | DIQMTQSPSSLSASVGDRVTITCSASQDI |
| | | SNYLNWYQQKPGKAPKVLIYFTSSLHSGV |
| | | PSRFSGSGSGTDFTLTISSLQPEDFATYY |
| | | CQQYSTVPWTFGQGTKVEIKRTVAAPSVF |
| | | IFPPSDEQLKSGTASVVCLLNNFYPREAK |
| | | VQWKVDNALQSGNSQESVTEQDSKDSTYS |
| | | LSSTLTLSKADYEKHKVYACEVTHQGLSS |
| | | PVTKSFNRGEC |

### Navicixizumab

Navicixizumab, an anti-VEGF/anti-DLL4 bispecific antibody, is described in detail, for example, in U.S. Pat. Nos. 9,376,488, 9,574,009 and 9,879,084, each of which is incorporated herein by reference in its entirety.

TABLE 11
Navicixizumab Sequences

| SEQ | | |
|---|---|---|
| ID | | |
| NO | Description | Sequence |
| 13 | VEGF VH CDR1 | NYWMH |
| 14 | VEGF VH CDR2 | DINPSNGRTSYKEKFKR |
| 15 | VEGF VH CDR3 | HYDDKYYPLMDY |
| 16 | DLL4 VH CDR1 | TAYYIH |
| 17 | DLL4 VH CDR2 | YISNYNRATNYNQKFKG |
| 18 | DLL4 V4 CDR3 | RDYDYDVGMDY |
| 19 | VL CDR1 | RASESVDNYGISFMK |
| 20 | VL CDR2 | AASNQGS |
| 21 | VL CDR3 | QQSKEVPWTFGG |
| 24 | VH | QVQLVQSGAEVKKPGASVKISCKASGY |
| | | SFTAYYIHWVKQAPGQGLEWIGYISNY |
| | | NRATNYNQKFKGRVTFTTDTSTSTAYM |
| | | ELRSLRSDDTAVYYCARDYDYDVGMDY |
| | | WGQGTLVTVSS |
| 25 | VL | DIVMTQSPDSLAVSLGERATISCRASE |
| | | SVDNYGISFMKWFQQKPGQPPKLLIYA |
| | | ASNQGSGVPDRFSGSGSGTDFTLTISS |
| | | LQAEDVAVYYCQQSKEVPWTFGGGTKV |
| | | EIK |

In some aspects, navicixizumab is administered to a patient with gastric cancer (e.g., locally advanced or metastatic gastric cancer) in a combination treatment further comprising chemotherapy (e.g., docetaxel, cabazitaxel, etc.) and an immune checkpoint inhibitor, e.g., an anti-PD-1 antibody (e.g., sintilimab, tislelizumab, pembrolizumab, or an antigen binding portion thereof).

In some aspects, navicixizumab is administered to a patient with breast cancer (e.g., locally advance or metastatic Her-2 negative breast cancer) or prostate cancer (e.g., castration-resistance metastatic prostate cancer) in a combination treatment further comprising chemotherapy (e.g., docetaxel, cabazitaxel, etc.) or a PARP inhibitor (e.g., Rucaparib, Olaparib, etc.).

In some aspects, navicixizumab is administered to a patient with colorectal cancer (e.g., advanced colorectal cancer metastatic to liver) in a combination treatment further comprising an anti-PD(L)1 therapy and an innate immune stimulating agent (e.g., the Dectin agonist Imprime PGG, the STING agonist BMS-986301, or the NLR agonist BMS-986299).

In some aspects, navicixizumab is administered to a patient with ovarian cancer (e.g., platinum-resistant ovarian cancer or platinum-sensitive recurrent ovarian cancer) in a combination treatment further comprising a PARP inhibitor therapy (e.g., Olaparib, Rucaparib, or Niraparib) plus an immune checkpoint inhibitor therapy (e.g., anti-PD-(L)1, i.e., an inhibitor to PD-1 or PD-L1).

### Varisacumab

Varisacumab, an anti-VEGF-A monoclonal antibody, is described in detail, for example, in U.S. Pat. Nos. 8,394,943, 9,421,256, and 8,034,905, each of which is incorporated herein by reference in its entirety.

TABLE 12
Varisacumab Sequences

| SEQ ID | | |
|---|---|---|
| NO | Description | Sequence |
| 7 | VH CDR1 | SYAIS |
| 8 | VH CDR2 | GFDPEDGETIYAQKFQG |
| 9 | VH CDR3 | GRSMVRGVIIPFNGMDV |
| 10 | VL CDR1 | RASQSISSYLN |
| 11 | VL CDR2 | AASSLQS |
| 12 | VL CDR3 | QQSYSTPLT |
| 26 | VH | QVQLVQSGAEVKKPGASVKVSCKASGGTF |
| | | SSYAISWVRQAPGQGLEWMGGFDPEDGET |
| | | IYAQKFQGRVTMTEDTSTDTAYMELSSLR |
| | | SEDTAVYYCATGRSMVRGVIIPFNGMDVW |
| | | GQGTTVTVSS |
| 27 | VL | DIRMTQSPSSLSASVGDRVTITCRASQSI |
| | | SSYLNWYQQKPGKAPKLLIYAASSLQSGV |
| | | PSRFSGSGSGTDFTLTISSLQPEDFATYY |
| | | CQQSYSTPLTFGGGTKVEIK |

In some aspects, the varisacumab molecule is administered in combination with a second antibody, e.g., an anti-PD-1 antibody (e.g., sintilimab, tislelizumab, pembrolizumab, or an antigen binding portion thereof). In some aspects, the varisacumab molecule is administered in combination with a chemotherapeutic, e.g., taxane, e.g, paclitaxel or docetaxel.

In some aspects, tyrosine kinase inhibitors (TKIs) are used in anti-angiogenic therapies. Examplary TKIs include cabozantinib, vandetanib, tivozanib, axitinib, lenvatinib, sorafenib, regorafenib, sunitinib, fruquitinib, and pazopanib. In some aspects, c-MET inhibitors can be used.

Specific therapeutic agents that can be administered as part of the TME-Class specific therapies disclosed herein as include in TABLE 13.

TABLE 13
Therapeutic agents for administration in TME phenotype class-specific therapies

| TME- | | | |
|---|---|---|---|
| Class | Therapy | Therapeutic | |
| Therapy | family | agent type | Specific examples |
| IA | CPM | Anti-GITR | TRX518, INCAGN01876, BMS-986156 |
| IA | CPM | Anti-OX40 | Oxelumab |
| IA | CPM | Anti-ICOS (CD278) | vopratelimab. XmAb23104 (anti-PD- |
| | | | 1/anti-ICOS) |
| IA | CPM | Anti-4-1BB (CD137) | urelumab, utomilumab, INBRX-105 |
| | | | (anti-PD-L1/anti-4-1BB), MCL A-145 |
| | | | (anti-PD-L1/anti-4-1BB) |
| IA | CPM | RORγ agonist | LYC-55716 (cintirorgon) |
| IA, IS, ID | CPI | Anti-PD-1 | nivolumab, pembrolizumab, |
| | | | cemiplimab, PDR001, CBT-501, CX-188, |
| | | | TSR-042, XmAb20717 (anti PD- |
| | | | 1/anti-CTLA-4), cetrelimab (JNJ- |
| | | | 63723283), Gilvetmab (for canine |
| | | | veterinarian use), sintilimab (IBI308), |
| | | | tilselizumab, pidilizumab, prolgolimab |
| | | | (BCD 100), camrelizumab (SHR-1210), |
| | | | XmAb23104 (anti-PD-1/anti-ICOS), |
| | | | AK104 (anti-PD-1/anti-CTLA-4), |
| | | | MGD019 (anti-PD-1/anti-CTLA-4), |
| | | | XmAb20717 (anti-PD-1/anti-CTLA-4), |
| | | | MEDI5752 (anti-PD-1/anti-CTLA-4), |
| | | | MGD013 (anti-PD-1/anti-LAG3), |
| | | | RO7121661 (RG7769) (anti-PD-1/anti- |
| | | | TIM3), IBI318 (anti-PD-1/undisclosed |
| | | | TAA) |
| IA, IS, ID | CPI | Anti-PD-L1 | atezolizumab, avelumab, durvalumab, |
| | | | CX-072, LY3300054, INBRX-105 |
| | | | (anti-PD-L1/anti-4-1BB), MCL A-145 |
| | | | (anti-PD-L1/anti-4-1BB), KN046 (anti- |
| | | | PD-L1/anti-CTLA4), FS118 (anti-PD- |
| | | | L1/anti-LAG3), LY3415244 (anti-PD- |
| | | | L1/anti-TIM3), YW243.55.570, |
| | | | MDPL3280A |
| IA, IS, ID | CPI | Anti-PD-(L)1 | AMP-224, AN-2005, BAT1308, BBI-801, |
| | | | BCD-217, BION-004, CCX4503, |
| | | | CX-188, dual TIM-3/PD-1 antibody, |
| | | | GX-P2, KY-1003, NLG PD1 Aptamer, |
| | | | PD-1 sd-rxRNA, PD1-41BB, PRS-332, |
| | | | STI-A1010, STI-A1110, TIGIT/PD-L1 |
| | | | inhibitor (Exelexis). |
| IA, IS, ID | CPI | Anti-PD-L2 | AMP-224 |
| IA, IS, ID | CPI | Anti-CTLA-4 | ipilimumab, XmAb20717 (anti |
| | | | PD-1/anti-CTLA-4), tremelimumab, AK104 |
| | | | (anti-PD-1/anti-CTLA-4), MGD019 |
| | | | (anti-PD-1/anti-CTLA-4), XmAb20717 |
| | | | (anti-PD-1/anti-CTLA-4), MEDI5752 |
| | | | (anti-PD-1/anti-CTLA-4), KN046 |
| | | | (anti-PD-L1/anti-CTLA4), |
| IA, IS | CPI, AIT | TIM-3 inhibitor | RO7121661 (RG7769) |
| | | | (anti-PD-1/anti-TIM3), LY3415244 |
| | | | (anti-PD-L1/anti-TIM3) |
| IA, IS | CPI, AIT | LAG-3 inhibitor | relatlimab, MGD013 (anti-PD-1/anti- |
| | | | LAG3), FS118 (anti-PD-L1/anti- |
| | | | LAG3), BMS-986016 |
| IA, IS | CPI, AIT | BTLA inhibitor | |
| IA, IS | CPI, AIT | TIGIT inhibitor | Etigilimab (OMP 313M32), AB-154, |
| | | | BGB-A1217 (ociperlimab) |
| IA, IS | CPI, AIT | VISTA inhibitor | |
| IA, IS | CPI, AIT | TGF-β inhibitor | LY2157299 (galunisertib) |
| IA, IS | CPI, AIT | TGF-β R1 inhibitor | LY3200882 |
| IA, IS | CPI, AIT | CD86 agonist | |
| IA, IS | CPI, AIT | LAIR1 inhibitor | |
| IA, IS | CPI, AIT | CD160 inhibitor | |
| IA, IS | CPI, AIT | 2B4 inhibitor | |
| IA, IS | CPI, AIT | GITR inhibitor | |
| IA, IS | CPI, AIT | OX40 inhibitor | |
| IA, IS | CPI, AIT | 4-1BB (CD137) | |
| | | inhibitor | |
| IA, IS | CPI, AIT | CD2 inhibitor | |
| IA, IS | CPI, AIT | CD27 inhibitor | |
| IA, IS | CPI, AIT | CDS inhibitor | |
| IA, IS | CPI, AIT | ICAM-1 inhibitor | |
| IA, IS | CPI, AIT | LFA-1 (CD11a/CD18) | |
| | | inhibitor | |
| IA, IS | CPI, AIT | ICOS (CD278) | |
| | | inhibitor | |
| IA, IS | CPI, AIT | CD30 inhibitor | |
| IA, IS | CPI, AIT | CD40 inhibitor | |
| IA, IS | CPI, AIT | BAFFR inhibitor | |
| IA, IS | CPI, AIT | HVEM inhibitor | |
| IA, IS | CPI, AIT | CD7 inhibitor | |
| IA, IS | CPI, AIT | LIGHT inhibitor | |
| IA, IS | CPI, AIT | NKG2C inhibitor | |
| IA, IS | CPI, AIT | SLAMF7 inhibitor | |
| IA, IS | CPI, AIT | NKp80 inhibitor | |
| IS, A | AAT | Anti-VEGF | varisacumab, bevacizumab, |
| | | | navicixizumab (OMP-305B83) |
| | | | (anti-DLL4/anti-VEGF), ABL101 |
| | | | (NOV1501)(anti-DLL4/anti-VEGF), |
| | | | ranibizumab, faricimab |
| | | | (anti-Ang2/anti-VEGFA), vanucizumab |
| | | | (anti-Ang2/Anti-VEGF), BI836880 |
| | | | (anti-Ang2/anti-VEGFA), ABT165 |
| | | | (anti-DLL4/anti-VEGF), |
| IS | AAT | Anti-VEGFR1 | icrucumab (IMC-18F1) |
| IS, A | AAT | Anti-VEGFR2 | ramucirumab, alacizumab, 33C3 |
| IS | AIT | Anti-PS targeting | Bavituximab |
| IS | AIT | Anti-β2-glycoprotein | Bavituximab |
| | | 1 | |
| IS, A, ID | AIT | PI3K inhibitor | LY3023414 (samotolisib), IPI-549, |
| | | | BKM120, BYL719 |
| IS | AIT | Adenosine pathway | AB-928 |
| | | inhibitor | |
| IS | AIT | IDO inhibitor | epacadostat (INCB24360), navoximod |
| | | | (GDC-0919), BMS-986205 |
| IS | AIT | CD47 inhibitor | magrolimab (5F9), TG-1801 (NI-1701) |
| | | | (anti-CD47/anti-CD19) |
| ID | IRIT | Cancer vaccines | IGV-001 (Imvax), ilixadence, IMM-2, |
| | | | TG4010 (MVA expressing MUC-1 and |
| | | | IL-2), TroVax (MVA expressing fetal |
| | | | oncogene 5T4 (MVA-5T4)), |
| | | | PROSTVAC (or PSA-TRICOM) (MVA |
| | | | expressing PSA), GVAX, recMAGE-A3 |
| | | | protein + AS15 immunostimulant, |
| | | | Rindopepimut with GM-CSF plus |
| | | | temozolomide, IMA901 (10 different |
| | | | synthetic tumor-associated peptides), |
| | | | Tecemotide (L-BLP25) (MUC-1- |
| | | | derived lipopeptide), a DC-based |
| | | | vaccine (expressing, e.g., a cytokine |
| | | | such as IL-12), a multiepitope vaccine |
| | | | composed of tyrosinase, gp100 and |
| | | | MART-1 peptides, a peptide vaccine |
| | | | (EGFRvIII, EphA2, Her2/neu peptide) |
| | | | (alone or in combination with |
| | | | bevacizumab), HSPPC-96 (personalized |
| | | | peptide-based vaccine) (alone or in |
| | | | combination with bevacizumab, Intuvax |
| | | | (allogenic cell-based therapy) (alone |
| | | | or in combination with Sunitinib), |
| | | | PF-06755990 (vaccine) (alone or in |
| | | | combination with sunitinib and/or |
| | | | tremelimumab), NeoVax (neoantigen |
| | | | peptide) (alone or in combination with |
| | | | pembrolizumab and/or radiotherapy), |
| | | | the peptide vaccine used in clinical trial |
| | | | NCT02600949 (alone or in combination |
| | | | with pembrolizumab), DPX-Survivac |
| | | | (encapsulated peptide) (alone or in |
| | | | combination with pembrolizumab |
| | | | and/or chemotherapy, e.g., with |
| | | | cyclophosphamide), pTVG-HP (DNA |
| | | | vaccine encoding PAP antigen) (alone |
| | | | or in combination with nivolumab |
| | | | and/or CM-CSF), GVAX (GM-CSF- |
| | | | secreting tumor cells) (alone or in |
| | | | combination with nivolumab and/or |
| | | | chemotherapy, e.g., with |
| | | | cyclophosphamide), PROSTVAC |
| | | | (poxviral vector expressing PSA) (alone |
| | | | or in combination with nivolumab), |
| | | | PROSTVAC (poxviral vector |
| | | | expressing PSA) (alone or in |
| | | | combination with ipilimumab), GVAX |
| | | | (GM-CSF-secreting tumor cells) (alone |
| | | | or in combination with nivolumab and |
| | | | ipilimumab, and in combination with |
| | | | CRS-207 and cyclophosphamide), |
| | | | Dendritic cell-based p53 vaccine (alone |
| | | | or in combination with nivolumab and |
| | | | ipilimumab), Neoantigen DNA vaccine |
| | | | (in combination with durvalumab), or |
| | | | CDX-1401 vaccine (DEC-205/NY-ESO-1 |
| | | | fusion protein) (alone or in |
| | | | combination with atezolizumab and |
| | | | chemotherapy, e.g., guadecitabine) |
| ID | IRIT | CAR-T therapies | IMM-3, axicabtagene ciloleucel, |
| | | | AUTO, Immunotox, sparX/ARC-T |
| | | | therapies, BCMA CAR-T |
| ID | IRIT | TLR-based therapies | poly(I:C), BCG (Bacillus Calmette |
| | | | Guerin), IPH 31XX, monophosphoryl |
| | | | lipid A (MPL), CBLB502 (entolimod), |
| | | | CBLB502, imiquimod (ALDARA), |
| | | | 852A (ssRNA), IMOxine (CpG-ODN), |
| | | | MGN1703 (dSLIM, CpG-ODN), |
| | | | PF3512676, 1018 ISS, lefitolimod, |
| | | | SD-101, VTX-2337, EMD 1201081, |
| | | | IMO-2125, DV281, CMP-001, or CPG7907 |
| A, IS | VTT/A | Angiopoietin 1 (Ang1) | |
| | | inhibitor | |
| A, IS | VTT/A | Angiopoietin 2 (Ang2) | vanucizumab (anti-Ang2/Anti-VEGF), |
| | | inhibitor | faricimab (anti-Ang2/anti-VEGFA), |
| | | | nesvacumab, BI836880 |
| | | | (anti-Ang2/anti-VEGFA) |
| A, IS | VTT/A | DLL4 inhibitor | |
| A, IS | VTT/A | TKI inhibitor | cabozantinib, vandetanib, tivozanib, |
| | | | axitinib, lenvatinib, sorafenib, |
| | | | regorafenib, sunitinib, fruquitinib, |
| | | | pazopanib, apatinib, 3D011, 4SC-203, |
| | | | A006, ACTB1003, Acurita, AEE788, |
| | | | AGN-745, AIV001, AIV007, AK109, |
| | | | altiratinib, AM-712, APL-102, APX004, |
| | | | BL-011256, BMS-690514, BMS-817378, |
| | | | BR55, brivanib, cabozantinib, |
| | | | cederinib, CDP 791, CEP-11981, |
| | | | CTx-0294886, CTx-0357927, CYC116, |
| | | | dovitinib lactate, elpamotide, famitinib, |
| | | | FLAG-003, foretinib, GEN-2, GFB-204, |
| | | | golvatinib, henatinib, HLX06, HLX12, |
| | | | HyNap-Sora, IBI302, Iclusig |
| | | | (ponatinib), ICP-033, icrucumab, |
| | | | IMC-1C11, IMC-3C5, INXN-4001, |
| | | | jintuximab, KD020, lucitanib, |
| | | | MSB0254, NEV-801, ningetinib, ODM-203, |
| | | | ofev, OPT-302, orantinib, OSI-632, |
| | | | pegdinetanib, PF-337, 210, RAF265, |
| | | | recentin, rivoceranib, Rydapt/PKC412, |
| | | | SAR402663, sitravatinib, STP355, SU-14813, |
| | | | surufatinib, TAS-115, telatinib, |
| | | | tesevatinib, TLK60404, TTAC-0001, |
| | | | vatalanib, V-DOS47, versavo, |
| | | | vorolanib, VXM01, XC001, |
| | | | XL092XL999. |
| A, IS, ID | VTT/A | c-MET inhibitor | |
| A, IS, ID | VTT/A | Anti-FGF | |
| A, IS, ID | VTT/A | anti-FGFR1 | BFKB8488A (RG7992) (anti- |
| | | | FGFR1/anti-KLB) |
| A, IS, ID | VTT/A | Anti-FGFR2 | bemarituzumab (FPA144), aprutumab |
| | | | (BAY 1179470) |
| A, IS, ID | VTT/A | FGFR1 inhibitor | |
| A, IS, ID | VTT/A | FGFR2 inhibitor | |
| A, IS | VTT/A | Anti-PLGF | |
| A, IS | VTT/A | PLGF inhibitor | |
| A, IS | VTT/A | Anti-VEGFB | |
| A, IS | VTT/A | Anti-VEGFC | |
| A, IS | VTT/A | Anti-VEGFD | |
| A, IS | VTT/A | Anti-VEGF/PLGF | ziv-aflibercept |
| | | trap | |
| A, IS | VTT/A | Anti-DLL4/anti- | navicixizumab (anti-DLL4/anti-VEGF), |
| | | VEGF | ABL101 (NOV1501) (anti-DLL4/anti- |
| | | | VEGF), ABT165 (anti-DLL4/anti-VEGF) |
| A, IS, ID | VTT/A | Anti-Notch | Brontictuzumab, tarextumab |
| A, IS | ATTT | Endoglin | |
| A, IS | ATTT | Angiopoietin | |
| A, IS | ATTT | Antagonist to endoglin | TRC105 |
| A, IS | VTT/A | Anti-DLL4 | navicixizumab (anti-DLL4/anti-VEGF), |
| | | | ABL101 (NOV1501) (anti-DLL4/anti- |
| | | | VEGF), ABT165 (anti-DLL4/anti-VEGF), |
| | | | demcizumab |
| IA, IS, ID, A | Chemo | Taxanes | Paclitaxel, docetaxel |
| IA, IS, ID, A | Chemo | Vinca alkaloyds | Vinblastine, vincristine |
| IA, IS, ID, A | Chemo | Anthracyclines | Daunorubicin, doxorubicin, |
| | | | aclacinomycin, dihydroxy anthracin |
| | | | dione, mitoxantrone, |
| IA, IS, ID, A | Chemo | Topoisomerase | camptothecin, topotecan, irinotecan, |
| | | inhibitor | 20-S camptothecin, 9-nitro-camptothecin, |
| | | | 9-amino-camptothecin, G1147211 |
| IA, IS, ID, A | Chemo | Antimetabolites | methotrexate, 6-mercaptopurine, |
| | | | 6-thioguanine, cytarabine, 5-fluorouracil |
| | | | decarbazine |
| IA, IS, ID, A | Chemo | Alkylating agents | mechlorethamine, thioepa chlorambucil, |
| | | | CC-1065, melphalan, carmustine |
| | | | (BSNU), lomustine (CCNU), |
| | | | cyclophosphamide, busulfan, |
| | | | dibromomannitol, streptozotocin, |
| | | | mitomycin C, cysplatin, |
| | | | cis-dichlorodiamine platinum (II) |
| | | | (DDP) cisplatin |
| IA, IS, ID, A | Chemo | Other | etoposide, hydroxyurea, cytochalasin B, |
| | | | gramicidin D, emetine, mitomycin, |
| | | | tenoposide, colchicine, mithramycin, |
| | | | actinomycin D, 1-dehydrotestosterone, |
| | | | glucocorticoids, maytansinoid (e.g., |
| | | | maytansinol or CC-1065) |
| ID | Chemo | Antibody-Drug | DS-8201a, glembatumumab vedotin, |
| | | Conjugates (ADC) | ABBV-085, IMMU-130, SGN-15, |
| | | | brentuximab vedotin, SYD985, |
| | | | BA3011, inotuzumab ozogamicin. |

CPM: Check Point Modulator;
CPI: Check Point Inhibitor;
AAT: Anti-Angiogenic Therapy;
AIT: Anti-Immunosuppression Therapy;
IRIT: Immune Response Initiation Therapy;
VTT/A: VEGF-targeted therapy/Other Antiogenics;
ATTT: Angiopoietin/TIE2-Targeted Therapy;
Chemo: Chemotherapy

### III.D ID TME Phenotype Class-Specific Therapies

In some aspects, the TME is dominated by lack of immune cells but vasculature is functional. Accordingly, such TME of a cancer tumor can be classified in the TD (Immune Desert) TME phenotype class by a classifier of the present disclosure such as the TME Panel-1 Classifier (i.e., an ID biomarker-positive patient). When this TME phenotype class is identified in a patient's sample, the tumor can be treated with an ID-class TME therapy.

In some aspects, the ID-class TME therapy comprises the administration of a checkpoint modulator therapy concurrently or after the administration of a therapy that initiates an immune response. In some aspects, the therapy that initiates an immune response is a vaccine, a CAR-T, or a neo-epitope vaccine. In some aspects, the checkpoint modulator therapy comprises the administration of an inhibitor of an inhibitory immune checkpoint molecule. In some aspects, the inhibitor of an inhibitory immune checkpoint molecule is an antibody against PD-1 (e.g., sintilimab, tislelizumab, pembrolizumab, or an antigen binding portion thereof), PD-L1, PD-L2, CTLA-4, or a combination thereof. In some aspects, the anti-PD-1 antibody comprises nivolumab, pembrolizumab, cemiplimab, PDR001, CBT-501, CX-188, sintilimab, tislelizumab, or TSR-042, or an antigen-binding portion thereof. In some aspects, the anti-PD-1 antibody cross-competes with nivolumab, pembrolizumab, cemiplimab, PDR001, CBT-501, CX-188, sintilimab, tislelizumab, or TSR-042, for binding to human PD-1. In some aspects, the anti-PD-1 antibody binds to the same epitope as nivolumab, pembrolizumab, cemiplimab, PDR001, CBT-501, CX-188, sintilimab, tislelizumab, or TSR-042. In some aspects, the anti-PD-L1 antibody comprises avelumab, atezolizumab, CX-072, LY3300054, durvalumab, or an antigen-binding portion thereof. In some aspects, the anti-PD-L1 antibody cross-competes with avelumab, atezolizumab, CX-072, LY3300054, or durvalumab for binding to human PD-L1. In some aspects, the anti-PD-L1 antibody binds to the same epitope as avelumab, atezolizumab, CX-072, LY3300054, or durvalumab. In some aspects, the anti-CTLA-4 antibody comprises ipilimumab or the bispecific antibody XmAb20717 (anti PD-1/anti-CTLA-4), or an antigen-binding portion thereof. In some aspects, the anti-CTLA-4 antibody cross-competes with ipilimumab or the bispecific antibody XmAb20717 (anti PD-1/anti-CTLA-4) for binding to human CTLA-4. In some aspects, the anti-CTLA-4 antibody binds to the same CTLA-4 epitope as ipilimumab or the bispecific antibody XmAb20717 (anti PD-1/anti-CTLA-4). In some aspects, the checkpoint modulator therapy comprises the administration of (i) an anti-PD-1 antibody selected from the group consisting of nivolumab, pembrolizumab, cemiplimab PDR001, CBT-501, CX-188, sintilimab, tislelizumab, and TSR-042; (ii) an anti-PD-L1 antibody selected from the group consisting of avelumab, atezolizumab, CX-072, LY3300054, and durvalumab; (iv) an anti-CTLA-4 antibody, which is ipilimumab or the bispecific antibody XmAb20717 (anti PD-1/anti-CTLA-4), or (iii) a combination thereof.

For the TME with no immune activity, such as a patient classified as the ID (Immune Desert) phenotype (i.e., an ID biomarker-positive patient), a patient with this biology would not respond to a monotherapy of checkpoint inhibitors, anti-angiogenics or other TME targeted therapies, and so should not be treated with anti-PD-1s, anti-PD-L1s, anti-CTLA-4s, or RORγ agonists as monotherapies. A patient with this biology can be treated with therapies that induce immune activity allowing them to then get benefit from checkpoint inhibitors or other TME targeted therapies. Therapies that might induce immune activity for these patients include vaccines, CAR-Ts, neo-epitope vaccines, including personalized vaccines, and Pattern Recognition Receptor (PRR) TLR-based therapies.

CAR-T therapy is a type of treatment in which a patient's T cells (a type of immune system cell) are changed in the laboratory so they will attack cancer cells. T cells are taken from a patient's blood. Then the gene for a special receptor that binds to a certain protein on the patient's cancer cells is added in the laboratory. The special receptor is called a chimeric antigen receptor (CAR). Large numbers of the CAR T-cells are grown in the laboratory and given to the patient by infusion. CAR T-cell therapy is being studied in the treatment of some types of cancer. Also called chimeric antigen receptor T-cell therapy. In some aspects, a CAR-T therapy comprises the administration of IMM-3, axicabtagene ciloleucel, AUTO, Immunotox, sparX/ARC-T therapies, or BCMA CAR-T.

Pattern Recognition Receptor Agonist Class: Toll-like receptors (TLRs), mammalian homolog of drosophila Toll protein, are regarded as critical pattern recognition receptors (PRRs) of innate immunity. Some TLR agonists have been found to induce strong antitumor activity by indirectly activating tolerant host immune system to destroy cancer cells. Therefore, specific agonists of TLRs can be used to treat cancer. Multiple TLR agonists have been considered for clinical application. BCG (Bacillus Calmette-Guerin)—an agonist of TLR2 and TLR4—can be used, e.g., for therapy of superficial bladder cancer or colorectal cancer. TLR3 (Toll-like receptor 3) ligand IPH-3102 (IPH-31XX) can be used to treat, e.g., breast cancer. TLR4 (Toll-like receptor 4) agonist monophosphoryl lipid A (MPL) can be used, e.g., for the treatment of colorectal cancer. In some aspects, MPL can be administered with CERVARIX™ vaccines as an adjuvant for the prophylaxis of HPV (human papilloma virus)-associated cervical cancer. In some aspects, flagellin-derived agonist CBLB502 (entolimod) can be used to treat advanced solid tumors.

In some aspects, the TLR-based therapy comprises the administration of BCG (Bacillus Calmette-Guerin), monophosphoryl lipid A (MPL), entolimod (CBLB502), imiquimod (ALDARA®), 852A (small molecule ssRNA), IMOxine (CpG-ODN), lefitolimod (MGN1703), dSLIM® (Double-Stem Loop ImmunoModulator), CpG oligodeoxynucleotides (CpG-ODN), PF3512676 (also known as CpG7909; alone or combined with chemotherapy), 1018 ISS (alone or in combination with chemotherapy or RITUXAN®), SD-101, motolimod (VTX-2337), IMO-2055 (IMOxine; EMID 1201081), tilsotolimod (IMO-2125), DV281, CMP-001, or CPG7907, VB-201, OPN-305, INNA-051, CBLB612, Ampligen, BO-102, Poly-ICLC, PrEP-001, YS-ON-001, ID-G100, ID-93, TARA-002, ALT-702, APR-003, BDB-001, BNT411, CV8102, DSP-0509, GS986, PRTX007, resiquimod, RG6115, RG7854, TRANSCON, Vesimune, Vesatolimod, Motolimod, SBT6050, SBT6290, SBT8230, Tallac/ALX, Cavrotilimod, EnanDIM, Heplisav-B, Kappaproct, and vidutolimod.

Agonists of other PRRs would also be expected to activate the innate immune system and thereby instigate a robust anti-cancer response inclusive of the adaptive immune system. Accordingly, these agents could be useful in the treatment of ID phenotype tumors as defined by the Xerna TME panel. These include agonists of the C-type Lectin Receptors (i.e. DECTIN-1, DECTIN-2, MINCLE) including Beta Glucans such as Imprime PGG; agonists of Retinoic Acid Inducible Gene-like receptors (RIG-I), such as CV8102, MK4621, Inarigivir, BO-112; agonists of the NOD-like receptors such as BMS-986299; and agonists of the cGAS-STING pathway including ADU-S100, BMS-986301, CRD-100, CRD-5500, E7766, exoSTING, GB492, GSK3745417, MAVU-104, MK-1454, NZ-STING, ONM-500, ONM-501, SB11285, SNX 281, SOMCL-18-202, STACT-TREX1, STI-001, SYNB1891, TAK676, TTI-10001, and XMT-2056.

Therapeutic cancer vaccines are based on specific stimulation of the immune system using tumor antigens to elicit an antitumor response. In some aspects, the cancer vaccine comprises, e.g., IGV-001 (IMVAX™), ilixadencel, IMM-2, TG4010 (MVA expressing MUC-1 and IL-2), TROVAX® (MVA expressing fetal oncogene 5T4 (MVA-5T4)), PROSTVAC® (or PSA-TRICOM®) (MVA expressing PSA), GVAX®, recMAGE-A3 (recombinant Melanoma-associated antigen 3) protein plus AS15 immunostimulant, rindopepimut with GM-CSF plus temozolomide, IMA901 (10 different synthetic tumor-associated peptides), recemotide (L-BLP25) (MUC-1-derived lipopeptide), a DC-based vaccine (expressing, e.g., a cytokine such as IL-12), a multiepitope vaccine composed of tyrosinase, gp100 and MART-1 peptides, a peptide vaccine (EGFRvIII, EphA2, Her2/neu peptide) (alone or in combination with bevacizumab), HSPPC-96 (personalized peptide-based vaccine) (alone or in combination with bevacizumab, INTUVAX® (allogenic cell-based therapy) (alone or in combination with sunitinib), PF-06755990 (vaccine) (alone or in combination with sunitinib and/or tremelimumab), NEOVAX® (neoantigen peptide) (alone or in combination with pembrolizumab and/or radiotherapy), the peptide vaccine used in clinical trial NCT02600949 (alone or in combination with pembrolizumab), DPX-Survivac (encapsulated peptide) (alone or in combination with pembrolizumab and/or chemotherapy, e.g., with cyclophosphamide), pTVG-HP (DNA vaccine encoding PAP antigen) (alone or in combination with nivolumab and/or CM-CSF), GVAX® (GM-CSF-secreting tumor cells) (alone or in combination with nivolumab and/or chemotherapy, e.g., with cyclophosphamide), PROSTVAC® (poxviral vector expressing PSA) (alone or in combination with nivolumab), PROSTVAC® (poxviral vector expressing PSA) (alone or in combination with ipilimumab), GVAX® (GM-CSF-secreting tumor cells) (alone or in combination with nivolumab and ipilimumab, and in combination with CRS-207 and cyclophosphamide), dendritic cell-based p53 vaccine (alone or in combination with nivolumab and ipilimumab), neoantigen DNA vaccine (in combination with durvalumab), or CDX-1401 vaccine (DEC-205/NY-ESO-1 fusion protein) (alone or in combination with atezolizumab and chemotherapy, e.g., guadecitabine).

Antibody-based activators of the immune response may also be useful to stimulate the immune response, especially in ID phenotype tumors which lack evidence of an ongoing anti-cancer immune response. These would include agonistic antibodies of CD27 such as varlilumab and CDX-527. These would also include antibody agonists of 4-1BB (CD137) such as FS222, ABL 503, INBRX-105, GEN1046, MCLA-145. These would also include agonists of CD40 such as CDX1140, selicrelumab, CP-870,893, dacetuzmumab, ChiLob7/4.

### IV. Adjuvant Therapies

The methods to select a patient with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) for treatment with a certain therapy and well as the methods of treatment disclosed herein can also comprise (i) the administration of additional therapies, for example, chemotherapy, hormonal therapy, or radiotherapy, (ii) surgery, or (iii) combinations thereof. In some aspects, additional (adjuvant) therapies can be administered simultaneously or sequentially (before or after) the administration of the TME phenotype class-specific therapies disclosed above or a combination thereof.

Adjuvant chemotherapy is effective in preventing the outgrowth of micrometastatic disease from cancer that has been removed surgically, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC). Studies have shown that fluorouracil is an effective adjuvant chemotherapy among patients with microsatellite stability or low-frequency microsatellite instability, but not in patients with high-frequency microsatellite instability.

When one or more adjuvant therapies are used in combination with a TME phenotype class-specific therapy as described herein or a combination thereof, there is no requirement for the combined results to be additive of the effects observed when each treatment is conducted separately. Although at least additive effects are generally desirable, any increased therapeutic effect or benefit (e.g., reduced side-effects) above one of the single therapies would be of value. Also, there is no particular requirement for the combined treatment to exhibit synergistic effects, although this is possible and advantageous.

“Neoadjuvant therapy” may be given as a first step to shrink a tumor before the main treatment, which is usually surgery, is given. Examples of neoadjuvant therapy include, e.g., chemotherapy and radiation therapy. It is a type of induction therapy.

In a particular aspect, A TME phenotype class therapy can be administered in combination with chemotherapeutics, e.g., taxanes such as paclitaxel or docetaxel. In some aspects, A TME phenotype class therapy can comprise chemotherapy (e.g., taxanes such as paclitaxel or docetaxel) combined with VEGF-targeted therapies and/or DLL-4-targeted therapies.

Chemotherapy can be administered as standard of care. Thus, if a cancer patient or a patient's cancer is assigned to a particular TME phenotype class or a combination thereof (i.e., the patient is biomarker-positive for one of more TME phenotype classes and/or biomarker-negative for one or more TME phenotype classes), the specific therapy for that TME phenotype class or combination thereof can be added to the standard of care chemotherapy.

In some aspects, the cancer is selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC).

### IV.A Chemotherapy

TME phenotype class-specific therapies as described herein can be administered to patients with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) in combination with one or more adjuvant chemotherapeutic agents or drugs.

The term “chemotherapy” refers to various treatment modalities affecting cell proliferation and/or survival. The treatment may include administration of alkylating agents, antimetabolites, anthracyclines, plant alkaloids, topoisomerase inhibitors, and other antitumor agents, including monoclonal antibodies and kinase inhibitors. The term “neoadjuvant chemotherapy” relates to a preoperative therapy regimen consisting of a panel of chemotherapeutic and/or antibody agents, which is aimed to shrink the primary tumor, thereby rendering local therapy (surgery or radiotherapy) less destructive or more effective enabling evaluation of responsiveness of tumor sensitivity towards specific agents in vivo.

Chemotherapeutic drugs can kill proliferating tumor cells, enhancing the necrotic areas created by the overall treatment. The drugs can thus enhance the action of the primary therapeutic agents of the present disclosure.

Chemotherapeutic agents used in cancer treatment can be divided into several groups, depending on their mechanism of action. Some chemotherapeutic agents directly damage DNA and RNA. By disrupting replication of the DNA such chemotherapeutics either completely halt replication, or result in the production of nonsense DNA or RNA. This category includes, for example, cisplatin (PLATINOL®), daunorubicin (CERUBIDINE®), doxorubicin (ADRIAMYCIN®), and etoposide (VEPESID®). Another group of cancer chemotherapeutic agents interferes with the formation of nucleotides or deoxyribonucleotides, so that RNA synthesis and cell replication is blocked. Examples of drugs in this class include methotrexate (ABITREXATE®), mercaptopurine (PURINETHOL®), fluorouracil (ADRUCIL®), and hydroxyurea (HYDREA®). A third class of chemotherapeutic agents affects the synthesis or breakdown of mitotic spindles, and, as a result, interrupts cell division. Examples of drugs in this class include vinblastine (VELBAN®), vincristine (ONCOVIN®) and taxenes, such as, paclitaxel (TAXOL®), and docetaxel (TAXOTERE®). Chemotherapeutic regimens such as FOLFOX (leucovorin “FOL”, fluorouracil (5-FU) “F”, and oxaliplatin (eloxatin) “OX”) or FOLFIRI—(leucovorin “FOL”, fluorouracil (5-FU) “F”, and irinotecan (camptosar) “IRI”) are also used in treatment of colorectal cancer or another type of cancer disclosed herein.

In some aspects, the methods disclosed herein include treatment of patients with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) with a taxane derivative, e.g., paclitaxel or docetaxel. In some aspects, the method disclosed herein includes treatment of patients with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) with an anthracycline derivative, such as, for example, doxorubicin, daunorubicin, and aclacinomycin. In some aspects, the method disclosed herein include treatment of patients with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) with a topoisomerase inhibitor, such as, for example, camptothecin, topotecan, irinotecan, 20-S camptothecin, 9-nitro-camptothecin, 9-amino-camptothecin, or water soluble camptothecin analog G1147211. Treatment with any combination of these and other chemotherapeutic drugs is specifically contemplated.

Patients with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) can receive chemotherapy immediately following surgical removal of a tumor. This approach is commonly referred to as adjuvant chemotherapy. However, chemotherapy can be administered also before surgery, as so-called neoadjuvant chemotherapy.

### IV.B Radiotherapy

TME phenotype class-specific therapies as described herein may be administered to a patient suffering from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) in combination with radiotherapy.

The terms “radiation therapy” and “radiotherapy” refers to the treatment of cancer with ionizing radiation, which comprises particles having sufficient kinetic energy to emit electrons from atoms or molecules and thereby generate ions. The term includes treatments with direct ionizing radiation, such as those produced by alpha particles (helium nuclei), beta particles (electrons), and atomic particles such as protons, and indirect ionizing radiation, such as photons (including gamma and x-rays). Examples of ionizing radiation used in radiation therapy include high energy X-rays, γ-irradiation, electron beams, UV irradiation, microwaves, and photon beams. The direct delivery of radioisotopes to tumor cells is also contemplated.

Most patients receive radiotherapy immediately following surgical removal of a tumor. This approach is commonly referred to as adjuvant radiotherapy. However, radiotherapy can be administered also before surgery, as so-called neoadjuvant radiotherapy.

### V. Colorectal Cancer (CRC)

Colorectal Cancer (CRC) is the third most common type of cancer, and is deadly in its advanced stages. While curative surgery is appropriate for early-stage disease, up to 30% of the patients experience recurrence within 2-5 years (Fatemi et al. 2015 Iran. J. Cancer Prev. 8; Duineveld et al. 2016 Ann. Fam. Med. 14:215-220). Certain targeted therapies are available for late-stage CRC, such as anti-EGFR depending on mutation status, anti-angiogenics, or checkpoint inhibitors in cases where patients are shown to be MSI-H/dMMR. Unfortunately, few diagnostic tools exist to match an individual with recurrent metastatic disease to the optimal therapy regime, and for the majority of patients with metastatic disease, the clinician must choose therapies without the benefit of precision tools that would indicate the best course of treatment.

The methods and compositions disclosed herein can be used for the treatment of colorectal cancer, e.g., to identify patients for treatment with specific therapies, to predict disease free probability and overall survival, or to predict the outcome of targeted therapies. Colorectal cancer (CRC), also known as bowel cancer, colon cancer, or rectal cancer, is the development of cancer from the colon or rectum (parts of the large intestine). A “cancer” refers to a broad group of various proliferative diseases characterized by the uncontrolled growth of abnormal cells in the body. Unregulated cell division and growth results in the formation of malignant tumors that invade neighboring tissues and can also metastasize to distant parts of the body through the lymphatic system or bloodstream.

Colorectal cancer is a disease originating from the epithelial cells lining the colon or rectum of the gastrointestinal tract, most frequently as a result of mutations in the Wnt signaling pathway that increase signaling activity. The mutations can be inherited or acquired, and most probably occur in the intestinal crypt stem cell. The most commonly mutated gene in all colorectal cancer is the APC gene, which produces the APC protein. The APC protein prevents the accumulation of β-catenin protein. Without APC, β-catenin accumulates to high levels and translocates (moves) into the nucleus, binds to DNA, and activates the transcription of proto-oncogenes. These genes are normally important for stem cell renewal and differentiation, but when inappropriately expressed at high levels, they can cause cancer. While APC is mutated in most colon cancers, some cancers have increased β-catenin because of mutations in β-catenin (CTNNB1) that block its own breakdown, or have mutations in other genes with function similar to APC such as AXIN1, AXIN2, TCF7L2, or NKD1.

Beyond the defects in the Wnt signaling pathway, other mutations must occur for the cell to become cancerous. The p53 protein, produced by the TP53 gene, normally monitors cell division and induces their programmed death if they have Wnt pathway defects. Eventually, a cell line acquires a mutation in the TP53 gene and transforms the tissue from a benign epithelial tumor into an invasive epithelial cell cancer. Sometimes the gene encoding p53 is not mutated, but another protective protein named BAX is mutated instead.

Other proteins responsible for programmed cell death that are commonly deactivated in colorectal cancers are TGF-β and DCC (Deleted in Colorectal Cancer). TGF-β has a deactivating mutation in at least half of colorectal cancers. Sometimes TGF-β is not deactivated, but a downstream protein named SMAD is deactivated. DCC commonly has a deleted segment of a chromosome in colorectal cancer.

Approximately 70% of all human genes are expressed in colorectal cancer, with just over 1% having increased expression in colorectal cancer compared to other forms of cancer. Some genes are oncogenes: they are overexpressed in colorectal cancer. For example, genes encoding the proteins KRAS, RAF, and PI3K, which normally stimulate the cell to divide in response to growth factors, can acquire mutations that result in over-activation of cell proliferation. The chronological order of mutations is sometimes important. If a previous APC mutation occurred, a primary KRAS mutation often progresses to cancer rather than a self-limiting hyperplastic or borderline lesion. PTEN, a tumor suppressor, normally inhibits PI3K, but can sometimes become mutated and deactivated.

Comprehensive, genome-scale analysis has revealed that colorectal carcinomas can be categorized into hypermutated and non-hypermutated tumor types. In addition to the oncogenic and inactivating mutations described for the genes above, non-hypermutated samples also contain mutated CTNNB1, FAM123B, SOX9, ATM, and ARID1A. Progressing through a distinct set of genetic events, hypermutated tumors display mutated forms of ACVR2A, TGFBR2, MSH3, MSH6, SLC9A9, TCF7L2, and BRAF. The common theme among these genes, across both tumor types, is their involvement in Wnt and TGF-β signaling pathways, which results in increased activity of MYC, a central player in colorectal cancer.

Mismatch repair (MMR) deficient tumors are characterized by a relatively high amount of poly-nucleotide tandem repeats. This is caused by a deficiency in MMR proteins—which are typically caused by epigenetic silencing and or inherited mutations (e.g. Lynch syndrome). 15 to 18 percent of colorectal cancer tumours have MMR deficiencies, with 3 percent developing due to Lynch syndrome. The role of the mismatch repair system is to protect the integrity of the genetic material within cells (i.e.: error detecting and correcting). Consequently, a deficiency in MMR proteins may lead to an inability to detect and repair genetic damage, allowing for further cancer-causing mutations to occur and colorectal cancer to progress.

The polyp to cancer progression sequence is the classical model of colorectal cancer pathogenesis. The polyp to cancer sequence describes the phases of transition from benign tumours into colorectal cancer over many years. Central to the polyp to CRC sequence are gene mutations, epigenetic alterations and local inflammatory changes. The polyp to CRC sequence can be used as an underlying framework to illustrate how specific molecular changes lead to various cancer subtypes.

In some aspects, the methods and compositions disclosed herein are used to reduce or decrease a size of a colorectal cancer tumor or inhibit a colorectal cancer tumor growth in a subject in need thereof.

Agents that can be used for the treatment of colorectal cancer include, e.g., semustine (methyl CCNU), raltitrexed (TOMUDEX®), fluorouracil (5 fluorouracil, 5 FU, fluouracil, fluorodeoxyuridine) (EFUDEX®, CARAC®, FLUOROPLEX®), floxuridine (prodrug) (FUDR®), doxifluridine, mitomycin (mitomycin C), docetaxel (TAXOTERE®), oxaliplatin (ELOXATIN®, MEDAC®), irinotecan (CPT-11), camptosar, cetuximab (anti-EGFR) (ERBITUX®), panitumumab (anti-EGFR) (VECTIBIX®), bevacizumab (anti-VEGF) (AVASTIN®), alloStim, AMP-224, ampligen, avicine, ColoAdl, CV-301, DCVax-Colon, ETBX-011, GI-4000, imlygic, IMM-101, imprime PGG, pembrolizumab (KEYTRUDA®), MelCancerVac, OncoVAX, nivolumab (OPDIVO®), Pexa-Vec, pidilizumab, PV-10, revlimid, tecemotide, tremelimumab, TroVax, Vigil vaccine, and combinations thereof.

In some aspects, subjects diagnosed with metastatic colorectal cancer have samples of their tumor tissue genotyped for mutations such as RAS (KRAS and NRAS) and/or BRAF, individually or as part of a gene panel, e.g., a next generation sequencing (NGS) panel. In some aspects, metastatic colorectal tumor samples are tested for universal mismatch repair (MMR) and/or microsatellite instability (MSI). In some aspects, metastatic colorectal tumor samples are tested for HER2 levels, e.g., via immunohistochemistry, fluorescence in situ hybridization (FISH), or NGS. In some aspects, metastatic colorectal tumor samples are further tested for NTRK fusions if the subject's tumor samples are positive for wild type KRAS, NRAS, BRAF. In some aspects, metastatic colorectal tumor samples are further tested for NTRK fusions if the subject's tumor samples are MMR deficient (dMMR)/MSI-H.

In some aspects, a subject with a colorectal cancer determined to be eligible for intensive therapy can be treated with (i) chemotherapy with or without bevacizumab (AVASTIN®), (ii) chemotherapy with or without anti-EGFR therapy such as cetuximab (ERBITUX®) or panitumumab (VECTIBIX®) if the subject's colorectal tumor samples have tested positive for KRAS/NRAS wild type and the colorectal cancer is a left sided tumor, or (iii) nivolumab (OPDIVO®) monotherapy, pembrolizumab (KEYTRUDA®) monotherapy, or nivolumab (OPDIVO®) and ipilimumab (YERVOY®) combined therapy if the subject's colorectal tumor samples have tested positive for dMMR/MSI-H.

In some aspects, a subject with a colorectal cancer who was determined to be eligible for intensive therapy but has progressed following one of the intensive therapy treatments disclosed above, can be treated with alternative therapies, e.g., (i) chemotherapy with or without bevacizumab (AVASTIN®), (ii) chemotherapy with or without anti-EGFR therapy such as cetuximab (ERBITUX®) or panitumumab (VECTIBIX®) if the subject's colorectal tumor samples have tested positive for KRAS/NRAS wild type and the colorectal cancer is a left sided tumor, (iii) regorafenib, or (iv) trifluridine plus tipiracil, with or without bevacizumab (AVASTIN®). In some aspects, if the subject with colorectal cancer who was determined to be eligible for intensive therapy but has progressed following one of the treatments disclosed above tested positive for dMMR/MSI-H colorectal cancer, the subject can be treated with nivolumab (OPDIVO®) monotherapy, pembrolizumab (KEYTRUDA®) monotherapy, or nivolumab (OPDIVO®) and ipilimumab (YERVOY®) combined therapy. In some aspects, if the subject with colorectal cancer who was determined to be eligible for intensive therapy but has progressed following one of the treatments disclosed above tested positive for HER2-amplified status and RAS and BRAF WT, the subject can be treated with (i) trastuzumab (HERCEPTIN®) with pertuzumab (PERJETA®) or lapatinib, or (ii) fam-trastuzumab deruxtecan-nxki (ENHERTU®).

In some aspects, a subject with a colorectal cancer deemed not eligible for intensive therapy can be treated, for example, less intensive chemotherapy, with or without bevacizumab. In some aspects, a subject with a colorectal cancer deemed not eligible for intensive therapy who has tested positive for KRAS/NRAS wild type and has a left sided tumors, can be treated with anti-EGFR therapy comprising, e.g., cetuximab (ERBITUX®) orpanitumumab (VECTIBIX®). In some aspects, a subject with a colorectal cancer deemed not eligible for intensive therapy who has tested positive for MSI-H/dMMR, can be treated with nivolumab (OPDIVO®) monotherapy, pembrolizumab (KEYTRUDA®) monotherapy, or nivolumab (OPDIVO®) and ipilimumab (YERVOY®) combined therapy. In some aspects, a subject with a colorectal cancer deemed not eligible for intensive therapy who has tested positive for HER2-amplified status and RAS and BRAF WT, can be treated with (i) trastuzumab (HERCEPTIN®) with pertuzumab (PERJETA®) or lapatinib, or (ii) fam-trastuzumab deruxtecan-nxki (ENHERTU®).

In some aspects, the functional status of a subject with a colorectal cancer deemed not eligible for intensive therapy but who has progressed following one of the treatments disclosed above is determined. If the subject has experienced an improvement in functional status, then the subject may be deemed eligible for intensive therapy, and be administered oe of the intensive therapies disclosed above, i.e., (i) chemotherapy with or without bevacizumab (AVASTIN®), (ii) chemotherapy with or without anti-EGFR therapy such as cetuximab (ERBITUX®) or panitumumab (VECTIBIX®) if the subject's colorectal tumor samples have tested positive for KRAS/NRAS wild type and the colorectal cancer is a left sided tumor, or (iii) nivolumab (OPDIVO®) monotherapy, pembrolizumab (KEYTRUDA®) monotherapy, or nivolumab (OPDIVO®) and ipilimumab (YERVOY®) combined therapy if the subject's colorectal tumor samples have tested positive for dMMR/MSI-H.

### VI. Kits and Articles of Manufacture

The present disclosure also provides kits and articles of manufacture comprising reagents and instructions to allow obtaining RNA expression data from a sample obtained from a patient with gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC). This data, in turn, would be used as input to the TME Panel-1 classifier disclosed herein, which would classify the tumor sample within one or more TME phenotype classes. Accordingly, the present disclosure provides a kit comprising (i) a plurality of oligonucleotide probes capable of specifically detecting an RNA encoding a gene biomarker from TABLE 1, and (ii) a plurality of oligonucleotide probes capable of specifically detecting an RNA encoding a gene biomarker from TABLE 2. Also provided is an article of manufacture comprising (i) a plurality of oligonucleotide probes capable of specifically detecting an RNA encoding a gene biomarker from TABLE 1, and (ii) a plurality of oligonucleotide probes capable of specifically detecting an RNA encoding a gene biomarker from TABLE 2, wherein the article of manufacture comprises a microarray.

In some aspects, the kit or article of manufacture can comprise (i) a plurality of oligonucleotide probes capable of specifically detecting RNAs encoding genes in a gene biomarker set from TABLE 3, and (ii) a plurality of oligonucleotide probes capable of specifically detecting RNAs encoding genes in a gene biomarker set from TABLE 4.

In some aspects, the kit or article of manufacture can comprise a plurality of oligonucleotide probes capable of specifically detecting RNAs encoding genes in a gene panel from TABLE 5.

In some aspects, the kit or article of manufacture can comprise a plurality of oligonucleotide probes capable of specifically detecting RNAs encoding genes in a gene panel (genesets) disclosed in FIG. 9A-G. In some aspects, the kits disclosed herein can comprise oligonucleotide probes to determine the dMMR or MSI-H status of the cancer patient. In some aspects, the kits disclosed herein can comprise oligonucleotide probes to determine the BRAF mutation status of the cancer patient. In some aspects, the kits disclosed herein can comprise oligonucleotide probes to determine the presence or absence of mutations in CTNNB1, FAM123B, SOX9, ATM, ARID1A, ACVR2A, TGFBR2, MSH3, MSH6, SLC9A9, TCF7L2, BRAF, and any combination thereof. The kit can also comprise oligonucleotides probes capable of detecting biomarkers specific for a cancer disclosed herein, e.g., gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC).

In some aspects, the kits disclosed herein can comprise oligonucleotide probes to determine the level of one or more biomarker in at least one sample obtained from a cancer patient, wherein the patient suffers from a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC).

Such kits and articles of manufacture can comprise containers, each with one or more of the various reagents (e.g., in concentrated form) utilized in the method, including, for example, one or more oligonucleotides (e.g., oligonucleotide capable of hybridizing to an mRNA corresponding to a biomarker gene disclosed herein), or antibodies (i.e., antibodies capable of detecting the protein expression product of a biomarker gene disclosed herein).

One or more oligonucleotides or antibodies, e.g., capture antibodies, can be provided already attached to a solid support. One or more oligonucleotides or antibodies can be provided already conjugated to a detectable label.

The kit can also provide reagents, buffers, and/or instrumentation to support the practice of the methods provided herein.

In some aspects, a kit comprises one or more nucleic acid probes (e.g., oligonucleotides comprising naturally occurring and/or chemically modified nucleotide units) capable of hybridizing a subsequence of the gene sequence of a biomarker gene disclosed herein, e.g., under high stringency conditions. In some aspects, one or more nucleic acid probes (e.g., oligonucleotides comprising naturally occurring and/or chemically modified nucleotide units) capable of hybridizing a subsequence of the gene sequence of a biomarker gene disclosed herein, e.g., under high stringency conditions are attached to a microarray, e.g., a microarray chip. In some aspects, the microarray is, e.g., an Affymetrix, Agilent, Applied Microarrays, Arrayjet, or Illumina microarray. In some aspects, the array is an RNA microarray. A kit provided according to this disclosure can also comprise brochures or instructions describing the methods disclosed herein or their practical application to classify a patient's cancer sample, e.g., a sample obtained from gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC). Instructions included in the kits can be affixed to packaging material or can be included as a package insert. While the instructions are typically written or printed materials they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some aspects, the kit is an HTG Molecular Edge-Seq sequencing kit. In other aspects, the kit is an Illumina sequencing kit, e.g., for the NovaSEq, NextSeq, of HiSeq 2500 platforms.

### VI. Companion Diagnostic System

The methods disclosed herein can be provided as a companion diagnostic, for example available via a web server, to inform the clinician or patient about potential treatment choices. The methods disclosed herein can comprise collecting or otherwise obtaining a biological sample and performing an analytical method, e.g., applying an ANN classifier disclosed herein (e.g., the TME Panel-1 classifier) to classify a sample from a patient's tumor, alone or in combination with other biomarkers, into a TME class, and based on the TME class assignment (e.g., presence or absence of a specific stromal phenotype, i.e., whether the subject is biomarker-positive and/or biomarker-negative for a stromal phenotype or a combination thereof) provide a suitable treatment (e.g., a TME class-specific therapy disclosed herein or a combination thereof) for administration to the patient. In some aspects, the cancer is selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC).

At least some aspects of the methods described herein, due to the complexity of the calculations involved, e.g., calculation of Signature scores, preprocessing of input data to apply a ANN model (e.g., the TME Panel-1 classifier), preprocessing of input data to train an ANN, post-processing the output of an ANN, training an ANN, or any combination thereof, can be implemented with the use of a computer. In some aspects, the computer system comprises hardware elements that are electrically coupled via bus, including a processor, input device, output device, storage device, computer-readable storage media reader, communications system, processing acceleration (e.g., DSP or special-purpose processors), and memory. The computer-readable storage media reader can be further coupled to computer-readable storage media, the combination comprehensively representing remote, local, fixed and/or removable storage devices plus storage media, memory, etc. for temporarily and/or more permanently containing computer-readable information, which can include storage device, memory and/or any other such accessible system resource.

A single architecture might be utilized to implement one or more servers that can be further configured in accordance with currently desirable protocols, protocol variations, extensions, etc. However, it will be apparent to those skilled in the art that aspects may well be utilized in accordance with more specific application requirements. Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system further comprises one or more devices for providing input data to the one or more processors. The system further comprises a memory for storing a dataset of ranked data elements. In another aspect, the device for providing input data comprises a detector for detecting the characteristic of the data element, e.g., such as a fluorescent plate reader, mass spectrometer, or gene chip reader.

The system additionally may comprise a database management system. User requests or queries can be formatted in an appropriate language understood by the database management system that processes the query to extract the relevant information from the database of training sets. The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests. The system can be in communication with an input device for providing data regarding data elements to the system (e.g., expression values). In one aspect, the input device can include a gene expression profiling system including, e.g., a mass spectrometer, gene chip or array reader, and the like.

In some aspects, the systems disclosed herein can be partially or completely implemented as a cloud-based service, e.g., only some components such as databases may be cloud-based and executable modules may be installed locally, or the entirety of the system could be cloud-based. The term “cloud-based service”, or more simply, “cloud service”, refers not only to a service provided through the cloud, but also to a service providing form in which a cloud customer contracts with a cloud service provider to deliver the service provided through the cloud online. A cloud service provider manages a public cloud, a private cloud, or a hybrid cloud for delivering cloud services to one or more cloud customers online. The term cloud-based service refers not only to services provided by the cloud, but also to cloud customers contracting with cloud service providers for online delivery of services provided by the cloud.

Some aspects described herein can be implemented so as to include a computer program product. A computer program product may include a computer readable medium having computer readable program code embodied in the medium for causing an application program to execute on a computer with a database. As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements.

Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents. In one aspect, a computer program product is provided to implement the treatment, diagnostic, prognostic, or monitoring methods disclosed herein, for example, to determine whether to administer a certain therapy based on the classification of a tumor sample or tumor microenvironment sample from a patient according, e.g., to an ANN classifier disclosed herein such as TME Panel-1.

The computer program product includes a computer readable medium embodying program code executable by a processor of a computing device or system, the program code comprising:
(a) code that retrieves data attributed to a biological sample from a subject, wherein the data comprises expression level data (or data otherwise derived from expression level values) corresponding to biomarkers genes in the biological sample (e.g., a panel of genes from TABLE 1 to derive an Angiogenesis Signature and a panel of genes from TABLE 2 to derive an Immune Signature; or a panel of Angiogenesis Signature genes from TABLE 3 and a panel of Immune Signature genes from TABLE 4; a geneset disclosed in TABLE 5, or any of the gene panels (Genesets) disclosed in FIG. 9A-G that has been used to train an ANN such as TME Panel-1). These values can also be combined with values corresponding, for example, the patient's current therapeutic regimen or lack thereof, and,
(b) code that executes a classification method that indicates, e.g., whether to administer a therapeutic agent to a patient with a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) based, e.g., on the TME classification of the patient's cancer according to the TME Panel-1 classifier disclosed herein.

While various aspects have been described as methods or apparatuses, it should be understood that aspects can be implemented through code coupled with a computer, e.g., code resident on a computer or accessible by the computer. For example, software and databases could be utilized to implement many of the methods discussed above. Thus, in addition to aspects accomplished by hardware, it is also noted that these aspects can be accomplished through the use of an article of manufacture comprised of a computer usable medium having a computer readable program code embodied therein, which causes the enablement of the functions disclosed in this description. Therefore, it is desired that aspects also be considered protected by this patent in their program code means as well.

Furthermore, some aspects can be code stored in a computer-readable memory of virtually any kind including, without limitation, RAM, ROM, magnetic media, optical media, or magneto-optical media. Even more generally, some aspects could be implemented in software, or in hardware, or any combination thereof including, but not limited to, software running on a general purpose processor, microcode, PLAs, or ASICs.

It is also envisioned that some aspects could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

### VIII. Additional Techniques and Tests

Factors known in the art for diagnosing and/or suggesting, selecting, designating, recommending or otherwise determining a course of treatment for a patient or class of patients suspected of having a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) can be employed, e.g., in combination with the methods disclosed herein. Accordingly, the methods disclosed herein can include additional techniques such as cytology, histology, ultrasound analysis, MRI results, CT scan results, and cancer-specific antigen measurements.

Certified tests for classifying disease status and/or designating treatment modalities can also be used in diagnosing, predicting, and/or monitoring the status or outcome of a cancer in a subject. A certified test can comprise a means for characterizing the expression levels of one or more of the target sequences of interest, and a certification from a government regulatory agency endorsing use of the test for classifying the disease status of a biological sample.

In some aspects, the certified test can comprise reagents for amplification reactions used to detect and/or quantitate expression of the target sequences to be characterized in the test. An array of probe nucleic acids can be used, with or without prior target amplification, for use in measuring target sequence expression.

The test can be submitted to an agency having authority to certify the test for use in distinguishing disease status and/or outcome. Results of detection of expression levels of the target sequences used in the test and correlation with disease status and/or outcome can be submitted to the agency. A certification authorizing the diagnostic and/or prognostic use of the test can be obtained.

Also provided are portfolios of expression levels comprising a plurality of normalized expression levels of any of the genesets disclosed herein. In some aspects, the genes in the geneset are selected from TABLE 1. In some aspects, the genes in the geneset are selected from TABLE 2. In some aspects, the genes in the geneset are selected from TABLE 1 and TABLE 2. In some aspects, the geneset is selected from the gene panels disclosed in TABLE 3. In some aspects, the geneset is selected from the gene panel disclosed in TABLE 4. In some aspects, the geneset is selected from the gene panels disclosed in TABLE 3 and TABLE 4. In some aspects, the geneset is selected from the genesets disclosed in TABLE 5. In some aspects, the geneset is selected from any of the genesets disclosed in FIG. 9A-G.

In some aspects, the geneset comprises at least one gene from TABLE 1 and at least one gene from TABLE 2. In some aspects, the geneset comprises a gene panel from TABLE 3 and a gene panel from TABLE 4. In some aspects, the geneset consists of a geneset from TABLE 5. In some aspects, the geneset consists of any of the genesets disclosed in FIG. 9A-G. Such portfolios can be provided by performing the methods described herein to obtain expression levels from an individual patient or from a group of patients. The expression levels can be normalized by any method known in the art; exemplary normalization methods that can be used in various aspects include Robust Multichip Average (RMA), probe logarithmic intensity error estimation (PLIER), non-linear fit (NLFIT) quantile-based and nonlinear normalization, and combinations thereof. Background correction can also be performed on the expression data; exemplary techniques useful for background correction include mode of intensities, normalized using median polish probe modeling and sketch-normalization.

In some aspects, genes can be included or excluded from gene panels or portfolios expression disclosed herein such that the ANN classifier resulting from training with the combination of genes in the gene panel exhibits improved sensitivity and specificity relative to known methods. In considering a group of genes for inclusion in a gene panel, a small standard deviation in expression measurements correlates with greater specificity. Other measurements of variation such as correlation coefficients can also be used in this capacity.

The disclosure also encompasses the above methods where the expression level determines the status or outcome of a cancer gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, or lung cancer (e.g., NSCLC) in the subject or the efficacy and/or outcome of the treatment the cancer patient with a personalized, e.g., TME-specific, therapy with at least about 45% specificity, at least about 50% specificity, at least about 55%, at least about 60% specificity, at least about 65% specificity, at least about 70% specificity, at least about 75% specificity, at least about 80% specificity, at least about 85% specificity, at least about 90% specificity, or at least about 95% specificity.

In some aspects, the accuracy of the TME Panel-1 classifier disclosed herein and its applications, e.g., for diagnosing, monitoring, and/or predicting a status or outcome of a cancer selected from the group consisting of gastric cancer (e.g., locally advanced, metastatic gastric cancer, or previously untreated gastric cancer), breast cancer (e.g., locally advanced or metastatic Her2-negative breast cancer), prostate cancer (e.g., castration-resistant metastatic prostate cancer), liver cancer (e.g., hepatocellular carcinoma such as advanced metastatic hepatocellular carcinoma), carcinoma of head and neck (e.g., recurrent or metastatic squamous cell carcinoma of head and neck), melanoma, colorectal cancer (e.g., advanced colorectal cancer metastatic to liver), ovarian cancer (e.g., platinum-resistant ovarian cancer or platinum-sensitive recurrent ovarian cancer), glioma (e.g., metastatic glioma), glioblastoma, and lung cancer (e.g., NSCLC) for predicting the efficacy and/or outcome of the cancer in a patient with a personalized, e.g., TME-specific, therapy, is at least about 45%, at least about 50%, at least about 55%, at least about 60%, at least about 65%, at least about 70%, at least about 75%, at least about 80%, at least about 85%, at least about 90%, or at least about 95%.

The accuracy of a classifier can be determined by the 95% confidence interval (CI). Generally, a classifier is considered to have good accuracy if the 95% CI does not overlap 1. In some aspects, the 95% CI of a classifier is at least about 1.08, at least about 1.10, at least about 1.12, at least about 1.14, at least about 1.15, at least about 1.16, at least about 1.17, at least about 1.18, at least about 1.19, at least about 1.20, at least about 1.21, at least about 1.22, at least about 1.23, at least about 1.24, at least about 1.25, at least about 1.26, at least about 1.27, at least about 1.28, at least about 1.29, at least about 1.30, at least about 1.31, at least about 1.32, at least about 1.33, at least about 1.34, or at least about 1.35 or more. The 95% CI of a classifier may be at least about 1.14, at least about 1.15, at least about 1.16, at least about 1.20, at least about 1.21, at least about 1.26, or at least about 1.28. The 95% CI of a classifier may be less than about 1.75, less than about 1.74, less than about 1.73, less than about 1.72, less than about 1.71, less than about 1.70, less than about 1.69, less than about 1.68, less than about 1.67, less than about 1.66, less than about 1.65, less than about 1.64, less than about 1.63, less than about 1.62, less than about 1.61, less than about 1.60, less than about 1.59, less than about 1.58, less than about 1.57, less than about 1.56, less than about 1.55, less than about 1.54, less than about 1.53, less than about 1.52, less than about 1.51, less than about 1.50 or less. The 95% CI of a classifier may be less than about 1.61, less than about 1.60, less than about 1.59, less than about 1.58, less than about 1.56, 1.55, or 1.53. The 95% CI of a classifier may be between about 1.10 to 1.70, between about 1.12 to about 1.68, between about 1.14 to about 1.62, between about 1.15 to about 1.61, between about 1.15 to about 1.59, between about 1.16 to about 1.160, between about 1.19 to about 1.55, between about 1.20 to about 1.54, between about 1.21 to about 1.53, between about 1.26 to about 1.63, between about 1.27 to about 1.61, or between about 1.28 to about 1.60.

In some aspects, the accuracy of a classifier is dependent on the difference in range of the 95% CI (e.g., difference in the high value and low value of the 95% CI interval). Generally, classifiers with large differences in the range of the 95% CI interval have greater variability and are considered less accurate than classifiers with small differences in the range of the 95% CI intervals. In some aspects, a classifier is considered more accurate if the difference in the range of the 95% CI is less than about 0.60, less than about 0.55, less than about 0.50, less than about 0.49, less than about 0.48, less than about 0.47, less than about 0.46, less than about 0.45, less than about 0.44, less than about 0.43, less than about 0.42, less than about 0.41, less than about 0.40, less than about 0.39, less than about 0.38, less than about 0.37, less than about 0.36, less than about 0.35, less than about 0.34, less than about 0.33, less than about 0.32, less than about 0.31, less than about 0.30, less than about 0.29, less than about 0.28, less than about 0.27, less than about 0.26, less than about 0.25 or less. The difference in the range of the 95% CI of a classifier may be less than about 0.48, less than about 0.45, less than about 0.44, less than about 0.42, less than about 0.40, less than about 0.37, less than about 0.35, less than about 0.33, or less than about 0.32. In some aspects, the difference in the range of the 95% CI for a classifier is between about 0.25 to about 0.50, between about 0.27 to about 0.47, or between about 0.30 to about 0.45.

In some aspects, the sensitivity of the TME Panel-1 classifier is at least about 45%. In some aspects, the sensitivity is at least about 50%. In some aspects, the sensitivity is at least about 55%. In some aspects, the sensitivity is at least about 60%. In some aspects, the sensitivity is at least about 65%. In some aspects, the sensitivity is at least about 70%. In some aspects, the sensitivity is at least about 75%. In some aspects, the sensitivity is at least about 80%. In some aspects, the sensitivity is at least about 85%. In some aspects, the sensitivity is at least about 90%. In some aspects, the sensitivity is at least about 95%.

In some aspects, the output from the TME Panel-1 classifier is clinically significant. In some aspects, the clinical significance of the classifier is determined by the AUC value. In order to be clinically significant, the AUC value is at least about 0.5, at least about 0.55, at least about 0.6, at least about 0.65, at least about 0.7, at least about 0.75, at least about 0.8, at least about 0.85, at least about 0.9, or at least about 0.95. The clinical significance of the classifier can be determined by the percent accuracy. For example, a classifier is determined to be clinically significant if the accuracy of the classifier is at least about 50%, at least about 55%, at least about 60%, at least about 65%, at least about 70%, at least about 72%, at least about 75%, at least about 77%, at least about 80%, at least about 82%, at least about 84%, at least about 86%, at least about 88%, at least about 90%, at least about 92%, at least about 94%, at least about 96%, or at least about 98%.

In other aspects, the clinical significance of the TME Panel-1 classifier is determined by the median fold difference (MDF) value. In order to be clinically significant, the MDF value is at least about 0.8, at least about 0.9, at least about 1.0, at least about 1.1, at least about 1.2, at least about 1.3, at least about 1.4, at least about 1.5, at least about 1.6, at least about 1.7, at least about 1.9, or at least about 2.0. In some aspects, the MDF value is greater than or equal to 1.1. In other aspects, the MDF value is greater than or equal to 1.2. Alternatively, or additionally, the clinical significance of the classifiers or biomarkers is determined by the t-test P-value. In some aspects, in order to be clinically significant, the t-test P-value is less than about 0.070, less than about 0.065, less than about 0.060, less than about 0.055, less than about 0.050, less than about 0.045, less than about 0.040, less than about 0.035, less than about 0.030, less than about 0.025, less than about 0.020, less than about 0.015, less than about 0.010, less than about 0.005, less than about 0.004, or less than about 0.003. The t-test P-value can be less than about 0.050. Alternatively, the t-test P-value is less than about 0.010.

In some aspects, the clinical significance of the TME Panel-1 classifier is determined by the clinical outcome. For example, different clinical outcomes can have different minimum or maximum thresholds for AUC values, MDF values, t-test P-values, and accuracy values that would determine whether the classifier is clinically significant. In another example, a classifier is considered clinically significant if the P-value of the t-test was less than about 0.08, less than about 0.07, less than about 0.06, less than about 0.05, less than about 0.04, less than about 0.03, less than about 0.02, less than about 0.01, less than about 0.005, less than about 0.004, less than about 0.003, less than about 0.002, or less than about 0.001.

In some aspects, the performance of the TME Panel-1 classifier is based on the odds ratio. A classifier may be considered to have good performance if the odds ratio is at least about 1.30, at least about 1.31, at least about 1.32, at least about 1.33, at least about 1.34, at least about 1.35, at least about 1.36, at least about 1.37, at least about 1.38, at least about 1.39, at least about 1.40, at least about 1.41, at least about 1.42, at least about 1.43, at least about 1.44, at least about 1.45, at least about 1.46, at least about 1.47, at least about 1.48, at least about 1.49, at least about 1.50, at least about 1.52, at least about 1.55, at least about 1.57, at least about 1.60, at least about 1.62, at least about 1.65, at least about 1.67, at least about 1.70 or more. In some aspects, the odds ratio of a classifier is at least about 1.33.

The clinical significance of a classifier may be based on Univariable Analysis Odds Ratio P-value (uvaORPval). The Univariable Analysis Odds Ratio P-value (uvaORPval) of the TME Panel-1 classifier may be between about 0 and about 0.4. The Univariable Analysis Odds Ratio P-value (uvaORPval) of the TME Panel-1 classifier may be between about 0 and about 0.3. The Univariable Analysis Odds Ratio P-value (uvaORPval)) of the TME Panel-1 classifier may be between about 0 and about 0.2. The Univariable Analysis Odds Ratio P-value (uvaORPval)) of the TME Panel-1 classifier may be less than or equal to 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11.

The Univariable Analysis Odds Ratio P-value (uvaORPval) of the TME Panel-1 classifier may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Univariable Analysis Odds Ratio P-value (uvaORPval) of the TME Panel-1 classifier may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of a classifier may be based on multivariable analysis Odds Ratio P-value (mvaORPval). The multivariable analysis Odds Ratio P-value (mvaORPval)) of the TME Panel-1 classifier may be between about 0 and about 1. The multivariable analysis Odds Ratio P-value (mvaORPval)) of the TME Panel-1 classifier may be between about 0 and about 0.9. The multivariable analysis Odds Ratio P-value (mvaORPval)) of the TME Panel-1 classifier may be between about 0 and about 0.8. The multivariable analysis Odds Ratio P-value (mvaORPval) of the TME Panel-1 classifier may be less than or equal to about 0.90, less than or equal to about 0.88, less than or equal to about 0.86, less than or equal to about 0.84, less than or equal to about 0.82, or less than or equal to about 0.80. The multivariable analysis Odds Ratio P-value (mvaORPval)) of the TME Panel-1 classifier may be less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The multivariable analysis Odds Ratio P-value (mvaORPval) of the TME Panel-1 classifier may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The multivariable analysis Odds Ratio P-value (mvaORPval)) of the TME Panel-1 classifier may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The multivariable analysis Odds Ratio P-value (mvaORPval)) of the TME Panel-1 classifier may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of a classifier may be based on the Kaplan Meier P-value (KM P-value). The Kaplan Meier P-value (KM P-value) of the TME Panel-1 classifier may be between about 0 and about 0.8. The Kaplan Meier P-value (KM P-value) of the TME Panel-1 classifier may be between about 0 and about 0.7. The Kaplan Meier P-value (KM P-value) of the TME Panel-1 classifier may be less than or equal to about 0.80, less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The Kaplan Meier P-value (KM P-value) of the TME Panel-1 classifier may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Kaplan Meier P-value (KM P-value) of the TME Panel-1 classifier may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Kaplan Meier P-value (KM P-value) of the TME Panel-1 classifier may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of a classifiers may be based on the survival AUC value (survAUC). The survival AUC value (survAUC) of the TME Panel-1 classifier may be between about 0-1. The survival AUC value (survAUC) of the TME Panel-1 classifier may be between about 0 to about 0.9. The survival AUC value (survAUC) of the TME Panel-1 classifier may be less than or equal to about 1, less than or equal to about 0.98, less than or equal to about 0.96, less than or equal to about 0.94, less than or equal to about 0.92, less than or equal to about 0.90, less than or equal to about 0.88, less than or equal to about 0.86, less than or equal to about 0.84, less than or equal to about 0.82, or less than or equal to about 0.80. The survival AUC value (survAUC) of the TME Panel-1 classifier may be less than or equal to about 0.80, less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The survival AUC value (survAUC) of the TME Panel-1 classifier may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The survival AUC value (survAUC) of the TME Panel-1 classifier may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The survival AUC value (survAUC) of the TME Panel-1 classifier may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001

The clinical significance of a classifier may be based on the Univariable Analysis Hazard Ratio P-value (uvaHRPval). The Univariable Analysis Hazard Ratio P-value (uvaHRPval) of the TME Panel-1 classifier may be between about 0 to about 0.4. The Univariable Analysis Hazard Ratio P-value (uvaHRPval) of the TME Panel-1 classifier may be between about 0 to about 0.3. The Univariable Analysis Hazard Ratio P-value (uvaHRPval) of the TME Panel-1 classifier may be less than or equal to about 0.40, less than or equal to about 0.38, less than or equal to about 0.36, less than or equal to about 0.34, or less than or equal to about 0.32. The Univariable Analysis Hazard Ratio P-value (uvaRPval) of the TME Panel-1 classifier may be less than or equal to about 0.30, less than or equal to about 0.29, less than or equal to about 0.28, less than or equal to about 0.27, less than or equal to about 0.26, less than or equal to about 0.25, less than or equal to about 0.24, less than or equal to about 0.23, less than or equal to about 0.22, less than or equal to about 0.21, or less than or equal to about 0.20. The Univariable Analysis Hazard Ratio P-value (uvaRPval) of the TME Panel-1 classifier may be less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Univariable Analysis Hazard Ratio P-value (uvaHRPval) of the TME Panel-1 classifier may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Univariable Analysis Hazard Ratio P-value (uvaRPval) of the TME Panel-1 classifier may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of a classifier may be based on the Multivariable Analysis Hazard Ratio P-value (mvaHRPval)mva HRPval. The Multivariable Analysis Hazard Ratio P-value (mvaHRPval)mva HRPval of the TME Panel-1 classifier may be between about 0 to about 1. The Multivariable Analysis Hazard Ratio P-value (mvaHRPval)mva HRPval of the TME Panel-1 classifier may be between about 0 to about 0.9. The Multivariable Analysis Hazard Ratio P-value (mvaHRPval)mva HRPval of the TME Panel-1 classifier may be less than or equal to about 1, less than or equal to about 0.98, less than or equal to about 0.96, less than or equal to about 0.94, less than or equal to about 0.92, less than or equal to about 0.90, less than or equal to about 0.88, less than or equal to about 0.86, less than or equal to about 0.84, less than or equal to about 0.82, or less than or equal to about 0.80. The Multivariable Analysis Hazard Ratio P-value (mvaHRPval)mva HRPval of the TME Panel-1 classifier may be less than or equal to about 0.80, less than or equal to about 0.78, less than or equal to about 0.76, less than or equal to about 0.74, less than or equal to about 0.72, less than or equal to about 0.70, less than or equal to about 0.68, less than or equal to about 0.66, less than or equal to about 0.64, less than or equal to about 0.62, less than or equal to about 0.60, less than or equal to about 0.58, less than or equal to about 0.56, less than or equal to about 0.54, less than or equal to about 0.52, or less than or equal to about 0.50. The Multivariable Analysis Hazard Ratio P-value (mvaHRPval)mva HRPval of the TME Panel-1 classifier may be less than or equal to about 0.48, less than or equal to about 0.46, less than or equal to about 0.44, less than or equal to about 0.42, less than or equal to about 0.40, less than or equal to about 0.38, less than or equal to about 0.36, less than or equal to about 0.34, less than or equal to about 0.32, less than or equal to about 0.30, less than or equal to about 0.28, less than or equal to about 0.26, less than or equal to about 0.25, less than or equal to about 0.22, less than or equal to about 0.21, less than or equal to about 0.20, less than or equal to about 0.19, less than or equal to about 0.18, less than or equal to about 0.17, less than or equal to about 0.16, less than or equal to about 0.15, less than or equal to about 0.14, less than or equal to about 0.13, less than or equal to about 0.12, or less than or equal to about 0.11. The Multivariable Analysis Hazard Ratio P-value (mvaHRPval)mva HRPval of the TME Panel-1 classifier may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Multivariable Analysis Hazard Ratio P-value (mvaHRPval)mva HRPval of the TME Panel-1 classifier may be less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The clinical significance of a classifier may be based on the Multivariable Analysis Hazard Ratio P-value (mvaHRPval). The Multivariable Analysis Hazard Ratio P-value (mvaHRPval) of the TME Panel-1 classifier may be between about 0 to about 0.60. Significance of the TME Panel-1 classifier may be based on the Multivariable Analysis Hazard Ratio P-value (mvaHRPval). The Multivariable Analysis Hazard Ratio P-value (mvaHRPval) of the TME Panel-1 classifier may be between about 0 to about 0.50. Significance of the classifier may be based on the Multivariable Analysis Hazard Ratio P-value (mvaHRPval). The Multivariable Analysis Hazard Ratio P-value (mvaHRPval) of the TME Panel-1 classifier may be less than or equal to about 0.50, less than or equal to about 0.47, less than or equal to about 0.45, less than or equal to about 0.43, less than or equal to about 0.40, less than or equal to about 0.38, less than or equal to about 0.35, less than or equal to about 0.33, less than or equal to about 0.30, less than or equal to about 0.28, less than or equal to about 0.25, less than or equal to about 0.22, less than or equal to about 0.20, less than or equal to about 0.18, less than or equal to about 0.16, less than or equal to about 0.15, less than or equal to about 0.14, less than or equal to about 0.13, less than or equal to about 0.12, less than or equal to about 0.11, or less than or equal to about 0.10. The Multivariable Analysis Hazard Ratio P-value (mvaHRPval) of the TME Panel-1 classifier may be less than or equal to about 0.10, less than or equal to about 0.09, less than or equal to about 0.08, less than or equal to about 0.07, less than or equal to about 0.06, less than or equal to about 0.05, less than or equal to about 0.04, less than or equal to about 0.03, less than or equal to about 0.02, or less than or equal to about 0.01. The Multivariable Analysis Hazard Ratio P-value (mvaHRPval) of the TME Panel-1 classifier may be less than or equal to about 0.01, less than or equal to about 0.009, less than or equal to about 0.008, less than or equal to about 0.007, less than or equal to about 0.006, less than or equal to about 0.005, less than or equal to about 0.004, less than or equal to about 0.003, less than or equal to about 0.002, or less than or equal to about 0.001.

The TME Panel-1 classifier disclosed herein may outperform current classifiers (e.g., CMS for colorectal cancer) in providing clinically relevant analysis of a sample from a subject. In some aspects, TME Panel-1 may more accurately predict a clinical outcome or status as compared to current classifiers (e.g., CMS for colorectal cancer). For example, TME Panel-1 may more accurately predict metastatic disease. Alternatively, TME Panel-1 may more accurately predict no evidence of disease. In some aspects, TME Panel-1 may more accurately predict death from a disease. The performance of TME Panel-1 may be based on the AUC value, odds ratio, 95% CI, difference in range of the 95% CI, p-value or any combination thereof.

The performance of the TME Panel-1 classifier disclosed herein can be determined by AUC values and an improvement in performance may be determined by the difference in the AUC value of TME Panel-1 and the AUC value of current classifiers (e.g., CMS for colorectal cancer). In some aspects, TME Panel-1 outperforms current classifiers (e.g., CMS for colorectal cancer) when the AUC value of TME Panel-1 is greater than the AUC value of the current classifiers (e.g., CMS for colorectal cancer) by at least about 0.05, by at least about 0.06, by at least about 0.07, by at least about 0.08, by at least about 0.09, by at least about 0.10, by at least about 0.11, by at least about 0.12, by at least about 0.13, by at least about 0.14, by at least about 0.15, by at least about 0.16, by at least about 0.17, by at least about 0.18, by at least about 0.19, by at least about 0.20, by at least about 0.022, by at least about 0.25, by at least about 0.27, by at least about 0.30, by at least about 0.32, by at least about 0.35, by at least about 0.37, by at least about 0.40, by at least about 0.42, by at least about 0.45, by at least about 0.47, or by at least about 0.50 or more. In some aspects, the AUC value of TME Panel-1 herein is greater than the AUC value of the current classifiers (e.g., CMS for colorectal cancer) by at least about 0.10. In some aspects, the AUC value of TME Panel-1 is greater than the AUC value of the current classifiers (e.g., CMS for colorectal cancer) by at least about 0.13. In some aspects, the AUC value of TME Panel-1 is greater than the AUC value of the current classifiers (e.g., CMS for colorectal cancer) by at least about 0.18.

The performance of TME Panel-1 can be determined by the odds ratios and an improvement in performance can be determined by comparing the odds ratio of TME Panel-1 and the odds ratio of current classifiers (e.g., CMS for colorectal cancer). Comparison of the performance of two or more classifiers can generally be based on the comparison of the absolute value of (1-odds ratio) of a first classifier to the absolute value of (1-odds ratio) of a second classifier. Generally, the classifier with the greater absolute value of (1-odds ratio) can be considered to have better performance as compared to the classifier with a smaller absolute value of (1-odds ratio).

In some aspects, the TME Panel-1 Classifier disclosed herein is more accurate than a current classifier (e.g., CMS for colorectal cancer). In some aspects, TME Panel-1 is more accurate than a current classifier (e.g., CMS) when difference in range of the 95% CI of TME Panel-1 herein is about 0.70, about 0.60, about 0.50, about 0.40, about 0.30, about 0.20, about 0.15, about 0.14, about 0.13, about 0.12, about 0.10, about 0.09, about 0.08, about 0.07, about 0.06, about 0.05, about 0.04, about 0.03, or about 0.02 times less than the difference in range of the 95% CI of the current classifier (e.g., CMS for colorectal cancer). In some aspects, TME Panel-1 is more accurate than a current classifier (e.g., CMS for colorectal cancer) when difference in range of the 95% CI of TME Panel-1 between about 0.20 to about 0.04 times less than the difference in range of the 95% CI of the current classifier (e.g., CMS for colorectal cancer).

### EMBODIMENTS

In the listing below, embodiments are abbreviated as E, thus, E1 to E82 represent Embodiment 1 to Embodiment 82.

E1. A method for treating a human subject afflicted with a cancer comprising administering a TME phenotype class-specific therapy to the subject, wherein, prior to the administration, a TME phenotype class is determined by applying an Artificial Neural Network (ANN) classifier to a plurality of RNA expression levels obtained from a gene panel from a cancer tumor sample obtained from the subject, wherein the cancer tumor is assigned a TME phenotype class selected from the group consisting of IS (immune suppressed), A (angiogenic), IA (immune active), ID (immune desert), and combinations thereof.

E2. A method for treating a human subject afflicted with a cancer comprising
(i) applying an ANN classifier to a plurality of RNA expression levels obtained from a gene panel from a cancer tumor sample obtained from the subject, wherein the cancer tumor is assigned a TME phenotype class selected from the group consisting of IS, A, IA, ID, and combinations thereof; and,
(ii) administering a TME phenotype class-specific therapy to the subject.

E3. A method for identifying a human subject afflicted with a cancer suitable for treatment with a TME phenotype class-specific therapy, the method comprising applying an ANN classifier to a plurality of RNA expression levels obtained from a gene panel from a cancer tumor sample obtained from the subject, wherein the cancer tumor is assigned a TME phenotype class selected from the group consisting of IS, A, IA, ID, and combinations thereof, and wherein the assigned TME phenotype class indicates that a TME phenotype class-specific therapy can be administered to treat the cancer.

E4. The method of any one of embodiments E1 to E3, wherein the ANN classifier comprises an input layer, a hidden layer, and an output layer.

E5. The method of embodiment E4, wherein the input layer comprises between 2 and 100 nodes.

E6. The method of embodiment E5, wherein each node in the input layer corresponds to a gene in a gene panel selected from the genes presented in TABLE 1 and TABLE 2, wherein the gene panel comprises (i) between 1 and 63 genes selected from TABLE 1, and between 1 and 61 genes selected from TABLE 2, (ii) a gene panel comprising genes selected from TABLE 3 and TABLE 4, (iii) a gene panel of TABLE 5, or (iv) any of the gene panels (Genesets) disclosed in FIG. 9A-G.

E7. The method of any one of embodiments E1 to E6, wherein the sample comprises intratumoral tissue.

E8. The method of any one of embodiments E1 to E7, wherein the RNA expression levels are transcribed RNA expression levels determined using Next Generation Sequencing (NGS) such as RNA-Seq, EdgeSeq, PCR, Nanostring, WES, or combinations thereof.

E9. The method of any one of embodiments E4 to E8, wherein the hidden layer comprises 2 nodes and the output layer comprises 4 output nodes, wherein each one of the 4 output nodes in the output layer corresponds to a TME phenotype class, wherein the 4 TME phenotype classes are IA, IS, ID, and A.

E10. The method of any one of embodiments E4 to E9, further comprising applying a logistic regression classifier comprising a Softmax function to the output of the ANN, wherein the Softmax function assigns probabilities to each TME phenotype class.

E11. The method of any of one of embodiments E1 to E10, wherein the TME phenotype-class specific therapy is an IA, IS, ID or A TME phenotype class-specific therapy or a combination thereof.

E12. The method of any one of embodiments E1 to E11, wherein the TME phenotype class-specific therapy is an IA TME phenotype class-specific therapy comprising a checkpoint modulator therapy.

E13. The method of embodiment E12, wherein the checkpoint modulator therapy comprises administering
(i) an activator of a stimulatory immune checkpoint molecule such as an antibody molecule against GITR, OX-40, ICOS, 4-1BB, or a combination thereof,
(ii) a RORγ agonist; or,
(iii) an inhibitor of an inhibitory immune checkpoint molecule such as an antibody against PD-1 (such as nivolumab, pembrolizumab, cemiplimab, PDR001, CBT-501, CX-188, sintilimab, tislelizumab, TSR-042 or an antigen-binding portion thereof), an antibody against PD-L1 (such as avelumab, atezolizumab, durvalumab, CX-072, LY3300054, or an antigen-binding portion thereof), an antibody against PD-L2, or an antibody against CTLA-4, alone or a combination thereof, or in combination with an inhibitor of TIM-3, LAG-3, BTLA, TIGIT, VISTA, TGF-β, LAIR1, CD160, 2B4, GITR, OX40, 4-1BB, CD2, CD27, CDS, ICAM-1, LFA-1, ICOS, CD30, CD40, BAFFR, HVEM, CD7, LIGHT, NKG2C, SLAMF7, NKp80, or CD86.

E14. The method of embodiment E12, where the checkpoint modulator therapy comprises administering (i) an anti-PD-1 antibody selected from the group consisting of nivolumab, pembrolizumab, cemiplimab, PDR001, CBT-501, CX-188, sintilimab, tislelizumab, or TSR-042; (ii) an anti-PD-L1 antibody selected from the group consisting of avelumab, atezolizumab, CX-072, LY3300054, and durvalumab; or (iii) a combination thereof.

E15. The method of any one of embodiments E1 to E14, wherein the TME phenotype class-specific therapy is an IS-class TME therapy comprising administering (1) a checkpoint modulator therapy and an anti-immunosuppression therapy, and/or (2) an antiangiogenic therapy.

E16. The method of embodiment E15, wherein the checkpoint modulator therapy comprises administering an inhibitor of an inhibitory immune checkpoint molecule.

E17. The method of embodiment E16, wherein the inhibitor of an inhibitory immune checkpoint molecule is
(i) an antibody against PD-1 selected from the group consisting of pembrolizumab, nivolumab, cemiplimab, PDR001, CBT-501, CX-188, sintilimab, tislelizumab, TSR-042, an antigen-binding portion thereof, and a combination thereof,
(ii) an antibody against PD-L1 selected from the group consisting of avelumab, atezolizumab, CX-072, LY3300054, durvalumab, an antigen-binding portion thereof, and a combination thereof,
(iii) an antibody against PD-L2 or an antigen binding portion thereof,
(iv) an antibody against CTLA-4 selected from ipilimumab and the bispecific antibody XmAb20717 (anti PD-1/anti-CTLA-4); or
(v) a combination thereof.

E18. The method of any one of embodiments E15 to E17, wherein the antiangiogenic therapy comprises administering
(i) an anti-VEGF antibody selected from the group consisting of varisacumab, bevacizumab, navicixizumab (anti-DLL4/anti-VEGF bispecific), ABL101 (NOV1501) (anti-DLL4/anti-VEGF), ABT165 (anti-DLL4/anti-VEGF), and a combination thereof,
(ii) an anti-VEGFR2 antibody, wherein the anti-VEGFR2 antibody comprises ramucirumab; or,
(iii) a combination thereof.

E19. The method of any one of embodiment E15 to E18, wherein the anti-immunosuppression therapy comprises administering an anti-PS antibody, anti-PS targeting antibody, antibody that binds β2-glycoprotein 1, inhibitor of PI3Kγ, adenosine pathway inhibitor, inhibitor of IDO, inhibitor of TIM, inhibitor of LAG3, inhibitor of TGF-β, CD47 inhibitor, or a combination thereof, wherein
(i) the anti-PS targeting antibody is bavituximab, or an antibody that binds β2-glycoprotein 1;
(ii) the PI3Kγ inhibitor is LY3023414 (samotolisib) or IPI-549;
(iii) the adenosine pathway inhibitor is AB-928;
(iv) the TGFβ inhibitor is LY2157299 (galunisertib) or the TGFβR1 inhibitor is LY3200882;
(v) the CD47 inhibitor is magrolimab (5F9); and,
(vi) the CD47 inhibitor targets SIRPα.

E20. The methods of any one of embodiment E15 to E19, wherein the anti-immunosuppression therapy comprises administering an inhibitor of TIM-3, LAG-3, BTLA, TIGIT, VISTA, TGF-β or its receptors, an inhibitor of LAIR1, CD160, 2B4, GITR, OX40, 4-1BB, CD2, CD27, CDS, ICAM-1, LFA-1, ICOS, CD30, CD40, BAFFR, HVEM, CD7, LIGHT, NKG2C, SLAMF7, NKp80, an agonist of CD86, or a combination thereof.

E21. The method of any one embodiment E1 to E20, wherein the TME phenotype class-specific therapy is an A TME phenotype class-specific therapy comprising administering a VEGF-targeted therapy, an inhibitor of angiopoietin 1 (Ang1), an inhibitor of angiopoietin 2 (Ang2), an inhibitor of DLL4, a bispecific of anti-VEGF and anti-DLL4, a TKI inhibitor, an anti-FGF antibody, an anti-FGFR1 antibody, an anti-FGFR2 antibody, a small molecule that inhibits FGFR1, a small molecule that inhibits FGFR2, an anti-PLGF antibody, a small molecule against a PLGF receptor, an antibody against a PLGF receptor, an anti-VEGFB antibody, an anti-VEGFC antibody, an anti-VEGFD antibody, an antibody to a VEGF/PLGF trap molecule such as aflibercept, or ziv-aflibercet, an anti-DLL4 antibody, an anti-Notch therapy such as an inhibitor of gamma-secretase, or any combination thereof.

E22. The method of embodiment E21, wherein the TKI inhibitor is selected from the group consisting of cabozantinib, vandetanib, tivozanib, axitinib, lenvatinib, sorafenib, regorafenib, sunitinib, fruquitinib, pazopanib, and any combination thereof.

E23. The method of embodiment E21, wherein the VEGF-targeted therapy comprises administering
(i) an anti-VEGF antibody comprising varisacumab, bevacizumab, an antigen-binding portion thereof, or a combination thereof;
(ii) an anti-VEGFR2 antibody comprising ramucirumab or an antigen-binding portion thereof, or,
(iii) a combination thereof.

E24. The method of any one of embodiments E21 to E23, wherein the A TME phenotype class-specific therapy comprises administering an angiopoietin/TIE2-targeted therapy comprising endoglin and/or angiopoietin.

E25. The method of any one of embodiments E21 to E24, wherein the A TME phenotype class-specific therapy comprises administering a DLL4-targeted therapy comprising navicixizumab, ABL101 (NOV1501), ABT165, or a combination thereof.

E26. The method of any one of embodiments E1 to E25, wherein the TME phenotype class-specific therapy is an ID TME phenotype class-specific therapy comprising administering a of a checkpoint modulator therapy concurrently or after the administration of a therapy that initiates an immune response.

E27. The method of embodiment E26, wherein the therapy that initiates an immune response is a vaccine, a CAR-T, or a neo-epitope vaccine.

E28. The method of embodiment E26, wherein the checkpoint modulator therapy comprises the administration of an inhibitor of an inhibitory immune checkpoint molecule.

E29. The method of embodiment E28, wherein the inhibitor of an inhibitory immune checkpoint molecule is an antibody against PD-1, PD-L1, PD-L2, CTLA-4, or a combination thereof.

E30. The method of embodiment E29, wherein the anti-PD-1 antibody comprises nivolumab, pembrolizumab, cemiplimab, PDR001, CBT-501, CX-188, sintilimab, tislelizumab, or TSR-042, or an antigen-binding portion thereof.

E31. The method of embodiment E29, wherein the anti-PD-L1 antibody comprises avelumab, atezolizumab, CX-072, LY3300054, durvalumab, or an antigen-binding portion thereof.

E32. The method of embodiment E29, wherein the anti-CTLA-4 antibody comprises ipilimumab or the bispecific antibody XmAb20717 (anti PD-1/anti-CTLA-4), or an antigen-binding portion thereof.

E33. The method of embodiment E26, wherein the checkpoint modulator therapy comprises the administration of (i) an anti-PD-1 antibody selected from the group consisting of nivolumab, pembrolizumab, cemiplimab PDR001, CBT-501, CX-188, sintilimab, tislelizumab, and TSR-042; (ii) an anti-PD-L1 antibody selected from the group consisting of avelumab, atezolizumab, CX-072, LY3300054, and durvalumab; (iv) an anti-CTLA-4 antibody, which is ipilimumab or the bispecific antibody XmAb20717 (anti PD-1/anti-CTLA-4), or (iii) a combination thereof.

E34. The method of any one of embodiments E1 to E33, further comprising
(a) administering chemotherapy;
(b) performing surgery;
(c) administering radiation therapy; or,
(d) any combination thereof.

E35. The method of any one of embodiments E1 to E34, wherein the cancer is relapsed, refractory, metastatic, dMMR, or a combination thereof.

E36. The method of embodiment E27, wherein the cancer is refractory following at least one prior therapy comprising administration of at least one anticancer agent.

E37. The method of any one of embodiments E1 to E36, wherein the cancer is selected from the group consisting of gastric cancer, breast cancer, prostate cancer, liver cancer, carcinoma of head and neck, melanoma, colorectal cancer, ovarian cancer, glioma, lung cancer, and glioblastoma.

E38. The method of embodiment E37, wherein the gastric cancer is locally advanced, metastatic gastric cancer, or previously untreated gastric cancer.

E39. The method of embodiment E37, wherein the breast cancer is locally advanced or metastatic Her2-negative breast cancer.

E40. The method of embodiment E37, wherein the prostate cancer is castration-resistant metastatic prostate cancer.

E41. The method of embodiment E37, wherein the liver cancer is hepatocellular carcinoma such as advanced metastatic hepatocellular carcinoma.

E42. The method of embodiment E37, wherein the carcinoma of head and neck is recurrent or metastatic squamous cell carcinoma of head and neck.

E43. The method of embodiment E37, wherein the colorectal cancer is advanced colorectal cancer metastatic to liver.

E44. The method of embodiment E37, wherein the ovarian cancer is platinum-resistant ovarian cancer or platinum-sensitive recurrent ovarian cancer.

E45. The method of embodiment E37, wherein the glioma is a metastatic glioma.

E46. The method of embodiment E37, wherein the lung cancer is non-small cell lung cancer (NSCLC).

E47. The method of any one of embodiments E1 to E46, wherein administering a TME phenotype class-specific therapy reduces the cancer burden by at least about 10%, 20%, 30%, 40%, or 50% compared to the cancer burden prior to the administration.

E48. The method of any one of embodiments E1 to E47, wherein the subject exhibits progression-free survival of at least about 1, 2, 3, 4, 5, 6, 7, 8, 9, 10, 11, or 12 months, or at least about 1, 2, 3, 4 or 5 years after the initial administration of the TME phenotype class-specific therapy.

E49. The method of any one of embodiments E1 to E48, wherein the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy.

E50. The method of any one of embodiments E1 to E49, wherein the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy.

E51. The method of any one of embodiments E1 to E50, wherein the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy.

E52. The method of any one of embodiments E1 to E50, wherein administering the TME phenotype class-specific therapy improves progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject who has not received a TME phenotype class-specific therapy assigned using an ANN classifier such as TME Panel-1.

E53. The method of any one of embodiments E1 to E50, wherein administering the TME phenotype class-specific therapy improves overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject who has not received a TME phenotype class-specific therapy assigned using an ANN classifier such as TME Panel-1.

E54. A method of assigning a TME phenotype class to a cancer in a subject in need thereof, the method comprising
(i) generating an ANN classifier by training an ANN with a training set comprising RNA expression levels for each gene in a gene panel in a plurality of samples obtained from a plurality of subjects, wherein each sample is assigned a TME phenotype classification; and,
(ii) assigning, using the ANN classifier, a TME phenotype class to the cancer in the subject, wherein the input to the ANN classifier comprises RNA expression levels for each gene in the gene panel in a test sample obtained from the subject.

E55. A method of assigning a TME phenotype class to a cancer in a subject in need thereof, the method comprising generating an ANN classifier by training an ANN with a training set comprising RNA expression levels for each gene in a gene panel in a plurality of samples obtained from a plurality of subjects, wherein each sample is assigned a TME phenotype classification; wherein the ANN classifier assigns a TME phenotype class to the cancer in the subject using as input RNA expression levels for each gene in the gene panel in a test sample obtained from the subject.

E56. A method of assigning a TME phenotype class to a cancer in a subject in need thereof, the method comprising using an ANN classifier to predict the TME phenotype class of the cancer in the subject, wherein the ANN classifier is generated by training an ANN with a training set comprising RNA expression levels for each gene in a gene panel in a plurality of samples obtained from a plurality of subjects, wherein each sample is assigned a TME phenotype class or combination thereof.

E57. The method of any one of embodiments E54 to E56, where the method is implemented in a computer system comprising at least one processor and at least one memory, the at least one memory comprising instructions executed by the at least one processor to cause the at least one processor to implement the machine-learning model.

E58. The method of embodiment E57, further comprising
(i) inputting, into the memory of the computer system, the ANN classifier code;
(ii) inputting, into the memory of the computer system, the gene panel input data corresponding to the subject, wherein the input data comprises RNA expression levels;
(iii) executing the ANN classifier code; or,
(v) any combination thereof.

E59. A method to treat a subject having a locally advanced, metastatic gastric cancer with an IA TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject.

E60. A method to treat a subject having a locally advanced, metastatic gastric cancer with an A TME phenotype comprising administering an A TME phenotype class-specific therapy to the subject, wherein the TME phenotype class has been assigned by applying an ANN classifier to a plurality of RNA expression levels obtained from a gene panel from a cancer tumor sample obtained from the subject.

E61. A method to treat a subject having a locally advanced, metastatic gastric cancer with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject.

E62. A method to treat a subject having a previously untreated gastric cancer with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject.

E63. A method to treat a subject having a previously untreated gastric cancer with an A TME phenotype comprising administering an A TME phenotype class-specific therapy to the subject, wherein the TME phenotype class has been assigned by applying an ANN classifier to a plurality of RNA expression levels obtained from a gene panel from a cancer tumor sample obtained from the subject.

E64. A method to treat a subject having a locally advanced/metastatic HER2-negative breast Cancer with an A TME phenotype comprising administering an A TME phenotype class-specific therapy to the subject, wherein the TME phenotype class has been assigned by applying an ANN classifier to a plurality of RNA expression levels obtained from a gene panel from a cancer tumor sample obtained from the subject.

E65. A method to treat a subject having a locally advanced/metastatic HER2-negative breast cancer with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject.

E66. A method to treat a subject having a castration-resistant metastatic prostate cancer with an A TME phenotype comprising administering an A TME phenotype class-specific therapy to the subject, wherein the TME phenotype class has been assigned by applying an ANN classifier to a plurality of RNA expression levels obtained from a gene panel from a cancer tumor sample obtained from the subject.

E67. A method to treat a subject having a castration-resistant metastatic prostate cancer with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject.

E68. A method to treat a subject having a advanced metastatic hepatocellular carcinoma with an IA TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject.

E69. A method to treat a subject having a advanced metastatic hepatocellular carcinoma with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject.

E70. A method to treat a subject having a recurrent/metastatic squamous cell carcinoma of head and neck with an IA TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject.

E71. A method to treat a subject having a recurrent/metastatic squamous cell carcinoma of head and neck with an IS TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject.

E72. A method to treat a subject having a melanoma with an IA TME phenotype comprising administering an IA TME phenotype class-specific therapy to the subject, wherein the TME phenotype class has been assigned by applying an ANN classifier to a plurality of RNA expression levels obtained from a gene panel from a cancer tumor sample obtained from the subject.

E73. A method to treat a subject having a melanoma with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject.

E74. A method to treat a subject having an advanced colorectal cancer metastatic to liver with an ID TME phenotype comprising administering an ID TME phenotype class-specific therapy to the subject, wherein the TME phenotype class has been assigned by applying an ANN classifier to a plurality of RNA expression levels obtained from a gene panel from a cancer tumor sample obtained from the subject.

E75. A method to treat a subject having a platinum resistant or platinum-sensitive recurrent ovarian cancer with an IA, IS or A TME phenotype comprising administering an IA, IS, or A TME phenotype class-specific therapy to the subject, wherein the TME phenotype class has been assigned by applying an ANN classifier to a plurality of RNA expression levels obtained from a gene panel from a cancer tumor sample obtained from the subject.

E76. A method to treat a subject having platinum-resistant or platinum-sensitive recurrent triple negative breast Cancer with an IA, IS or A TME phenotype comprising administering an IA, IS or A TME phenotype class-specific therapy to the subject, wherein the TME phenotype class has been assigned by applying an ANN classifier to a plurality of RNA expression levels obtained from a gene panel from a cancer tumor sample obtained from the subject.

E77. A method to treat a subject having melanoma with an IS TME phenotype comprising administering an IS TME phenotype class-specific therapy to the subject, wherein the TME phenotype class has been assigned by applying an ANN classifier to a plurality of RNA expression levels obtained from a gene panel from a cancer tumor sample obtained from the subject.

E78. A method to treat a subject having metastatic colorectal cancer with an A or IS TME phenotype comprising administering an A or IS TME phenotype class-specific therapy to the subject, wherein the TME phenotype class has been assigned by applying an ANN classifier to a plurality of RNA expression levels obtained from a gene panel from a cancer tumor sample obtained from the subject.

E79. A method to treat a subject having glioma or glioblastoma with an IS or IA TME phenotype comprising administering an IS or IA TME phenotype class-specific therapy to the subject, wherein the TME phenotype class has been assigned by applying an ANN classifier to a plurality of RNA expression levels obtained from a gene panel from a cancer tumor sample obtained from the subject.

E80. A method to treat a subject having non-small cell lung cancer with an IS or IA TME phenotype comprising administering an IS or IA TME phenotype class-specific therapy to the subject, wherein the TME phenotype class has been assigned by applying an ANN classifier to a plurality of RNA expression levels obtained from a gene panel from a cancer tumor sample obtained from the subject.

E81. A kit comprising (i) a plurality of oligonucleotide probes capable of specifically detecting an RNA encoding a gene biomarker from TABLE 1, and (ii) a plurality of oligonucleotide probes capable of specifically detecting an RNA encoding a gene biomarker from TABLE 2.

E82. An article of manufacture comprising (i) a plurality of oligonucleotide probes capable of specifically detecting an RNA encoding a gene biomarker from TABLE 1 (or FIG. 9A-9G), and (ii) a plurality of oligonucleotide probes capable of specifically detecting an RNA encoding a gene biomarker from TABLE 2 (or FIG. 9A-9G), wherein the article of manufacture comprises a microarray.

### EXAMPLES

### Example 1

### TME Panel-1 Classifier

The classifier used in the methods of the present disclosure is a feed-forward artificial neural network (ANN) consisting of at least three layers of nodes: an input layer, a hidden layer, and an output layer. Except for the input nodes, each node is a neuron that uses a nonlinear activation function. The artificial neural network utilizes backpropagation for training.

Training set: The ACRG gene expression dataset was used as training set. The ACRG training set comprised 235 samples out of 298 available, as 63 samples were identified as lying close to the decision boundary of the class labels; these samples affected the robustness of the model, and therefore were not included in the training set. Also included were 98 continuous variables (a 98 gene panel which comprises a subset of the genes presented in the Angiogenesis Signature gene panel of TABLE 1 and the Immune Signature gene panel of TABLE 2), and corresponded to four target classes (A, IA, IS, and ID tumor microenvironments). Each sample included values (e.g., mRNA levels) for each gene in the gene panel and its classification into a specific Class assigned using a population method based on two Signatures disclosed in U.S. application Ser. No. 17/089,234, which is incorporated herein by reference in its entirety.

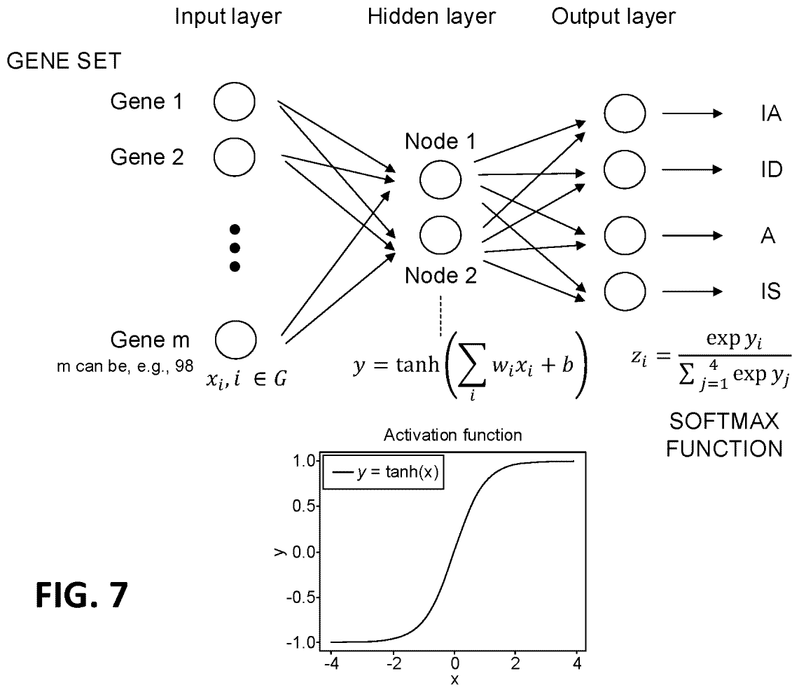

Neural Layer Architecture: The ANN used was a multi-layer perceptron (MLP) comprising an input layer, and output layer, and one hidden layer, as shown in a simplified form in FIG. 7. Each neuron in the input layer was connected to the two neurons in the hidden layer, and each of the neurons in the hidden layer was connected to each of the neurons in the output layer.

Training: A goal of the training process was to identify weights wi for each input and bias b in the hidden layer such that the neural network minimized the prediction error on the training set. See FIG. 7. As shown in FIG. 7, each gene in the gene panel (x₁ . . . xₙ) was used as input for each neuron in the hidden layer and a bias b value for the hidden layer was identified through the training process. The output from each neuron was a function of each gene expression level (xᵢ), weight (wᵢ) and bias (b) as shown in FIG. 7.

A hyperbolic tangent activation function (tanh) that ranged from −1 to 1 was used to generate an ANN classifier as described herein

y(vᵢ)=tanh(vᵢ)

wherein yᵢ was the output of the i th node (neuron) and vᵢ was the weighted sum of the input connections.

As described above, the artificial neural network classifier comprised gene expression values in the input layer (corresponding to a 98 gene panel), two neurons in the hidden layer that encoded the relation between the two stromal signatures, and four outputs which predicted the probability of four stromal phenotypes. See FIG. 8. Multi-class classification of the output layer values into four TME phenotype classes (IA, ID, A, and IS) was supported by applying a logistic regression classifier comprising the Softmax function. Softmax assigned decimal probabilities to each class that had to add up to 1.0. This additional constraint helped training converge more quickly. Softmax was implemented through a neural network layer just before the output layer and had the same number of nodes as the output layer.

As an additional refinement, various cut-offs were applied to the results of the Softmax function depending on the particular dataset used.

Inspection of the artificial neural network classifier revealed that the training algorithm has indeed learned the weights that represented the sign-based rule of the Angiogenesis Signature and Immune Signature. See TABLE 14. The rule was inferred from the training data automatically. The algorithm was not given any assumptions about the Angiogenesis Signature and Immune Signature except for the hidden layer to include two neurons. For each hidden neuron, the genes from the Angiogenesis Signature and Immune Signature contributed to at least some extent, either by a positive or negative gene weight, however one hidden neuron was more dominated by one signature, and vice versa.

TABLE 14
Artificial neural network weights on the output layer.

| | Output A | Output IA | Output ID | Output IS |
|---|---|---|---|---|
| Hidden Neuron 1 | 1.83 | −1.96 | 1.95 | −1.82 |
| Hidden Neuron 2 | −1.82 | 1.90 | 1.77 | −1.85 |

A list of parameters of the final Artificial Neural Network model fitted on the ACRG dataset is shown in TABLE 15.

TABLE 15
Parameters of the Final Artificial Neural Network Model.
MLP Classifier Parameters

| | |
|---|---|
| hidden_layer sizes | 2 |
| Alpha | 2 |
| Solver | Lbfgs |
| Activation | Tanh |
| learning_rate | Constant |

| | | | Output | Output | Output | Output |
|---|---|---|---|---|---|---|
| | | | TME | TME | TME | TME |
| | Hidden | Hidden | Class | Class | Class | Class |
| | Neuron 1 | Neuron 2 | A | IA | ID | IS |
| Intercept bias | 5.750706 | 6.132147 | −0.707687 | −0.641524 | −0.375602 | −0.413502 |

| | Hidden | Hidden | | | | | |
|---|---|---|---|---|---|---|---|
| Coefficients* | Neuron 1 | Neuron 2 | | Output A | Output IA | Output ID | Output IS |
| AFAP1L2 | −0.151264 | −0.117321 | Hidden | 1.83 | −1.96 | 1.95 | −1.82 |
| | | | Neuron 1 | | | | |
| AGR2 | −0.437438 | 0.049720 | Hidden | −1.82 | 1.90 | 1.77 | −1.85 |
| | | | Neuron 2 | | | | |
| BACE1 | −0.115562 | −0.271820 | | | | | |
| BGN | 0.029208 | −0.112965 | | | | | |

*Exemplary genes from a 98 gene set

The results of the application of the ANN model to 1200 patient samples sequences using RNA exome sequencing technology to 400 patients' samples, each of three different tumor types—colorectal, gastric, and ovarian and the consistency of the results across the probable TME phenotypes revealed that the ANN model of the present disclosure was agnostic to tumor type.

The ANN model was used on patient data (n=704) to retrospectively classify the TME phenotypes of tumors from at least 17 different origins in the body (TABLE 16). No outcome data was associated with the classification, but the distribution of the four TME phenotypes was similar to the distribution of the four TME phenotypes classified in an analysis of 1,099 samples, representing samples of ovarian (n=392), colorectal (n=370), and gastric cancers (n=337), sequenced by RNA exome techniques.

TABLE 16
TME phenotypes of 704 patients from at least 17 different origins.

| Biomarker Call | N/Total Patient Samples | Percentage |
|---|---|---|
| IA (Z-score) | 102/704 | 14.5% |
| IA (ANN) | 120/704 | 17.1% |
| IS_(Z-score) | 246/704 | 34.9% |
| IS_(ANN) | 234/704 | 33.2% |
| A (Z-score) | 108/704 | 15.3% |
| A_(ANN) | 104/704 | 14.7% |
| ID (Z-score) | 247/704 | 35.1% |
| ID (ANN) | 245/704 | 34.8% |

Projections of the probability function that resulted from the application of the ANN model to the data were plotted in a latent space, represented by disease scores glyphs (Complete Response, CR; Partial Response, PR; Stable Disease, SD; Progressive Disease, PD). The latent space visualizations provided a probability of the subtype call, which could be used to inform physicians of biomarker confidence to help with treatment decisions.

The curved contours observed in the latent space figures occurred due to interaction terms between features in the model. In the latent space plots, the features were a Angiogenesis Signature score (e.g., a signature in which gene activation was correlated with endothelial cell signature activation) and a Immune Signature score (e.g., a signature in which activation was correlated with inflammatory and immune cell signature activation). In this context, the term interaction refers to a situation in which the effect of one feature on the prediction depends on the value of the other feature, i.e., when effects of the two features are not additive. For example, adding or subtracting features in the model implies no interaction; however, multiplying, dividing, or pairing features in the model implies interaction.

The plots that predict the TME phenotype (four classes, corresponding to four TME) have curved contours because although the underlying model (a neuron) for each single TME phenotype class was equivalent to logistic regression, renormalization of the four TME phenotype class probabilities took place for the four logistic regressions, so the sum of the four TME phenotype class probabilities were equal to one. This was accomplished using the Softmax function, which is where interaction between the Angiogenesis Signature score and the Immune Signature score occurred. Consequently, this model produced curved contours.

### Example 2

### Colon Cancer Datasets

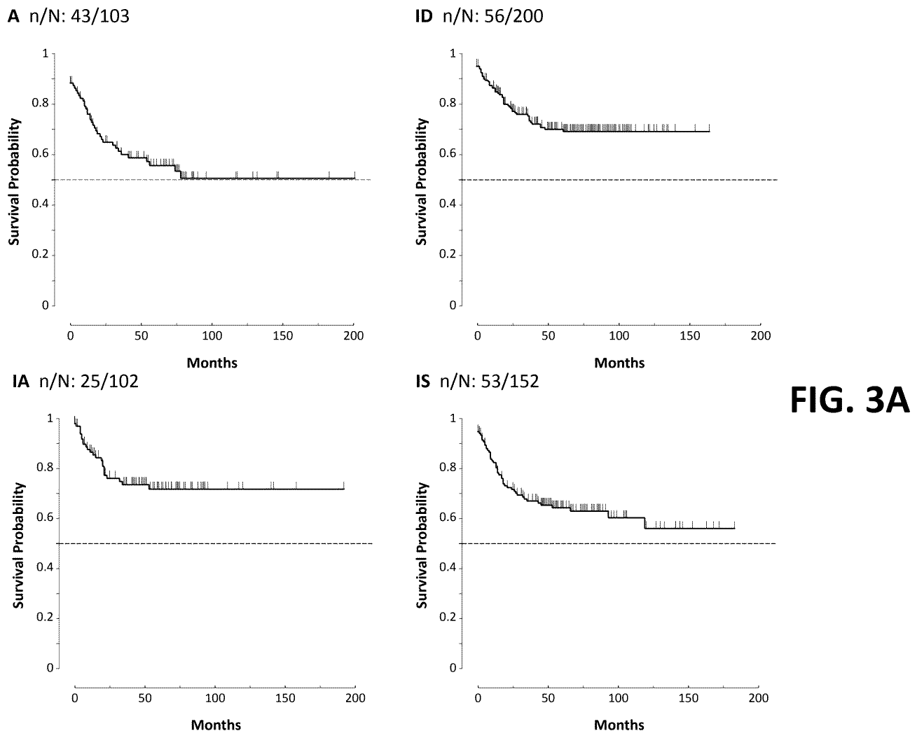

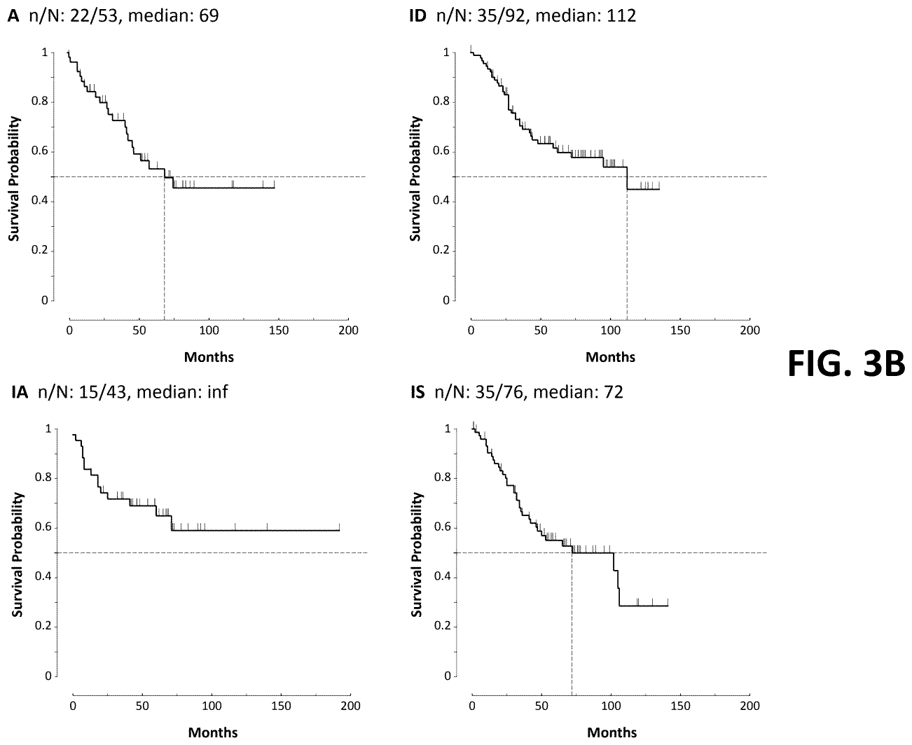

CIT French Consortium Colon Cancer: CIT (Cartes d'Identite des Tumeurs; GSE39582; Marisa et al. (2013) PLOS Medicine 10(5):e1001453) is a public dataset that contains 566 primary tumor samples from patients in stage 1-4 colorectal cancer CRC who had curative surgery between 1988 and 2007 in France. Dataset contains RNA expression (microarray), CMS classification, mutational status of KRAS, TP53, BRAF, MMR, and CIN status. Additionally, the disease-free interval (FIG. 3A), overall survival status (FIG. 3B), stage of disease at diagnosis and site of primary tumor was available in the dataset.

Patients in the CIT study had surgery to remove their tumors and the DNA and RNA genomics of these patients were analyzed and classified into the four CMS types (Guinney et al. (2015) Nature Medicine 21:1350-6).

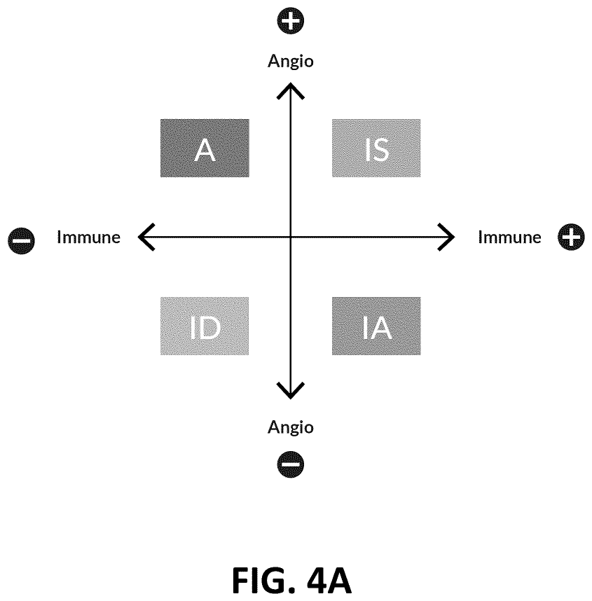

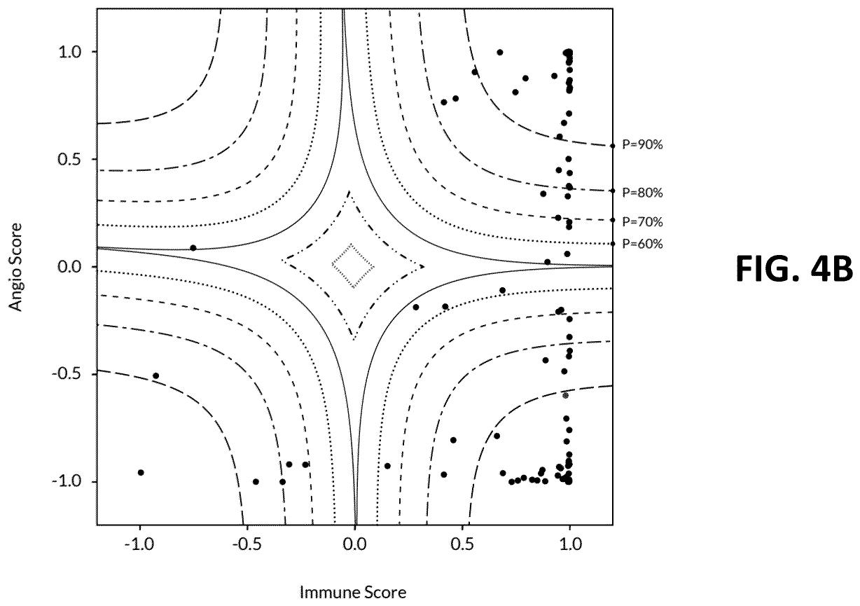

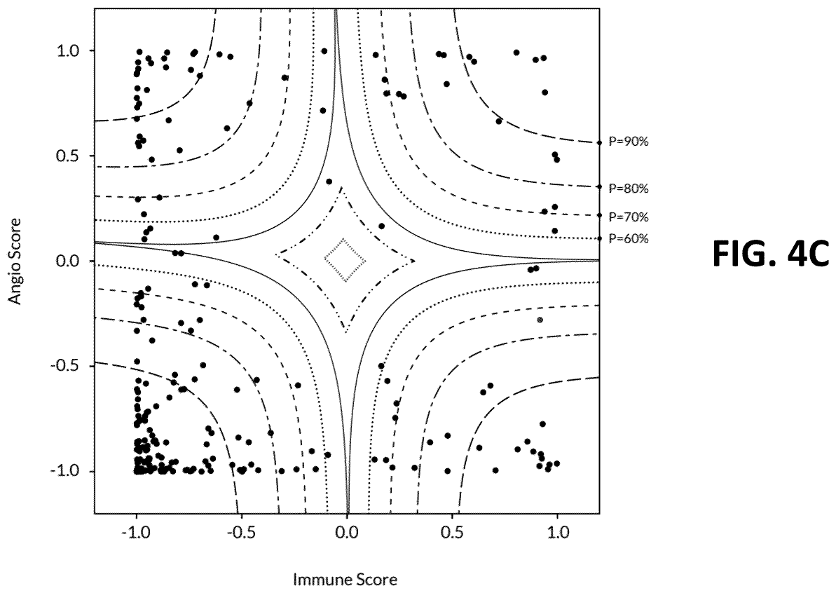

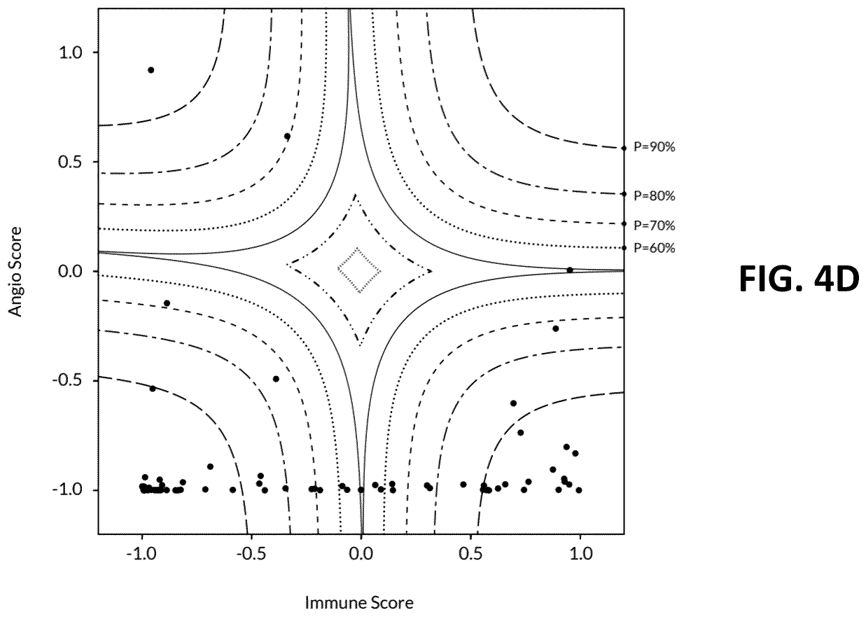

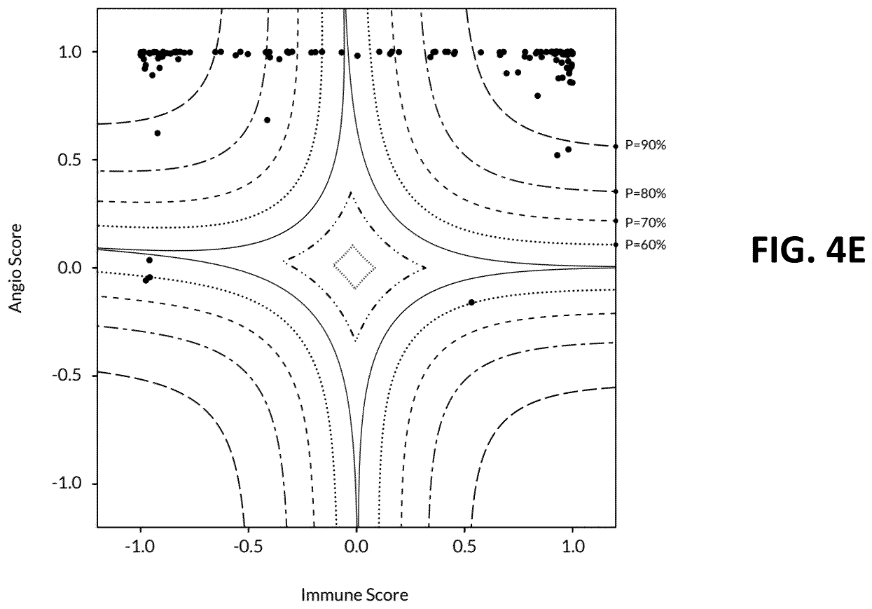

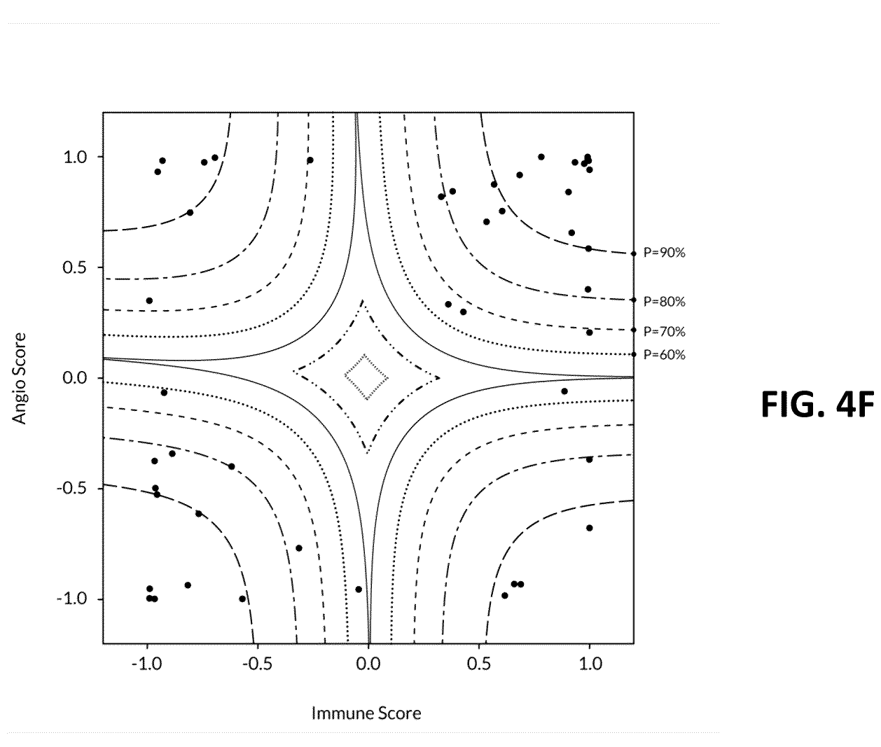

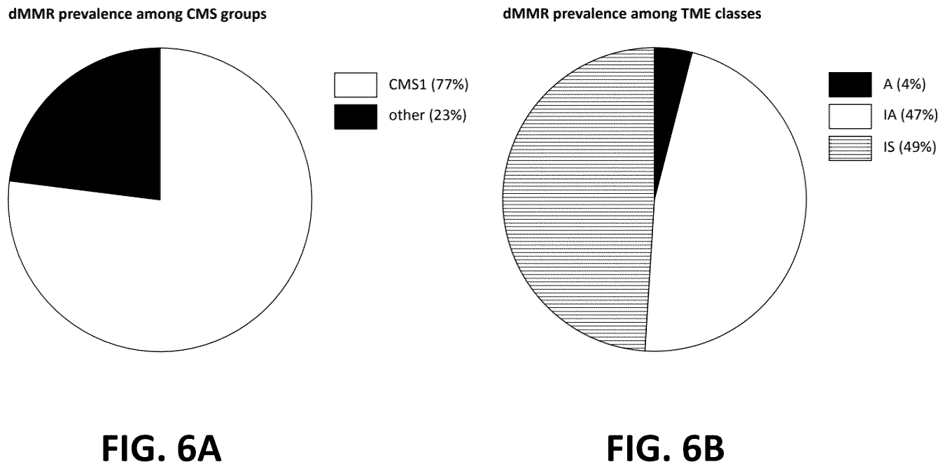

The publicly available CIT data (RNA gene expressions) were downloaded and analyzed using the TME Panel-1 ANN classifier. Using the angiogenesis signature scores and immune signature scores (FIG. 4A) provided by TME Panel-1, latent space plots were generated (FIGS. 4B, 4C, 4D, 4E and 4F). dMMR prevalence was calculated and is provided in FIG. 6A and FIG. 6B.

The advantage of having a data set comprising genomic analysis information derived from surgical treatment samples was that the outcome data was not influenced by the presence of therapeutics. Therefore, the TME phenotype classification resulting from the application of the TME Panel-1 classifier was prognostic.

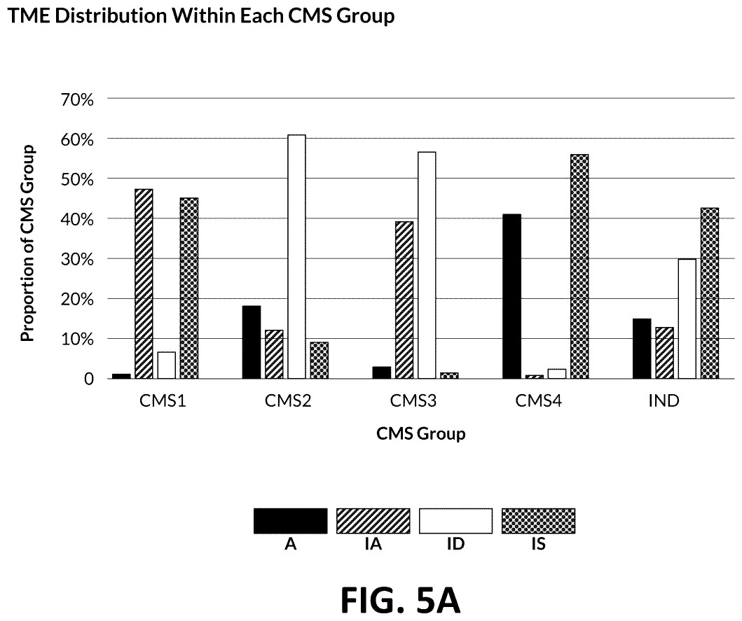

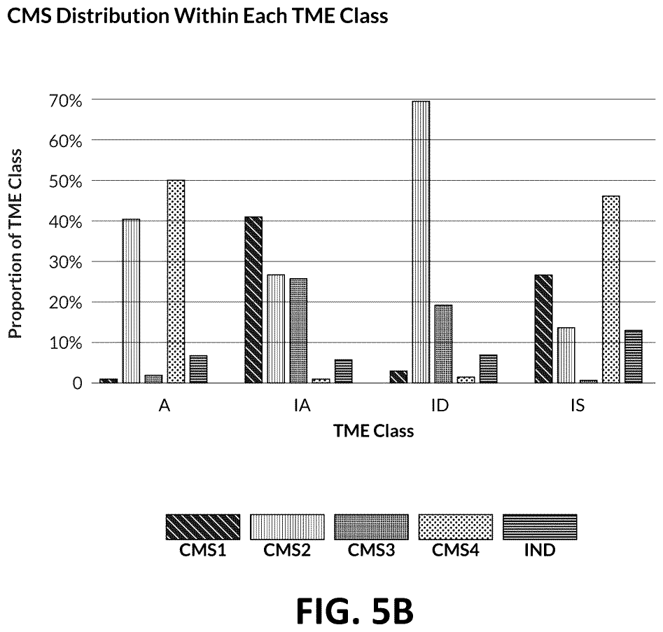

FIGS. 5A and 5B compare patient distribution in CIT according to CMS and TME Panel-1. FIG. 6A shows TME phenotype class distribution of the CIT dataset within each CMS group. For each CMS group, the proportion of patients of each TME class is shown, shaded according to the legend. FIG. 5B shows CMS distribution of the CIT dataset within each TME phenotype class. For each TME class, the proportion of patients of each CMS group is shown, shaded according to the legend. FIGS. 5A and 5B represent the same but converse tabulation analysis.

Wood Hudson Left and Right CRC TME Prevalence: The Wood Hudson dataset is a proprietary collection of 93 samples from the Wood Hudson Cancer Research Laboratory of patients with metastatic CRC that were treated with bevacizumab (AVASTIN®) at some point in their treatment history following surgery. RNA expression was measured by RNA-seq and each sample was evaluated for PD-L1. Additionally, stage of disease at diagnosis and site of primary tumor was available in the dataset. In general, left-sided (distal) colorectal cancer is found in the descending colon, and right-sided (proximal) colorectal cancer is found in the ascending colon.

RNA expression levels from FFPE samples from CRC patients were pre-processed and analyzed using the TME Panel-1 ANN classifier. The presence of TME phenotypes classes A and IA was lower in right (proximal) colorectal cancer that in left (distal) sided colorectal cancer.

As indicated above, all Wood Hudson (WH) and CIT samples were classified using the TME Panel-1 classifier into one of four TME phenotype classes (FIG. 1). This enabled tabulation of the prevalence of each TME phenotype classes by disease stage and Left (distal) or Right (proximal) tumor side. Survival analysis was performed on the CIT patients to evaluate the prognostic potential of TME Panel-1. Disease free survival (DFS) was evaluated on early stage (0-2) patients, inferred as months from surgery to recurrence, and overall survival (OS) on late stage (3-4) patents as time from recurrence to death. The relationship between the CMS and TME Panel-1 classifiers was explored by mapping CIT patients onto the latent space created by two hidden nodes of the TME Panel-1 classifier artificial neural network. In this manner, TME phenotype class was assigned to each patient, all of whom already had CMS group assignments.

TME Panel-1 has been shown to be both prognostic in gastric cancer, and predictive of targeted therapy outcome in gastric and ovarian cancer. Strand-Tibbitts et al. Working Towards Precision Medicine for the Tumor Microenvironment. SITC (2019); Strand-Tibbitts et al. (2020) Development of an RNA-based Diagnostic Platform Based on the Tumor Microenvironment Dominant Biology. SITC. Preliminary analysis of nearly 400 colorectal cancer patient samples suggested that TME Panel-1 classifier is suitable for colorectal cancer.

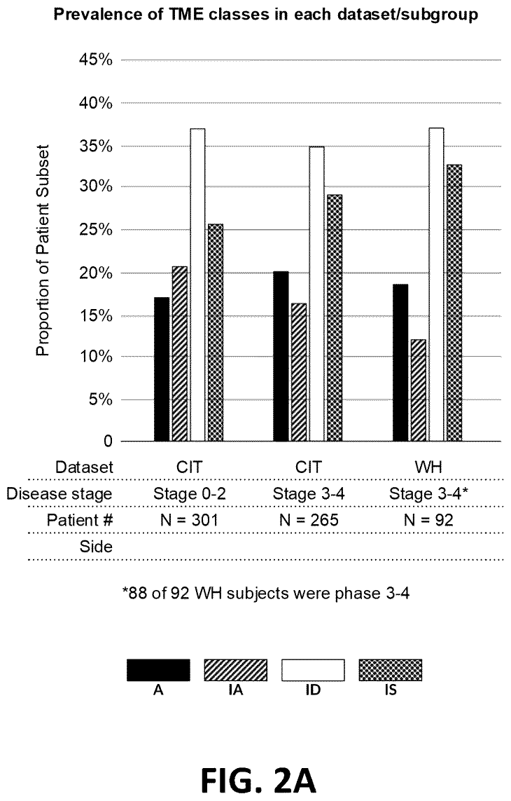

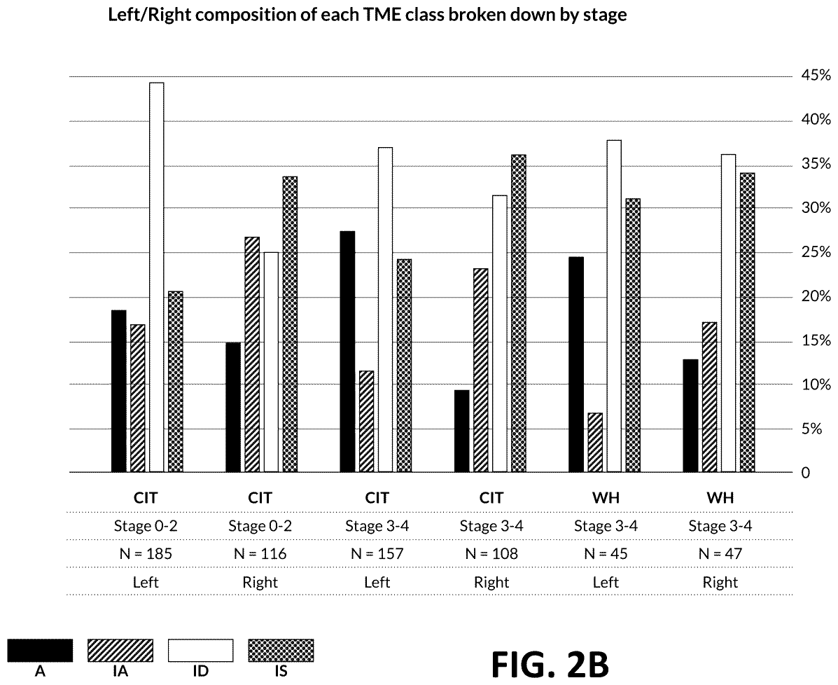

This analysis more directly addressed whether the TME Panel-1 classifier was applicable in colorectal cancer. Colorectal cancer is a heterogeneous disease with known differences in prognosis and tumor biology depending on, for example, the side of tumor origin left (distal) versus right (proximal) tumors, and the stage of disease. All patients from the CIT and WH datasets were classified according to the TME Panel-1 classifier into one of four TME phenotype classes: Angiogenic (A), Immune Suppressed (IS), Immune Active (IA) or Immune Desert (ID). The prevalence of patients in each TME phenotype class was tabulated based on disease stage (FIG. 2A) and tumor side (FIG. 2B).

A plurality of subjects were observed in the ID TME phenotype group, which was consistent with the notion that colorectal cancer has a “cold” tumor microenvironment. The next most prevalent TME phenotype was IS, indicating that many patients have tumors with high angiogenesis and high immune infiltration, but require therapy that can address the interaction between these biologies. Furthermore, the prevalence of the CIT stage 3-4 phenotypes were more similar to WH, for which 89 of the 93 patients were also stage 3-4, than it was to CIT stage 0-2 (FIG. 2A). When patients were further split by side of tumor into Left and Right (FIG. 2B), in all three data groups, the Left side was found to be more angiogenic (A), while the right side was more immune active (IA). Taken together, these results indicate that the TME Panel-1 classifications reflect biological attributes of the disease and confirm that it is generalizable across independent datasets.

Since TME Panel-1 recapitulated fundamental aspects of colorectal biology, TME phenotype classes were then evaluated to see if the classes were prognostic of the disease. Patients from the CIT dataset were analyzed for survival probability. The high angiogenic TME phenotype classes (A) and (IS) showed worse disease-free survival until recurrence, and worse overall survival among late-stage patents. In both early and late-stage analyses, immune active (IA) patients had the best prognosis

Numerous research teams have proposed prognostic and/or predictive signatures for colorectal cancer, recognizing this is a heterogeneous disease with distinct patient subsets. Most notable among these efforts is the Consensus Molecular Subtypes (CMS), a synthesis of six prevailing CRC stratification models. CMS describes four patient groups 1-4, and a fifth “indeterminant” catch-all (herein “IND”). To explore how TME Panel-1 related to CMS, patients from the CIT dataset were classified according to the TME Panel-1 algorithm, and projected on the latent space of the TME model defined by an immune X-axis and angiogenic Y-axis (FIG. 4A). Patients in each CMS group were then evaluated based on these biological axes, and more specifically, based on the TME phenotypes defined by the latent space quadrants. See FIG. 4B-4F.

Consistent with Guinney et al 2015 (Guinney et al. (2015) Nature Medicine 21:1350-6), CMS1 subjects were mostly high immune (positive on X-axis, top middle panel), and CMS4 were mostly angiogenic (positive on Y-axis, bottom right panel). CMS2 were distributed in all four quadrants, though enriched for ID, while CMS3 was low angiogenic. CMS-indeterminant patients were observed all over, but with a plurality in IS.

Despite the consistencies with the immune CMS1 and angiogenic CMS4, TME Panel-1 provides more granularity in terms of the molecular biological characteristics of the patients. For example, a considerable number of CMS1 patients were observed that had high TME angiogenic scores, and many of the CMS4 patients having high TME immune scores. The distribution of patients was quantified between CMS groups and TME classes to better appreciate how these classification approaches may lead to different conclusions about patient biology.

Unlike the meta-model synthesis of CMS, the TME Panel-1 was built to abstract the biology of the tumor microenvironment, and for all solid tumors, not just CRC. The Panel was designed to be predictive, i.e., classification based on those biologies allows matching TME phenotypes with appropriate therapies. This turned out to be the case when examined in other tumor types, such as Gastric and Ovarian (Strand-Tibbitts et al. (2020) Development of an RNA-based Diagnostic Platform Based on the Tumor Microenvironment Dominant Biology. SITC; Strand-Tibbitts et al. Working Towards Precision Medicine for the Tumor Microenvironment. SITC (2019)). The goal of this analysis was to understand how TME Panel-1 accords with known CRC biology such as differences in Left and Right sided cancers, and correlates with prior subtyping efforts in CRC, such as CMS. There are observed consistencies with prior analysis of immune and angiogenic biologies such as:
(i) Consistent enrichment for TME Angiogenesis (A) class and CMS4, particularly in Left sided tumors
(ii) Consistent enrichment for TME Immune Active (IA) class and CMS1
(iii) Similar prognostic relationships for TME (A) and CMS4 and TME (IA) and CMS1

Observed differences largely reflect the composition and distribution of TME phenotypes across CMS groups, where the TME panel identifies immune and angiogenic signal in patients outside of the canonical CMS definitions. For example, in this analysis of the CIT dataset, patients assigned to (A) and (IA) classes were dispersed more broadly than just CMS4 and CMS1. In fact, only half of the (A) patients are CMS4, and half of (IA) are CMS1.

dMMR/MSI-H are mostly captured by CMS1 and this is the most validated group of CRC patients to CPI (30-50% response). Andre et al. 2020 N. Engl. J. Med. 383:2207-2218. However, recent analysis of the MSS population through HLA mutation analysis and immune cell infiltration studies has suggested there is another 20% of MSS CRC that may be appropriate for CPI treatment. Giannakis et al. 2016 Cell Rep. 15:857-865. Similar to the relationship between TME (A) and CMS4, the TME (IA) class is made up of 41% CMS1 and then significant contributions from CMS2 and CMS3.

TME Panel-1 defines an Immune Suppressed (IS) class. CRC is characterized as “cold,” but this could be for either a lack of immune activity or immune suppression blocking CPI activity. Of note, almost half of the dMMR patients in the CIT dataset are classified as TME (IS) (FIG. 6B). Emerging therapies focused on immunosuppressive cells and cytokines, such as myeloid targeting agents or next-generation immune modulators such as anti-TIM3 or LAG3, may be able to “warm up” the IS group and further enhance immune therapy opportunities in CRC.

### Example 3

Analysis of dMMR Patients in the CIT Dataset

The lack of mismatch repair (MMR) genes in tumor cells can result in the accumulation of Microsatellite (MS) sequences, also known as Short Tandem Repeats (STRs) or Simple Sequence Repeats (SSRs). Also referred to as dMMR, patients that have accumulated MS sequences are called MSI-High, for the high levels of microsatellite instability. The MSI-High/dMMR biomarkers (usually analyzed by PCR and capillary electrophoresis, or NGS sequencing). The MSI-High/dMMR status for patients in the CIT database was analyzed by Marisa et. al. (Marisa et al. (2013) PLOS Medicine 10(5):e1001453), and the CMS classifications of the same patients were determined by Guinney et al. (Guinney et al. (2015) Nature Medicine 21:1350-6), and are shown in FIG. 6A.

MSI-High/dMMR status is an approved biomarker for checkpoint inhibitor (CPI) therapy in colorectal cancer, yet there is only a 30-50% response rate to CPI treatment. As can be seen in FIG. 6A, 77% of the MSI-High/dMMR patients in the CIT dataset are in CMS1, the MSI Immune group, and the rest are in the other CMS groups. When the same analysis is done with the ANN classifier (TME Panel-1), FIG. 6B, 96% of the MSI-High/dMMR patients fall within either the IA or IS TME phenotype classes. Colorectal cancer patients who are classified with the ANN classifier (TME Panel-1) and found to be in the IA TME phenotype class, i.e., are predicted to have the best response to CPI, like gastric cancer patients. Because neither dMMR nor CMS1 can distinguish immune active from immune suppressed, using TME phenotype class IA as a predictor for CPI would further improve on predicting response.

### Example 4

Colorectal Cancer Tumor Microenvironment RNA Signature Correlates to Clinical Response in Checkpoint Inhibitor Use in Patients with Mismatch Repair Deficiency (dMMR) or MSI-H Patients

A clinical trial is run to determine whether colorectal cancer tumor microenvironment phenotypes correlate to clinical responses when patients with dMMR or MSI-High status are treated with a checkpoint inhibitor. The analysis includes 40 colorectal cancer tumor samples. Data indicate that the immune active (IA) TME phenotype is enriched for response to checkpoint inhibition treatment in this patient population.

CRC patients with dMMR or MSI-H have the option of anti-PD-(L)1 (i.e., an inhibitor to PD-1 or PD-L1) therapy after advancement on appropriate front-line therapy. The use of an anti-PD-(L)1 checkpoint inhibitor increases progression-free survival (PFS) and overall survivor (OS) in dMMR or MSI-H patients with advanced colorectal cancer compared to chemotherapy. RNA gene signatures are analyzed from biopsy samples prior to treatment with an anti-PD-(L)1. The TME phenotypes are correlated to ORR, and 20-week PFS, and predict which patients benefit and which do not. Forty (40) patients are enrolled, 10 in each of the 4 stromal phenotypes. The correlation between each TME phenotype is tested against clinical outcome data. In immune active (IA) patients the use of anti-PD-(L)1 therapy confers benefit in comparison to patients classified to angiogenic (A), immune suppressed (IS) and immune desert (ID) TME phenotype classes as shown in the TABLE 17.

TABLE 17
Progression-free survival and overall response rate for the four
TME phenotype classes in a trial of an anti- PD-(L)1 checkpoint
inhibitor in colorectal cancer patients that are MSI-High or dMMR.

| | IA (n = 10) | IS (n = 10) | ID (n = 10) | A (n = 10) |
|---|---|---|---|---|
| ORR (%) | 90 | 50 | 0 | 0 |
| PFS % at | 100 | 70 | 0 | 0 |
| 20 weeks | | | | |

### Example 5

Colorectal Cancer Tumor Microenvironment RNA Signature Correlates to Clinical Response in Mismatch Repair Deficient (dMMR) or MSI-H Advanced Colorectal Cancer Patients Treated with Combination Checkpoint and Phosphatidylserine Inhibitors

A clinical trial is run to determine whether colorectal cancer TME phenotypes correlate to clinical responses when patients with dMMR or MSI-H status are treated with a combination of a checkpoint inhibitor and bavituximab. The analysis includes 40 colorectal cancer tumor samples. Data indicate that the immune active (IA) and (IS) TME phenotypes are the appropriate cohort of patients to treat with this combination.

An anti-PD-1 checkpoint inhibitor is known to increase PFS and OS in dMMR or MSI-H status patients with advanced colorectal cancer (CRC), compared to chemotherapy. CRC patients with dMMR or MSI-H have the option of anti-PD-(L)1 after advancement on appropriate front-line therapy. Patients are classified according to an ANN method such as the TME Panel-1 classifier. Some patients in the IS subgroup do not do as well with monotherapy, and so are subsequently treated with a phosphotidylserine-targeting antibody such as bavituximab in combination anti-PD-1 to improve responses in the IS group and to further optimize the immune therapy treatment paradigm for CRC. RNA gene signatures are analyzed from biopsy samples prior to treatment with anti-PD-1. The TME phenotypes are correlated with ORR and with 20-week PFS. The assigned TME phenotype classes are predictive of which patients benefit and which do not. 40 patients are enrolled, 10 in each of the 4 TME phenotypes. The correlation between each tumor TME phenotype is tested against clinical outcome data. In IA or IS patients the use of bavituximab and anti-PD-(L)1 confers gains in comparison to patients classified to angiogenic (A), and immune desert (ID) TME phenotypes as shown in TABLE 18.

TABLE 18
Progression-free Survival and overall response rate for the 4 TME
phenotypes in a trial of bavituximab and an anti-PD-1 checkpoint
inhibitor in colorectal cancer in dMMR or MSI-H status patients.

| | IA (n = 10) | IS (n = 10) | ID (n = 10) | A (n = 10) |
|---|---|---|---|---|
| ORR (%) | 90 | 90 | 0 | 0 |
| PFS % at | 100 | 100 | 0 | 0 |
| 20 weeks | | | | |

### Example 6

Colorectal Cancer Tumor Microenvironment RNA Signature Correlates to Clinical Response in Metastatic Colorectal Cancer Patients Treated with Anti-Angiogenic Therapy

A retrospective data analysis indicates that colorectal cancer TME phenotypes correlate to clinical responses when patients are treated with targeted therapies, including angiogenesis inhibitors. The analysis includes 60 colorectal cancer tumor samples. Data indicate that the angiogenic (A) and immune suppressed (IS) phenotypes are most responsive to anti-angiogenic therapy, such as bevacizumab, relative to the immune active (IA) and immune desert (ID) phenotypes.

Bevacizumab in combination with chemotherapy increases PFS and OS in patient with advanced colorectal cancer (Snyder, 2018). The overall response rate (ORR) in previously untreated metastatic colorectal cancer patients was reported as 80% in left-sided tumors and 83% in right-sided tumors. Median time to progression (PFS) and overall survival (OS) in both left- and right-sided tumors was 13 months and 37 months, respectively.

To test if TME phenotypes correlate with clinical outcomes when patients are treated with an angiogenesis inhibitor, tumor stroma RNA gene signatures are analyzed from archival tissues collected from 60 colorectal cancer patients (30 left-sided, 30 right-sided) using an ANN classifier such as the TME Panel-1 classifier. The correlation between each TME phenotype is tested against clinical outcome data. In A and IS patients, the use of bevacizumab confers gains in comparison to patients classified to IA and ID TME phenotypes: in A and IS patients median PFS and OS shifts to 15 months and 39 months, respectively. Progression-free survival and OS data in IA and ID patients are consistent with historical values. Overall, the A and IS TME phenotypes correlate specifically with improved clinical outcomes with angiogenesis inhibitors and has a predictive effect with respect to PFS.

### Example 7

First-Line Colorectal Cancer Stromal Phenotypes Correlate with Clinical Response to Navicixizumab and Chemotherapy

The standard of care for second-line treatment for mCRC is the anti-angiogenic ramucirumab plus the chemotherapeutic FOLFIRI. A comparison of ramucirumab plus FOLFIRI vs. placebo plus FOLFIRI in a phase II single arm study resulted in 58% ORR, PFS of 11.5 months and OS of 20.4 mos (Garcia-Carbanero, R. et al. 2014. An Open-Label Phase II Study Evaluating the Safety and Efficacy of Ramucirumab combined with mFOLFOX-6 as First-Line Therapy for Metastatic Colorectal Cancer. The Oncologist, V. 19, pp. 350-1).

A clinical trial is run to show benefit of anti-angiogenesis therapy in CRC by identifying patients based on their stromal phenotypes. RNA gene signatures are analyzed from biopsy samples prior to treatment with navicixizumab and chemotherapy (such as paclitaxel, FOLFOX, FOLFIRI, etc.). The stromal phenotypes are correlated with ORR, PFS, and OS. The analysis includes 40 colorectal cancer patients with treated with navicizumab and chemotherapy in the first-line setting. Data indicate that the angiogenic (A) and immune suppressed (IS) TME phenotypes are the most responsive to navicixizumab and chemotherapy relative to the immune active (IA) and immune desert (ID) TME phenotypes as shown in TABLE 19.

TABLE 19
Overall response rate, overall survival, and progression-
free survival in a retrospective analysis of navicixizumab
and chemotherapy in second-line mCRC.

| | IA | IS | ID | A |
|---|---|---|---|---|
| | (n = 10) | (n = 10) | (n = 10) | (n = 10) |
| ORR (%) | 30 | 80 | 45 | 90 |
| PFS (months) | 10 | 16 | 13 | 15 |
| OS (months) | 15 | 25 | 20 | 26 |

### Example 8

### Anti-VEGF Therapy Phase I/II Trial

The present example concerns the use of anti-angiogenic antibodies (e.g., monoclonal antibodies specific to VEGF or anti-DLL4 monoclonal antibodies) and/or bispecifics antibodies (e.g., the anti-VEGF/anti-DLL4 bispecific navicixizumab) with one component associated with VEGF to enhance the activity as a single agent or in combination with standard of care such as chemotherapy, based on a patient's TME phenotypes according to the present disclosure.

The present example describes an open-label, Phase I/II trial of anti-VEGF therapy alone or in combination with standard of care in patients with refractory adenocarcinoma of the colon or rectum after least two prior regimens of standard chemotherapies (e.g., 3rd line). The trial is conducted at approximately 10 centers world-wide, including the United States, European Union, and Asia. The goals of the trial are to see if the monotherapy anti-VEGF treatment or combination treatment is safe and a clinically meaningful improvement compared to historical results. Including potential predictive outcome in a biomarker positive subgroup (A and IS) to the VEGF treatment or combination treatment with VEGF is clinically meaningful in a RUO (research use only) scenario.

The test product, dose, and mode of administration are as follows: will be administered as an intravenous (IV) infusion according to the clinical protocol.

Formalin-fixed tissue from a recent biopsy is used for generating RNA sequences according to the protocol established by an RNA sequencing technology company, such as HTG Molecular Diagnostics (Tucson, Arizona, USA), QIAGEN (Manchester, UK), Exact Sciences (Madison, WI), or Almac (Craigavon, Northern Ireland, UK). The patient whose TME phenotype is A or IS will receive benefit from the anti-VEGF treatment or anti-VEGF bispecific or combination treatment.

### Example 9

### Anti-VEGF Therapy Phase III Trial

The present example describes a Phase III, pivotal trial for one of the indications of the previous example with anti-VEGF therapy (e.g., with monoclonal antibodies specific to VEGF or anti-DLL4 monoclonal antibodies, and/or with bispecifics antibodies, e.g., the anti-VEGF/anti-DLL4 bispecific navicixizumab) alone or in combination with standard of care in patients with refractory adenocarcinoma of the colon or rectum after least one prior regimen of standard chemotherapies (e.g., 2^{nd} or 3rd line), using the methods of the present disclosure as a stratification tool, i.e., an IUO (Investigator Use Only).

### Example 10

Locally Advanced/Metastatic Gastric Cancer Tumor Microenvironment RNA Signature Correlates to Clinical Response in Maintenance Setting with Immune Checkpoint Inhibitors and Anti-Angiogenic Agents

A clinical trial is run to determine whether locally advanced or metastatic gastric cancer tumor microenvironment phenotypes correlate to clinical responses when patients are treated with an immune checkpoint inhibitor or anti-angiogenic therapy in the maintenance setting following initial chemotherapy. The analysis includes samples from 240 patients across 4 treatment arms (60 patients in each arm). All patients receive first-line chemotherapy and those who achieve stable disease or better are randomized into 4 treatment groups for follow-on therapy: 1) Surveillance only (no therapy given), 2) chemotherapy, 3) immune checkpoint inhibitor, 4) chemotherapy+anti-angiogenic agent. RNA gene signatures are analyzed from tumor samples acquired before any treatments and TME phenotypes are determined. Patients are followed for clinical response, progression-free survival, and overall survival. Data indicate that the immune active (IA) TME phenotype is enriched for response and clinical benefit to immune checkpoint inhibition treatment in this patient population and that the Angiogenic (A) TME phenotype is enriched for response and clinical benefit to chemotherapy+anti-angiogenic therapy.

Gastric cancer patients are randomized to surveillance only or to receiving continued chemotherapy (e.g., capecitabine), an immune checkpoint inhibitor (anti-PD-(L)1—i.e., an inhibitor to PD-1 or PD-L1) therapy, or a chemotherapy combination with anti-angiogenic therapy (e.g., anti-VEGF or anti-VEGFR2) after stabilization of disease or response on first-line chemotherapy (e.g., platinum/fluoruracil). The use of either an immune checkpoint inhibitor or the combination of chemotherapy and an anti-angiogenic agent increases overall response rate (ORR), progression-free survival (PFS) and overall survival (OS) in patients with advanced gastric cancer compared to chemotherapy or surveillance alone. The TME phenotypes are correlated to ORR, and 12-week PFS, and overall survival and predict which patients benefit and which do not. Two hundred forty (240) patients are enrolled, 60 in each of the treatment groups with equal representation across the 4 stromal phenotypes (15 in each phenotype per treatment group). The correlation between each TME phenotype is tested against clinical outcome data. In immune active (IA) patients the use of immune checkpoint therapy confers benefit in comparison to patients classified to angiogenic (A), immune suppressed (IS) and immune desert (ID) TME phenotype classes as shown in the TABLE 20. In angiogenic (A) patients the use of a combination of chemotherapy and anti-angiogenic therapy confers benefit in comparison to patients classified to immune active (IA), immune suppressed (IS) and immune desert (ID) TME phenotype classes as shown in the TABLE 21.

TABLE 20
Progression-free survival and overall response rate for
the four TME phenotype classes in the group receiving
an immune checkpoint inhibitor in gastric cancer patients
in the maintenance setting following chemotherapy.

| | IA | IS | ID | A |
|---|---|---|---|---|
| | (n = 15) | (n = 15) | (n = 15) | (n = 15) |
| ORR (%) | 20 | 7 | 0 | 0 |
| PFS % at 12 weeks | 80 | 53 | 40 | 27 |

TABLE 21
Progression-free survival and overall response rate for the four
TME phenotype classes in the group receiving a combination of
chemotherapy and anti-angiogenic inhibitor in gastric cancer
patients in the maintenance setting following chemotherapy.

| | IA | IS | ID | A |
|---|---|---|---|---|
| | (n = 15) | (n = 15) | (n = 15) | (n = 15) |
| ORR (%) | 7 | 13 | 0 | 27 |
| PFS % at 12 weeks | 53 | 60 | 13 | 73 |

### Example 11

Tumor Microenvironment RNA Signature from Previously Untreated Gastric Cancer Patients Correlates to Clinical Response in Perioperative Setting with an Anti-Angiogenic Agent

A clinical trial is run to determine whether locally advanced or metastatic gastric cancer tumor microenvironment phenotypes correlate to clinical responses when patients are treated with chemotherapy with or without anti-angiogenic therapy in the perioperative setting. The analysis includes samples from 200 patients across 2 treatment arms (100 patients in each arm). Patients receive either chemotherapy or a combination of chemotherapy and an anti-angiogenic agent 9 weeks before surgery and an additional 9 weeks after surgical resection of their primary gastric tumors. RNA gene signatures are analyzed from tumor resection samples and TME phenotypes are determined. Patients are followed for clinical response, progression-free survival, and overall survival. Data indicate that the biomarker positive angiogenic TME phenotypes (A and IS) are enriched for response and clinical benefit to chemotherapy in combination with an anti-angiogenic therapy.

Gastric cancer patients are randomized to a 9 week pre-surgical/9 week post-surgical regimen of chemotherapy (e.g., epirubicin/cisplatin/capecitabine etc.) or a combination of chemotherapy and anti-angiogenic therapy (e.g., anti-VEGF or anti-VEGFR2). The use of chemotherapy and an anti-angiogenic agent increases overall response rate (ORR), progression-free survival (PFS) and overall survival (OS) in patients with advanced gastric cancer compared to chemotherapy alone. The TME phenotypes are correlated to perioperative RECIST ORR, pathological response and overall survival and predict which patients benefit and which do not. Two hundred (200) patients are enrolled, 100 in each of the treatment groups with equal representation across the 4 stromal phenotypes (25 in each phenotype per treatment group). The correlation between each TME phenotype is tested against clinical outcome data. In tumors that are biomarker positive, represented by angiogenic (A) and immune suppressed (IS) phenotypes, the use of a combination of chemotherapy and anti-angiogenic therapy confers benefit in comparison to patients classified as biomarker negative, represented by immune active (IA) and immune desert (ID) TME phenotype classes as shown in the TABLE 22.

TABLE 22
Perioperative RECIST overall response rate (ORR), pathological
response rate and 3 year overall survival rate for the four
TME phenotype classes and by biomarker status in the group
receiving chemotherapy and an anti-angiogenic agent.

| | Biomarker | Biomarker | IA | IS | ID | A |
|---|---|---|---|---|---|---|
| | Positive | Negative | (n = | (n = | (n = | (n = |
| | (n = 50) | (n = 50) | 25) | 25) | 25) | 25) |
| Perioperative | 60 | 20 | 28 | 48 | 12 | 72 |
| ORR (%) | | | | | | |
| Pathological | 48 | 12 | 20 | 32 | 4 | 64 |
| Response Rate | | | | | | |
| (%) | | | | | | |
| OS % at 3 years | 66 | 30 | 40 | 60 | 20 | 72 |

### Example 12

Locally Advanced/Metastatic Gastric Cancer Tumor Microenvironment RNA Signature Correlates to Clinical Response in First Line Setting with Chemotherapy, Immune Checkpoint Inhibitors and Bavituximab

A clinical trial is run to determine whether locally advanced or metastatic gastric cancer tumor microenvironment phenotypes correlate to clinical responses when patients are treated with chemotherapy, an immune checkpoint inhibitor, and Bavituximab in the first-line setting. The analysis includes samples from 120 patients across 2 treatment arms (60 patients in each arm). Patients are randomized to receive either (i) chemotherapy and an immune checkpoint inhibitor, or (ii) chemotherapy, an immune checkpoint inhibitor, and bavituximab. RNA gene signatures are analyzed from tumor samples acquired before any treatments and TME phenotypes are determined. Patients are followed for clinical response, progression-free survival, and overall survival. Data indicate that the immune active (IA) and immune suppressed (IS) TME phenotypes are enriched for response and clinical benefit to the regimen consisting of chemotherapy, immune checkpoint inhibition, and Bavituximab treatment in this patient population.

Gastric cancer patients are randomized to receiving a regimen of chemotherapy (i.e. capecitabine, 5-FU, cisplatin etc.) plus an immune checkpoint inhibitor (anti-PD-(L)1—i.e., an inhibitor to PD-1 or PD-L1) therapy, or a regimen of chemotherapy/immune checkpoint inhibitor/Bavituximab. The combination of chemotherapy/immune checkpoint inhibitor/Bavituximab increases overall response rate (ORR), 6 month progression-free survival (PFS) and overall survival (OS) patients with advanced gastric cancer compared to the regimen of chemotherapy/immune checkpoint inhibitor alone as shown in TABLE 23. The TME phenotypes are correlated to ORR, and 6 month PFS, and overall survival (OS) and predict which patients benefit and which do not. One hundred and twenty (120) patients are enrolled, 60 in each of the treatment groups with equal representation across the 4 stromal phenotypes (15 in each phenotype per treatment group). The correlation between each TME phenotype is tested against clinical outcome data. In immune active (IA) and immune suppressed (IS) patients the use of the regimen of chemotherapy/immune checkpoint inhibitor/Bavituximab confers benefit in comparison to patients classified to angiogenic (A) and immune desert (ID) TME phenotype classes as shown in the TABLE 24.

TABLE 23
Overall response rate (ORR), 6 month progression free survival
rate, overall survival (OS) for the treatment groups.

| | | Chemo/Immune |
|---|---|---|
| | Chemo/Immune | Checkpoint |
| | Checkpoint Inhibitor | Inhibitor/Bavituximab |
| | (n = 60) | (n = 60) |
| ORR (%) | 48 | 60 |
| 6 month PFS (%) | 50 | 65 |
| OS (months) | 12 | 15 |

TABLE 24
Overall response rate (ORR), 6-month progression free
survival rate, overall survival (OS) for the four
TME phenotype classes in the group receiving chemotherapy/immune
checkpoint inhibitor/Bavituximab.

| | IA | IS | ID | A |
|---|---|---|---|---|
| | (n = 15) | (n = 15) | (n = 15) | (n = 15) |
| ORR (%) | 73.3 | 66.7 | 53.3 | 46.7 |
| 6 Month PFS (%) | 80.0 | 73.3 | 60.0 | 46.7 |
| OS (months) | 20.2 | 19.8 | 11.8 | 8.2 |

### Example 13

Locally Advanced/Metastatic Gastric Cancer Tumor Microenvironment RNA Signature Correlates to Clinical Response in First Line Setting with Chemotherapy, Immune Checkpoint Inhibitors and Navicixizumab

A clinical trial is run to determine whether locally advanced or metastatic gastric cancer tumor microenvironment phenotypes correlate to clinical responses when patients are treated with chemotherapy, an immune checkpoint inhibitor, and Navicixizumab in the first-line setting. The analysis includes samples from 120 patients across 2 treatment arms (60 patients in each arm). Patients are randomized to receive either (i) chemotherapy and an immune checkpoint inhibitor or (ii) chemotherapy, an immune checkpoint inhibitor and Navicixizumab. RNA gene signatures are analyzed from tumor samples acquired before any treatments and TME phenotypes are determined. Patients are followed for clinical response, progression-free survival, and overall survival. Data indicate that the immune active (IA), immune suppressed (IS) and Angiogenic (A) TME phenotypes are enriched for response and clinical benefit to the regimen consisting of chemotherapy, immune checkpoint inhibition, and Navicixizumab treatment in this patient population.

Gastric cancer patients are randomized to receiving a regimen of chemotherapy (e.g., capecitabine, 5-FU, cisplatin etc.) plus an immune checkpoint inhibitor (anti-PD-(L)1—e.g., an inhibitor to PD-1 or PD-L1) therapy, or a regimen of chemotherapy/immune checkpoint inhibitor/Navicixizumab. The combination of chemotherapy/immune checkpoint inhibitor/Navicixizumab increases overall response rate (ORR), 6-month progression-free survival (PFS) and overall survival (OS) patients with advanced colorectal cancer compared to the regimen of chemotherapy/immune checkpoint inhibitor alone as shown in TABLE 25. The TME phenotypes are correlated to ORR, and 6-month PFS, and overall survival (OS) and predict which patients benefit and which do not. One hundred and twenty (120) patients are enrolled, 60 in each of the treatment groups with equal representation across the 4 stromal phenotypes (15 in each phenotype per treatment group). The correlation between each TME phenotype is tested against clinical outcome data. In immune active (IA), immune suppressed (IS) and angiogenic (A) patients the use of the regimen of chemotherapy/immune checkpoint inhibitor/Navicixizumab confers benefit in comparison to patients classified to the immune desert (ID) TME phenotype class as shown in the TABLE 26.

TABLE 25
Overall response rate (ORR), 6-month progression free survival
rate, overall survival (OS) for the treatment groups.

| | | Chemo/Immune |
|---|---|---|
| | Chemo/Immune | Checkpoint |
| | Checkpoint Inhibitor | Inhibitor/Navicixizumab |
| | (n = 60) | (n = 60) |
| ORR (%) | 48 | 63 |
| 6 month PFS (%) | 50 | 67 |
| OS (months) | 12 | 16 |

TABLE 26
Overall response rate (ORR), 6-month progression free
survival rate, overall survival (OS) for the four
TME phenotype classes in the group receiving chemotherapy/immune
checkpoint inhibitor/Navicixizumab.

| | IA | IS | ID | A |
|---|---|---|---|---|
| | (n = 15) | (n = 15) | (n = 15) | (n = 15) |
| ORR (%) | 66.7 | 73.3 | 46.7 | 60.0 |
| 6 Month PFS (%) | 73.3 | 73.3 | 53.3 | 66.7 |
| OS (months) | 17.4 | 19.7 | 11.5 | 15.4 |

### Example 14

Locally Advanced/Metastatic HER2-Negative Breast Cancer Tumor Microenvironment RNA Signature Correlates to Clinical Response in Second Line Setting with Combination of Navicixizumab/Chemotherapy or Navicixizumab/PARP Inhibitor

A clinical trial is run to determine whether locally advanced/metastatic HER2-negative breast cancer tumor microenvironment phenotypes correlate to clinical responses when patients are treated with a combination of Navicixizumab/chemotherapy or Navicixizumab/PARP inhibitor in the second-line setting. The analysis includes samples from 120 patients across 2 treatment arms (60 patients in each arm). Patients receive either Navicixizumab/chemotherapy (BRCA WT and hormone receptor-positive) or Navicixizumab/PARP inhibitor (BRCA mutant or triple-negative for ER/PR/HER2). RNA gene signatures are analyzed from tumor samples acquired before any treatments and TME phenotypes are determined. Patients are followed for clinical response and progression-free survival. Data indicate that the biomarker positive group, comprising the immune suppressed (IS) and Angiogenic (A) TME phenotypes, are enriched for response and clinical benefit to the Navicixizumab regimens.

BRCA WT/hormone receptor-positive/HER2-negative breast cancer patients receive a regimen of Navicixizumab plus chemotherapy (e.g., capecitabine, etc.). BRCA mutant or triple-negative for ER/PR/HER2 breast cancer patients receive a regimen of Navicixizumab plus a PARP inhibitor (e.g., Rucaparib, Olaparib, etc.). The TME phenotypes are correlated to ORR and PFS and predict which patients benefit and which do not. One hundred and twenty (120) patients are enrolled, 60 in each of the treatment groups with equal representation across the 4 stromal phenotypes (15 in each phenotype per treatment group). The correlation between each TME phenotype is tested against clinical outcome data. In biomarker positive tumors, represented by angiogenic (A) and immune suppressed (IS) phenotypes, the use of Navicixizumab combinations confers benefit in comparison to patients classified as biomarker negative, represented by immune active (IA) and immune desert (ID) TME phenotype classes as shown in the TABLE 27 and TABLE 28.

TABLE 27
Overall response rate (ORR) and progression free survival (PFS)
for the four TME phenotype classes and by biomarker status
in the group receiving Navicixizumab plus chemotherapy.

| | Biomarker | Biomarker | IA | IS | ID | A |
|---|---|---|---|---|---|---|
| | Positive | Negative | (n = | (n = | (n = | (n = |
| | (n = 30) | (n = 30) | 15) | 15) | 15) | 15) |
| ORR (%) | 63.4 | 20 | 26.7 | 66.7 | 13.3 | 60 |
| PFS (months) | 12.3 | 5.7 | 6.0 | 13.1 | 5.4 | 11.5 |

TABLE 28
Overall response rate (ORR) and progression free survival (PFS)
for the four TME phenotype classes and by biomarker status
in the group receiving Navicixizumab plus PARP inhibitor.

| | Biomarker | Biomarker | IA | IS | ID | A |
|---|---|---|---|---|---|---|
| | Positive | Negative | (n = | (n = | (n = | (n = |
| | (n = 30) | (n = 30) | 15) | 15) | 15) | 15) |
| ORR (%) | 80 | 60 | 60 | 80 | 60 | 80 |
| PFS (months) | 13.7 | 6.3 | 7.1 | 14.2 | 5.5 | 13.2 |

### Example 15

Castration-Resistant Metastatic Prostate Cancer Tumor Microenvironment RNA Signature Correlates to Clinical Response in Third Line Setting with Combination of Navicixizumab/Chemotherapy or Navicixizumab/PARP Inhibitor

A clinical trial is run to determine whether castration-resistant metastatic prostate cancer tumor microenvironment phenotypes correlate to clinical responses when patients are treated with a combination of Navicixizumab/chemotherapy or Navicixizumab/PARP inhibitor in the third-line setting. The analysis includes samples from 80 patients across 2 treatment arms (40 patients in each arm). Patients receive either Navicixizumab/chemotherapy (BRCA WT) or Navicixizumab/PARP inhibitor (BRCA mutant). RNA gene signatures are analyzed from tumor samples acquired before any treatments and TME phenotypes are determined. Patients are followed for clinical response, progression-free survival and overall survival. Data indicate that the biomarker positive group, comprising the immune suppressed (IS) and Angiogenic (A) TME phenotypes, is enriched for response and clinical benefit to the Navicixizumab regimens.

BRCA WT prostate cancer patients receive a regimen of Navicixizumab plus chemotherapy (e.g., docetaxel, cabazitaxel etc.). BRCA mutant prostate cancer patients receive a regimen of Navicixizumab plus a PARP inhibitor (e.g., Rucaparib, Olaparib, etc.). The TME phenotypes are correlated to ORR, PFS and OS and predict which patients benefit and which do not. Eighty (80) patients are enrolled, 40 in each of the treatment groups with equal representation across the 4 stromal phenotypes (10 in each phenotype per treatment group). The correlation between each TME phenotype is tested against clinical outcome data. In tumors that are biomarker positive, represented by angiogenic (A) and immune suppressed (IS) phenotypes, the use of Navicixizumab combinations confers benefit in comparison to patients classified as biomarker negative, represented by immune active (IA) and immune desert (ID) TME phenotype classes as shown in the TABLE 29 and TABLE 30.

TABLE 29
Overall response rate (ORR), progression free survival
(PFS) and overall survival (OS) for the four TME
phenotype classes and by biomarker status in the
group receiving Navicixizumab plus chemotherapy.

| | Biomarker | Biomarker | IA | IS | ID | A |
|---|---|---|---|---|---|---|
| | Positive | Negative | (n = | (n = | (n = | (n = |
| | (n = 20) | (n = 20) | 10) | 10) | 10) | 10) |
| ORR (%) | 56 | 34 | 37 | 58 | 31 | 54 |
| PFS (months) | 14.2 | 9.8 | 10.9 | 14.7 | 8.7 | 13.5 |
| OS (months) | 20.1 | 11.9 | 12.0 | 21.2 | 11.8 | 19.0 |

TABLE 30
Overall response rate (ORR), progression free survival
(PFS) and overall survival (OS) for the four TME
phenotype classes and by biomarker status in the
group receiving Navicixizumab plus PARP inhibitor.

| | Biomarker | Biomarker | IA | IS | ID | A |
|---|---|---|---|---|---|---|
| | Positive | Negative | (n = | (n = | (n = | (n = |
| | (n = 20) | (n = 20) | 10) | 10) | 10) | 10) |
| ORR (%) | 55 | 30 | 30 | 60 | 30 | 50 |
| PFS (months) | 13.8 | 9.6 | 10.2 | 14.2 | 9.0 | 13.4 |
| OS (months) | 23.3 | 17.3 | 18.6 | 26.3 | 16.0 | 20.3 |

### Example 16

Advanced Metastatic Hepatocellular Carcinoma Tumor Microenvironment RNA Signature Correlates to Clinical Response in First Line Setting with Bavituximab and Immune Checkpoint Inhibitor

A single arm phase 2 clinical trial is run to determine whether advanced metastatic hepatocellular carcinoma tumor microenvironment phenotypes correlate to clinical responses when patients are treated with Bavituximab and an immune checkpoint inhibitor in the first-line setting. The analysis includes samples from 28 patients. RNA gene signatures are analyzed from tumor samples acquired before any treatments and TME phenotypes are determined. Patients are followed for clinical response, progression-free survival, and overall survival. Data indicate that the biomarker positive tumors, comprised of the immune active (IA) and immune suppressed (IS) TME phenotypes, are enriched for response and clinical benefit to the regimen consisting of Bavituximab and immune checkpoint inhibitor in this patient population.

Hepatocellular carcinoma patients receive a regimen of Bavituximab plus an immune checkpoint inhibitor (anti-PD-(L)1—i.e., an inhibitor to PD-1 or PD-L1) therapy. The combination of Bavituximab/immune checkpoint inhibitor increases overall response rate (ORR-˜32%), 6 month progression-free survival (6-month PFS-˜57.2%) and overall survival (OS-˜20 months) patients with advanced hepatocellular cancer compared to historical data with immune checkpoint inhibitor alone (15% ORR, ˜40% 6 month PFS, and 17 month OS). The TME phenotypes are correlated to ORR, and 6-month PFS, and overall survival (OS) and predict which patients benefit and which do not. Twenty-eight (28) patients are enrolled, with equal representation across the 4 stromal phenotypes (7 in each phenotype). The correlation between each TME phenotype is tested against clinical outcome data. In patients that are biomarker positive, represented by immune active (IA) and immune suppressed (IS) phenotypes, the use of the regimen of Bavituximab/immune checkpoint inhibitor confers benefit in comparison to patients classified as biomarker negative, comprised of angiogenic (A) and immune desert (ID) TME phenotype classes, as shown in the TABLE 31.

TABLE 31
Overall response rate (ORR), 6-month progression free survival
(PFS) rate, and overall survival (OS) for the four TME phenotype
classes and by biomarker status in the group receiving Bavituximab
plus immune checkpoint inhibitor.

| | Biomarker | Biomarker | IA | IS | ID | A |
|---|---|---|---|---|---|---|
| | Positive | Negative | (n = | (n = | (n = | (n = |
| | (n = 14) | (n = 14) | 7) | 7) | 7) | 7) |
| ORR (%) | 50 | 14 | 43 | 57 | 14 | 14 |
| 6-month PFS (%) | 78.6 | 35.7 | 71.4 | 85.7 | 28.6 | 42.8 |
| OS (months) | 23.8 | 16.2 | 22.4 | 25.2 | 15.7 | 16.7 |

### Example 17

Recurrent/Metastatic Squamous Cell Carcinoma of Head and Neck Tumor Microenvironment RNA Signature Correlates to Clinical Response with Bavituximab and Immune Checkpoint Inhibitor Following Progression on an Immune Checkpoint Inhibitor

A single arm phase 2 clinical trial is run to determine whether recurrent/metastatic squamous carcinoma of head and neck (HNSCC) tumor microenvironment phenotypes correlate to clinical responses when patients are treated with Bavituximab and an immune checkpoint inhibitor following progression on an immune checkpoint inhibitor (e.g., nivolumab, pembrolizumab, durvalumab, atezolizumab, etc.). The analysis includes samples from 28 patients. RNA gene signatures are analyzed from tumor samples acquired before any treatments and TME phenotypes are determined. Patients are followed for clinical response, progression-free survival, and overall survival. Data indicate that the tumors that are biomarker positive, comprised of the immune active (IA) and immune suppressed (IS) TME phenotypes, are enriched for response and clinical benefit to the regimen consisting of Bavituximab and immune checkpoint inhibitor in this patient population.

HNSCC patients receive a regimen of Bavituximab plus an immune checkpoint inhibitor (anti-PD-(L)1—i.e., an inhibitor to PD-1 or PD-L1) therapy. The TME phenotypes are correlated to ORR, and PFS, and overall survival (OS) and predict which patients benefit and which do not. Twenty-eight (28) patients are enrolled, with equal representation across the 4 stromal phenotypes (7 in each phenotype). The correlation between each TME phenotype is tested against clinical outcome data. In biomarker positive patients, represented by immune active (IA) and immune suppressed (IS) phenotypes, the use of the regimen of Bavituximab/immune checkpoint inhibitor confers benefit in comparison to patients classified as biomarker negative, comprised of angiogenic (A) and immune desert (ID) TME phenotype classes, as shown in the TABLE 32.

TABLE 32
Overall response rate (ORR), progression free survival
(PFS) rate, and overall survival (OS) for the four TME
phenotype classes and by biomarker status in the group
receiving Bavituximab plus immune checkpoint inhibitor.

| | Biomarker | Biomarker | IA | IS | ID | A |
|---|---|---|---|---|---|---|
| | Positive | Negative | (n = | (n = | (n = | (n = |
| | (n = 14) | (n = 14) | 7) | 7) | 7) | 7) |
| ORR (%) | 36.4 | 14.3 | 28.6 | 42.9 | 14.3 | 14.3 |
| PFS (months) | 5.5 | 2.5 | 5.3 | 5.7 | 2.4 | 2.6 |
| OS (months) | 11.2 | 7.8 | 11.0 | 11.4 | 7.0 | 8.6 |

### Example 18

Melanoma Tumor Microenvironment RNA Signature Correlates to Clinical Response with Adjuvant Bavituximab and Radiation Following Neoadjuvant Treatment with an Immune Checkpoint Inhibitor

A phase 2 clinical trial is run to determine whether melanoma tumor microenvironment phenotypes correlate to clinical responses when patients are treated with adjuvant Bavituximab and radiation following neoadjuvant immune checkpoint inhibitor treatment and surgical resection of stage 2b lymph nodes. The analysis includes samples from 20 patients. RNA gene signatures are analyzed from tumor samples acquired as surgical resections and TME phenotypes are determined. Patients are followed for ORR. Data indicate that the tumors that are biomarker positive, comprised of the immune active (IA) and immune suppressed (IS) TME phenotypes, are enriched for response and clinical benefit to the regimen consisting of Bavituximab and radiation in this patient population.

Melanoma patients receive a course of neoadjuvant immune checkpoint inhibitor (anti-PD-(L)1—i.e., an inhibitor to PD-1 or PD-L1) therapy and their stage 2b lymph nodes are resected. Radiation and Bavituximab are given as adjuvant therapy. The TME phenotypes from resected tumor samples are correlated to ORR. Twenty-eight (20) patients are enrolled, with equal representation across the 4 stromal phenotypes (5 in each phenotype). The correlation between each TME phenotype is tested against clinical outcome data. In biomarker positive patients, represented by immune active (IA) and immune suppressed (IS) phenotypes, the use of the regimen of Bavituximab/radiation confers benefit in comparison to patients classified as biomarker negative, comprised of angiogenic (A) and immune desert (ID) TME phenotype classes, as shown in the TABLE 33.

TABLE 33
Overall response rate (ORR), progression free survival (PFS)
rate for the four TME phenotype classes and by biomarker
status in the group receiving Bavituximab plus radiation.

| | Biomarker | Biomarker | IA | IS | ID | A |
|---|---|---|---|---|---|---|
| | Positive | Negative | (n = | (n = | (n = | (n = |
| | (n = 10) | (n = 10) | 5) | 5) | 5) | 5) |
| ORR (%) | 90 | 60 | 80 | 100 | 60 | 60 |

### Example 19

The Tumor Microenvironment RNA Signature for Advanced Colorectal Cancers Metastatic to Liver Correlates with Clinical Benefit from a Triplet Combination of Navicixizumab, an Innate Immune Activating Agent and an Immune Checkpoint Inhibitor

A phase 2 clinical trial is run to determine clinical benefit in patients with advanced colorectal cancers metastatic to liver (mCRC) treated with a triplet combination of navicixizumab, an innate immune activating agent, and a PD-(L)1 agent. In this setting, regorafenib or trifluoridine/tipiracil therapy typically yield a median PFS (mPFS) of ˜2 months and median OS (mOS) of ˜7 months. Data indicate that as many as 40% of third line or later (“3L+”) mCRC patients with liver metastases, who have failed prior becavizumab (AVASTIN®) and/or EGFR-targeted therapies and are MSS+(i.e., microsatellite stable), have the ID phenotype and may not benefit from either standard chemotherapeutic or immunologic approaches. The analysis includes enrollment of 60 patients with prospectively defined, retrospective analysis of liver metastatic lesions by the classifying the TME phenotypes as A, IA, IS or ID.

MSS+ advanced colorectal cancer patients (3L+) with liver metastases are treated with the triplet combination of navicixizumab, an anti-PD(L)1 therapy and an innate immune stimulating agent, such as the Dectin agonist Imprime PGG, the STING agonist BMS-986301 and the NLR agonist BMS-986299. ORR, mPFS, 3-month PFS rate, mOS and OS rate at 9 months are assessed across all TME phenotypes. TABLE 34 reports the result of the prospective analysis. ID-class patients received a greater clinical benefit than the standard of care for 3L+ mCRC with regorafenib or trifluoridine/tipiracil.

TABLE 34
Prospective Analysis of mCRC in 3L+ patients.

| | IA | IS | ID | A |
|---|---|---|---|---|
| | (n = 12) | (n = 12) | (n = 24) | (n = 12) |
| ORR (%) | 35% | 30% | 20% | 25% |
| mPFS | 6 | 5 | 4 | 6 |
| PFS rate (3 month) | 30% | 30% | 30% | 30% |
| mOS | 12 mos | 11 mos | 10 mos | 12 mos |
| OS rate (9 month) | 50% | 45% | 40% | 455 |

### Example 20

Platinum-Resistant or Platinum-Sensitive Recurrent Ovarian Cancer Tumor Microenvironment RNA Signature Correlates to Clinical Response with PARP Inhibitor, and Navicixizumab

A clinical trial is run to determine whether ovarian cancer tumor microenvironment phenotypes correlate to clinical responses when patients are treated with a PARP inhibitor, and navicixizumab in PARP naïve patients or patients that previously progressed on PARP inhibitor. The analysis includes samples from two different cohorts (one consisting of PARP inhibitor naïve patients and one consisting of PARP resistant patients). 40 patients from each cohort are randomized to 2 different treatment arms (20 patients in each arm). Patients are randomized to receive either a PARP inhibitor in combination with navicixizumab or PARP inhibitor monotherapy (PARP naïve cohort) or PARP inhibitor in combination with navicixizumab or navicixizumab monotherapy (PARP resistant). RNA gene signatures are analyzed from tumor samples acquired before the treatments and TME phenotypes are determined. Patients are followed for clinical response. Data indicate that the suppressed (IS), and angiogenic (A) TME phenotypes are enriched for response to the regimen consisting of Navicixizumab monotherapy or in combination with PARP inhibitor treatment in PARP naive patients and PARP resistant patients.

Ovarian cancer patients are randomized to receiving a regimen of PARP inhibitor (Olaparib, Rucaparib, Niraparib, etc.) plus navicixizumab, PARP monotherapy (PARP naive) or Navicixizumab monotherapy. The combination of PARP inhibitor/Navicixizumab increases overall response rate in patients with recurrent ovarian cancer compared to navicixizumab alone in both the PARP naive and PARP resistant cohorts as shown in TABLE 35. The TME phenotypes are correlated to ORR and predict which patients benefit and which do not. Eighty (80) total patients are enrolled, 20 in each of the treatment groups across the two cohorts with equal representation across the 4 stromal phenotypes (5 in each phenotype per treatment group). The correlation between each TME phenotype is tested against clinical outcome data. In immune suppressed (IS), and angiogenic (A) patients the use of the regimen of PARPi/Navicixizumab confers benefit in comparison to patients classified to immune active (IA) and immune desert (ID) TME phenotype classes as shown in the TABLE 36.

TABLE 35
Overall response rate (ORR) for the cohorts and treatment groups.

| | PARP naiive | | PARP resistant | |
|---|---|---|---|---|
| | PARPi/ | PARP | PARPi/Navicixizumab | |
| | Navicixizumab(n = 20) | i(n = 20) | (n = 20) | Navixicizumab(n = 20) |
| ORR (%) | 65 | 40 | 40% | 25% |

TABLE 36
Overall response rate (ORR) for the four TME phenotype
classes in the groups receiving PARPi/Navicixumab.

| | IA | IS | ID | A |
|---|---|---|---|---|
| | (n = 5) | (n = 5) | (n = 5) | (n = 5) |
| PARP naive cohort | 40 | 80 | 40 | 100 |
| PARP resistant cohort | 20 | 60 | 20 | 60 |

### Example 21

Platinum-Resistant or Platinum-Sensitive Recurrent Triple Negative Breast Cancer Microenvironment RNA Signature Correlates to Clinical Response with PARP Inhibitor, Immune Checkpoint Inhibitors and Navicixizumab

A clinical trial is run to determine whether the triple negative breast cancer (TNBC) tumor microenvironment phenotypes correlate to clinical responses when patients are treated with a PARP inhibitor, an immune checkpoint inhibitor, and Navicixizumab after recurrence on platinum-based chemotherapy. The analysis includes samples from two different cohorts (one consisting of platinum-sensitive patients and one consisting of platinum-resistant patients). 40 patients from each cohort are randomized to 2 different treatment arms (20 patients in each arm). Patients are randomized to receive either (i) a PARP inhibitor and an immune checkpoint inhibitor or (ii) a PARP inhibitor, an immune checkpoint inhibitor and Navicixizumab. RNA gene signatures are analyzed from tumor samples acquired before the treatments and TME phenotypes are determined. Patients are followed for clinical response. Data indicate that the immune active (IA), immune suppressed (IS), and angiogenic (A) TME phenotypes are enriched for response to the regimen consisting of PARP inhibitor, immune checkpoint inhibition, and Navicixizumab treatment in both platinum-sensitive and platinum-resistant patient cohorts.

TNBC patients are randomized to receiving a regimen of PARP inhibitor (olaparib, rucaparib, niraparib, etc.) plus an immune checkpoint inhibitor (anti-PD-(L)1—i.e., an inhibitor to PD-1 or PD-L1) therapy, or a regimen of PARP inhibitor/immune checkpoint inhibitor/Navicixizumab. The combination of PARP inhibitor/immune checkpoint inhibitor/Navicixizumab increases overall response rate in patients with TNBC compared to the regimen of PARP inhibitor/immune checkpoint inhibitor alone in both the platinum-sensitive and platinum-resistant cohorts as shown in TABLE 37. The TME phenotypes are correlated to ORR and predict which patients benefit and which do not. Eighty (80) total patients are enrolled, 20 in each of the treatment groups across the two cohorts with equal representation across the 4 stromal phenotypes (5 in each phenotype per treatment group). The correlation between each TME phenotype is tested against clinical outcome data. In immune active (IA), immune suppressed (IS), and angiogenic (A) patients the use of the regimen of PARPi/immune checkpoint inhibitor/Navicixizumab confers benefit in comparison to patients classified to immune desert (ID) TME phenotype classes as shown in the TABLE 38.

TABLE 37
Overall response rate (ORR) for the cohorts and treatment groups.

| | Platinum-sensitive cohort | | Platinum-resistant cohort | |
|---|---|---|---|---|
| | PARPi/Immune | PARPi/Immune | PARPi/Immune | PARPi/Immune |
| | Checkpoint | Checkpoint | Checkpoint | Checkpoint |
| | Inhibitor | Inhibitor/Navicixizumab | Inhibitor | Inhibitor/ |
| | (n = 20) | (n = 20) | (n = 20) | Navixicizumab(n = 20) |
| ORR (%) | 65 | 80 | 20 | 45 |

TABLE 38
Overall response rate (ORR) for the four TME
phenotype classes in the groups receiving PARPi/immune
checkpoint inhibitor/Navicixumab.

| | IA | IS | ID | A |
|---|---|---|---|---|
| | (n = 5) | (n = 5) | (n = 5) | (n = 5) |
| Platinum-sensitive cohort | 80 | 80 | 60 | 100 |
| Platinum-resistant cohort | 60 | 80 | 0 | 40 |

### Example 22

### Vidutolimod and CPI Combination Therapy in Melanoma

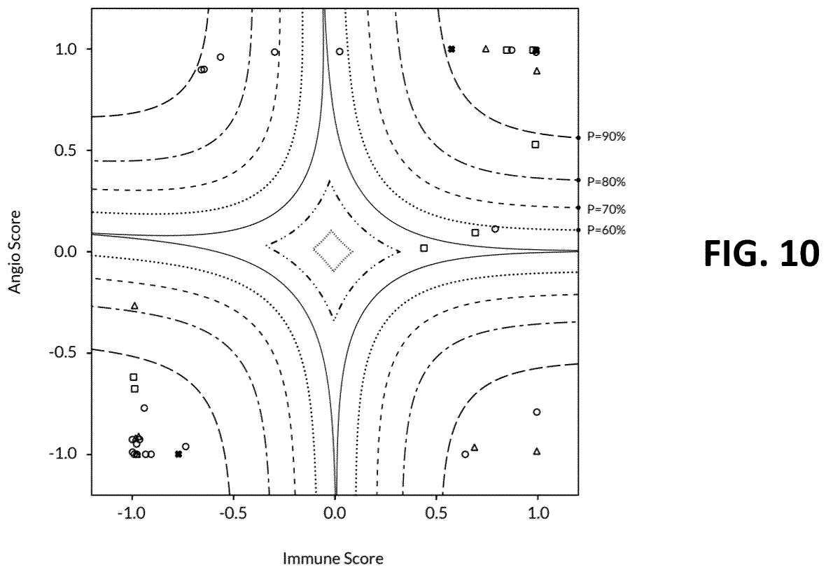

A phase 1 clinical trial in melanoma was conducted. Patients were refractory and had progressed on at least one line of CPI-targeted therapy. A cohort received a combination of vidutolimod, a TLR-9 agonist (CMP-001, Checkmate Pharmaceuticals, was Cyt003 from Cytos Ag) and pembrolizumab, and samples were taken pre-treatment via a core needle biopsy and stored as an FFPE slide prior to processing for RNA extraction. Retrospectively, TME Panel-1 calls were determined via the ANN method (TABLE 39, TABLE 40, and FIG. 10).

A therapeutic hypothesis is that many patients that have been heavily pretreated with CPIs and who were refractory have TMEs that are immunosuppressed (with the TME call IS) and would benefit from a restoration of the immune response. The immune modulator vidutolimod can initiate an innate immune response and alter the immune components of the tumor microenvironment of an IS patient to be able to respond. Thus, the immune modulator vidutolimod and CPI combination therapy in the IS group will have the most response (TABLE 40). Example additional immune modulators in this class are ProMune CpG 7909 (PF3512676), SD-101, 1018 ISS, IMO-2123, Litenimod, MIS416, Cobitolimod, ImprimePGG (odetiglucan), imiquimod, fingolimod, tilsotolimod, and BL-7040.

TABLE 39
ANN Model Performance.

| | | AUC | | Sensi- | Speci- | | |
|---|---|---|---|---|---|---|---|
| | ACC | ROC | F1 | tivity | ficity | PPV | NPV |
| TME IS | 0.76 | 0.75 | 0.61 | 0.70 | 0.79 | 0.54 | 0.88 |
| | (29/38) | | | (7/10) | (22/28) | (7/13) | (22/25) |
| Random | 0.62 ± | 0.5 | 0.27 ± | 0.27 ± | 0.74 ± | 0.27 ± | 0.74 ± |
| | 0.066 | | 0.125 | 0.125 | 0.044 | 0.125 | 0.044 |

TABLE 40
Best Objective Response versus TME Biomarker Status
for 38 Patients Retrospectively Determined.

| TME\BOR | PD | SD | PR | CR |
|---|---|---|---|---|
| A | 4 | 0 | 0 | 0 |
| IS | 4 | 2 | 5 | 2 |
| IA | 2 | 2 | 0 | 0 |
| ID | 10 | 4 | 2 | 1 |

### Example 23

DLL4 and VEGF Antagonists with FOLFOX, FOLFIRI, or Irinotecan in Metastatic CRC

A clinical trial in metastatic CRC is conducted with an anti-DLL4/anti-VEGF antagonist, such as navicixizumab, ABT-165, or CTX-009 in combination with investigator's choice of irinotecan, FOLFOX, or FOLFIRI, or another chemotherapeutic agent which is a standard of care. Based on the patients' TME Panel-1 biomarker status, A or IS patients, or patients defined as being biomarker positive for having a TME score that is A or IS, or above the angiogenic axis in a latent plot, receive the most clinical benefit from an anti-DLL4/anti-VEGF antagonist in combination with a chemotherapeutic agent, whereas most IA or ID patients are predicted to progress, and thus receive treatments appropriate to those TME groups (TABLE 41).

TABLE 41
Best Objective Response versus TME Biomaker
Status for DLL4 and VEGF Antagonists with
Chemotherapeutic Agents in Metastatic CRC.

| TME\BOR | PD | SD | PR | CR |
|---|---|---|---|---|
| A | 0 | 4 | 2 | 2 |
| IS | 0 | 4 | 2 | 2 |
| IA | 4 | 2 | 1 | 1 |
| ID | 6 | 2 | 1 | 1 |

### Example 24

### Bavituximab, CPI, and Radiation in Gliomas and Glioblastoma

A clinical trial is run to treat metastatic gliomas and glioblastoma with a combination of radiation, the anti-phosphatidylserine (anti-PS) targeting antibody bavituximab, and a checkpoint inhibitor (CPI), such as a PD-(L)1, e.g. pembrolizumab or nivolumab. Tumor tissue samples from surgical resections of glioblastoma are RNA sequenced and TME phenotypes are determined. Patients are followed for ORR. Data indicate that the tumors that are biomarker positive, comprised of the immune active (IA) and immune-suppressed (IS) TME phenotypes, are enriched for response and clinical benefit to the regimen consisting of bavituximab, checkpoint inhibitor, and radiation in this patient population (TABLE 42).

TABLE 42
Best Objective Response versus TME Biomaker
Status in Gliomas and Glioblastoma.

| TME\BOR | PD | SD | PR | CR |
|---|---|---|---|---|
| A | 4 | 1 | 1 | 0 |
| IS | 4 | 4 | 4 | 0 |
| IA | 4 | 2 | 3 | 0 |
| ID | 6 | 1 | 1 | 0 |

### Example 25

### Anti-Angiogenics and Checkpoint Inhibitor Combination Therapy/Basket Trials

A basket trial is initiated in solid tumors in second, third, or fourth line, or more settings. Tumor tissue samples from surgical resections of solid tumors are RNA sequenced and TME phenotypes are determined. Patients are followed for ORR. Data indicate that the tumors that are biomarker positive, comprised of the immune active (IA), immune suppressed (IS) and angiogenic (A) phenotypes are enriched for response and clinical benefit for the patients treated with anti-DLL4/anti-VEGF antagonist, such as navicixizumab, ABT-165, or CTX-009, or an anti-VEGF antagonist, such as bevacizumab, ramucirumab, or varisacumab, in combination with an anti-PD-1 or an anti-PD-L1 checkpoint inhibitor (CPI), or a bispecific immunoglobulin or modified immunoglobulin of an anti-VEGF antagonist and a CPI.

### Example 26

Tumor Vaccines and/or Chemotherapeutic Standard of Care in Immune Desert Patients

A basket clinical trial for patients who have progressed on one or two lines of prior therapies in colorectal, breast, triple-negative breast, prostate, liver, melanoma, head and neck cancer, or gastric cancer (primary tumors and metastatic) are selected based on their TME status after progression. Patients whose TME Panel-1 status is immune desert (ID) are treated with an investigator's choice of standard of care chemotherapeutics and/or tumor vaccines, the latter such as AST-301(pNGVL3-hICD), NeoVax, Proscavax, a personalized vaccine, α-lactalbumin vaccine, P10s-PADRE, OncoVax, PVX-410, Galinpepimut-S, GRT-C903/GRT-C904, KRAS peptide vaccine, pING-hHER3FL, GVAX, INCAGN01876, or a non-genetically-manipulated, living immune cell immunotherapy, a non-limiting example is AlloStim. A biopsy is taken 2 months after treatment and the patient's TME Panel-1 status is reassessed. Patients with transition from ID to IA are treated with an immunotherapy and responds. Patients that remain in the ID group are spared from more-futile therapies to which they are unlikely to respond, such as immunotherapy or antiangiogenic therapy.

### Example 27

### Stratification Strategies in a Basket Trial or Complex Trial Under a Master Protocol

In a basket trial or complex trial for solid tumors, under a master protocol, patients are assigned to a treatment arm (i.e., a substudy), using either an approach such as prespecified randomization ratio, or by prioritizing biomarkers. An example of the prespecified randomization ratio would be to use a reverse prevalence ratio in which patients who have low-prevalence biomarkers have a greater likelihood of being assigned to a substudy for the lower prevalence population. An example of biomarker-prioritizing approach is for the investigators to rank biomarker groups based on their predictive value and assign patients to the treatment group for which the patients' biomarker profile has the highest predictive value. The TME phenotype or biomarker status (i.e., IA, IS, ID, A, A+IA, A+IS, or biomarker positive) is prioritized over other biomarkers, or used in combination with other biomarkers such as MSS status or PD-L1.

### Example 28

A Phase 2, Multicenter, Open-Label Basket Study of Navicixizumab Monotherapy or in Combination with Paclitaxel or Irinotecan in Patients with Select Advanced Solid Tumors

A retrospective analysis of a signal-finding Phase 2 clinical trial is conducted. Patient samples from thirty patients in a phase 2, multicenter, open-label basket study of navicixizumab monotherapy or in combination with the chemotherapeutics paclitaxel or irinotecan in patients with advanced solid tumors in colorectal, triple-negative breast cancer, gastric, and ovarian cancers. Patients in the combined A and IS groups that received navicixizumab monotherapy have an ORR greater than 40%. Patients in the combined A and IS groups that receive navicixizumab and chemotherapy have an ORR greater than 50%.

### Example 29

NSCLC Tumor Microenvironment Signature Correlates to Clinical Response with Tislelizumab and Chemotherapeutic Agents

A retrospective analysis of 100 RNA signatures from a Phase 3 clinical trial of non-small cell lung cancer (NSCLC) with the anti-PD-1 checkpoint inhibitor tislelizumab in combination with the chemotherapeutic agents pemetrexed and a platinum agent (cisplatin or carboplatin) is run using the ANN method. Prior to the retrospective analysis (without any stratification into stromal phenotypes), the PFS was significantly longer with tislelizumab plus chemotherapy compared with chemotherapy alone (median PFS: 9.7 versus 7.6 mo; hazard ratio=0.645) (Lu et al., 2021, Journal of Thoracic Oncology, V. 16, pp. 1512-1522). After the retrospective ANN analysis, the biomarker positive group (IA or IS) is shown to have a PFS of 15.0 months with the combination therapy of tislelizumab and chemotherapy.

It is to be appreciated that the Detailed Description section, and not the Summary and Abstract sections, is intended to be used to interpret the embodiments. The Summary and Abstract sections can set forth one or more but not all exemplary embodiments of the present invention as contemplated by the inventor(s), and thus, are not intended to limit the present invention and the appended embodiments in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following embodiments and their equivalents.

The contents of all cited references (including literature references, patents, patent applications, and websites) that may be cited throughout this application are hereby expressly incorporated by reference in their entirety for any purpose, as are the references cited therein, in the versions publicly available on Mar. 25, 2021. Protein and nucleic acid sequences identified by database accession number and other information contained in the subject database entries (e.g., non-sequence related content in database entries corresponding to specific Genbank accession numbers) are incorporated by reference, and correspond to the corresponding database release publicly available on Mar. 25, 2021.

1. A method for treating a human subject afflicted with a cancer, the method comprising administering a TME phenotype class-specific therapy to the subject, wherein, prior to the administration, a TME phenotype class is determined by applying an Artificial Neural Network (ANN) classifier to a plurality of RNA expression levels obtained from a gene panel from a cancer tumor sample obtained from the subject, wherein the cancer tumor is assigned a TME phenotype class selected from the group consisting of IS (immune suppressed), A (angiogenic), IA (immune active), ID (immune desert), and combinations thereof.

2. A method for treating a human subject afflicted with a cancer, the method comprising:
(i) applying an ANN classifier to a plurality of RNA expression levels obtained from a gene panel from a cancer tumor sample obtained from the subject, wherein the cancer tumor is assigned a TME phenotype class selected from the group consisting of IS, A, IA, ID, and combinations thereof, and
(ii) administering a TME phenotype class-specific therapy to the subject.

3. A method for identifying a human subject afflicted with a cancer suitable for treatment with a TME phenotype class-specific therapy, the method comprising applying an ANN classifier to a plurality of RNA expression levels obtained from a gene panel from a cancer tumor sample obtained from the subject, wherein the cancer tumor is assigned a TME phenotype class selected from the group consisting of IS, A, IA, ID, and combinations thereof, and wherein the assigned TME phenotype class indicates that a TME phenotype class-specific therapy can be administered to treat the cancer.

4. The method of claim 1, wherein the ANN classifier comprises;
(a) an input layer comprising between 2 and 100 nodes, wherein each node in the input layer corresponds to a gene in a gene panel selected from the genes presented in TABLE 1 and TABLE 2, wherein the gene panel comprises (i) between 1 and 63 genes selected from TABLE 1, and between 1 and 61 genes selected from TABLE 2, (ii) a gene panel comprising genes selected from TABLE 3 and TABLE 4, (iii) a gene panel of TABLE 5, or (iv) any of the gene panels (Genesets) disclosed in FIG. 9A-G;
(b) a hidden layer comprising 2 nodes; and
(c) an output layer comprising 4 output nodes, wherein each one of the 4 output nodes in the output layer corresponds to a TME phenotype class, wherein the 4 TME phenotype classes are IA, IS, ID, and A.

5. The method of claim 1, wherein the TME phenotype class-specific therapy is an IA TME phenotype class-specific therapy comprising a checkpoint modulator therapy comprising:
(i) an activator of a stimulatory immune checkpoint molecule such as an antibody molecule against GITR, OX-40, ICOS, 4-IBB, or a combination thereof,
(ii) a RORγ agonist;
(iii) an inhibitor of an inhibitory immune checkpoint molecule such as an antibody against PD-1 (such as nivolumab, pembrolizumab, cemiplimab, PDR001, CBT-501, CX-188, sintilimab, tislelizumab, TSR-042 or an antigen-binding portion thereof), an antibody against PD-L1 (such as avelumab, atezolizumab, durvalumab, CX-072, LY3300054, or an antigen-binding portion thereof), an antibody against PD-L2, or an antibody against CTLA-4, alone or a combination thereof, or in combination with an inhibitor of TIM-3, LAG-3, BTLA, TIGIT, VISTA, TGF-β, LAIR1, CD160, 2B4, GITR, OX40, 4-1BB, CD2, CD27, CDS, ICAM-1, LFA-1, ICOS, CD30, CD40, BAFFR, HVEM, CD7, LIGHT, NKG2C, SLAMF7, NKp80, or CD86; or
(iv) a combination thereof.

6. The method of any one of claim 1, wherein the TME phenotype class-specific therapy is an IS-class TME therapy comprising:
(1) a checkpoint modulator therapy and an anti-immunosuppression therapy, and/or
(2) an antiangiogenic therapy;
wherein the checkpoint modulator therapy comprises an inhibitor of an inhibitory immune checkpoint molecule comprising:
(a) an antibody against PD-1 selected from the group consisting of pembrolizumab, nivolumab, cemiplimab, PDR001, CBT-501, CX-188, sintilimab, tislelizumab, TSR-042, an antigen-binding portion thereof, and a combination thereof,
(b) an antibody against PD-L1 selected from the group consisting of avelumab, atezolizumab, CX-072, LY3300054, durvalumab, an antigen-binding portion thereof, and a combination thereof,
(c) an antibody against PD-L2 or an antigen binding portion thereof,
(d) an antibody against CTLA-4 selected from ipilimumab and the bispecific antibody XmAb20717 (anti PD-1/anti-CTLA-4); or
(e) a combination thereof;
wherein the antiangiogenic therapy comprises:
(a) an anti-VEGF antibody selected from the group consisting of varisacumab, bevacizumab, navicixizumab (anti-DLL4/anti-VEGF bispecific), ABL101 (NOV1501) (anti-DLL4/anti-VEGF), ABT165 (anti-DLL4/anti-VEGF), and a combination thereof,
(b) an anti-VEGFR2 antibody, wherein the anti-VEGFR2 antibody comprises ramucirumab; or
(c) a combination thereof; and
wherein the anti-immunosuppression therapy comprises:
(a) an anti-PS antibody, anti-PS targeting antibody, antibody that binds 2-glycoprotein 1, inhibitor of PI3Kγ, adenosine pathway inhibitor, inhibitor of IDO, inhibitor of TIM, inhibitor of LAG3, inhibitor of TGF-β, CD47 inhibitor, or a combination thereof, wherein the anti-PS targeting antibody is bavituximab, or an antibody that binds A32-glycoprotein 1; the PI3Kγ inhibitor is LY3023414 (samotolisib) or IPI-549; the adenosine pathway inhibitor is AB-928; the TGFβ inhibitor is LY2157299 (galunisertib) or the TGFβR1 inhibitor is LY3200882; the CD47 inhibitor is magrolimab (5F9); and the CD47 inhibitor targets SIRPα;
(b) an inhibitor of TIM-3, LAG-3, BTLA, TIGIT, VISTA, TGF-β or its receptors, an inhibitor of LAIR1, CD160, 2B4, GITR, OX40, 4-1BB, CD2, CD27, CDS, ICAM-1, LFA-1, ICOS, CD30, CD40, BAFFR, HVEM, CD7, LIGHT, NKG2C, SLAMF7, NKp80, an agonist of CD86, or a combination thereof; or
(c) a combination thereof.

7. The method of claim 1, wherein the TME phenotype class-specific therapy is an A TME phenotype class-specific therapy comprising:
(i) a VEGF-targeted therapy, an inhibitor of angiopoietin 1 (Ang1), an inhibitor of angiopoietin 2 (Ang2), an inhibitor of DLL4, a bispecific of anti-VEGF and anti-DLL4, a TKI inhibitor, an anti-FGF antibody, an anti-FGFR1 antibody, an anti-FGFR2 antibody, a small molecule that inhibits FGFR1, a small molecule that inhibits FGFR2, an anti-PLGF antibody, a small molecule against a PLGF receptor, an antibody against a PLGF receptor, an anti-VEGFB antibody, an anti-VEGFC antibody, an anti-VEGFD antibody, an antibody to a VEGF/PLGF trap molecule such as aflibercept, or ziv-aflibercet, an anti-DLL4 antibody, an anti-Notch therapy such as an inhibitor of gamma-secretase, or any combination thereof, wherein the TKI inhibitor is selected from the group consisting of cabozantinib, vandetanib, tivozanib, axitinib, lenvatinib, sorafenib, regorafenib, sunitinib, fruquitinib, pazopanib, and any combination thereof, and wherein the VEGF-targeted therapy comprises administering (a) an anti-VEGF antibody comprising varisacumab, bevacizumab, an antigen-binding portion thereof, or a combination thereof, (b) an anti-VEGFR2 antibody comprising ramucirumab or an antigen-binding portion thereof; or (c) a combination thereof;
(ii) an angiopoietin/TIE2-targeted therapy comprising endoglin and/or angiopoietin; or
(iii) a DLL4-targeted therapy comprising navicixizumab, ABL101 (NOV1501), ABT165, or a combination thereof.

8. The method of claim 1, wherein the TME phenotype class-specific therapy is an ID TME phenotype class-specific therapy comprising:
a checkpoint modulator therapy concurrently or after the administration of a therapy that initiates an immune response, wherein the checkpoint modulator therapy comprises an inhibitor of an inhibitory immune checkpoint molecule such as an antibody against PD-1, PD-L1, PD-L2, CTLA-4, or a combination thereof, and wherein the therapy that initiates an immune response is a vaccine, a CAR-T, or a neo-epitope vaccine, wherein:
(i) the anti-PD-1 antibody comprises nivolumab, pembrolizumab, cemiplimab, PDR001, CBT-501, CX-188, sintilimab, tislelizumab, or TSR-042, or an antigen-binding portion thereof;
(ii) the anti-PD-L1 antibody comprises avelumab, atezolizumab, CX-072, LY3300054, durvalumab, or an antigen-binding portion thereof; or
(iii) the anti-CTLA-4 antibody comprises ipilimumab or the bispecific antibody XmAb20717 (anti PD-1/anti-CTLA-4), or an antigen-binding portion thereof.

9. The method of claim 1, the method further comprising (a) administering chemotherapy to the subject; (b) performing surgery on the subject; (c) administering radiation therapy to the subject; or (d) any combination thereof.

10. The method of claim 1, wherein the cancer is relapsed, refractory, metastatic, dMMR, or a combination thereof.

11. The method of claim 1, wherein the cancer is selected from the group consisting of:
(i) gastric cancer, such as locally advanced, metastatic gastric cancer, or previously untreated gastric cancer;
(ii) breast cancer, such as locally advanced, triple negative breast cancer, or metastatic Her2-negative breast cancer;
(iii) prostate cancer, such as castration-resistant metastatic prostate cancer;
(iv) liver cancer, such as advanced metastatic hepatocellular carcinoma;
(v) carcinoma of head and neck, such as recurrent or metastatic squamous cell carcinoma of head and neck;
(vi) melanoma, such as metastatic melanoma;
(vii) colorectal cancer, such as advanced colorectal cancer metastatic to liver;
(viii) ovarian cancer, such as platinum-resistant ovarian cancer or platinum-sensitive recurrent ovarian cancer;
(ix) glioma, such as metastatic glioma;
(x) lung cancer, such non-small cell lung cancer (NSCLC); and
(xi) glioblastoma.

12. The method of claim 1, wherein administering a TME phenotype class-specific therapy results in:
(i) reduction of the cancer burden by at least about 10%, 20%, 30%, 40%, or 50% compared to the cancer burden prior to the administration;
(ii) progression-free survival of at least about 1, 2, 3, 4, 5, 6, 7, 8, 9, 10, 11, or 12 months, or at least about 1, 2, 3, 4 or 5 years after the initial administration of the TME phenotype class-specific therapy;
(iii) stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy;
(iv) partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy;
(v) complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the TME phenotype class-specific therapy;
(vi) improved progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject who has not received a TME phenotype class-specific therapy assigned using an ANN classifier such as TME Panel-1;
(vii) improved overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject who has not received a TME phenotype class-specific therapy assigned using an ANN classifier such as TME Panel-1; or
(viii) a combination thereof.

13. A method of assigning a TME phenotype class to a cancer in a subject in need thereof, the method comprising:
(i) generating an ANN classifier by training an ANN with a training set comprising RNA expression levels for each gene in a gene panel in a plurality of samples obtained from a plurality of subjects, wherein each sample is assigned a TME phenotype classification; and assigning, using the ANN classifier, a TME phenotype class to the cancer in the subject, wherein the input to the ANN classifier comprises RNA expression levels for each gene in the gene panel in a test sample obtained from the subject;
(ii) generating an ANN classifier by training an ANN with a training set comprising RNA expression levels for each gene in a gene panel in a plurality of samples obtained from a plurality of subjects, wherein each sample is assigned a TME phenotype classification; wherein the ANN classifier assigns a TME phenotype class to the cancer in the subject using as input RNA expression levels for each gene in the gene panel in a test sample obtained from the subject; or
(iii) using an ANN classifier to predict the TME phenotype class of the cancer in the subject, wherein the ANN classifier is generated by training an ANN with a training set comprising RNA expression levels for each gene in a gene panel in a plurality of samples obtained from a plurality of subjects, wherein each sample is assigned a TME phenotype class or combination thereof.

14. The method of claim 13, where the method is implemented in a computer system comprising at least one processor and at least one memory, the at least one memory comprising instructions executed by the at least one processor to cause the at least one processor to implement the machine-learning model.

15. The method of claim 14, the method further comprising (i) inputting, into the memory of the computer system, the ANN classifier code; (ii) inputting, into the memory of the computer system, the gene panel input data corresponding to the subject, wherein the input data comprises RNA expression levels; (iii) executing the ANN classifier code; or (v) any combination thereof.

16. A method to treat a subject having a cancer with a specific TME phenotype, the method comprising administering a TME phenotype class-specific therapy to the subject wherein:
(i) the cancer is locally advanced, metastatic gastric cancer and the TME phenotype is IA, A, or IS;
(ii) the cancer is untreated gastric cancer and the TME phenotype is IS or A;
(iii) the cancer is advanced/metastatic HER2-negative breast Cancer and the TME phenotype is A or IS;
(iv) the cancer is castration-resistant metastatic prostate cancer and the TME phenotype is A or IS;
(v) the cancer is advanced metastatic hepatocellular carcinoma and the TME phenotype is IA or IS;
(vi) the cancer is recurrent/metastatic squamous cell carcinoma of head and neck and the TME phenotype is IA or IS;
(vii) the cancer is melanoma and the TME phenotype is IA or IS;
(viii) the cancer is advanced colorectal cancer metastatic to liver and the TME phenotype is ID;
(ix) the cancer is platinum resistant or platinum-sensitive recurrent ovarian cancer and the TME phenotype is IA, IS or A;
(x) the cancer is platinum-resistant or platinum-sensitive recurrent triple negative breast cancer and the TME phenotype is IA, IS or A;
(xi) the cancer is metastatic colorectal cancer and the TME phenotype is A or IS;
(xii) the cancer is glioma or glioblastoma and the TME phenotype is IS or IA; or
(xiii) the cancer is non-small cell lung cancer and the TME phenotype is IS or IA;
wherein the TME phenotype class has been assigned by applying an ANN classifier to a plurality of RNA expression levels obtained from a gene panel from a cancer tumor sample obtained from the subject, wherein the ANN classifier comprises;
(a) an input layer comprising between 2 and 100 nodes, wherein each node in the input layer corresponds to a gene in a gene panel selected from the genes presented in TABLE 1 and TABLE 2, wherein the gene panel comprises (i) between 1 and 63 genes selected from TABLE 1, and between 1 and 61 genes selected from TABLE 2, (ii) a gene panel comprising genes selected from TABLE 3 and TABLE 4, (iii) a gene panel of TABLE 5, or (iv) any of the gene panels (Genesets) disclosed in FIG. 9A-G; a hidden layer comprising 2 nodes; and
(b) an output layer comprising 4 output nodes, wherein each one of the 4 output nodes in the output layer corresponds to a TME phenotype class, wherein the 4 TME phenotype classes are IA, IS, ID, and A.

17. A kit or article of manufacture comprising (i) a plurality of oligonucleotide probes capable of specifically detecting an RNA encoding a gene biomarker from TABLE 1 (or FIG. 9A-9G), and (ii) a plurality of oligonucleotide probes capable of specifically detecting an RNA encoding a gene biomarker from TABLE 2 (or FIG. 9A-9G), wherein the article of manufacture comprises a microarray.

18. The method of claim 4, the method further comprising applying a logistic regression classifier comprising a Softmax function to the output of the ANN, wherein the Softmax function assigns probabilities to each TME phenotype class.

19. The method of claim 16, the method further comprising a logistic regression classifier comprising a Softmax function to the output of the ANN, wherein the Softmax function assigns probabilities to each TME phenotype class.


SEQUENCE LISTING (43 sequences)

SEQ ID NO: 1 - protein, 5 aa, artificial sequence
  VH CDR1
GYNMN

SEQ ID NO: 2 - protein, 7 aa, Artificial Sequence
  VH CDR2
HIDPYYG

SEQ ID NO: 3 - protein, 8 aa, Artificial Sequence
  VH CDR3
YCVKGGYY

SEQ ID NO: 4 - protein, 11 aa, Artificial Sequence
  VL CDR1
RASQDIGSSLN

SEQ ID NO: 5 - protein, 7 aa, Artificial Sequence
  VL CDR2
ATSSLDS

SEQ ID NO: 6 - protein, 9 aa, Artificial Sequence
  VL CDR3
LQYVSSPPT

SEQ ID NO: 7 - protein, 5 aa, Artificial Sequence
  VH CDR1
SYAIS

SEQ ID NO: 8 - protein, 17 aa, Artificial Sequence
  VH CDR2
GFDPEDGETIYAQKFQG

SEQ ID NO: 9 - protein, 17 aa, Artificial Sequence
  VH CDR3
GRSMVRGVIIPFNGMDV

SEQ ID NO: 10 - protein, 11 aa, Artificial Sequence
  VL CDR1
RASQSISSYLN

SEQ ID NO: 11 - protein, 7 aa, Artificial Sequence
  VL CDR2
AASSLQS

SEQ ID NO: 12 - protein, 9 aa, Artificial Sequence
  VL CDR3
QQSYSTPLT

SEQ ID NO: 13 - protein, 5 aa, Artificial Sequence
  VEGF VH CDR1
NYWMH

SEQ ID NO: 14 - protein, 17 aa, Artificial Sequence
  VEGF VH CDR2
DINPSNGRTSYKEKFKR

SEQ ID NO: 15 - protein, 12 aa, Artificial Sequence
  VEGF VH CDR3
HYDDKYYPLMDY

SEQ ID NO: 16 - protein, 6 aa, Artificial Sequence
  DLL4 VH CDR1
TAYYIH

SEQ ID NO: 17 - protein, 17 aa, Artificial Sequence
  DLL4 VH CDR2
YISNYNRATNYNQKFKG

SEQ ID NO: 18 - protein, 11 aa, Artificial Sequence
  DLL4 V4 CDR3
RDYDYDVGMDY

SEQ ID NO: 19 - protein, 15 aa, Artificial Sequence
  VL CDR1
RASESVDNYGISFMK

SEQ ID NO: 20 - protein, 7 aa, Artificial Sequence
  VL CDR2
AASNQGS

SEQ ID NO: 21 - protein, 12 aa, Artificial Sequence
  VL CDR3
QQSKEVPWTFGG

SEQ ID NO: 22 - protein, 120 aa, artificial sequence
  Heavy Chain
EVQLQQSGPELEKPGASVKLSCKASGYSFTGYNMNWVKQSHGKSLEWIGHIDPYYGDTSYNQKFRGKATLTVDKSSSTAYMQLKSLTSEDSAVYYCVKGGYYGHWYFDVWGAGTTVTVSS

SEQ ID NO: 23 - protein, 107 aa, artificial sequence
  Light Chain
DIQMTQSPSSLSASLGERVSLTCRASQDIGSSLNWLQQGPDGTIKRLIYATSSLDSGVPKRFSGSRSGSDYSLTISSLESEDFVDYYCLQYVSSPPTFGAGTKLELK

SEQ ID NO: 24 - protein, 119 aa, artificial sequence
  Heavy Chain
QVQLVQSGAEVKKPGASVKISCKASGYSFTAYYIHWVKQAPGQGLEWIGYISNYNRATNYNQKFKGRVTFTTDTSTSTAYMELRSLRSDDTAVYYCARDYDYDVGMDYWGQGTLVTVSS

SEQ ID NO: 25 - protein, 111 aa, artificial sequence
  Light Chain
DIVMTQSPDSLAVSLGERATISCRASESVDNYGISFMKWFQQKPGQPPKLLIYAASNQGSGVPDRFSGSGSGTDFTLTISSLQAEDVAVYYCQQSKEVPWTFGGGTKVEIK

SEQ ID NO: 26 - protein, 126 aa, artificial sequence
  Heavy Chain
QVQLVQSGAEVKKPGASVKVSCKASGGTFSSYAISWVRQAPGQGLEWMGGFDPEDGETIYAQKFQGRVTMTEDTSTDTAYMELSSLRSEDTAVYYCATGRSMVRGVIIPFNGMDVWGQGTTVTVSS

SEQ ID NO: 27 - protein, 107 aa, artificial sequence
  Light Chain
DIRMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 28 - protein, 8 aa, artificial sequence
  Heavy Chain CDR1
GFSLTSYG

SEQ ID NO: 29 - protein, 7 aa, artificial sequence
  Heavy Chain CDR2
IYADGST

SEQ ID NO: 30 - protein, 12 aa, artificial sequence
  Heavy Chain CDR3
ARAYGNYWYIDV

SEQ ID NO: 31 - protein, 6 aa, artificial sequence
  Light Chain CDR1
ESVSND

SEQ ID NO: 32 - protein, 3 aa, artificial sequence
  Light Chain CDR2
YAF

SEQ ID NO: 33 - protein, 9 aa, artificial sequence
  Light Chain CDR3
HQAYSSPYT

SEQ ID NO: 34 - protein, 118 aa, artificial sequence
  Heavy Chain
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 35 - protein, 107 aa, artificial sequence
  Light Chain
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 36 - protein, 8 aa, artificial sequence
  Heavy Chain CDR1
GGTFSSYA

SEQ ID NO: 37 - protein, 8 aa, artificial sequence
  Heavy Chain CDR2
IIPMFDTA

SEQ ID NO: 38 - protein, 13 aa, artificial sequence
  Heavy Chain CDR3
ARAEHSSTGTFDY

SEQ ID NO: 39 - protein, 6 aa, artificial sequence
  Light Chain CDR1
QGISSW

SEQ ID NO: 40 - protein, 3 aa, artificial sequence
  Light Chain CDR2
AAS

SEQ ID NO: 41 - protein, 9 aa, artificial sequence
  Light Chain CDR3
QQANHLPFT

SEQ ID NO: 42 - protein, 120 aa, artificial sequence
  Heavy Chain
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 43 - protein, 107 aa, artificial sequence
  Light Chain
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK